(19) United States
(12) Patent Application Publication
(10) Pub. No.: US 2026/0273110 A1
(43) Pub. Date: Sep. 17, 2026

(54) Radiolabeled Fibroblast Activation Protein Inhibitors and Methods of Making Same

(71) Applicant: REGENTS OF THE UNIVERSITY OF MICHIGAN, Ann Arbor, MI (US)

(72) Inventors: Allen F. Brooks, Ann Arbor, MI (US); Peter J.H. Scott, Ypsilanti, MI (US); Benjamin L. Viglianti, Ann Arbor, MI (US); Jason Witek, Ann Arbor, MI (US)

(21) Appl. No.: 19/167,133

(22) PCT Filed: Mar. 21, 2024
(86) PCT No.: PCT/US24/20846

§ 371 Date: Sep. 19, 2025

Related U.S. Application Data
(60) Provisional application No. 63/453,943, filed on Mar. 22, 2023.

(51) Int. Cl. A61K 51/04; C07B 59/00; C07D 207/16; C07D 401/12; C07D 403/12

(52) U.S. Cl. CPC A61K 51/0455; A61K 51/0446; C07B 59/002; C07D 207/16; C07D 401/12; C07D 403/12

(57) ABSTRACT

Fibroblast activation inhibitors having a structure according to formula (I), formula (II), or formula (III): wherein X is located at carbon 1′, 2′, or 3′ and is selected from the group of I, ¹⁸F, ²¹¹At, ¹²³I, ¹²⁴I, ¹²⁵I, ¹³¹I, ⁷⁶Br, ⁷⁷Br, H₃¹¹CO, CF₂ ¹⁸F, CHF¹⁸F, OCF₂¹⁸F, OCHF¹⁸F, SCF₂¹⁸F, SCHF¹⁸F, ¹¹CN, ¹¹CH₃, and H₃¹¹CS; each R is independently selected from H, F, ²H, CH₃, and cyclopentyl; with the proviso that in formula (I), when X is H₃¹¹CO and both R are H or both R are F, then X is not located at CN carbon 3′, and methods of making and using same.

### STATEMENT OF GOVERNMENT SUPPORT

This invention was made with government support under AWD018348 awarded by the National Institutes of Health. The government has certain rights in the invention.

### FIELD

The disclosure relates generally to fibroblast activation protein inhibitors. More specifically, the disclosure relates to radiolabeled fibroblast activation protein inhibitors having a glycine-cyano-proline pharmacophore attached to a quinoline, naphthalene, or indole core, wherein the radiolabel is provided directly on the quinoline, naphthalene, or indole core.

### BACKGROUND

Cancer is the second leading cause of death in the United States, with over 600K Americans dying in 2020 according to the CDC. Better methods of detection and treatment requires the continual elucidation of mechanisms involved in cancer progression. One requirement for the growth of solid tumors is the recruitment of normal stromal cells, providing the scaffold for their continued growth. The key type of cells that provides this scaffold are fibroblasts. During cancer growth, these cancer-associated fibroblasts are activated and express Fibroblast Activation Protein (FAP) on their cell surface.

Fibroblast activation protein (FAP) is a transmembrane protein that is a serine protease expressed on fibroblasts in up to 90% of epithelial tumors with subsequent limited FAP expression in normal tissues, unless undergoing wound healing. Additionally, some tumors themselves from mesenchymal origin express FAP, sarcoma and mesothelioma being the most notable. Given this expression, FAP has become a target for radiotherapeutic localization. As a result, multiple family of ligands/inhibitors of FAP (FAPI) have been developed and tested as therapeutics. Most of these inhibitors share a similar scaffold with substitution variations to enhance binding properties or improve pharmacokinetics. Testing of these inhibitors as pharmacologic agents (and FAP-directed antibodies) for the treatment of cancer have had limited clinical success to date. These inhibitors do accumulate/localize in tumors to varying degrees however, leading to interest in their use for diagnostic imaging and as a radiotherapeutic, representing a theranostic pair.

The small molecule quinoline-based FAP targeted radiotracer, FAPI-04 has demonstrated encouraging results with high uptake in over 80 patients across a variety of cancers. FAPI-04 and nearly identical analog FAPI-46 are based on a series of FAP inhibitors previously developed. Researchers observed that modifications at the 6 position of the quinoline ring had minimal impact on binding. Building off the 6-methoxy modification, investigators utilized an ether linkage at that position to connect the pharmacophore for FAP to DOTA for chelation of a radiometal FAPI-04 (FIG. 1). ⁶⁸Ga is utilized for imaging and radiometals with different decay properties for therapy can be utilized, e.g., ¹⁷⁷Lu for beta radiotherapy analogous to how [¹⁷⁷Lu]-PSMA-617 complements ⁶⁸Ga labelled PSMA diagnostic agents. The cyclic peptide FAP-2286 represents an alternative to the scaffold of FAPI-04/FAPI-46.

FAP-2286 was developed to increase the biological half life of the agent with the hope that it would slow the washout from target tissue to increase radiation dose to tumor of administered agent. FAP-2286 features a DOTA chelator linked to a cyclic peptide required for targeting FAP. This resulted in similar uptake to FAPI-46 from injection at 3 h for both the Ga and Lu agents. Although, the absolute [¹⁷⁷Lu] uptake at 24 and 72 h was increased in the tumor with FAP-2286, the tumor to kidney ratio was better for FAPI-46 at 3 h (21.0 vs 9.6), identical at 24 hours (12.7 vs 13.1), and better for FAP-2286 at 72 h (27.3 vs 8). Similarly, [⁶⁸Ga]-FAPI-46 had similar uptake in the tumor as FAP-2286, but FAPI-46 exhibited faster clearance in the background tissue providing improved tumor to background ratio of 3-5 times more than FAP-2286 at early time points. The prior work demonstrated the importance of FAP as a target with elevated expression in multiple cancers; the use of labeled FAPI for the identification and localization of tumors; the potential to treat cancer using radiolabeled FAPI; preclinical data demonstrating that smaller FAPIs directly labelled on the pharmacophore have increased tumor to background uptake compared to the FAPI that utilize a linker/chelator construct. However, the prior work also indicates that most FAPIs are based on a non-optimized molecule where tumor to background can be furthered improved for both imaging and treatment; the use of ⁶⁸Ga or ⁶⁴Cu as imaging agent although adequate are inferior to ¹⁸F based on the physical decay properties of the isotopes, imaging resolution, radiation dosimetry considerations, and current commercial production facilities.

There exists a need in the art to provide alternative FAPI compounds with improved pharmacokinetics and improved tumor to background selectivity for therapeutic advantages and advantages for imaging and detection of cancer that express FAP.

### SUMMARY

One aspect of the disclosure provides a fibroblast activation protein inhibitor having a structure according to formula (I), formula (II), or formula (III):

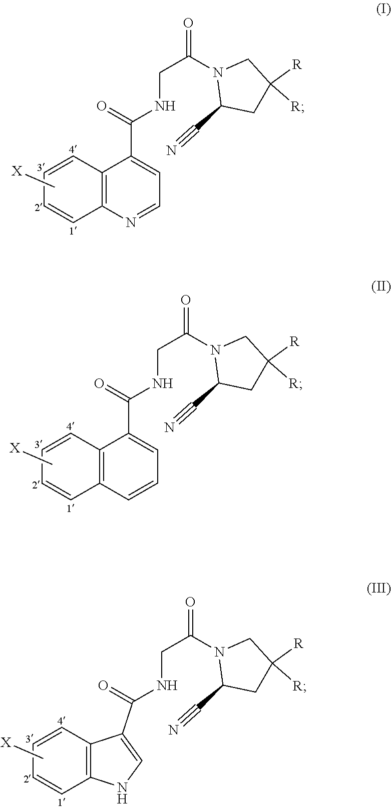

wherein X is located at carbon 1′, 2′, or 3′ and is selected from the group of I, ¹⁸F, ²¹¹At, ¹²³I, ¹²⁴I, ¹²⁵I, ¹³¹I, ⁷⁶Br, ⁷⁷Br, H₃¹¹CO, CF₂¹⁸F, CHF¹⁸F, OCF₂¹⁸F, OCHF¹⁸F, SCF₂¹⁸F, SCHF¹⁸F, ¹¹CN, ¹¹CH₃, and H₃¹¹CS; each R is independently selected from H, F, ²H, CH₃, and cyclopentyl; with the proviso that in formula (I), when X is H₃¹¹CO and both R are H or both R are F, then X is not located at carbon 3′.

Another aspect of the disclosure provides methods of preparing a fibroblast activation protein inhibitor comprising admixing a compound having a structure according to formula (IV) with a salt, MX, to form a compound having a structure according to formula (I):

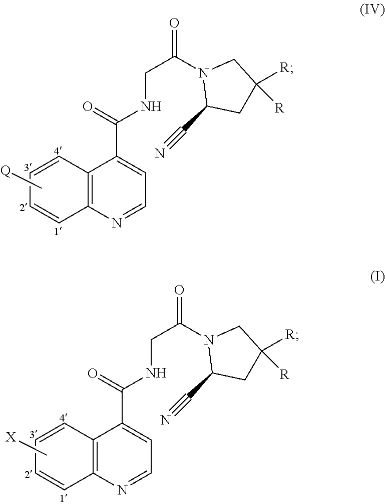

wherein M is selected from the group of K, Li, Na, Rb, Cs, tetraethylammonium, and tetrabutylammonium; in the salt, KX, and in formula (I), X is selected from the group of ¹²³I, ¹²⁴I, ¹²⁵I, ¹³¹I, ²¹¹At, ⁷⁶Br, ⁷⁷Br, ¹⁸F, H₃¹¹CO, CF₂¹⁸F, CHF¹⁸F, OCF₂¹⁸F, OCHF¹⁸F, SCF₂¹⁸F, SCHF¹⁸F, ¹¹CN, ¹¹CH₃, and H₃¹¹CS; and X is the same in the salt and in formula (I); Q is selected from trialkyl stannyl, trialkyl germyl, and trialkyl silyl; in formula (IV) and formula (I), Q and X are located at carbon 1′, 2′, or 3′, and the location of Q in formula (IV) and the location of X in formula (I) are the same; and each R is independently selected from H, F, ²H, CH₃, and cyclopentyl, and the R in formula (I) are the same as the R in formula (IV).

Another aspect of the disclosure provides methods of preparing a fibroblast activation protein inhibitor comprising admixing a compound having a structure according to formula (VI) with a salt, MX, to form a compound having a structure according to formula (II):

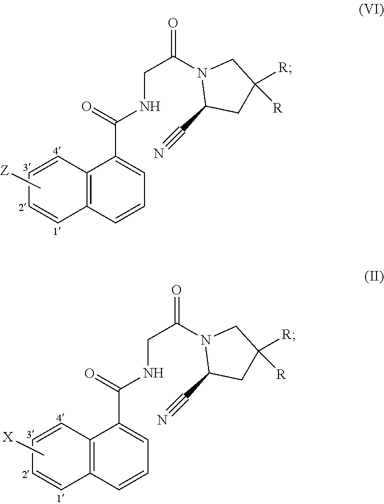

wherein M is selected from the group of K, Li, Na, Rb, Cs, tetraethylammonium, and tetrabutylammonium; in the salt, KX, and in formula (II), X is selected from the group of ¹²³I, ¹²⁴I, ¹²⁵I, ¹³¹I, ²¹¹At, ⁷⁶Br, ⁷⁷Br, ¹⁸F, H₃¹¹CO, CF₂¹⁸F, CHF¹⁸F, OCF₂¹⁸F, OCHF¹⁸F, SCF₂¹⁸F, SCHF¹⁸F, ¹¹CN, ¹¹CH₃, and H₃¹¹CS, and X is the same in the salt and in formula (II); Q is selected from trialkyl stannyl, trialkyl germyl, or trialkyl silyl; in formula (VI) and formula (II), Q and X are located at carbon 1′, 2′, or 3′, and the location of Q in formula (VI) and the location of X in formula (II) are the same; and each R is independently selected from H, F, ²H, CH₃, and cyclopentyl, and the R in formula (II) are the same as the R in formula (VI).

Another aspect of the disclosure provides methods of preparing a fibroblast activation protein inhibitor comprising admixing a compound having a structure according to formula (VII) with a salt, MX, to form a compound having a structure according to formula (III):

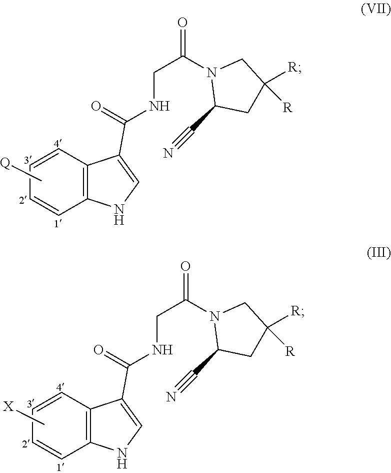

wherein M is selected from the group of K, Li, Na, Rb, Cs, tetraethylammonium, and tetrabutylammonium; in the salt, MX, and in formula (III), X is selected from the group of ¹²³I, ¹²⁴I, ¹²⁵I, ¹³¹I, ²¹¹At, ⁷⁶Br, ⁷⁷Br, ¹⁸F, H₃¹¹CO, CF₂¹⁸F, CHF¹⁸F, OCF₂¹⁸F, OCHF¹⁸F, SCF₂¹⁸F, SCHF¹⁸F, ¹¹CN, ¹¹CH₃, and H₃¹¹CS, and X is the same in the salt and in formula (III); Q is selected from trialkyl stannyl, trialkyl germyl, or trialkyl silyl; in formula (VII) and formula (III), Q and X are located at carbon 1′, 2′, or 3′, and the location of Q in formula (VII) and the location of X in formula (III) are the same; and each R is independently selected from H, F, ²H, CH₃, and cyclopentyl, and the R in formula (III) are the same as the R in formula (VII).

Another aspect of the disclosure provides methods of preparing a fibroblast activation protein inhibitor comprising admixing a compound having a structure according to formula (VIII) with a radiolabeled salt, MX, to form a compound having a structure according to formula (I):

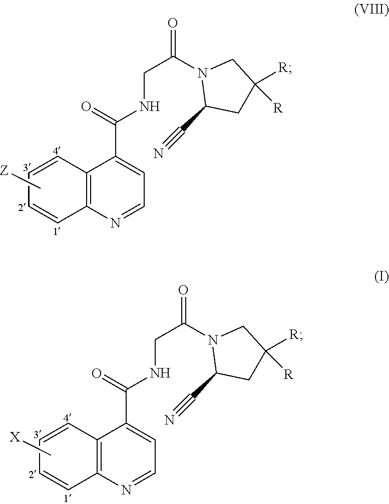

wherein Z has a structure according selected from the group of:

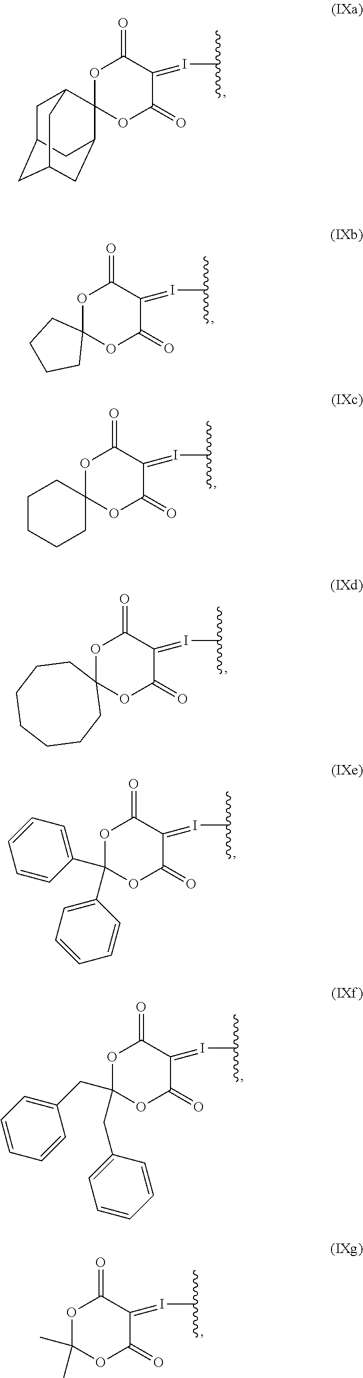

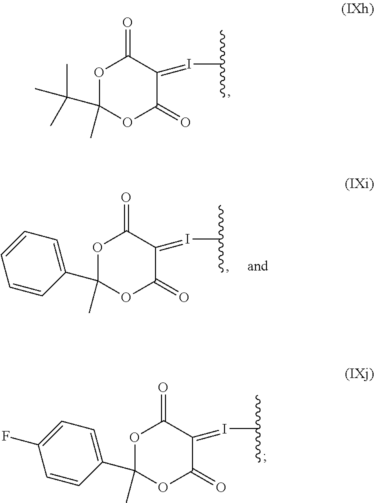 [SMILES: *I=C1C(=O)OC(C)(C(C)(C)C)OC1=O ; *I=C1C(=O)OC(C)(c2ccccc2)OC1=O ; *I=C1C(=O)OC(C)(c2ccc(F)cc2)OC1=O]

in formula (VIII) and formula (I), Z and X are located at carbon 1′, 2′, or 3′, and the location of Z in formula (VIII) and the location of X in formula (I) are the same; M is selected from the group of K, Li, Na, Rb, Cs, tetraethylammonium, and tetrabutylammonium; in the salt, MX, and in formula (I), X is selected from the group of ¹⁸F, ²¹¹At, ¹²³I, ¹²⁴I, ¹²⁵I, ¹³¹I, ⁷⁶Br, ⁷⁷Br; and each R is independently selected from H, F, ²H, CH₃, and cyclopentyl, and the R in formula (I) are the same as the R in formula (VIII).

Another aspect of the disclosure provides methods of preparing a fibroblast activation protein inhibitor comprising admixing a compound having a structure according to formula (X) with a radiolabeled salt, MX, to form a compound having a structure according to formula (II):

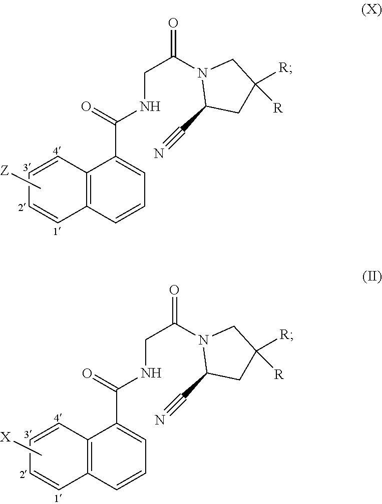

wherein Z has a structure selected from the group of:

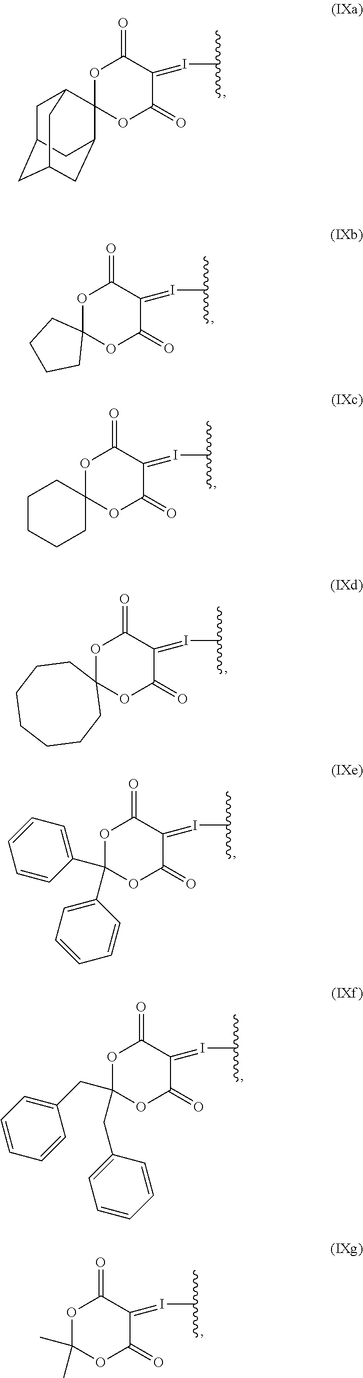

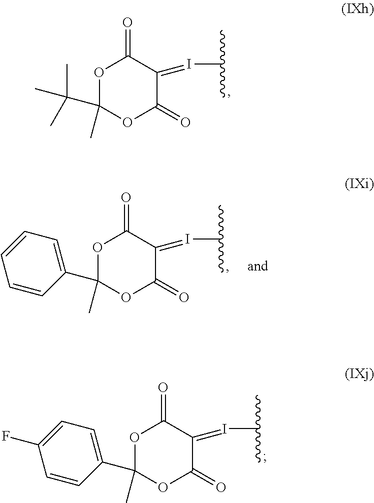 [SMILES: *I=C1C(=O)OC(C)(C(C)(C)C)OC1=O ; *I=C1C(=O)OC(C)(c2ccccc2)OC1=O ; *I=C1C(=O)OC(C)(c2ccc(F)cc2)OC1=O]

in formula (X) and formula (II), Z and X are located at carbon 1′, 2′, or 3′, and the location of Z in formula (X) and the location of X in formula (II) are the same; M is selected from the group of K, Li, Na, Rb, Cs, tetraethylammonium, and tetrabutylammonium; in the salt, MX, and in formula (II), X is selected from the group of ¹⁸F, ²¹¹At, ¹²³I, ¹²⁴I, ¹²⁵I, ¹³¹I, ⁷⁶Br, ⁷⁷Br; and each R is independently selected from H, F, ²H, CH₃, and cyclopentyl, and the R in formula (II) are the same as the R in formula (X).

Another aspect of the disclosure provides methods of preparing a fibroblast activation protein inhibitor comprising admixing a compound having a structure according to formula (XI) with a radiolabeled salt, MX, to form a compound having a structure according to formula (III):

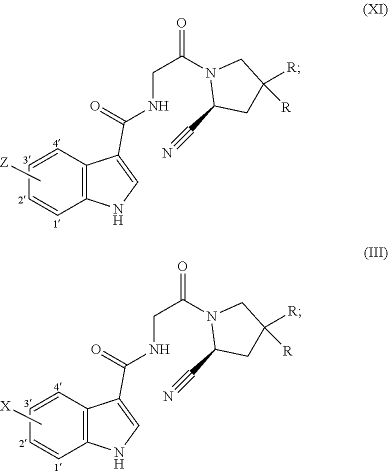

wherein Z has a structure selected from the group of:

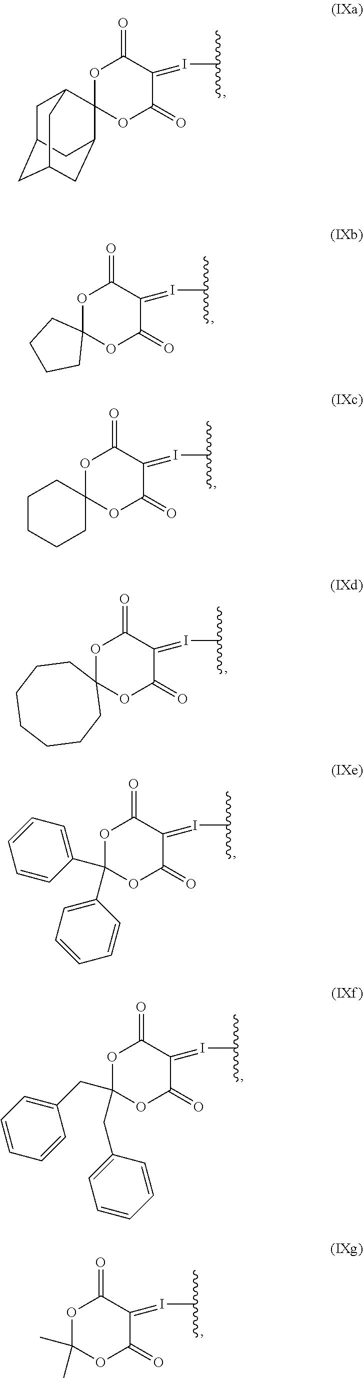

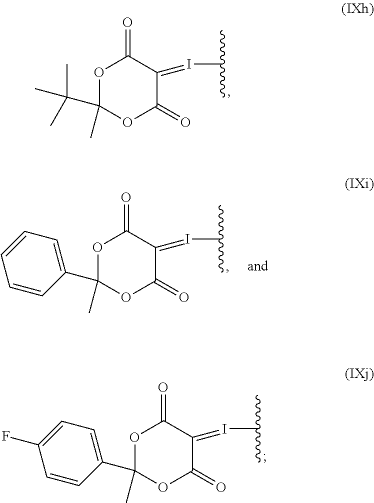 [SMILES: *I=C1C(=O)OC(C)(C(C)(C)C)OC1=O ; *I=C1C(=O)OC(C)(c2ccccc2)OC1=O ; *I=C1C(=O)OC(C)(c2ccc(F)cc2)OC1=O]

in formula (XI) and formula (III), Z and X are located at carbon 1′, 2′, or 3′, and the location of Z in formula (XI) and the location of X in formula (III) are the same; M is selected from the group of K, Li, Na, Rb, Cs, tetraethylammonium, and tetrabutylammonium; in the salt, MX, and in formula (III), X is selected from the group of ¹⁸F, ²¹¹At, ¹²³I, ¹²⁴I, ¹²⁵I, ¹³¹I, ⁷⁶Br, ⁷⁷Br; and each R is independently selected from H, F, ²H, CH₃, and cyclopentyl, and the R in formula (III) are the same as the R in formula (XI).

Another aspect of the disclosure provides methods of treating, detecting, or imaging cancer comprising, administering to a patient a fibroblast activation protein inhibitor according to the disclosure.

Another aspect of the disclosure provides methods of detecting fibroblast activation protein expression in human development, growth, wound healing, or a combination thereof comprising administering to a patient a fibroblast activation protein inhibitor according to the disclosure.

Another aspect of the disclosure provides methods of detecting a cardiovascular pathology comprising, administering to a patient a fibroblast activation protein inhibitor according to the disclosure.

Another aspect of the disclosure provides methods of detecting a pulmonary pathology comprising, administering to a patient a fibroblast activation protein inhibitor according to the disclosure.

Further aspects and advantages will be apparent to those of ordinary skill in the art from a review of the following detailed description. While the compositions and methods are susceptible of embodiments in various forms, the description hereafter includes specific embodiments with the understanding that the disclosure is illustrative and is not intended to limit the disclosure to the specific embodiments described herein.

### BRIEF DESCRIPTION OF THE DRAWINGS

While the specification concludes with claims particularly pointing out and distinctly claiming the subject matter, which is regarded as forming the present invention, it is believed that the invention will be better understood from the following description taken in conjunction with the accompanying drawings.

FIG. 1 is a scheme of various prior art FAP targeting agents.

### DETAILED DESCRIPTION

The disclosure provides fibroblast activation protein inhibitors (FAPIs) having a structure according to formula (I), (II), or (III):

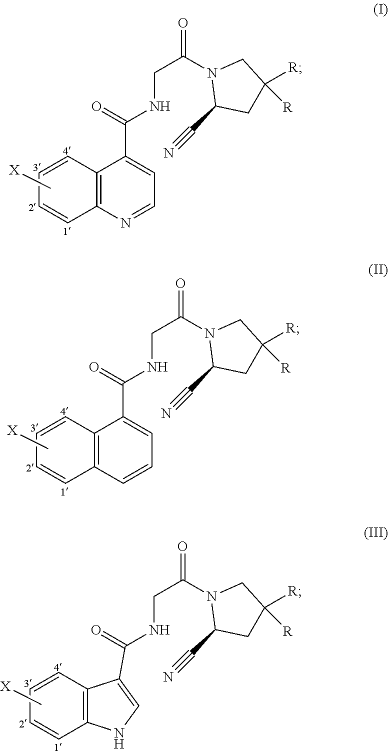

wherein X is located at carbon 1′, 2′, or 3′ and is selected from the group of I, ¹⁸F, ²¹¹At, ¹²³I, ¹²⁴I, ¹²⁵I, ¹³¹I, ⁷⁶Br, ⁷⁷Br, H₃¹¹CO, CF₂¹⁸F, CHF¹⁸F, OCF₂¹⁸F, OCHF¹⁸F, SCF₂¹⁸F, SCHF¹⁸F, ¹¹CN, ¹¹CH₃, and H₃¹¹CS; each R is independently selected from H, F, ²H, CH₃, and cyclopentyl; with the proviso that in formula (I), when X is H₃¹¹CO and both R are H or both R are F, then X is not located at carbon 3′.

As used herein the phrases “compound(s) of formula,” “compound(s) having a structure of formula,” “compound(s) according to formula,” “compound(s) having a structure according to formula,” and the like are used interchangeably. Similarly, the phrases “fibroblast activation protein inhibitor(s) of formula,” “fibroblast activation protein inhibitor(s) having a structure of formula,” “fibroblast activation protein inhibitor(s) according to formula,” “fibroblast activation protein inhibitor(s) having a structure according to formula,” and the like are used interchangeably.

The compounds of the disclosure are fibroblast activation protein inhibitors and are generically represented by formula (I), (II), and (III). Specific compounds within each class are identified by X group, position of X group, and R groups. For example, a compound having a structure according to formula (I), wherein X is I is referred to herein as a compound of formula (Ia). In the case wherein the I is located at position 3′ and both R are H, the compound is also referred to as a compound having a structure according to formula (Ia-3). For a similar compound where both R are F, the compound is referred to as a compound having a structure according to formula (Ia-3F). The related compound where one R is H and one R is F is a compound having a structure according to formula (Ia-3HF). Thus, in the naming scheme, the letter following the roman numeral 1, 11, or Ill identifies the X group, the number following the “-” provides the position of X, and the letters following the position number identifies the R groups, where no letter is provided for two hydrogen, one F is provided for two fluorine, one “cy” is provided for two cyclohexyl, one “²H” is provided for two deuterium atoms, one “Me” is provided for two methyl groups, and one H and one F when one R group is H and one R group is F, for example. The X group and corresponding letters are as follows: a=1, b=¹⁸F, c=¹²³I d=¹²⁴I e=¹²⁵I, f=¹³¹I, g=⁷⁶Br, h=⁷⁷Br, i=²¹¹At, j=H₃¹¹CO, k=H₃¹¹CS, m=¹¹CH₃, n=¹¹CN, o=CF₂¹⁸F, p=CHF¹⁸F, q=OCF₂¹⁸F, r=OCHF¹⁸F, s=SCF₂¹⁸F and t=SCHF¹⁸F.

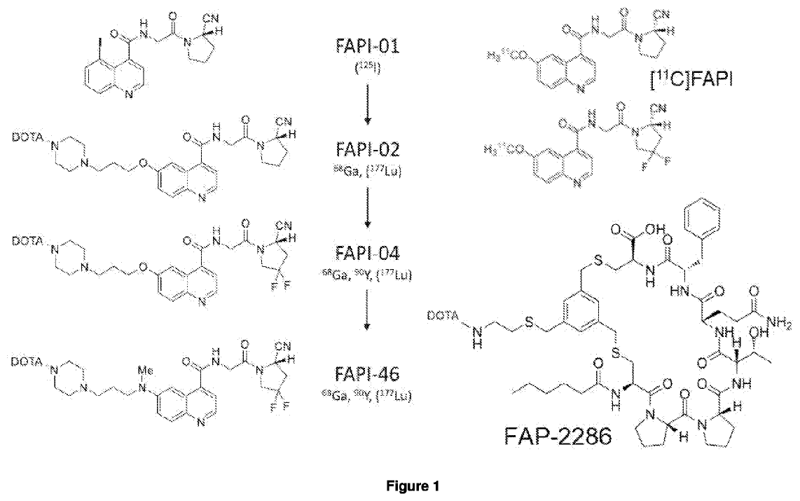

The disclosed compounds generally have smaller molecular size than FAPI compounds by directly labeling the quinoline core with the radiometal. Prior art FAPI imaging agents used ether linkages at the 6 position to a DOTA for radiometal labeling. By adding the linker and DOTA the molecular weight of the imaging agent greatly increased and changed its pharmacokinetics. This direct labeling of the binding motif can reduce the molecular weight of the FAPI by ~50% compared to FAPI-04/FAPI-46 and ~75% compared to FAP-2286. Wang et al., found that a FAPI compound having [¹¹C]CH₃O linked to the 6 position on the quinoline core (FIG. 1) demonstrated a ~30% increase in the injected dose/gm tissue (% ID/g) going to the tumor compared to FAPI-04 and the off target dose % ID/g to the kidneys was reduced >70%. Wang et al. Radiosynthesis and First Preclinical Evaluation of the Novel (11)C-Labeled FAP Inhibitor (11)C-FAPI: A Comparative Study of (11)C-FAPIs and ((68)Ga) Ga-DOTA-FAPI-04 in a High-FAP-Expression Mouse Model. Front Chem 2022; 10:939160. Combined, this resulted in an ~300% increase in tumor to background targeting of [¹¹C]FAPI. However, it should be noted that the short half-life of ¹¹C (20 min) limits the imaging window. Delayed imaging is limited with the agent, but imaging studies revelated that tumor uptake persisted whereas off target uptake cleared as fast or faster than FAPI-04/FAPI-46. Although the work by Wang et al. using ¹¹C is promising and shows significant improvements over FAPI-04/FAPI-46, ¹¹C is not a widespread viable option for clinical use given its short physical half-life an inability to develop a concurrent therapeutic pair. The compounds of the disclosure include FAPI with ¹⁸F, ¹²³I, ¹²⁴I, and ¹²⁵I for imaging (Fluro-FAPI), and iodine labeled FAPI for a therapeutic pair with ¹³¹I for beta therapy or ²¹¹At for alpha therapy, thus providing an imaging agent ready to be translated into humans with a lower molecular weight and improved pharmacokinetics that is suitable for clinical production utilizing existing infrastructure from the manufacture of FDG with a direct pathway for making a subsequent therapeutic pair.

The methods of the disclosure advantageously allow direct radiolabeling of the quinoline ring of the FAPI core structure. Methods to directly label the core structure were previously unknown. No attempt to directly radiolabel, e.g., the 6-Fluoro-FAPI, has been reported and is likely due to the fact that a method to prepare the required iodo intermediate (a compound of formula (Ia-3), herein) for generation of radiolabeling precursors has not previously been developed The ability to directly label the FAPI core structure advantageously provides FAPI compounds having small molecular sizes which can provide advantages for imaging and detection of cancer that express FAP and can be designed to provide therapeutic advantages by controlling the tumor-to-background ratio for the decay time of the therapeutic radioisotope, thus avoiding unnecessary radiation does to healthy tissue and organs.

### Compounds of Formula (I)

In some embodiments, the disclosure provides a compound having a structure according to formula (I):

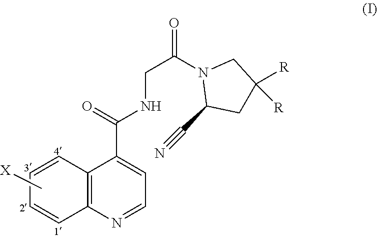

wherein X is located at carbon 1′, 2′, or 3′ and is selected from the group of I, ¹⁸F, ²¹¹At, ¹²³I, ¹²⁴I, ¹²⁵I, ¹³¹I, ⁷⁶Br, ⁷⁷Br, H₃¹¹CO, CF₂¹⁸F, CHF¹⁸F, OCF₂¹⁸F, OCHF¹⁸F, SCF₂¹⁸F, SCHF¹⁸F, ¹¹CN, ¹¹CH₃, and H₃¹¹CS; each R is independently selected from H, F, ²H, CH₃, and cyclopentyl; with the proviso that when X is H₃¹¹CO and both R are H or both R are F, then X is not located at carbon 3′.

In some embodiments, X is selected from the group of ¹⁸F, ²¹¹At, ¹²³I, ¹²⁴I, ¹²⁵I, ¹³¹I, ⁷⁶Br, ⁷⁷Br, H₃¹¹CO, CF₂¹⁸F, CHF¹⁸F, OCF₂¹⁸F, OCHF¹⁸F, SCF₂¹⁸F, SCHF¹⁸F, ¹¹CN, ¹¹CH₃, and H₃¹¹CS. In embodiments, X is selected from the group of ¹⁸F, ²¹¹At, ¹²³I, ¹²⁴I, ¹²⁵I, ¹³¹I, ⁷⁶Br, ⁷⁷Br, and H₃¹¹CO.

In some embodiments, X is located at the 3′ carbon. In some embodiments, X is located at the 2′ carbon. In some embodiments, X is located at the 1′ carbon.

In some embodiments, X is located at the 3′ carbon and X is 1. In some embodiments, X is located at the 3′ carbon and X is ¹⁸F. In some embodiments, X is located at the 3′ carbon and X is ²¹¹At. In some embodiments, X is located at the 3′ carbon and X is ¹²³I. In some embodiments, X is located at the 3′ carbon and X is ¹²⁴I. In some embodiments, X is located at the 3′ carbon and X is ¹²⁵I. In some embodiments, X is located at the 3′ carbon and X is ¹³¹I. In some embodiments, X is located at the 3′ carbon and X is ⁷⁶Br. In some embodiments, X is located at the 3′ carbon and X is ⁷⁷Br. In some embodiments, X is located at the 3′ carbon and X is H₃¹¹CO. In some embodiments, X is located at the 3′ carbon and X is CF₂¹⁸F. In some embodiments, X is located at the 3′ carbon and X is CHF¹⁸F. In some embodiments, X is located at the 3′ carbon and X is OCF₂¹⁸F. In some embodiments, X is located at the 3′ carbon and X is OCHF¹⁸F. In some embodiments, X is located at the 3′ carbon and X is SCF₂¹⁸F. In some embodiments, X is located at the 3′ carbon and X is SCHF¹⁸F. In some embodiments, X is located at the 3′ carbon and X is ¹¹CN. In some embodiments, X is located at the 3′ carbon and X is ¹¹CH₃. In some embodiments, X is located at the 3′ carbon and X is H₃¹¹CS.

In some embodiments, X is located at the 2′ carbon and X is 1. In some embodiments, X is located at the 2′ carbon and X is ¹⁸F. In some embodiments, X is located at the 2′ carbon and X is ²¹¹At. In some embodiments, X is located at the 2′ carbon and X is ¹²³I. In some embodiments, X is located at the 2′ carbon and X is ¹²⁴I. In some embodiments, X is located at the 2′ carbon and X is ¹²⁵I. In some embodiments, X is located at the 2′ carbon and X is ¹³¹I. In some embodiments, X is located at the 2′ carbon and X is ⁷⁶Br. In some embodiments, X is located at the 2′ carbon and X is ⁷⁷Br. In some embodiments, X is located at the 2′ carbon and X is H₃¹¹CO. In some embodiments, X is located at the 2′ carbon and X is CF₂¹⁸F. In some embodiments, X is located at the 2′ carbon and X is CHF¹⁸F. In some embodiments, X is located at the 2′ carbon and X is OCF₂¹⁸F. In some embodiments, X is located at the 2′ carbon and X is OCHF¹⁸F. In some embodiments, X is located at the 2′ carbon and X is SCF₂¹⁸F. In some embodiments, X is located at the 2′ carbon and X is SCHF¹⁸F. In some embodiments, X is located at the 2′ carbon and X is ¹¹CN. In some embodiments, X is located at the 2′ carbon and X is ¹¹CH₃. In some embodiments, X is located at the 2′ carbon and X is H₃¹¹CS.

In some embodiments, X is located at the 1′ carbon and X is 1. In some embodiments, X is located at the 1′ carbon and X is ¹⁸F. In some embodiments, X is located at the 1′ carbon and X is ²¹¹At. In some embodiments, X is located at the 1′ carbon and X is ¹²³I. In some embodiments, X is located at the 1′ carbon and X is ¹²⁴I. In some embodiments, X is located at the 1′ carbon and X is ¹²⁵I. In some embodiments, X is located at the 1′ carbon and X is ¹³¹I. In some embodiments, X is located at the 1′ carbon and X is ⁷⁶Br. In some embodiments, X is located at the 1′ carbon and X is ⁷⁷Br. In some embodiments, X is located at the 1′ carbon and X is H₃¹¹CO. In some embodiments, X is located at the 1′ carbon and X is CF₂¹⁸F. In some embodiments, X is located at the 1′ carbon and X is CHF¹⁸F. In some embodiments, X is located at the 1′ carbon and X is OCF₂¹⁸F. In some embodiments, X is located at the 1′ carbon and X is OCHF¹⁸F. In some embodiments, X is located at the 1′ carbon and X is SCF₂¹⁸F. In some embodiments, X is located at the 1′ carbon and X is SCHF¹⁸F. In some embodiments, X is located at the 1′ carbon and X is ¹¹CN. In some embodiments, X is located at the 1′ carbon and X is ¹¹CH₃. In some embodiments, X is located at the 1′ carbon and X is H₃¹¹CS.

In general, each R is independently selected from H, F, ²H, CH₃, and cyclopentyl. In embodiments, each R is independently selected from H and F. In embodiments, at least one R is H. In embodiments, both R are H. In embodiments, at least one R is F. In embodiments, both R are F. In embodiments, at least one R is ²H. In embodiments, both R are 2H. In embodiments, at least one R is CH₃. In embodiments, both R are CH₃. In embodiments, at least one R is cyclopentyl. In embodiments, both R are cyclopentyl. In embodiments, X is located at carbon 3′ and both R are H. In embodiments, X is located at carbon 3′ and both R are F. In embodiments, X is located at carbon 2′ and both R are H. In embodiments, X is located at carbon 2′ and both R are F. In embodiments, X is located at carbon 1′ and both R are H. In embodiments, X is located at carbon 1′ and both R are F.

In embodiments, the fibroblast activation protein inhibitor has a structure according to one of the following formulae:

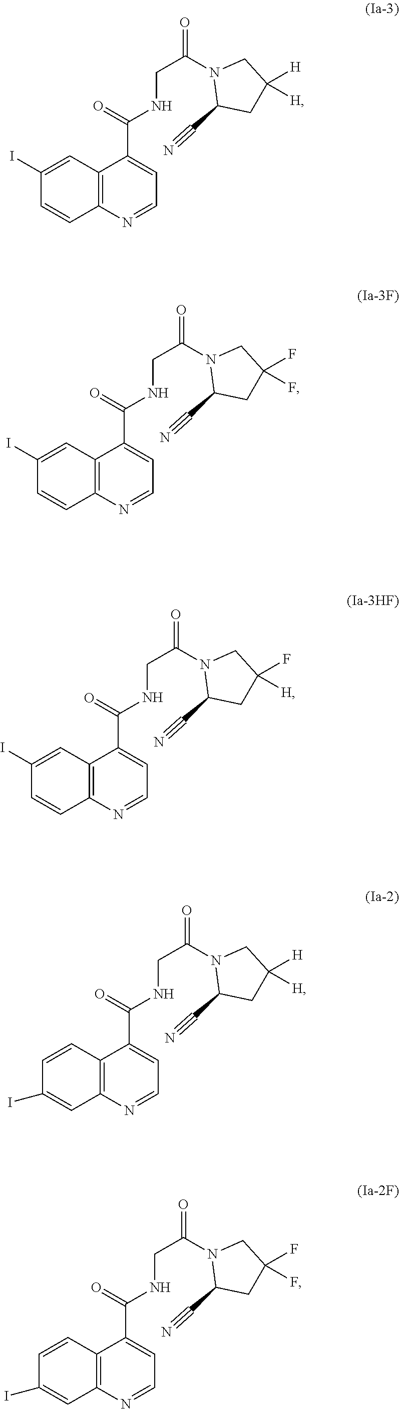

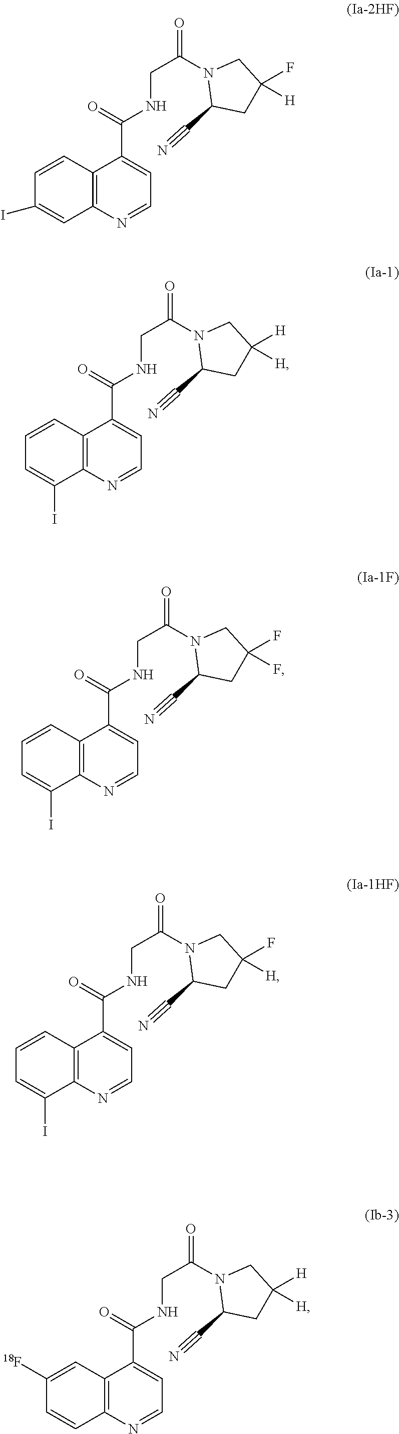

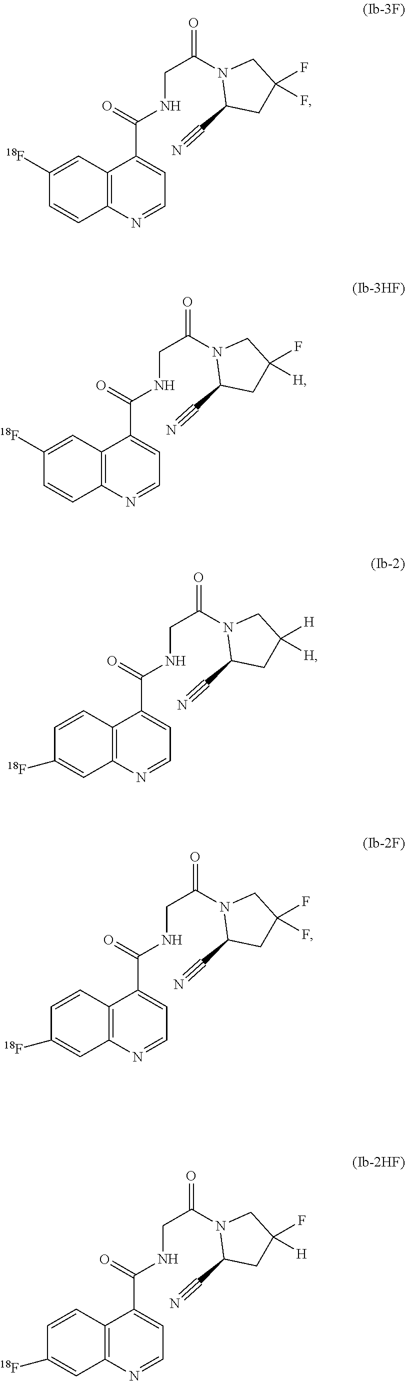

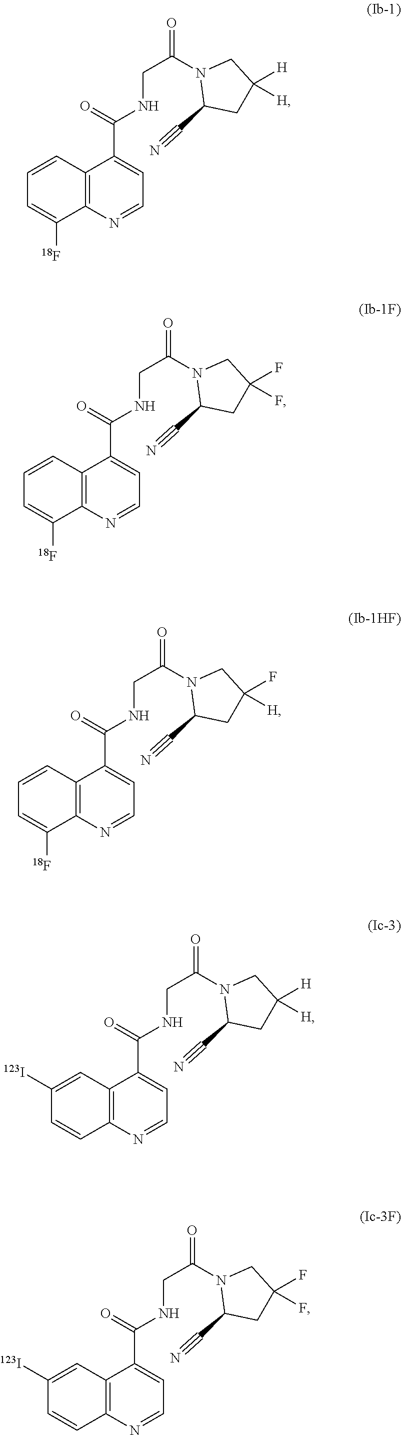

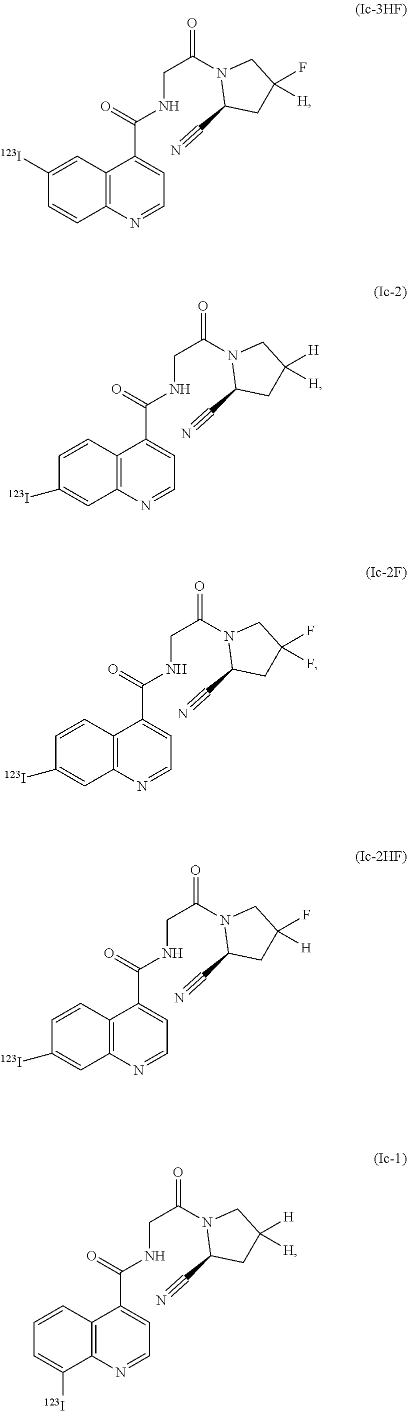

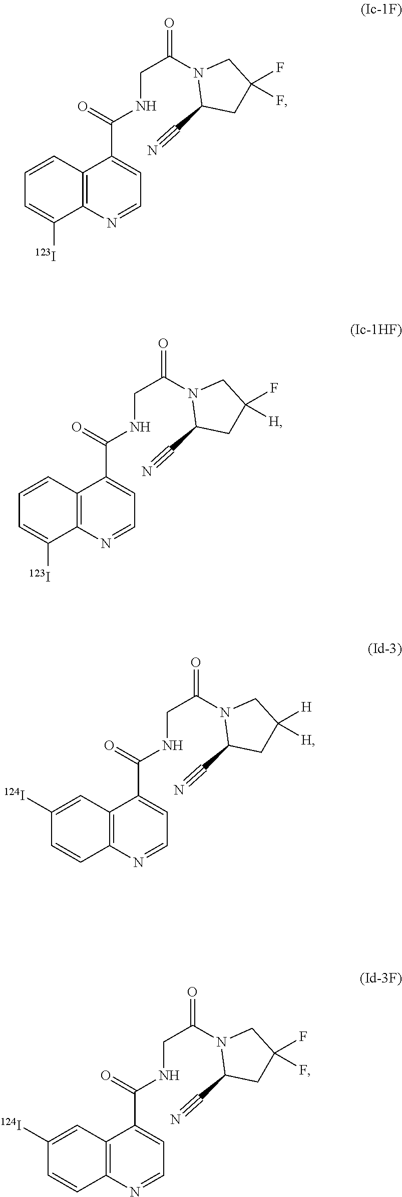

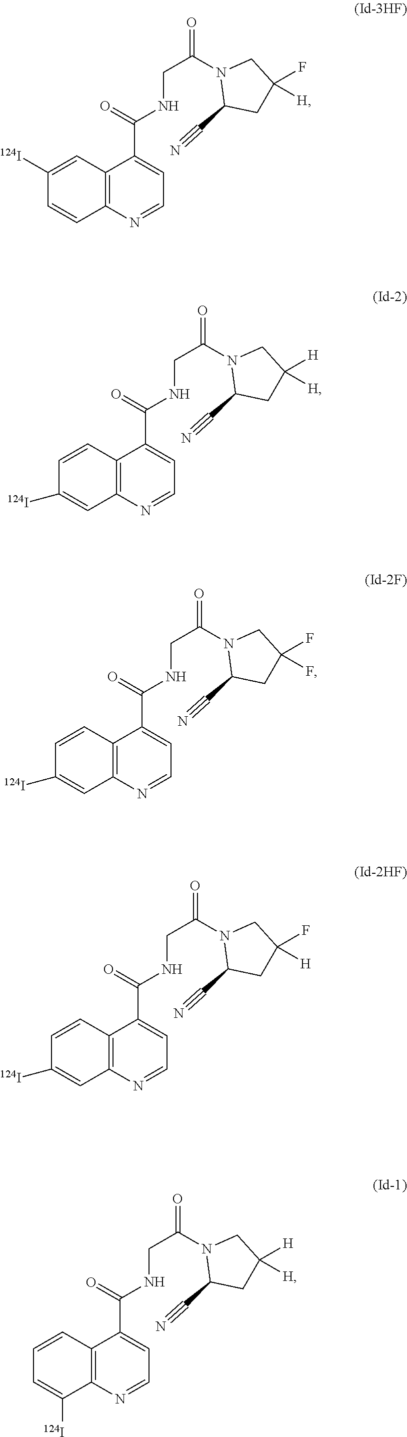

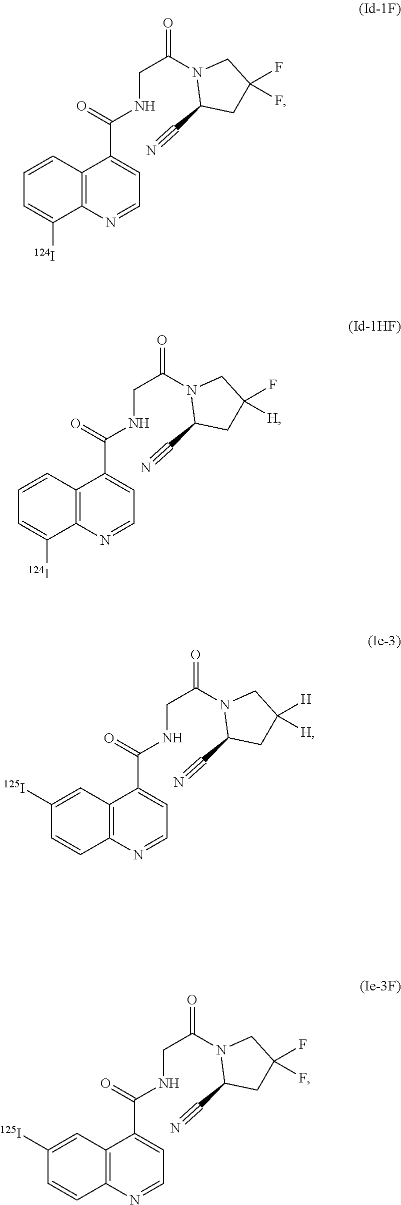

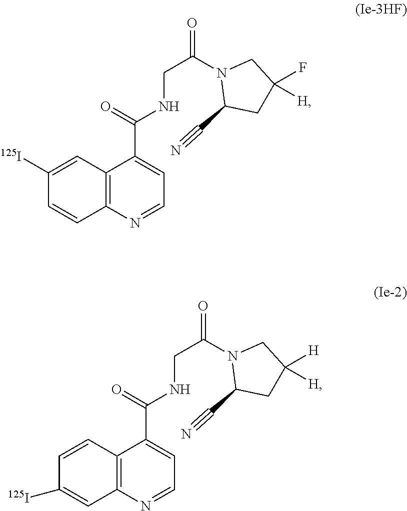

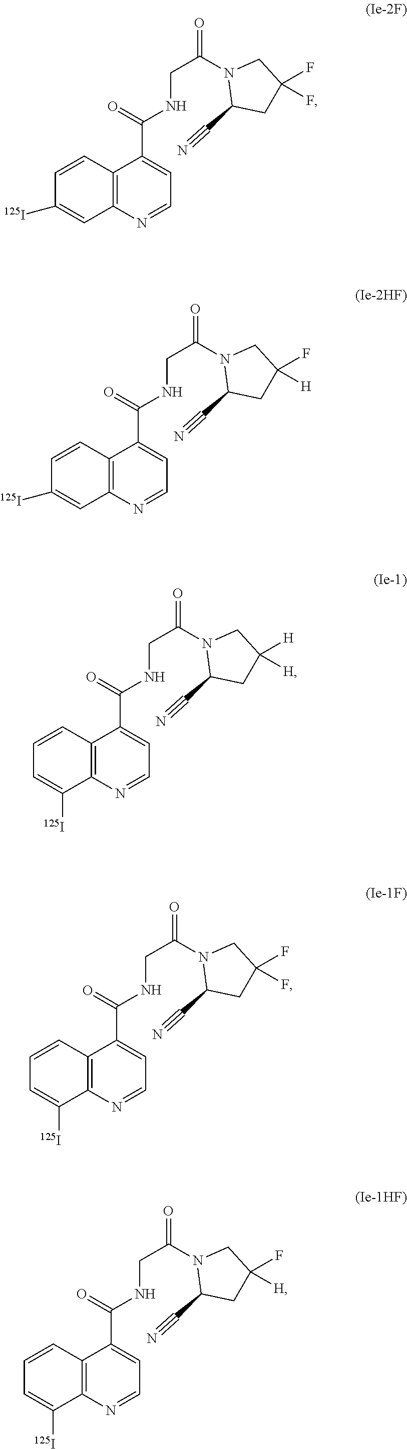

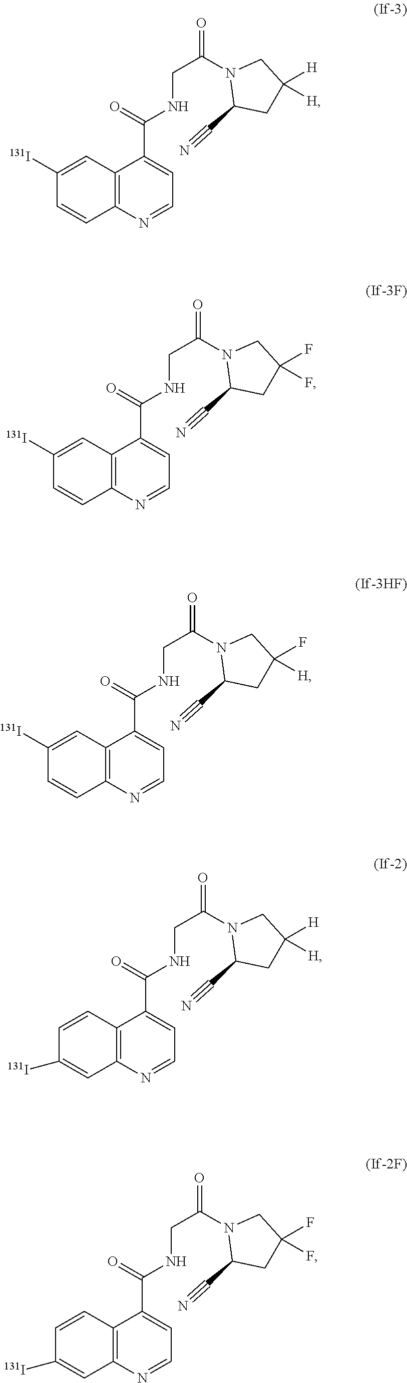

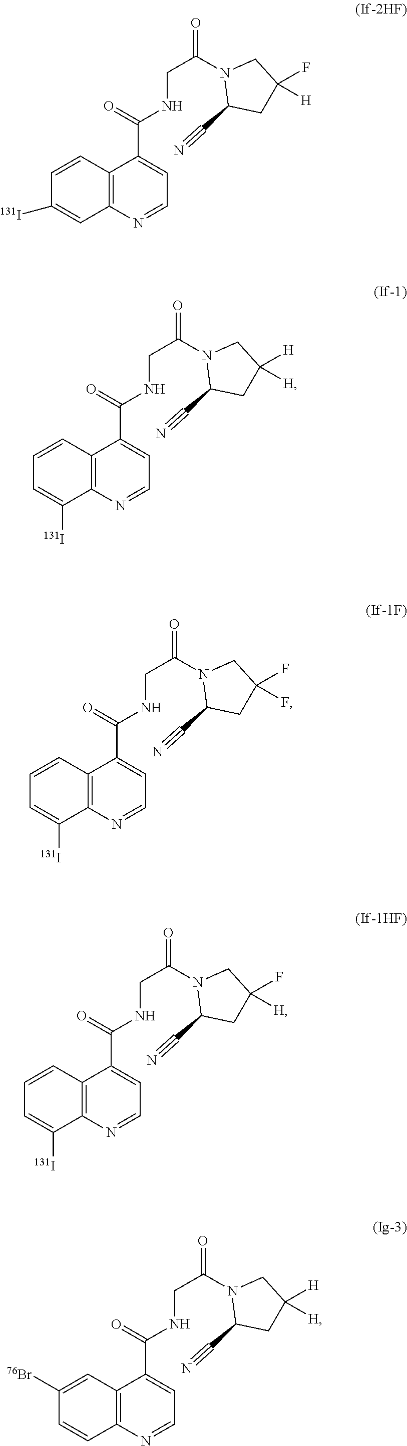

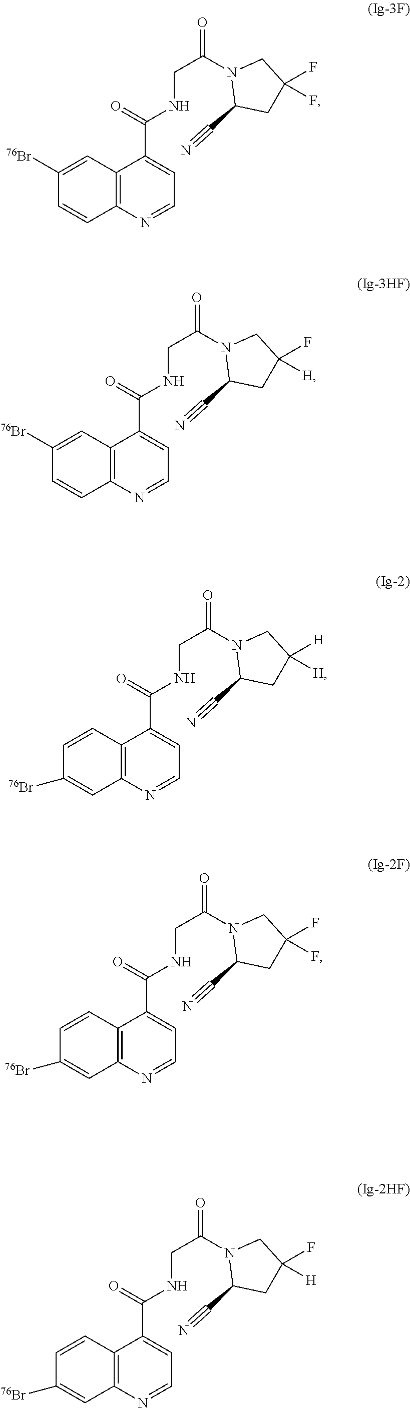

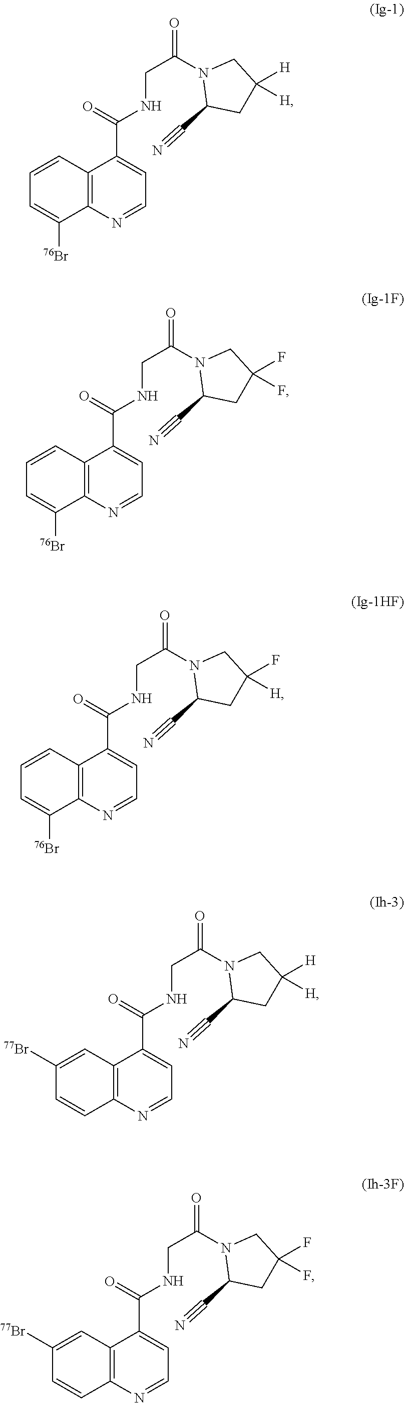

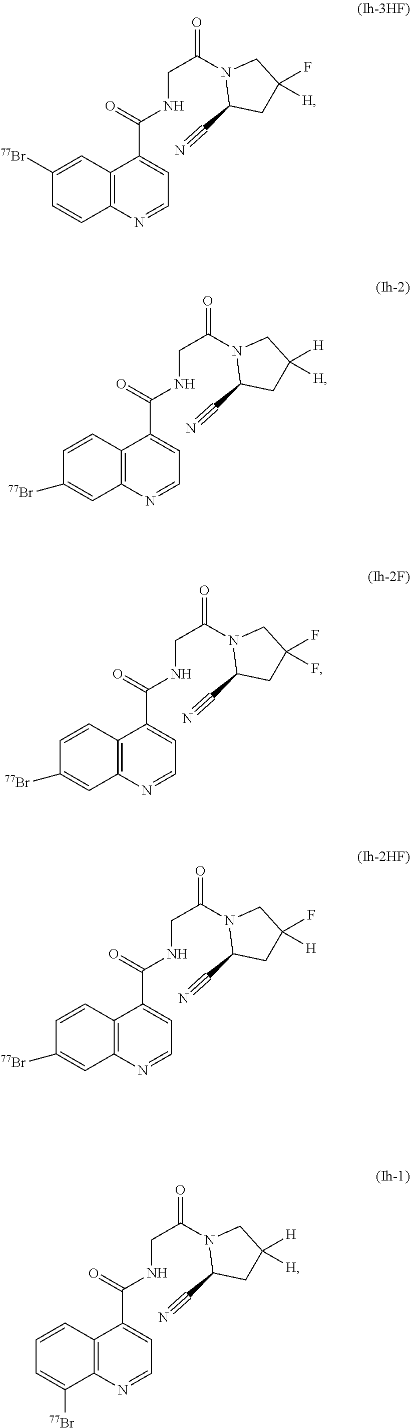

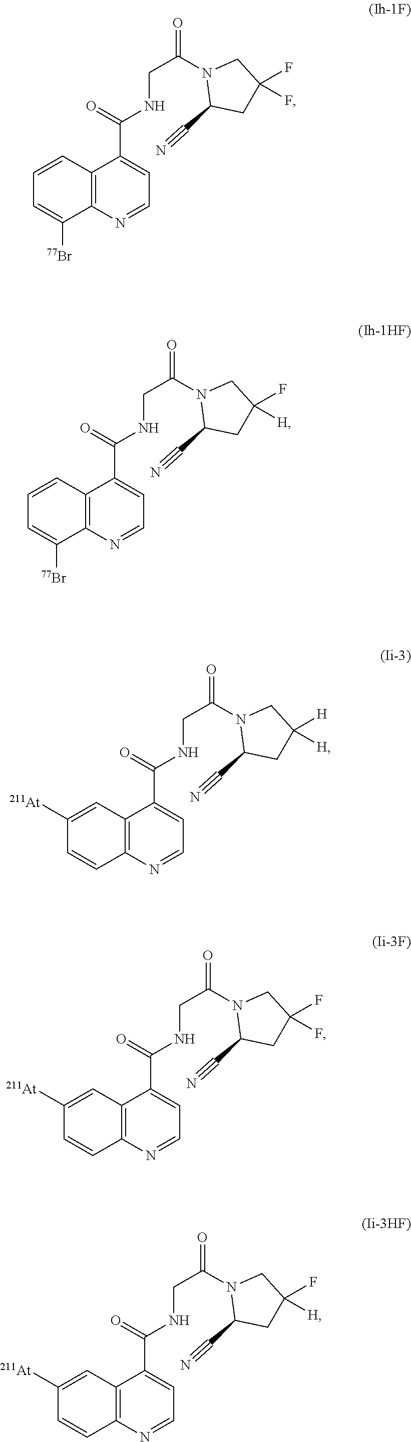

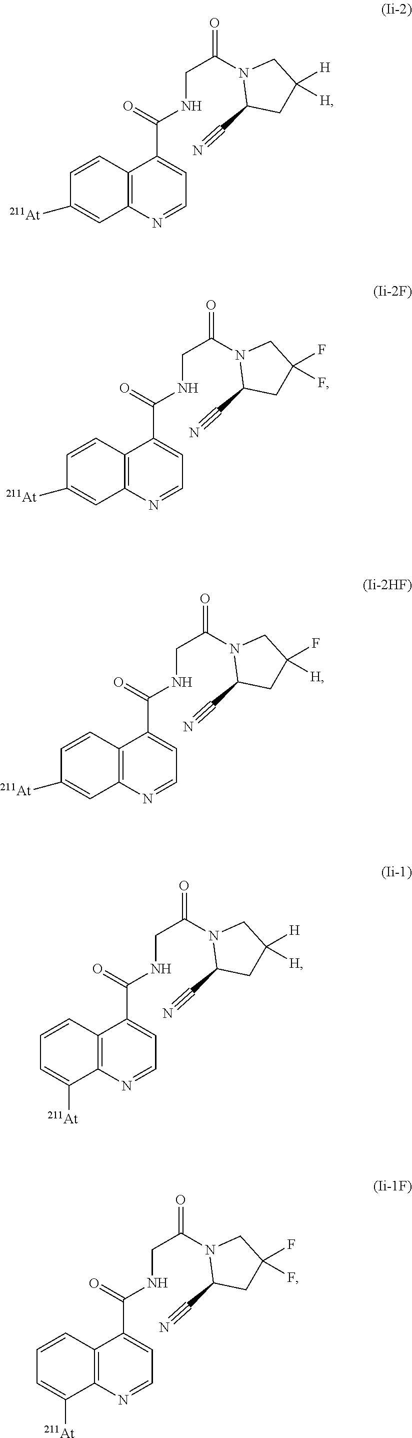

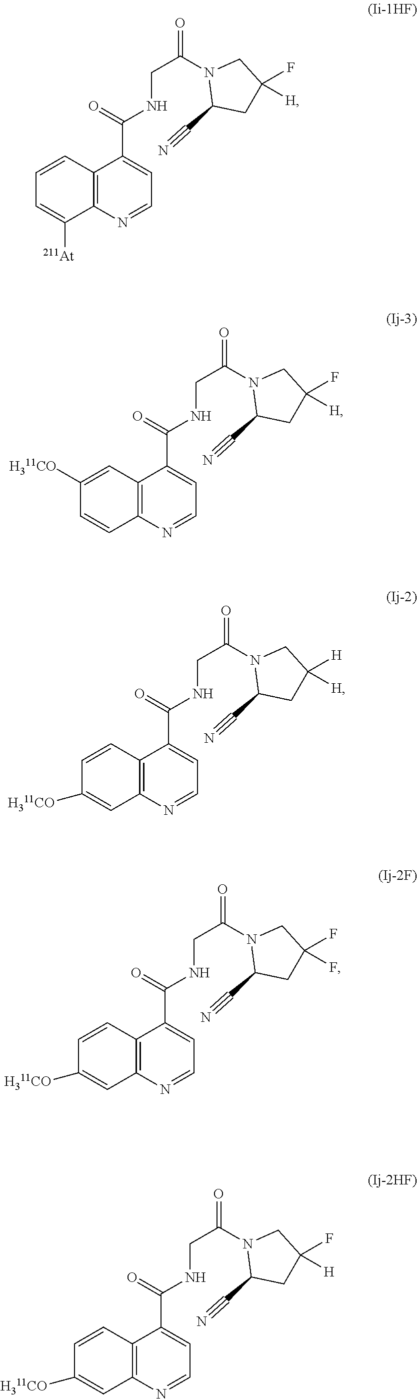

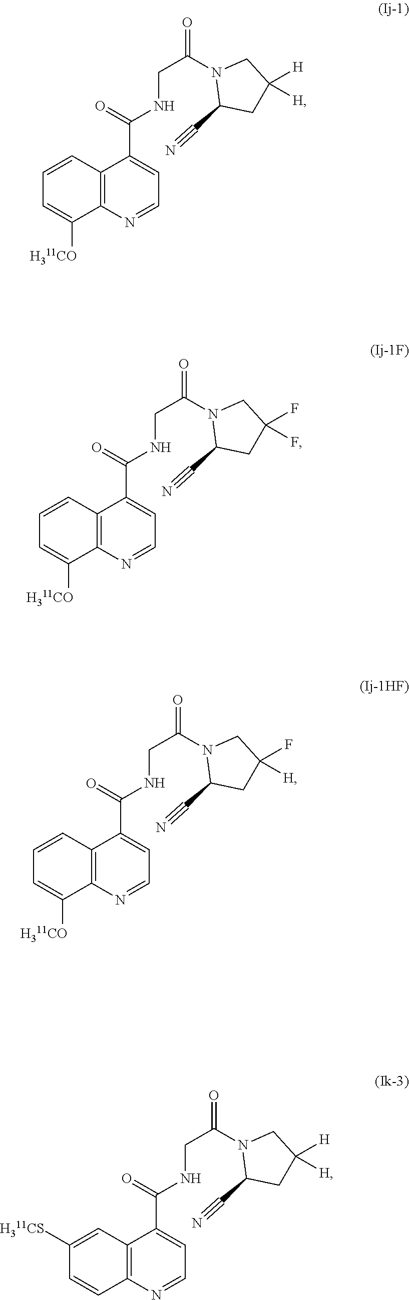

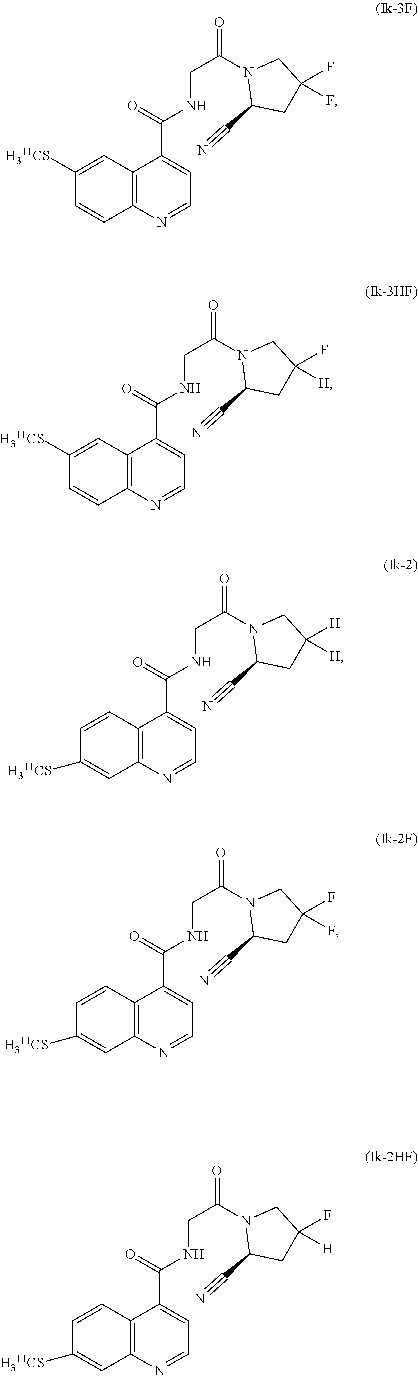

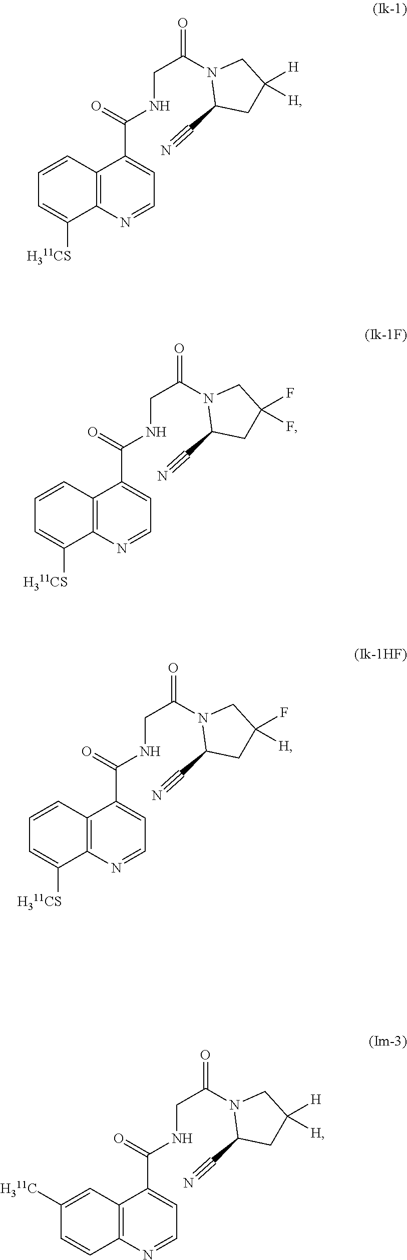

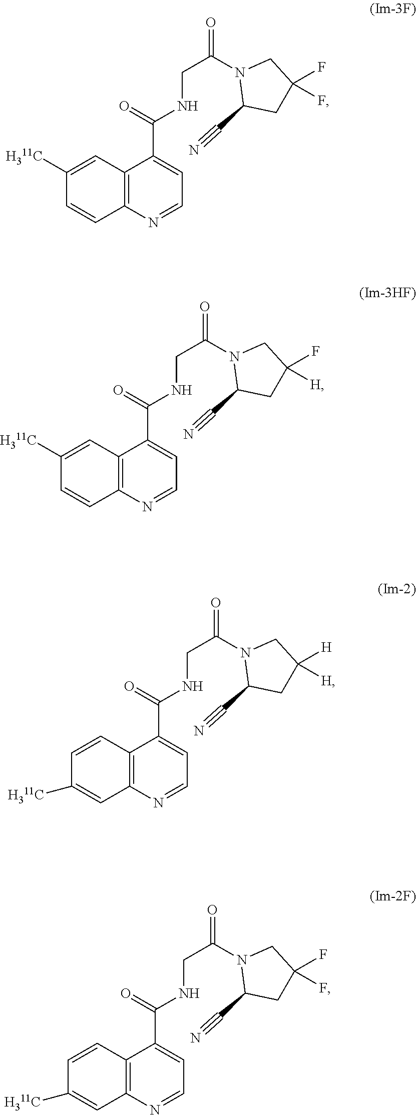

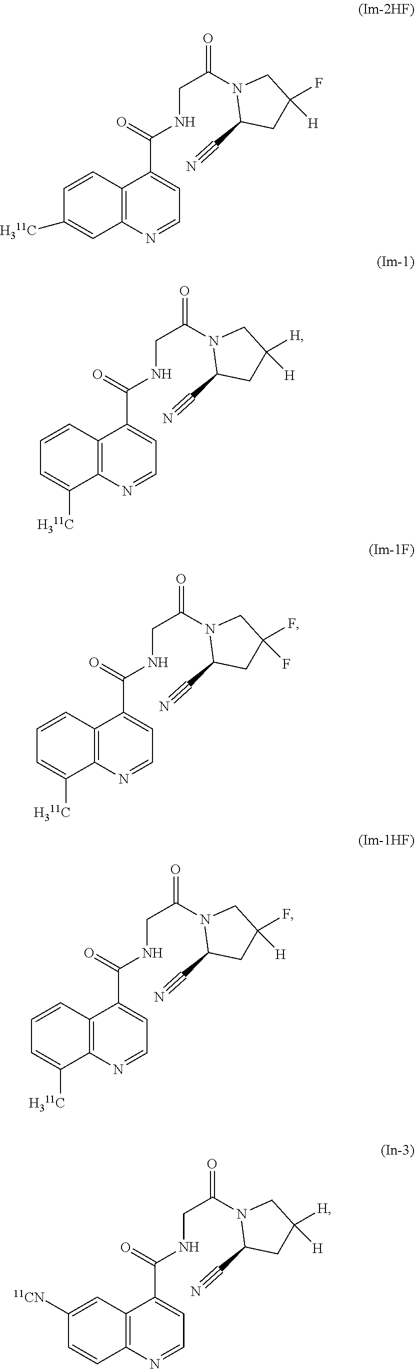

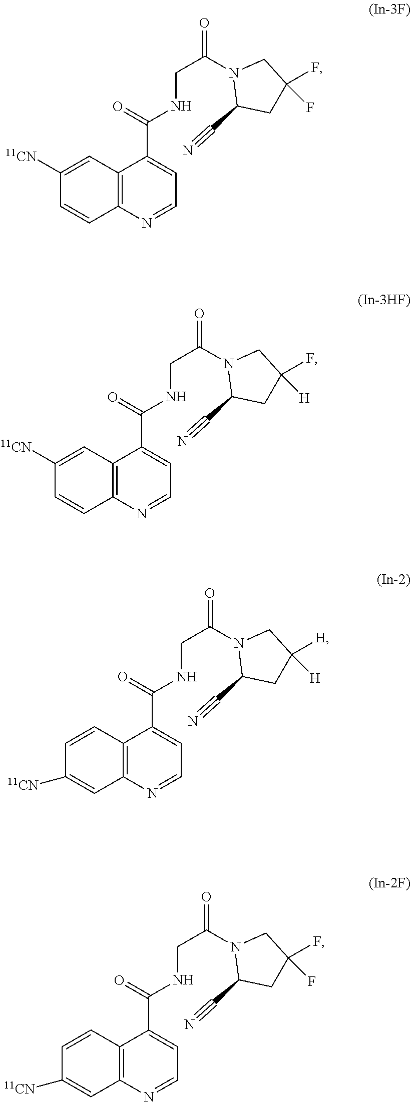

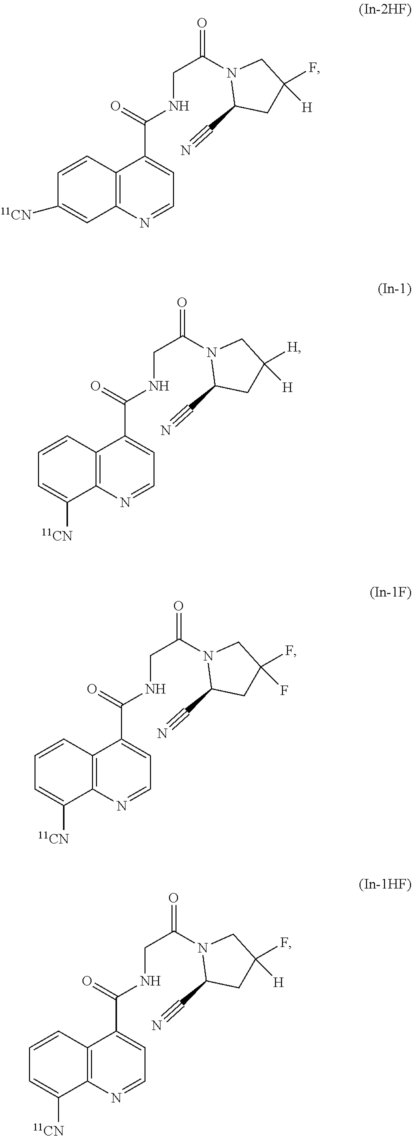

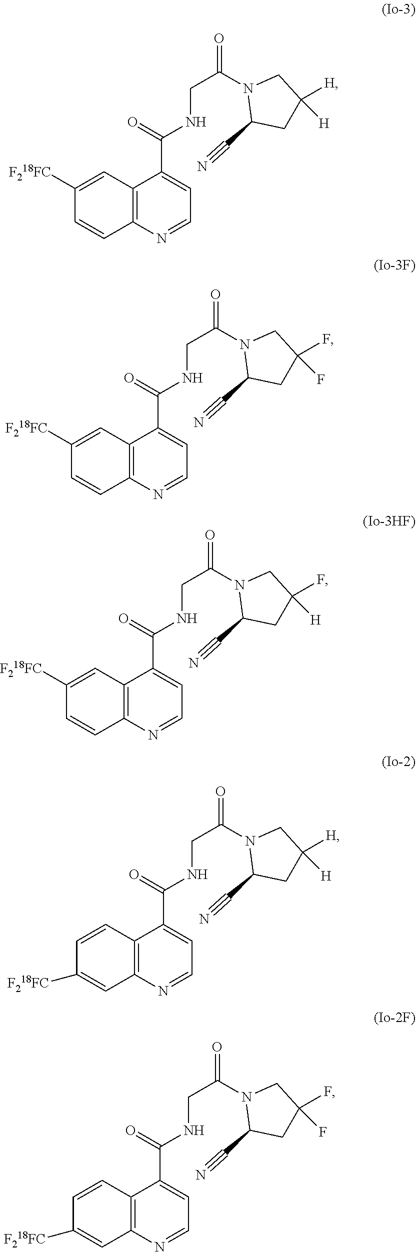

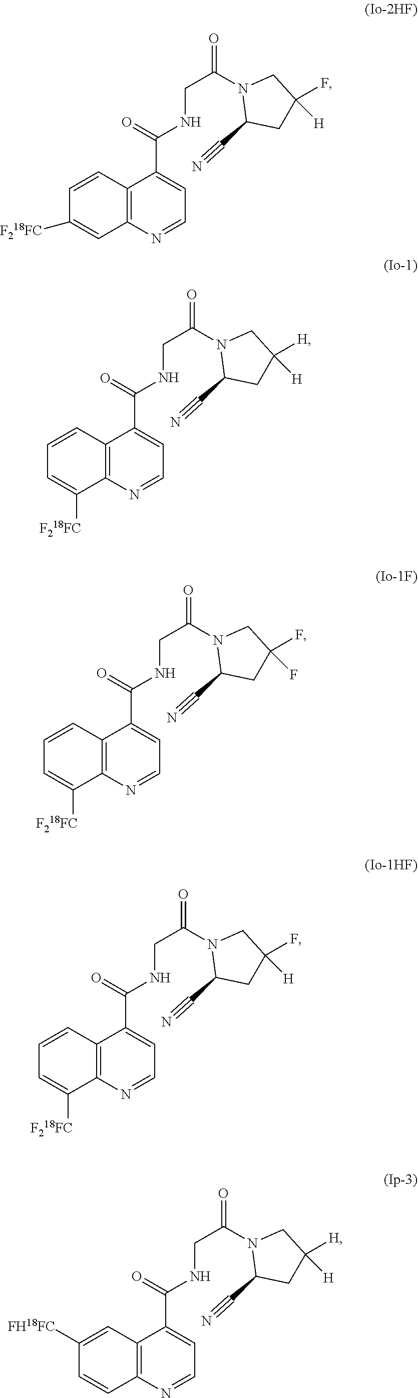

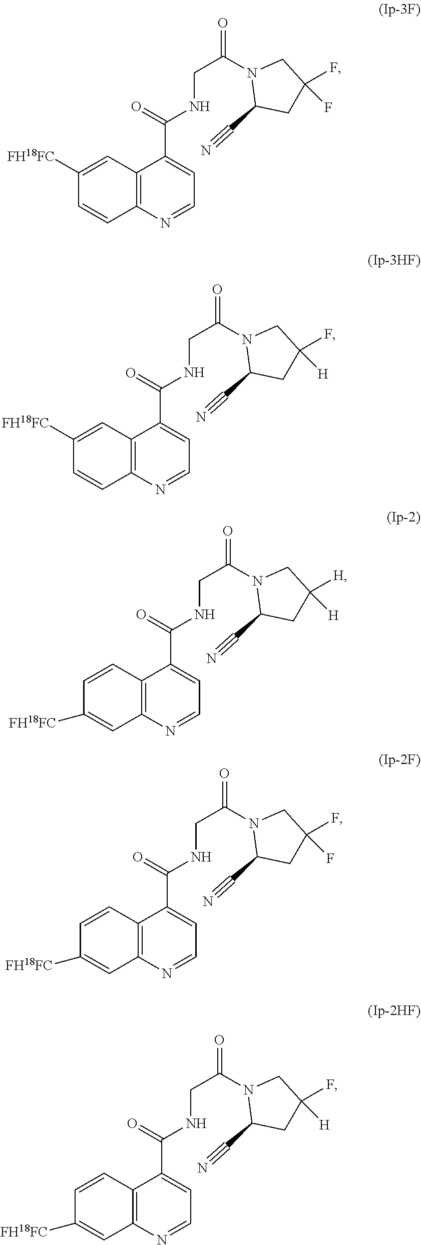

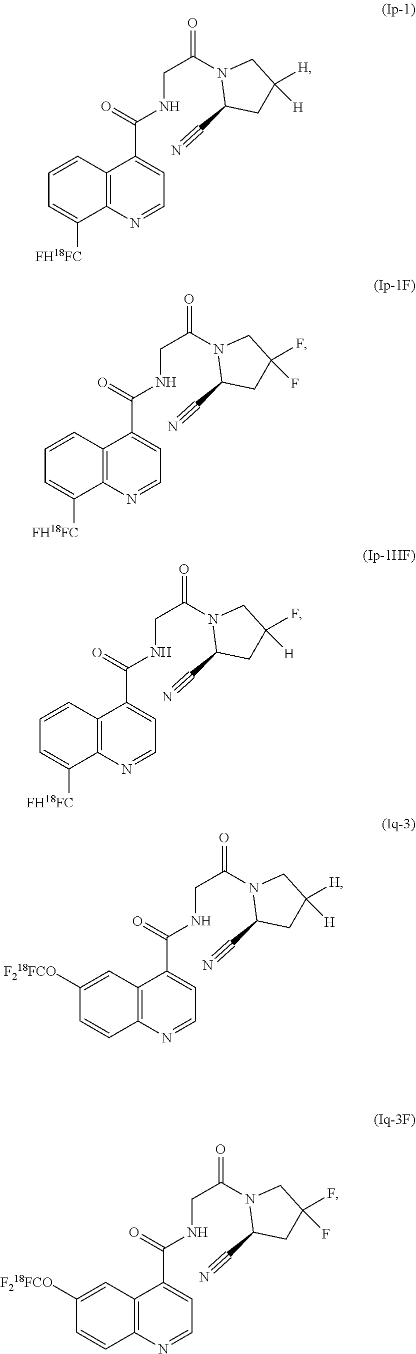

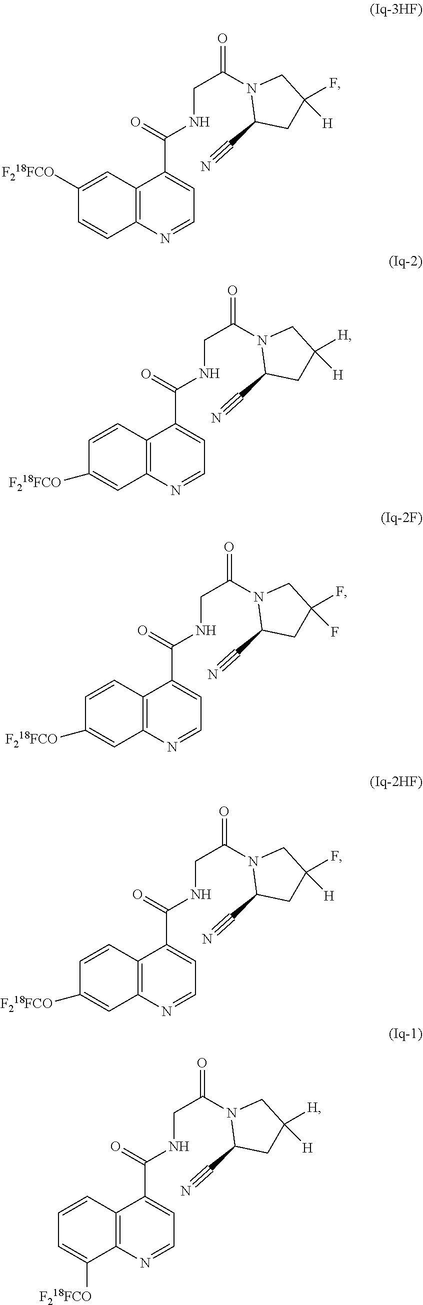

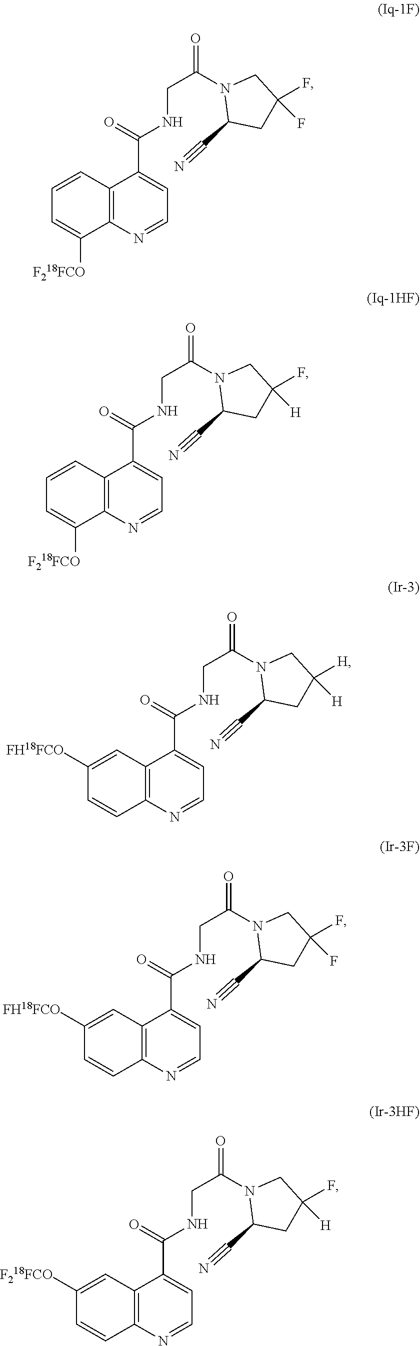

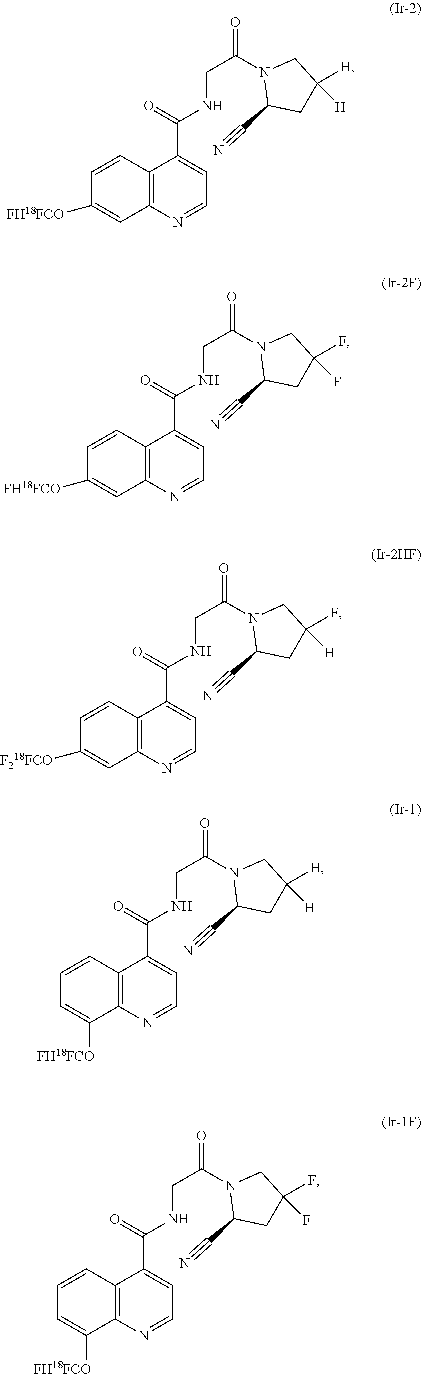

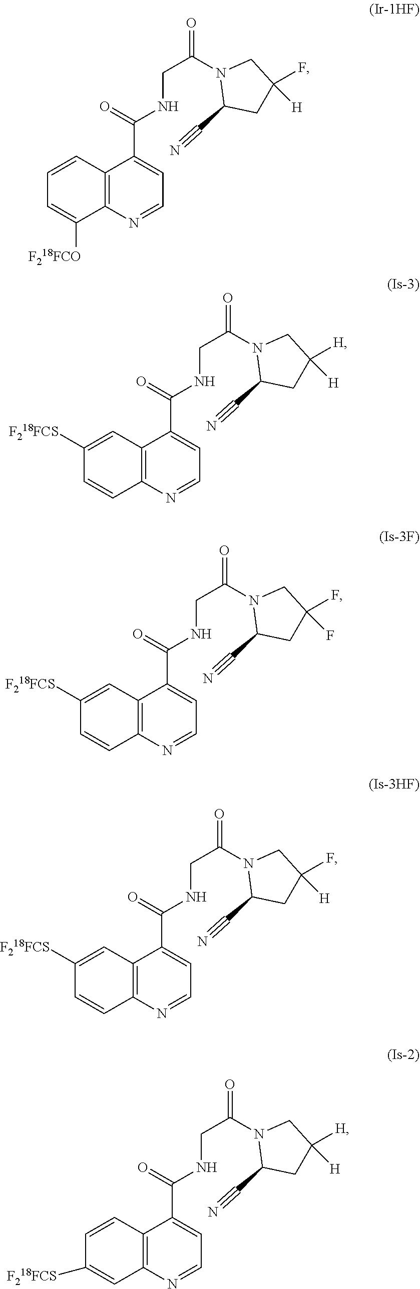

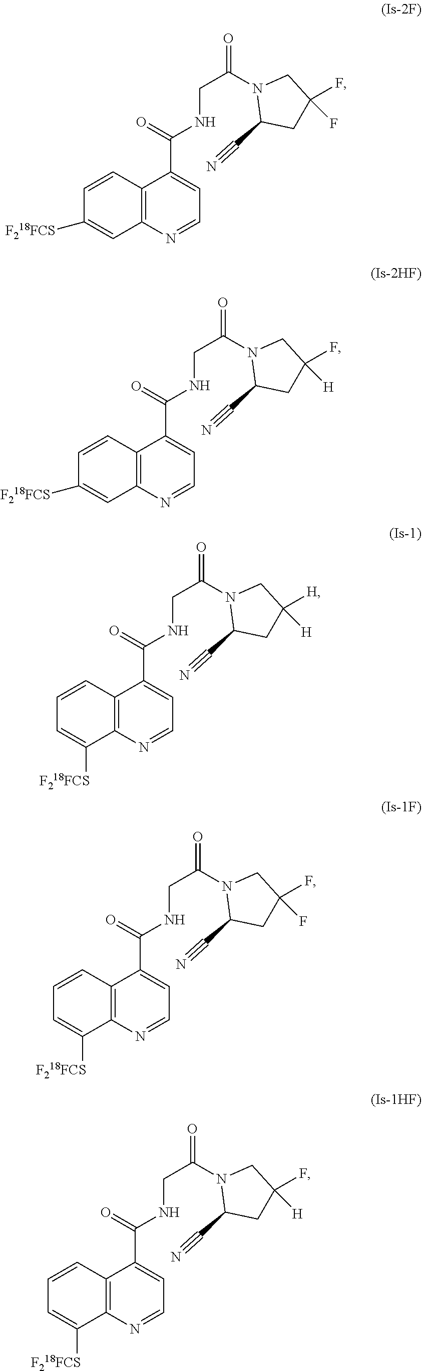

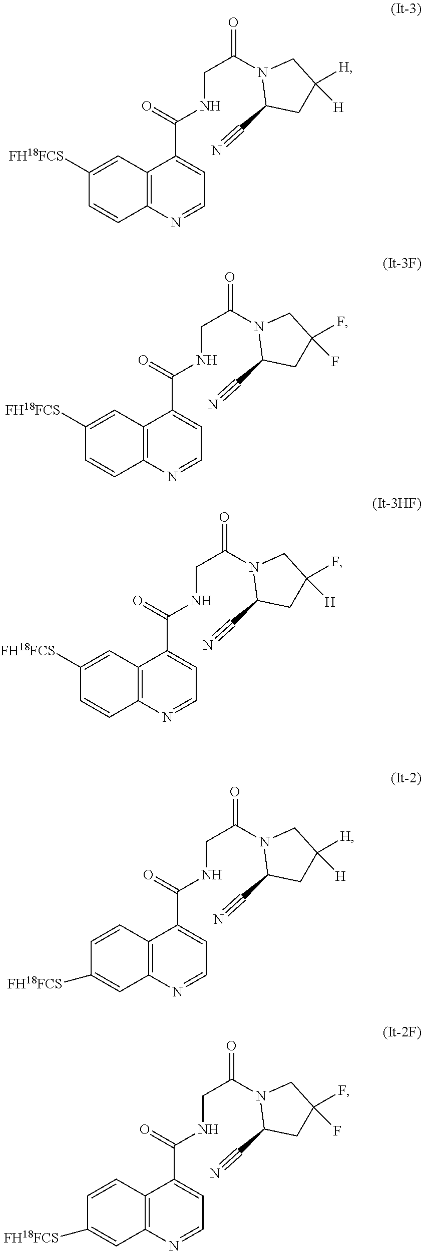

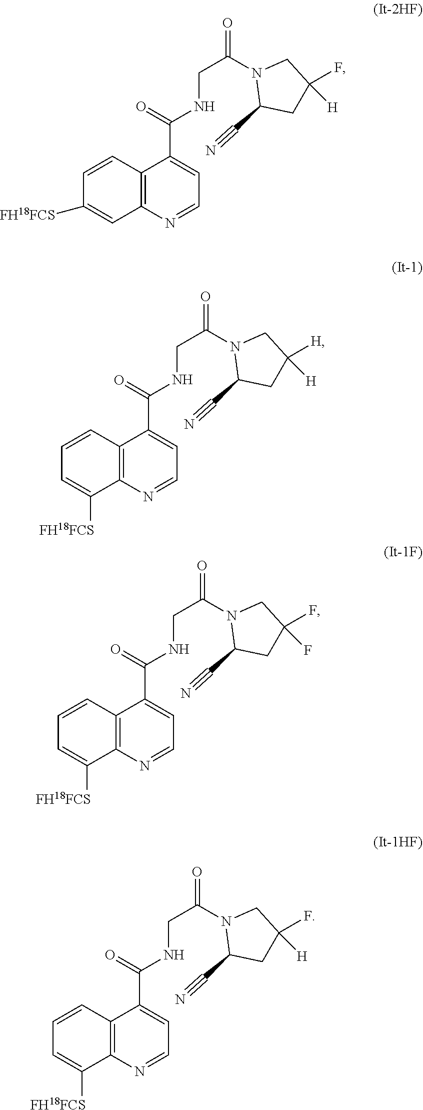

### Compounds of Formula (II)

In some embodiments, the disclosure provides a compound having a structure according to formula (II):

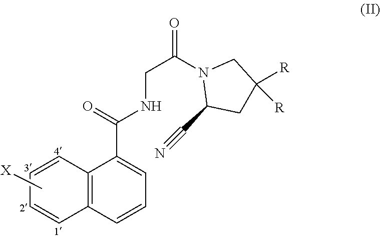

wherein X is located at carbon 1′, 2′, or 3′ and is selected from the group of I, ¹⁸F, ²¹¹At, ¹²³I, ¹²⁴I, ¹²⁵I, ¹³¹I, ⁷⁶Br, ⁷⁷Br, H₃¹¹CO, CF₂¹⁸F, CHF¹⁸F, OCF₂¹⁸F, OCHF¹⁸F, SCF₂¹⁸F, SCHF¹⁸F, ¹¹CN, ¹¹CH₃, and H₃¹¹CS; each R is independently selected from H, F, ²H, CH₃, and cyclopentyl.

In some embodiments, X is selected from the group of ¹⁸F, ²¹¹At, ¹²³I, ¹²⁴I, ¹²⁵I, ¹³¹I, ⁷⁶Br, ⁷⁷Br, H₃¹¹CO, CF₂¹⁸F, CHF¹⁸F, OCF₂¹⁸F, OCHF¹⁸F, SCF₂¹⁸F, SCHF¹⁸F, ¹¹CN, ¹¹CH₃, and H₃¹¹CS. In embodiments, X is selected from the group of ¹⁸F, ²¹¹At, ¹²³I, ¹²⁴I, ¹²⁵I, ¹³¹I, ⁷⁶Br, ⁷⁷Br, and H₃¹¹CO.

In some embodiments, X is located at the 3′ carbon. In some embodiments, X is located at the 2′ carbon. In some embodiments, X is located at the 1′ carbon.

In some embodiments, X is located at the 3′ carbon and X is I. In some embodiments, X is located at the 3′ carbon and X is ¹⁸F. In some embodiments, X is located at the 3′ carbon and X is ²¹¹At. In some embodiments, X is located at the 3′ carbon and X is ¹²³I. In some embodiments, X is located at the 3′ carbon and X is ¹²⁴I. In some embodiments, X is located at the 3′ carbon and X is ¹²⁵I. In some embodiments, X is located at the 3′ carbon and X is ¹³¹I. In some embodiments, X is located at the 3′ carbon and X is ⁷⁶Br. In some embodiments, X is located at the 3′ carbon and X is ⁷⁷Br. In some embodiments, X is located at the 3′ carbon and X is H₃¹¹CO. In some embodiments, X is located at the 3′ carbon and X is CF₂¹⁸F. In some embodiments, X is located at the 3′ carbon and X is CHF¹⁸F. In some embodiments, X is located at the 3′ carbon and X is OCF₂¹⁸F. In some embodiments, X is located at the 3′ carbon and X is OCHF¹⁸F. In some embodiments, X is located at the 3′ carbon and X is SCF₂¹⁸F. In some embodiments, X is located at the 3′ carbon and X is SCHF¹⁸F. In some embodiments, X is located at the 3′ carbon and X is ¹¹CN. In some embodiments, X is located at the 3′ carbon and X is ¹¹CH₃. In some embodiments, X is located at the 3′ carbon and X is H₃¹¹CS.

In some embodiments, X is located at the 2′ carbon and X is I. In some embodiments, X is located at the 2′ carbon and X is ¹⁸F. In some embodiments, X is located at the 2′ carbon and X is ²¹¹At. In some embodiments, X is located at the 2′ carbon and X is ¹²³I. In some embodiments, X is located at the 2′ carbon and X is ¹²⁴I. In some embodiments, X is located at the 2′ carbon and X is ¹²⁵I. In some embodiments, X is located at the 2′ carbon and X is ¹³¹I. In some embodiments, X is located at the 2′ carbon and X is ⁷⁶Br. In some embodiments, X is located at the 2′ carbon and X is ⁷⁷Br. In some embodiments, X is located at the 2′ carbon and X is H₃¹¹CO. In some embodiments, X is located at the 2′ carbon and X is CF₂¹⁸F. In some embodiments, X is located at the 2′ carbon and X is CHF¹⁸F. In some embodiments, X is located at the 2′ carbon and X is OCF₂¹⁸F. In some embodiments, X is located at the 2′ carbon and X is OCHF¹⁸F. In some embodiments, X is located at the 2′ carbon and X is SCF₂¹⁸F. In some embodiments, X is located at the 2′ carbon and X is SCHF¹⁸F. In some embodiments, X is located at the 2′ carbon and X is ¹¹CN. In some embodiments, X is located at the 2′ carbon and X is ¹¹CH₃. In some embodiments, X is located at the 2′ carbon and X is H₃¹¹CS.

In some embodiments, X is located at the 1′ carbon and X is I. In some embodiments, X is located at the 1′ carbon and X is ¹⁸F. In some embodiments, X is located at the 1′ carbon and X is ²¹¹At. In some embodiments, X is located at the 1′ carbon and X is ¹²³I. In some embodiments, X is located at the 1′ carbon and X is ¹²⁴I. In some embodiments, X is located at the 1′ carbon and X is ¹²⁵I. In some embodiments, X is located at the 1′ carbon and X is ¹³¹I. In some embodiments, X is located at the 1′ carbon and X is ⁷⁶Br. In some embodiments, X is located at the 1′ carbon and X is ⁷⁷Br. In some embodiments, X is located at the 1′ carbon and X is H₃¹¹CO. In some embodiments, X is located at the 1′ carbon and X is CF₂¹⁸F. In some embodiments, X is located at the 1′ carbon and X is CHF¹⁸F. In some embodiments, X is located at the 1′ carbon and X is OCF₂¹⁸F. In some embodiments, X is located at the 1′ carbon and X is OCHF¹⁸F. In some embodiments, X is located at the 1′ carbon and X is SCF₂¹⁸F. In some embodiments, X is located at the 1′ carbon and X is SCHF¹⁸F. In some embodiments, X is located at the 1′ carbon and X is ¹¹CN. In some embodiments, X is located at the 1′ carbon and X is ¹¹CH₃. In some embodiments, X is located at the 1′ carbon and X is H₃¹¹CS.

In general, each R is independently selected from H, F, ²H, CH₃, and cyclopentyl. In embodiments, each R is independently selected from H and F. In embodiments, at least one R is H. In embodiments, both R are H. In embodiments, at least one R is F. In embodiments, both R are F. In embodiments, at least one R is ²H. In embodiments, both R are 2H. In embodiments, at least one R is CH₃. In embodiments, both R are CH₃. In embodiments, at least one R is cyclopentyl. In embodiments, both R are cyclopentyl. In embodiments, X is located at carbon 3′ and both R are H. In embodiments, X is located at carbon 3′ and both R are F. In embodiments, X is located at carbon 2′ and both R are H. In embodiments, X is located at carbon 2′ and both R are F. In embodiments, X is located at carbon 1′ and both R are H. In embodiments, X is located at carbon 1′ and both R are F.

In embodiments, the fibroblast activation protein inhibitor has a structure according to one of the following formulae:

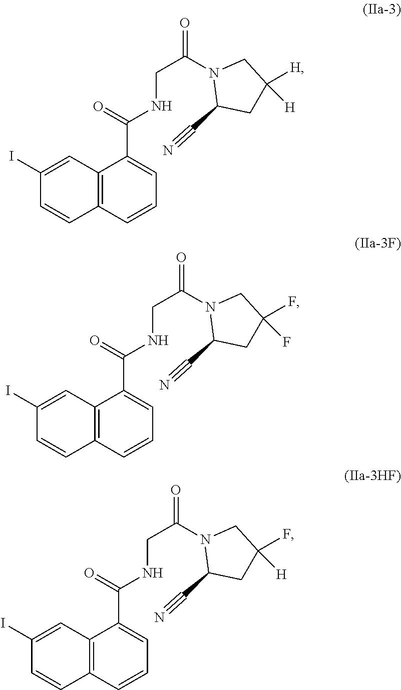

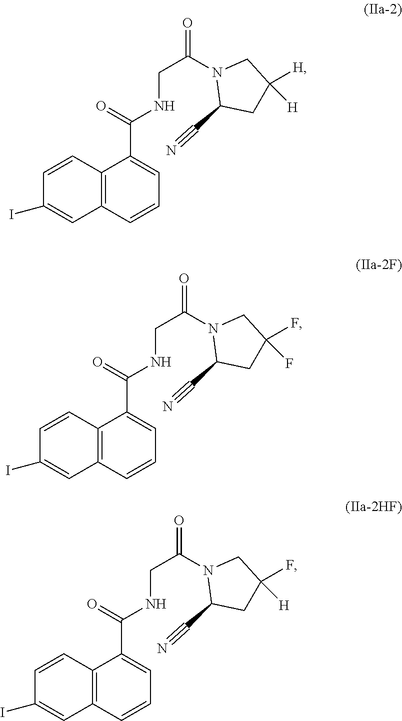

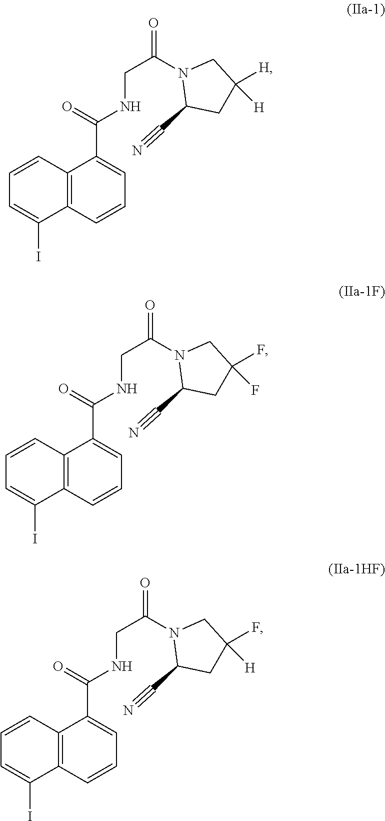

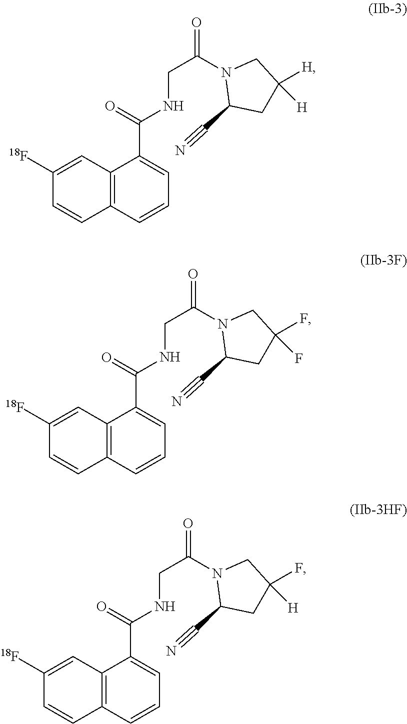

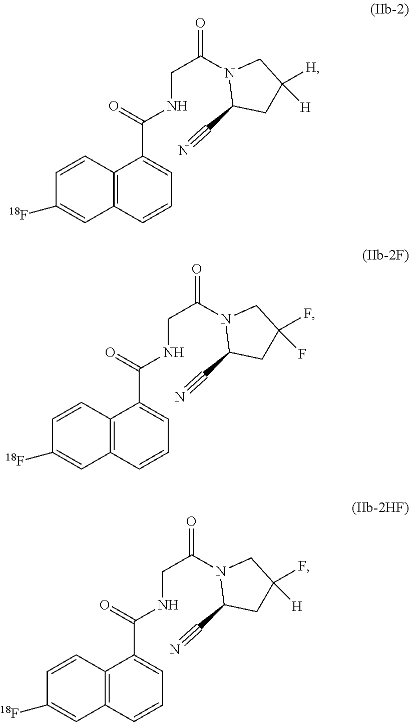

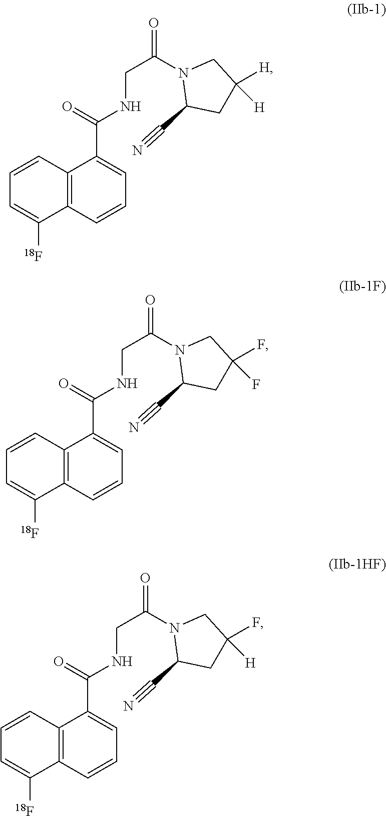

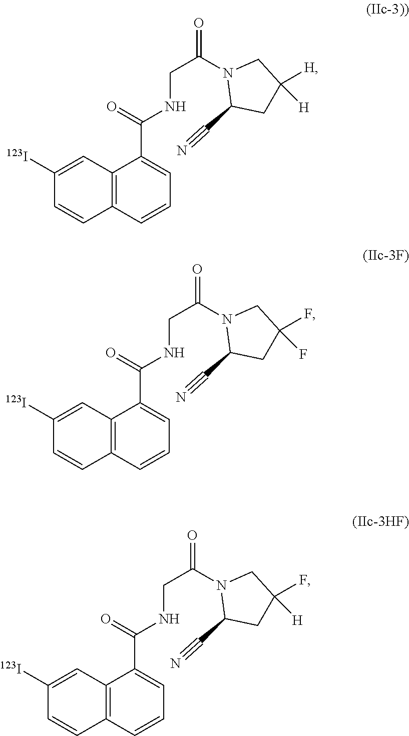

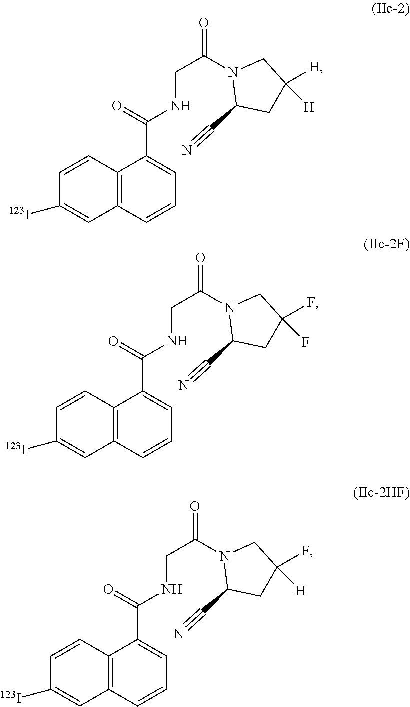

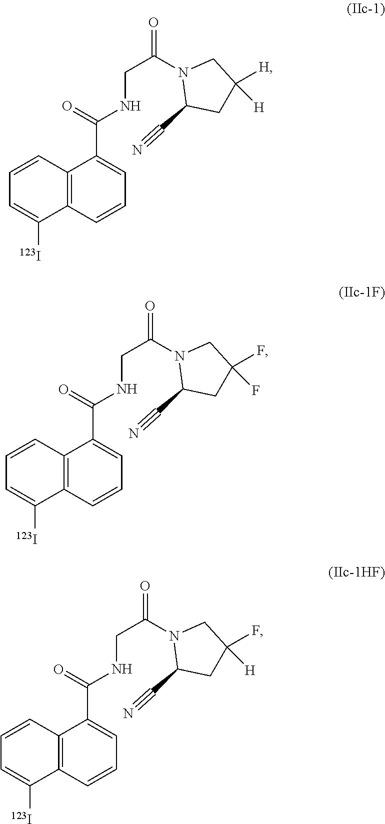

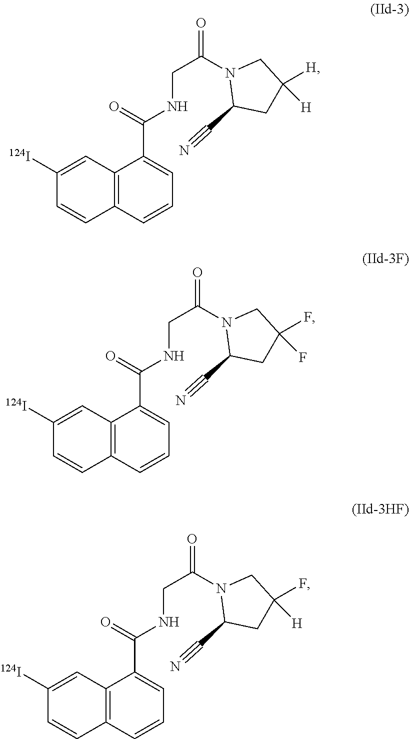

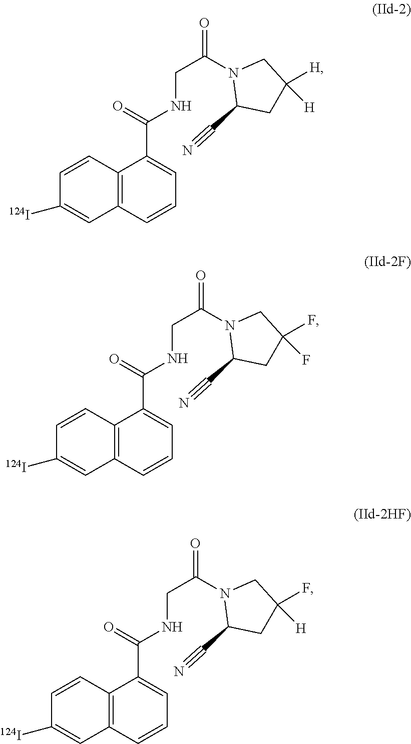

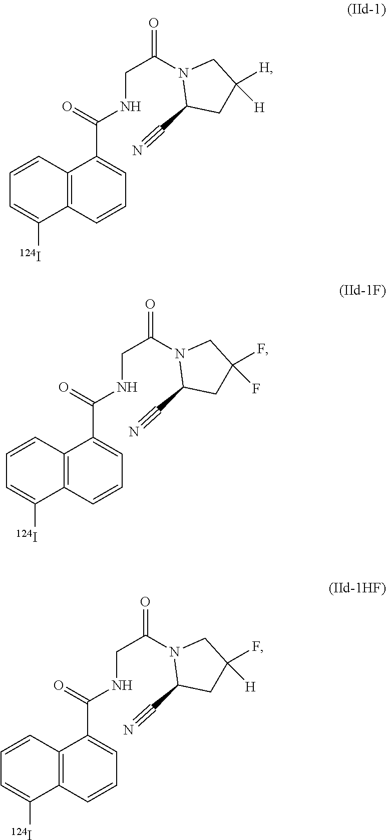

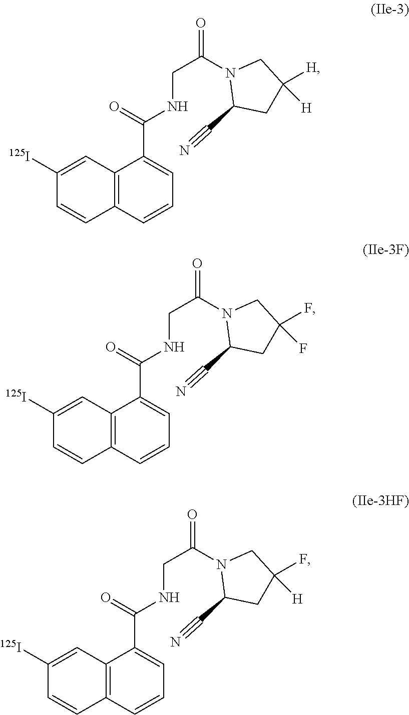

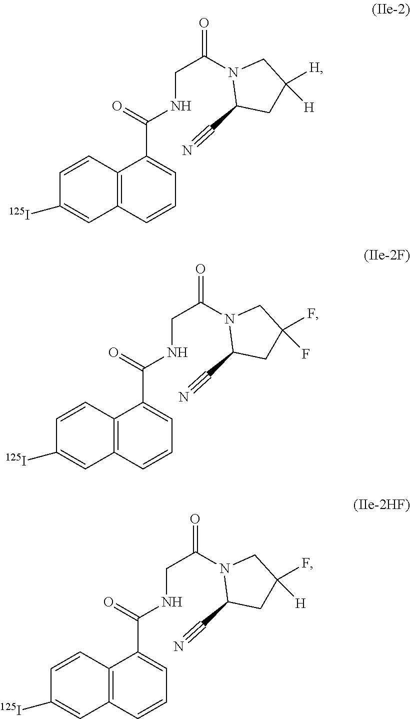

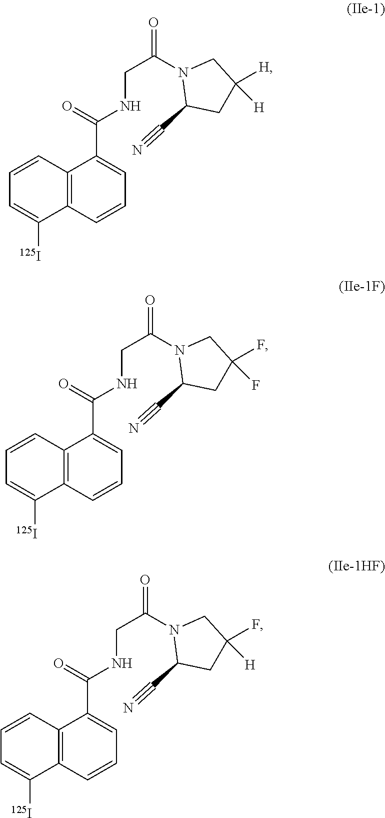

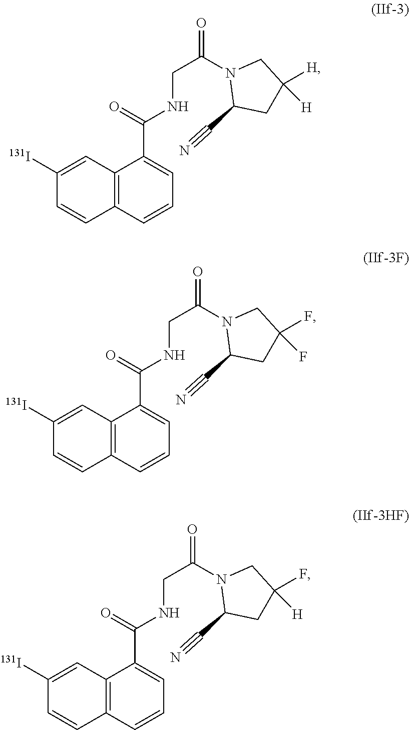

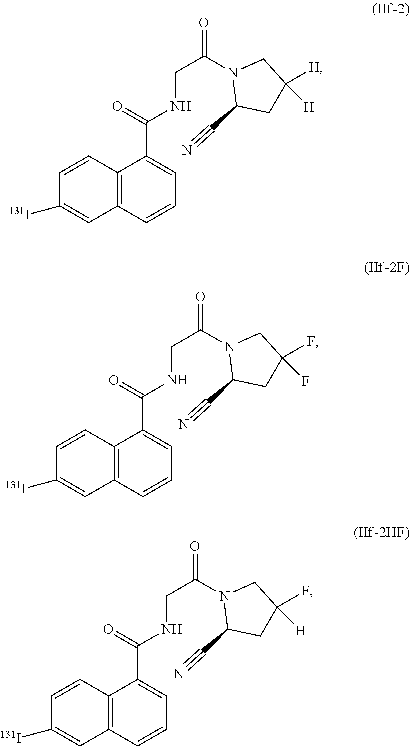

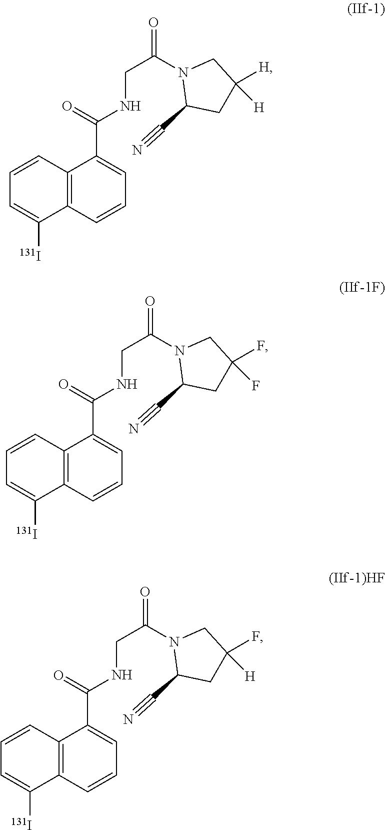

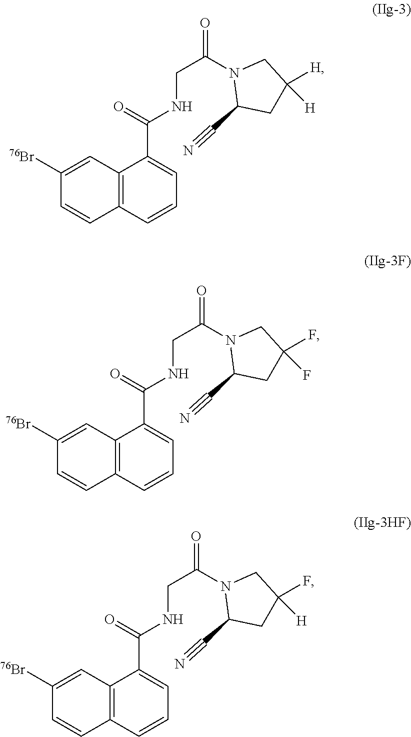

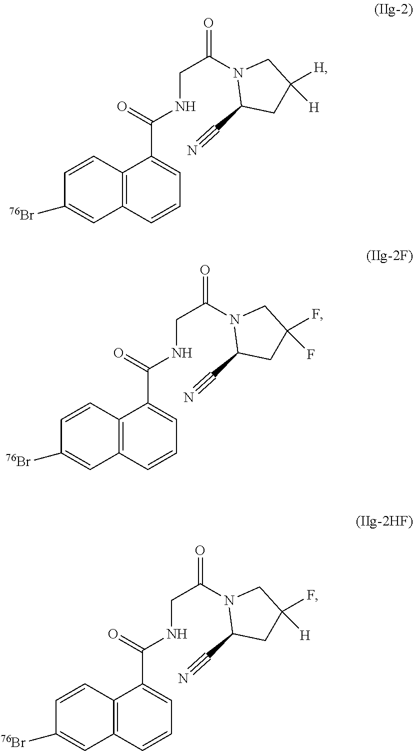

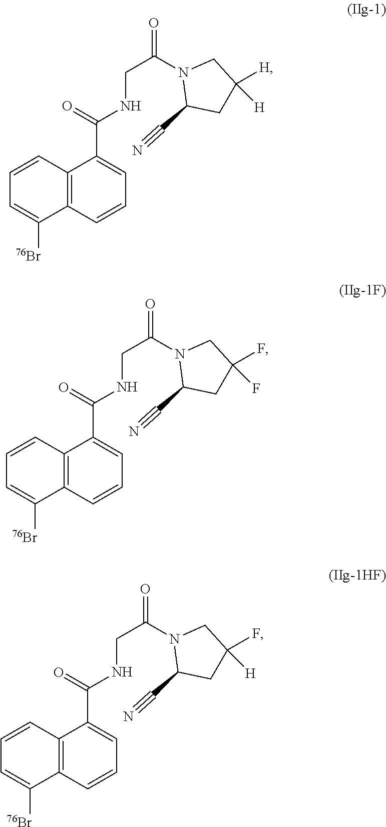

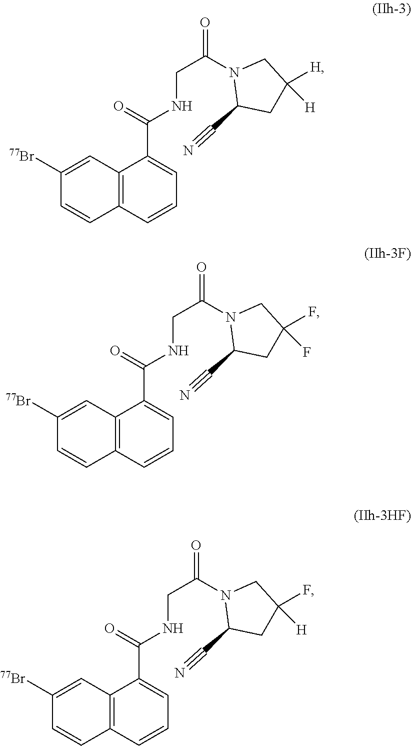

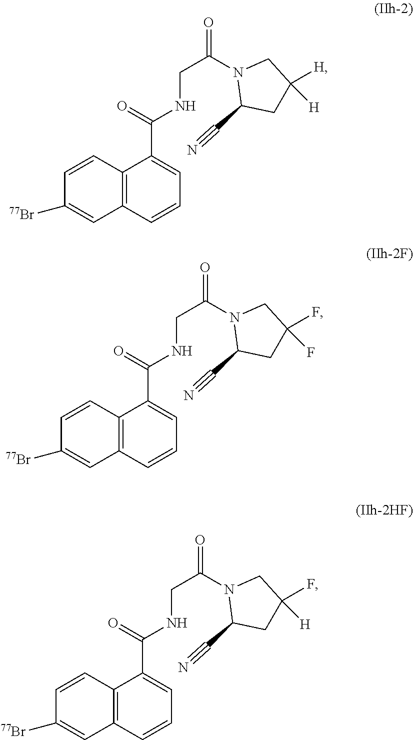

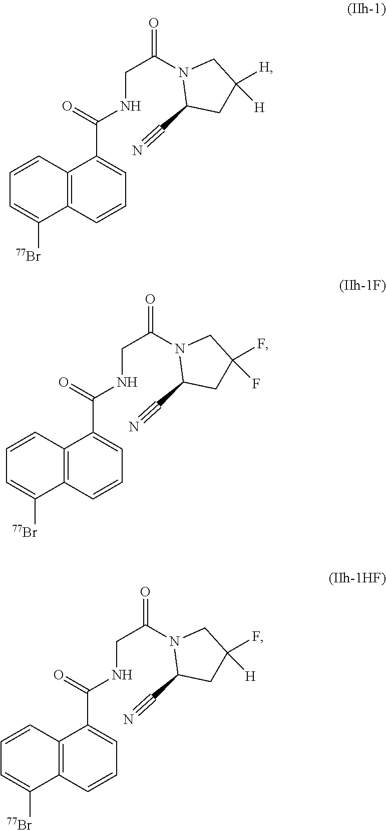

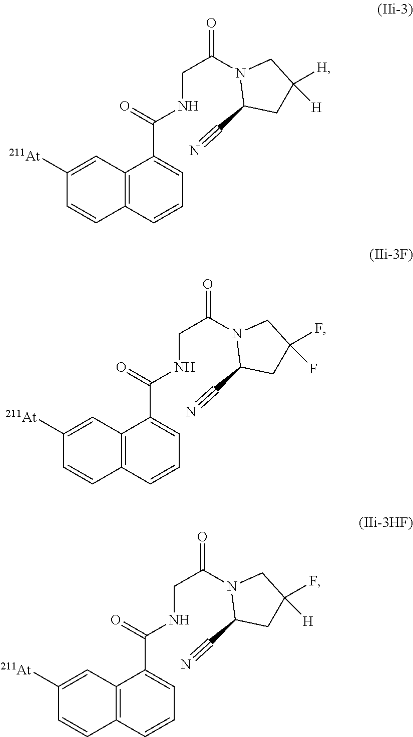

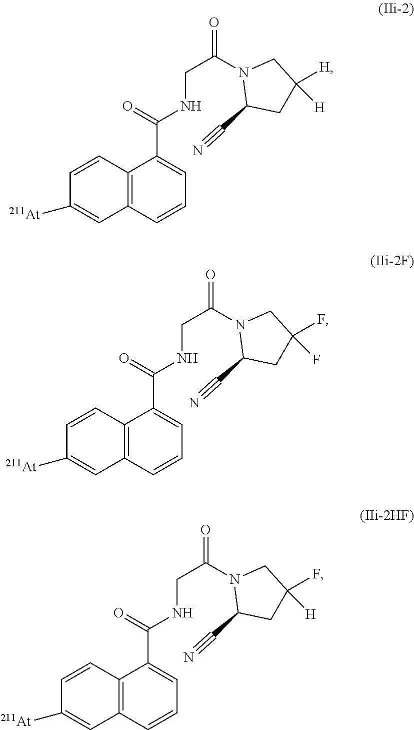

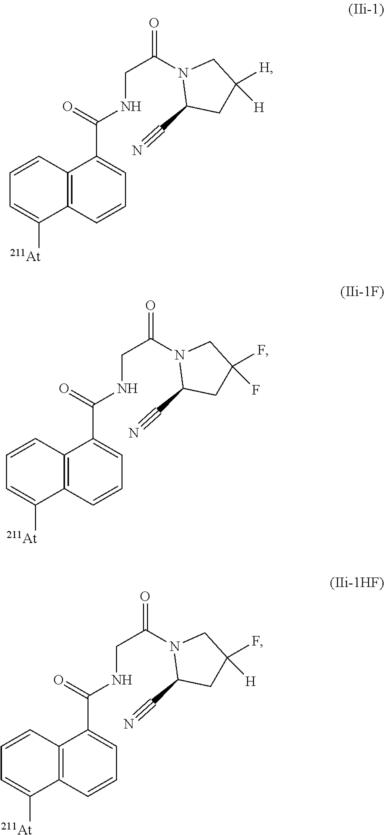

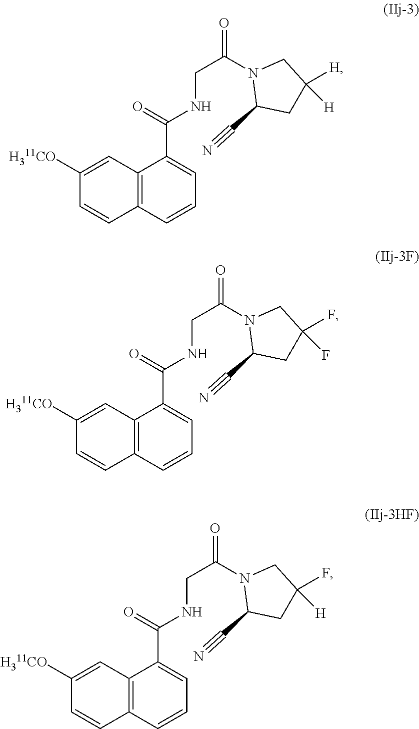

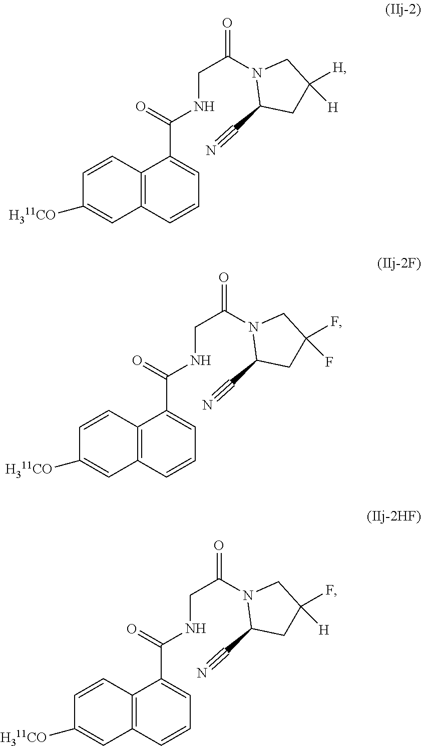

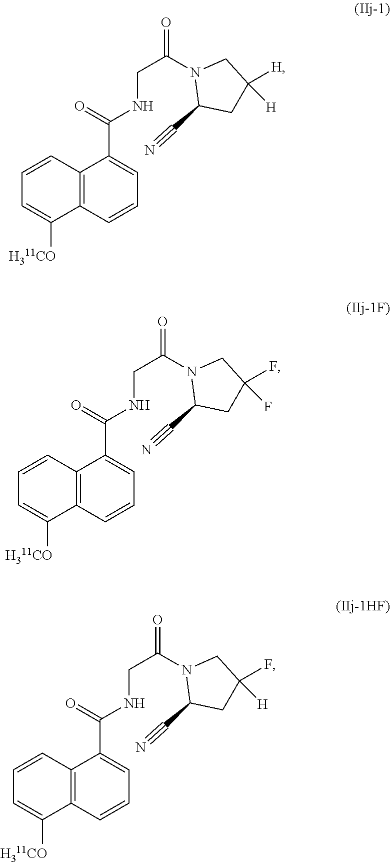

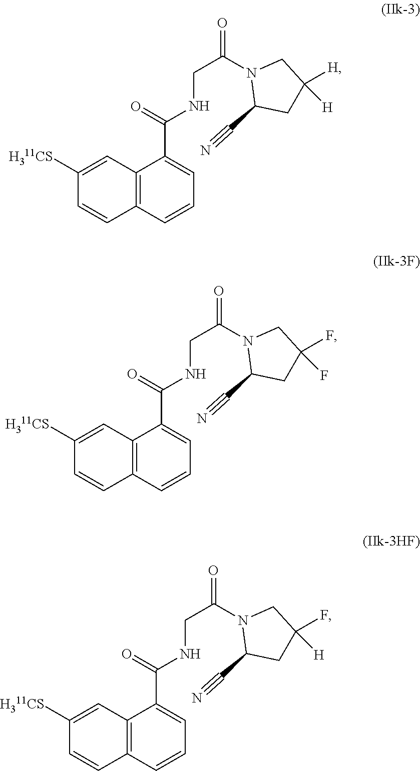

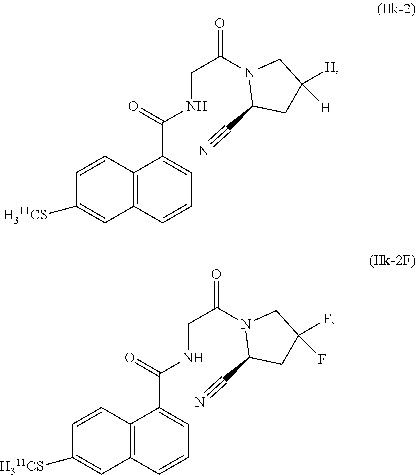

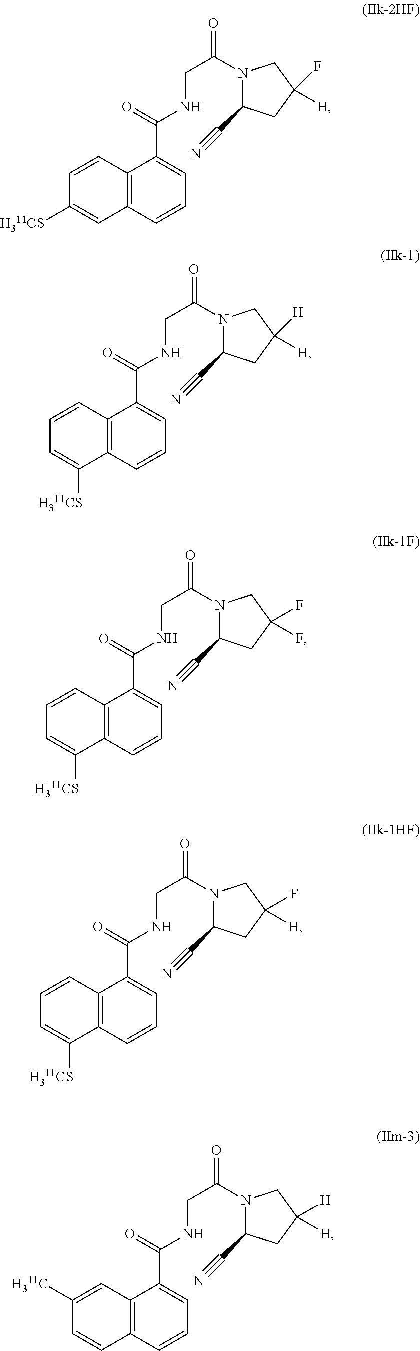

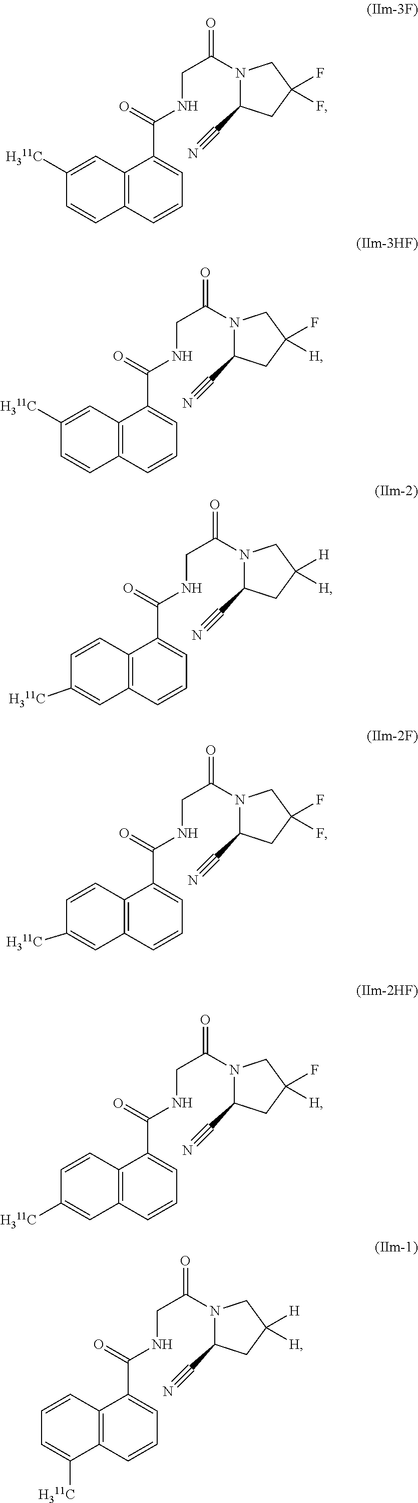

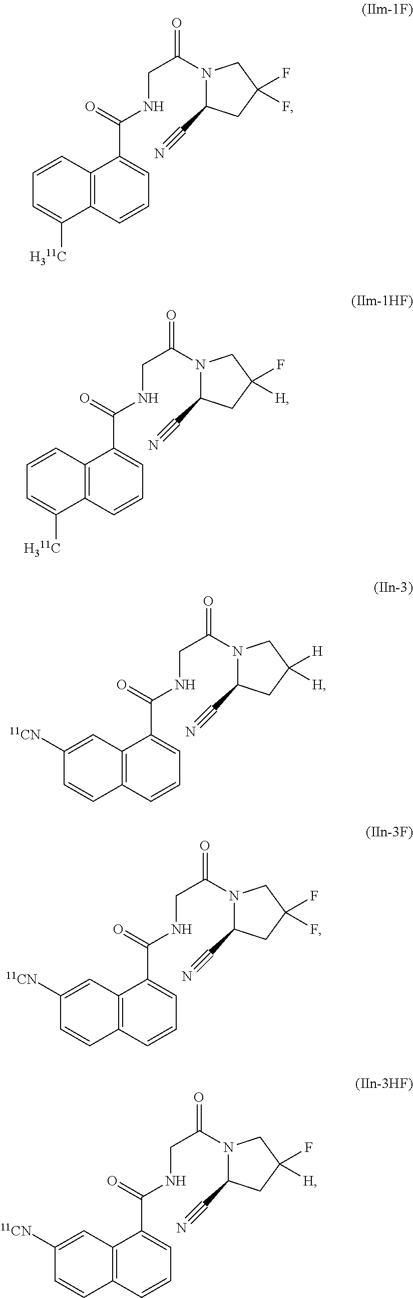

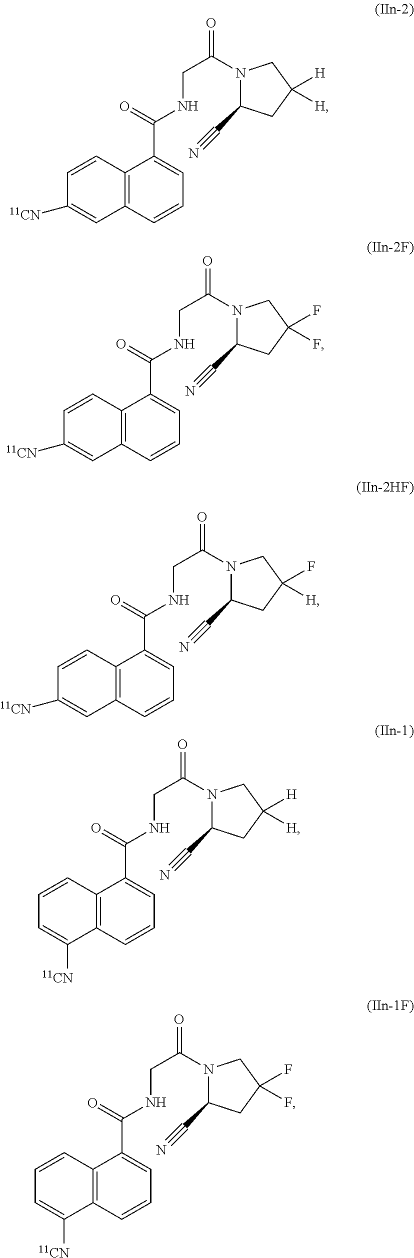

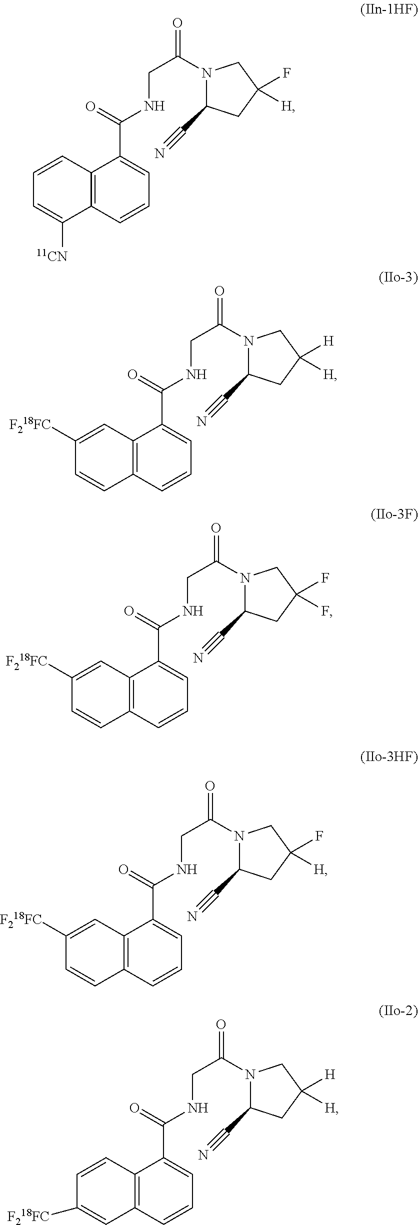

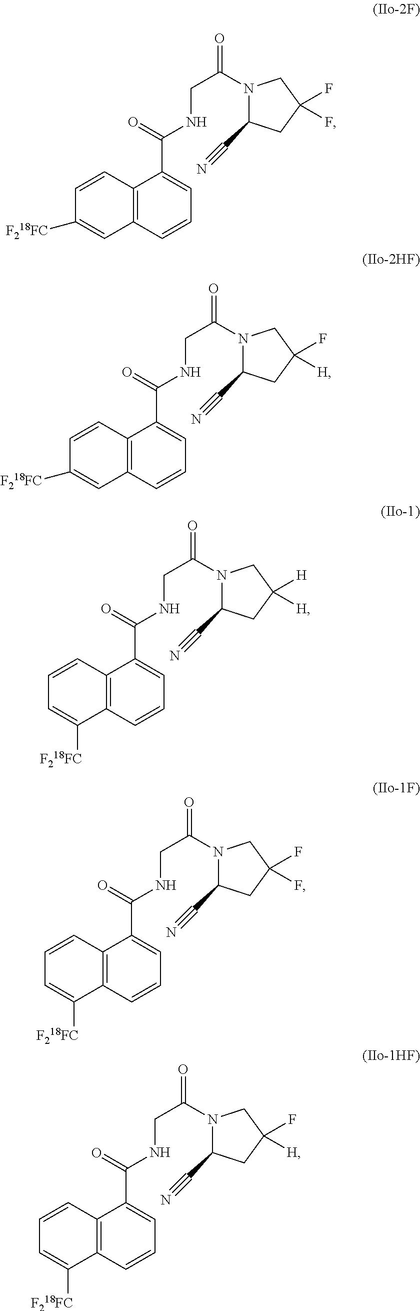

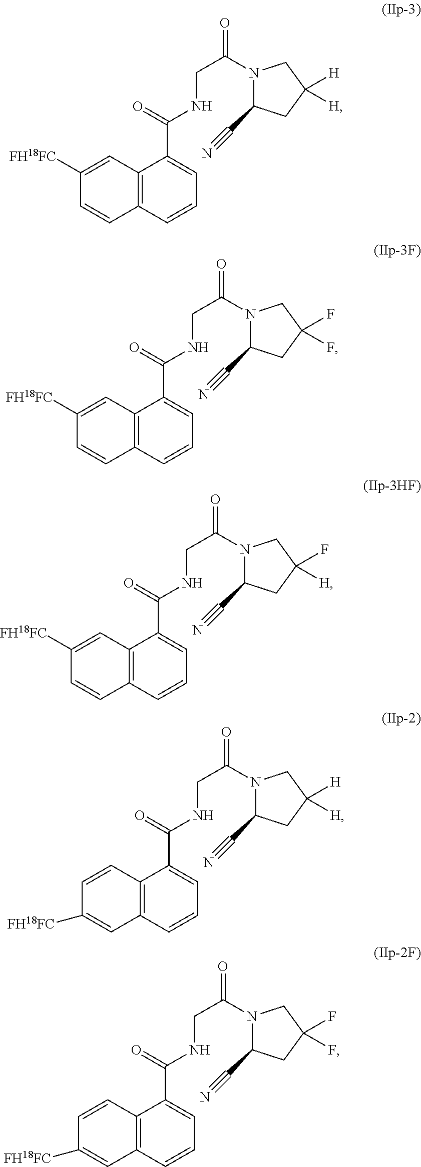

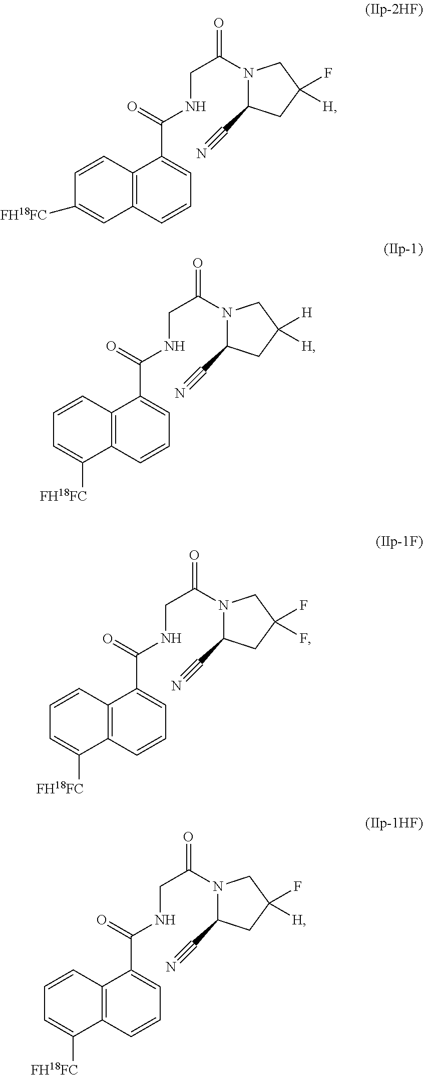

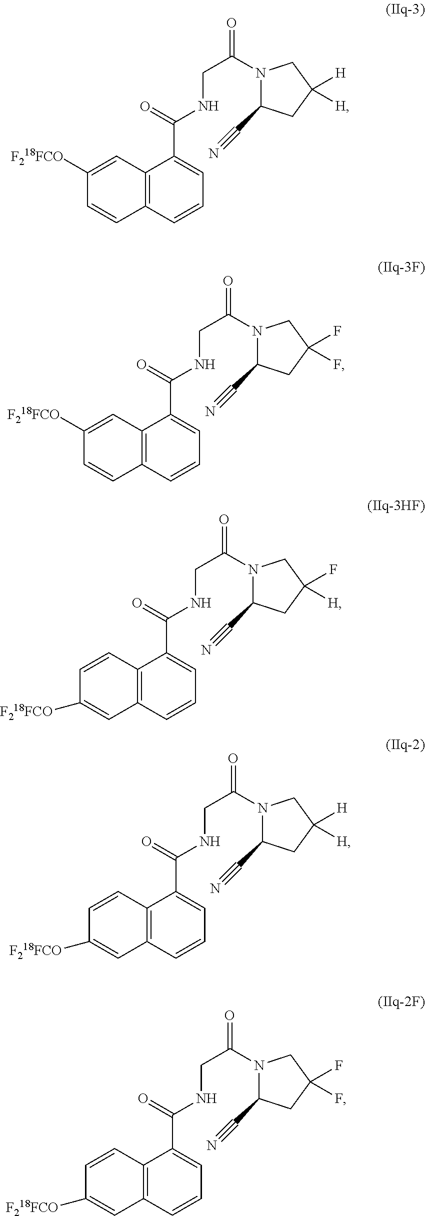

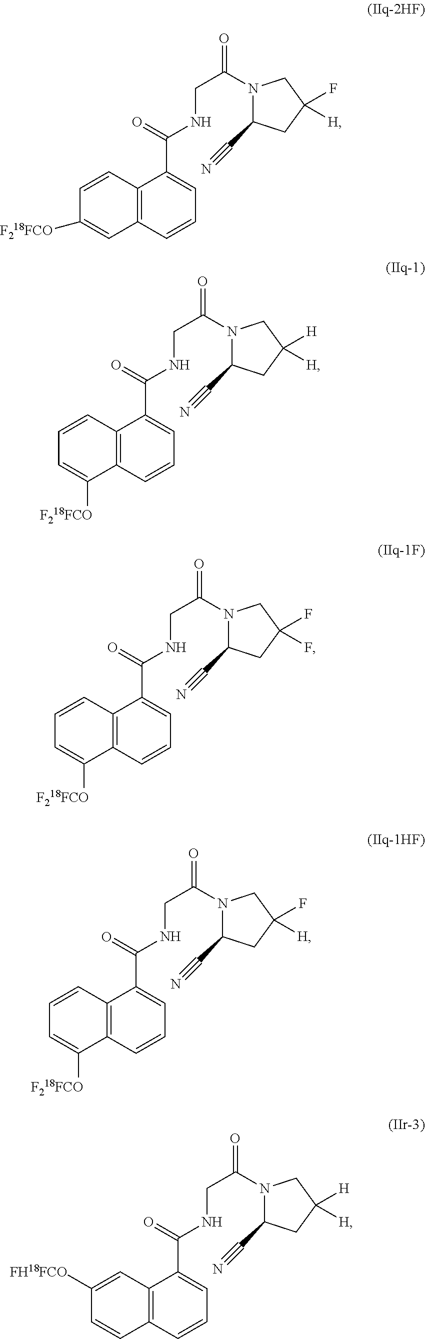

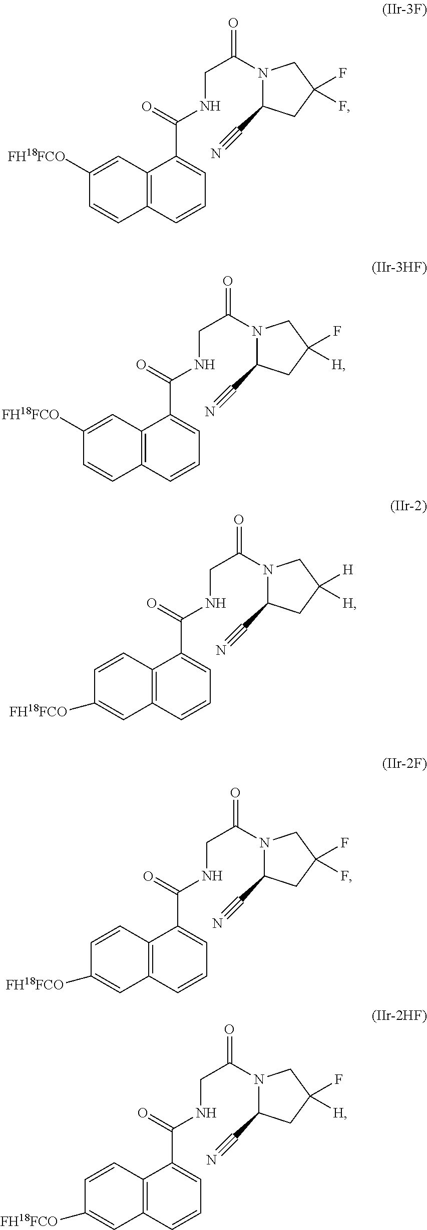

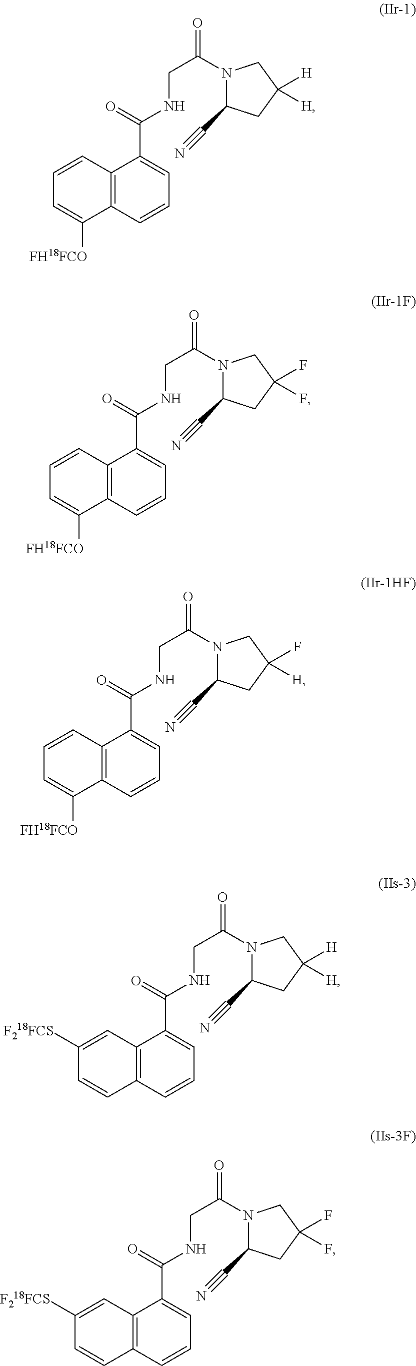

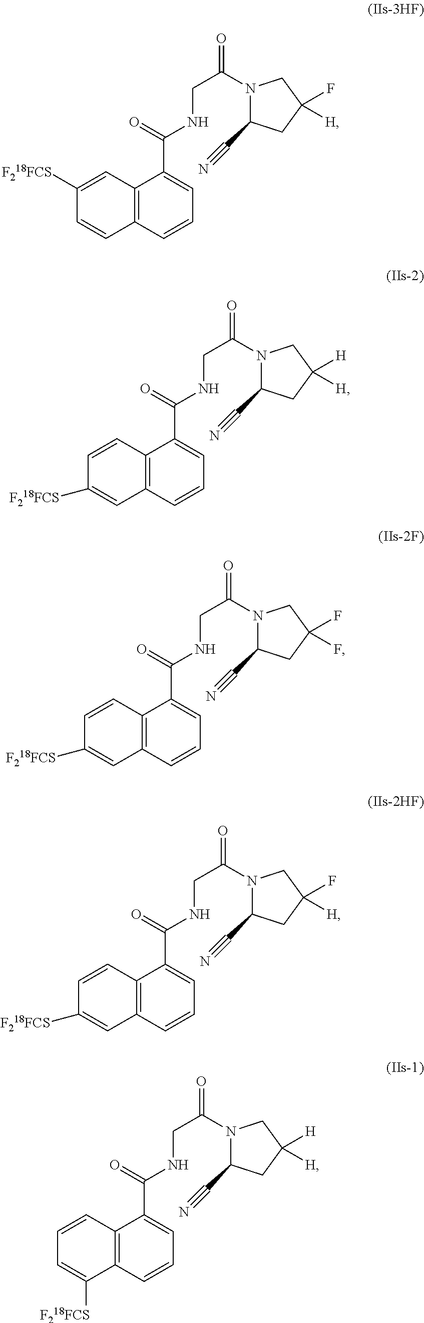

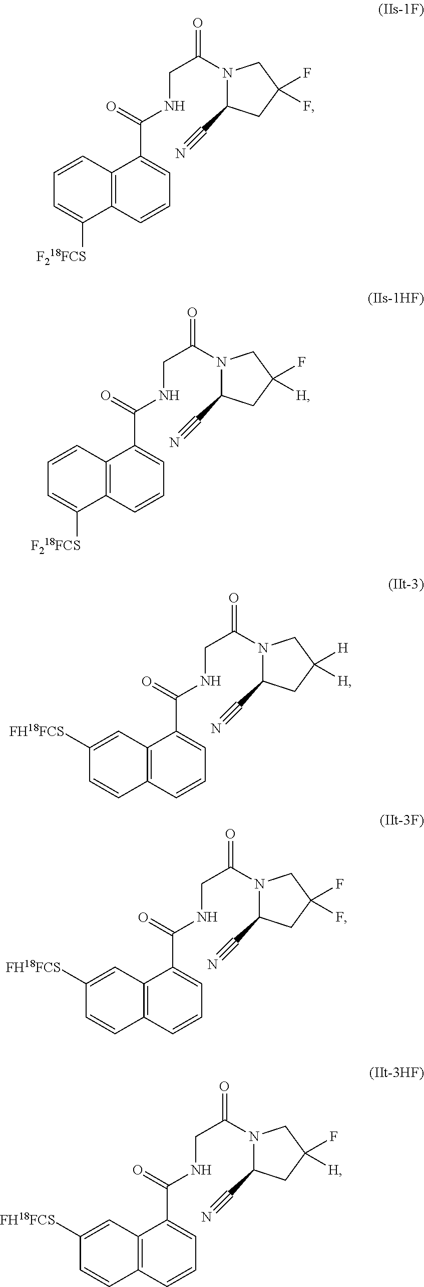

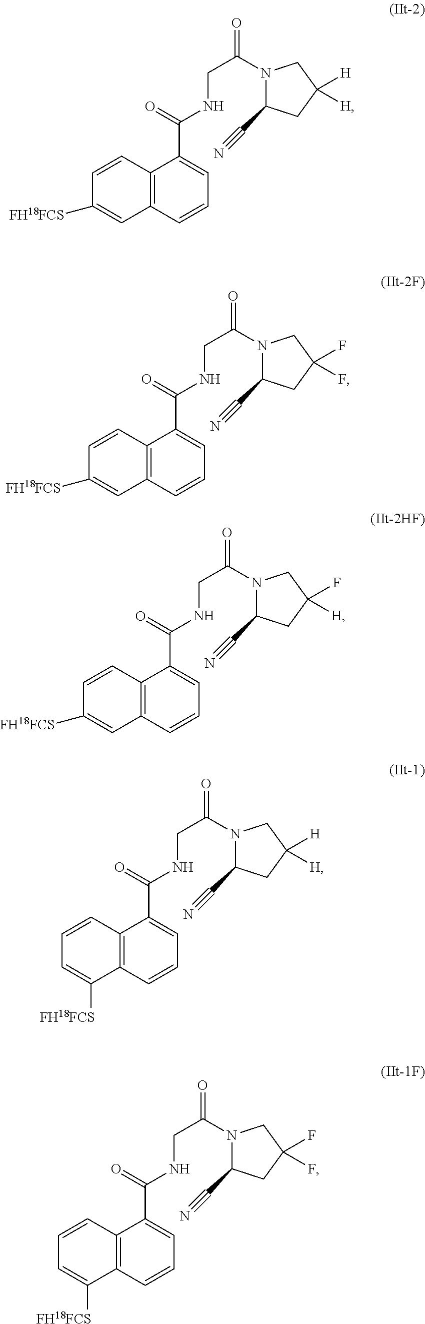

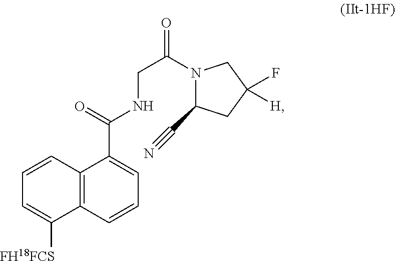

### Compounds of Formula (III)

In some embodiments, the disclosure provides a compound having a structure according to formula (III):

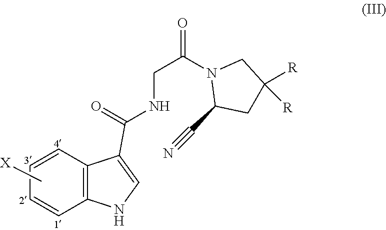

wherein X is located at carbon 1′, 2′, or 3′ and is selected from the group of I, ¹⁸F, ²¹¹At, ¹²³I, ¹²⁴I, ¹²⁵I, ¹³¹I, ⁷⁶Br, ⁷⁷Br, H₃¹¹CO, CF₂¹⁸F, CHF¹⁸F, OCF₂¹⁸F, OCHF¹⁸F, SCF₂¹⁸F, SCHF¹⁸F, ¹¹CN, ¹¹CH₃, and H₃¹¹CS; each R is independently selected from H, F, ²H, CH₃, and cyclopentyl.

In some embodiments, X is selected from the group of ¹⁸F, ²¹¹At, ¹²³I, ¹²⁴I, ¹²⁵I, ¹³¹I, ⁷⁶Br, ⁷⁷Br, H₃¹¹CO, CF₂¹⁸F, CHF¹⁸F, OCF₂¹⁸F, OCHF¹⁸F, SCF₂¹⁸F, SCHF¹⁸F, ¹¹CN, ¹¹CH₃, and H₃¹¹CS. In embodiments, X is selected from the group of ¹⁸F, ²¹¹At, ¹²³I, ¹²⁴I, ¹²⁵I, ¹³¹I, ⁷⁶Br, ⁷⁷Br, and H₃¹¹CO.

In some embodiments, X is located at the 3′ carbon. In some embodiments, X is located at the 2′ carbon. In some embodiments, X is located at the 1′ carbon.

In some embodiments, X is located at the 3′ carbon and X is I. In some embodiments, X is located at the 3′ carbon and X is ¹⁸F. In some embodiments, X is located at the 3′ carbon and X is ²¹¹At. In some embodiments, X is located at the 3′ carbon and X is ¹²³I. In some embodiments, X is located at the 3′ carbon and X is ¹²⁴I. In some embodiments, X is located at the 3′ carbon and X is ¹²⁵I. In some embodiments, X is located at the 3′ carbon and X is ¹³¹I. In some embodiments, X is located at the 3′ carbon and X is ⁷⁶Br. In some embodiments, X is located at the 3′ carbon and X is ⁷⁷Br. In some embodiments, X is located at the 3′ carbon and X is H₃¹¹CO. In some embodiments, X is located at the 3′ carbon and X is CF₂¹⁸F. In some embodiments, X is located at the 3′ carbon and X is CHF¹⁸F. In some embodiments, X is located at the 3′ carbon and X is OCF₂¹⁸F. In some embodiments, X is located at the 3′ carbon and X is OCHF¹⁸F. In some embodiments, X is located at the 3′ carbon and X is SCF₂¹⁸F. In some embodiments, X is located at the 3′ carbon and X is SCHF¹⁸F. In some embodiments, X is located at the 3′ carbon and X is ¹¹CN. In some embodiments, X is located at the 3′ carbon and X is ¹¹CH₃. In some embodiments, X is located at the 3′ carbon and X is H₃¹¹CS.

In some embodiments, X is located at the 2′ carbon and X is I. In some embodiments, X is located at the 2′ carbon and X is ¹⁸F. In some embodiments, X is located at the 2′ carbon and X is ²¹¹At. In some embodiments, X is located at the 2′ carbon and X is ¹²³I. In some embodiments, X is located at the 2′ carbon and X is ¹²⁴I. In some embodiments, X is located at the 2′ carbon and X is ¹²⁵I. In some embodiments, X is located at the 2′ carbon and X is ¹³¹I. In some embodiments, X is located at the 2′ carbon and X is ⁷⁶Br. In some embodiments, X is located at the 2′ carbon and X is ⁷⁷Br. In some embodiments, X is located at the 2′ carbon and X is H₃¹¹CO. In some embodiments, X is located at the 2′ carbon and X is CF₂¹⁸F. In some embodiments, X is located at the 2′ carbon and X is CHF¹⁸F. In some embodiments, X is located at the 2′ carbon and X is OCF₂¹⁸F. In some embodiments, X is located at the 2′ carbon and X is OCHF¹⁸F. In some embodiments, X is located at the 2′ carbon and X is SCF₂¹⁸F. In some embodiments, X is located at the 2′ carbon and X is SCHF¹⁸F. In some embodiments, X is located at the 2′ carbon and X is ¹¹CN. In some embodiments, X is located at the 2′ carbon and X is ¹¹CH₃. In some embodiments, X is located at the 2′ carbon and X is H₃¹¹CS.

In some embodiments, X is located at the 1′ carbon and X is I. In some embodiments, X is located at the 1′ carbon and X is ¹⁸F. In some embodiments, X is located at the 1′ carbon and X is ²¹¹At. In some embodiments, X is located at the 1′ carbon and X is ¹²³I. In some embodiments, X is located at the 1′ carbon and X is ¹²⁴I. In some embodiments, X is located at the 1′ carbon and X is ¹²⁵I. In some embodiments, X is located at the 1′ carbon and X is ¹³¹I. In some embodiments, X is located at the 1′ carbon and X is ⁷⁶Br. In some embodiments, X is located at the 1′ carbon and X is ⁷⁷Br. In some embodiments, X is located at the 1′ carbon and X is H₃¹¹CO. In some embodiments, X is located at the 1′ carbon and X is CF₂¹⁸F. In some embodiments, X is located at the 1′ carbon and X is CHF¹⁸F. In some embodiments, X is located at the 1′ carbon and X is OCF₂¹⁸F. In some embodiments, X is located at the 1′ carbon and X is OCHF¹⁸F. In some embodiments, X is located at the 1′ carbon and X is SCF₂¹⁸F. In some embodiments, X is located at the 1′ carbon and X is SCHF¹⁸F. In some embodiments, X is located at the 1′ carbon and X is ¹¹CN. In some embodiments, X is located at the 1′ carbon and X is ¹¹CH₃. In some embodiments, X is located at the 1′ carbon and X is H₃¹¹CS.

In general, each R is independently selected from H, F, ²H, CH₃, and cyclopentyl. In embodiments, each R is independently selected from H and F. In embodiments, at least one R is H. In embodiments, both R are H. In embodiments, at least one R is F. In embodiments, both R are F. In embodiments, at least one R is ²H. In embodiments, both R are ²H. In embodiments, at least one R is CH₃. In embodiments, both R are CH₃. In embodiments, at least one R is cyclopentyl. In embodiments, both R are cyclopentyl. In embodiments, X is located at carbon 3′ and both R are H. In embodiments, X is located at carbon 3′ and both R are F. In embodiments, X is located at carbon 2′ and both R are H. In embodiments, X is located at carbon 2′ and both R are F. In embodiments, X is located at carbon 1′ and both R are H. In embodiments, X is located at carbon 1′ and both R are F.

In embodiments, the fibroblast activation protein inhibitor has a structure according to one of the following formulae:

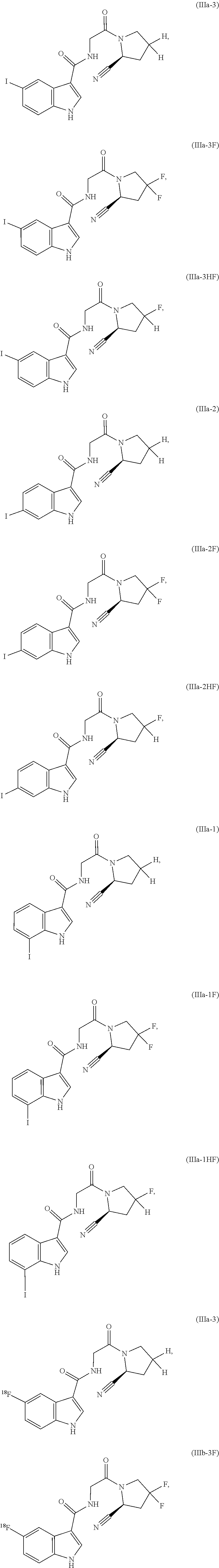

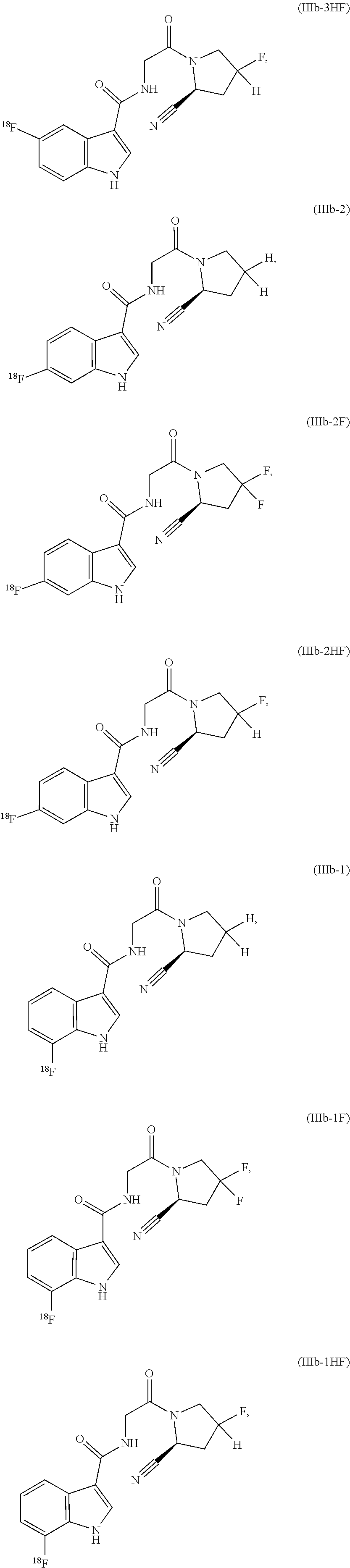

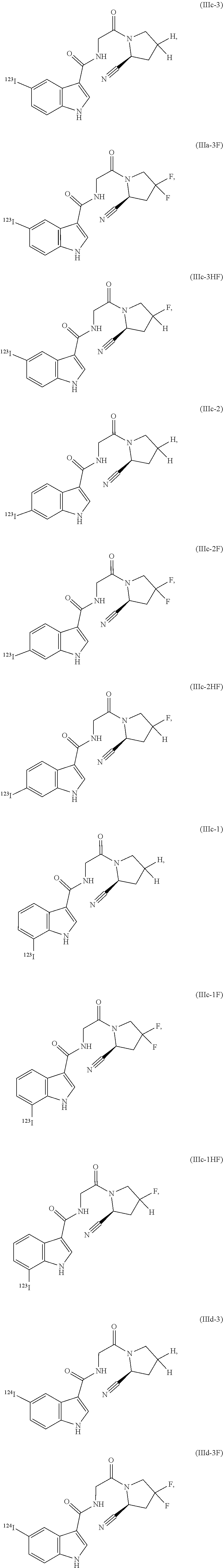

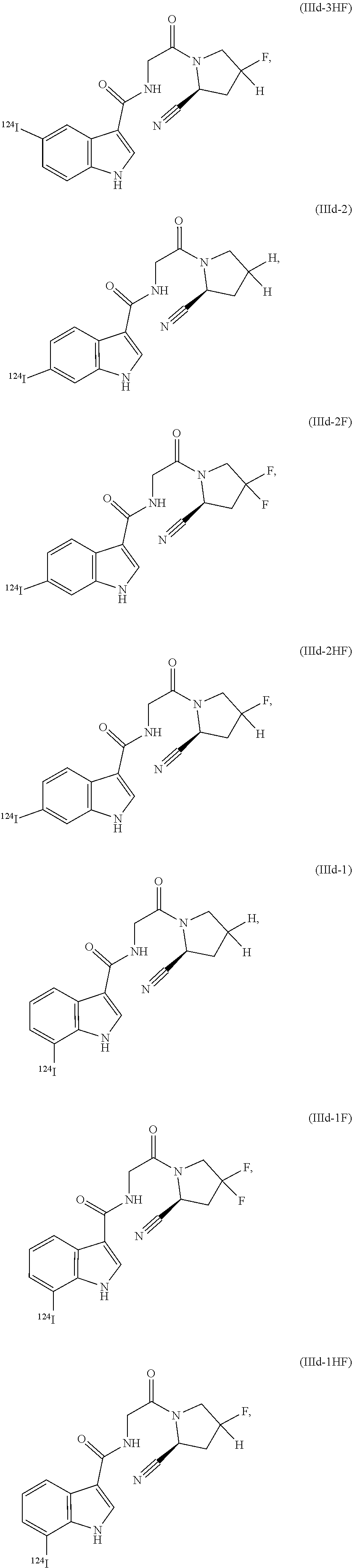

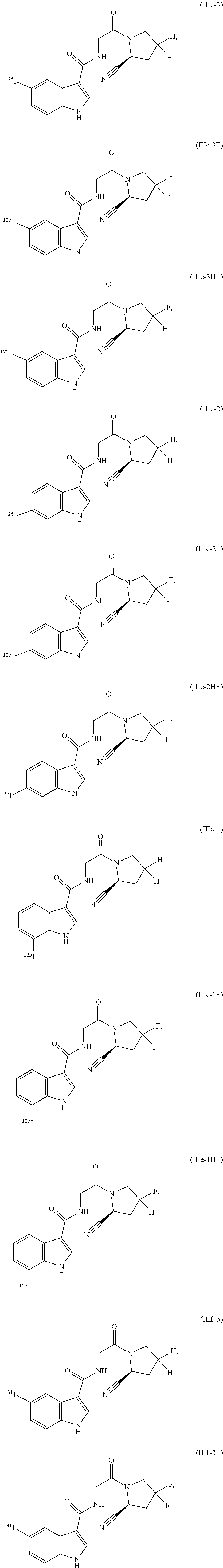

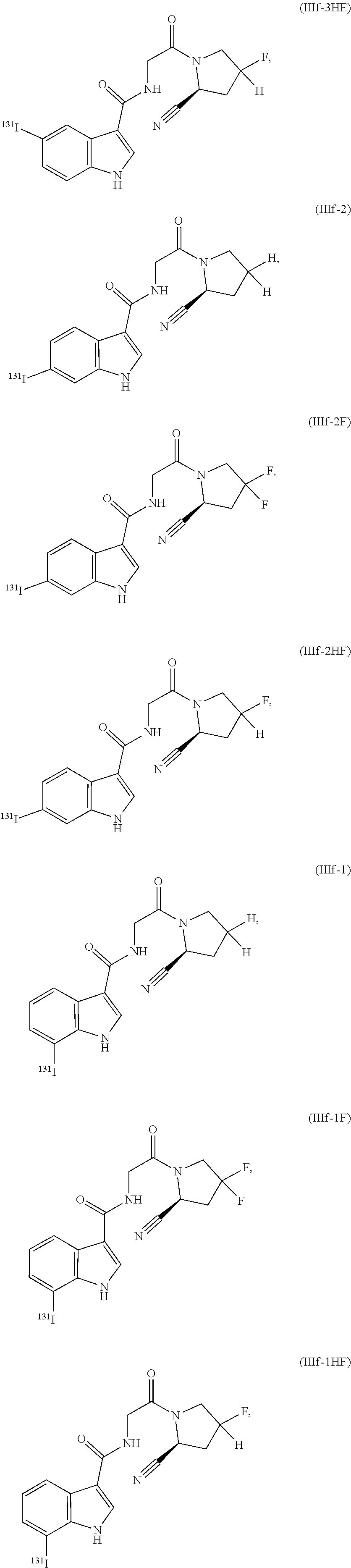

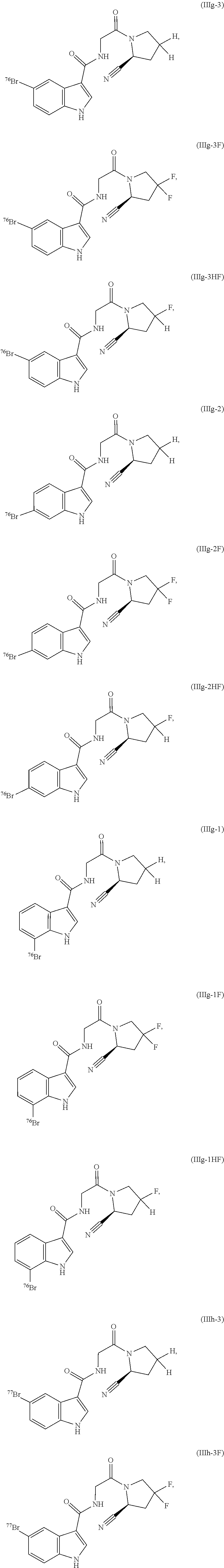

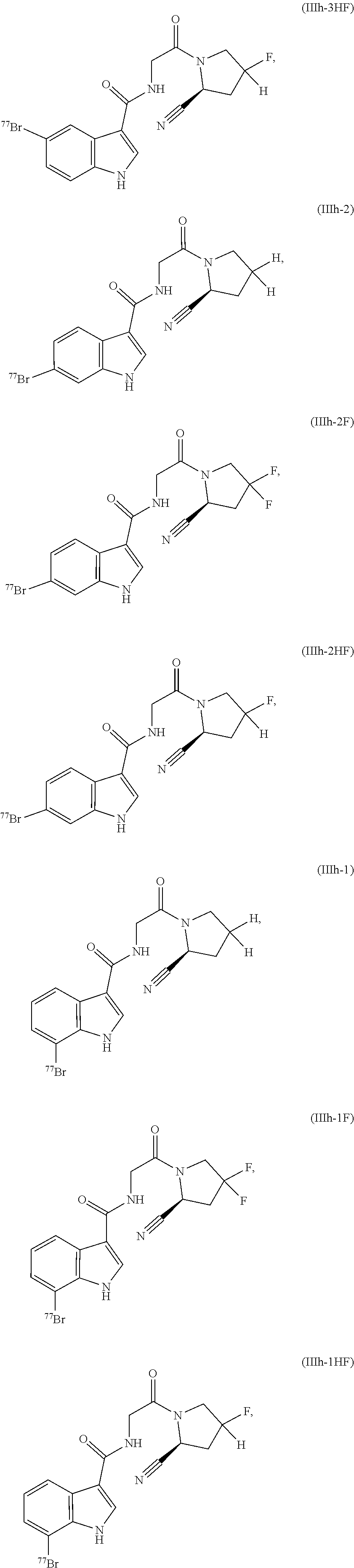

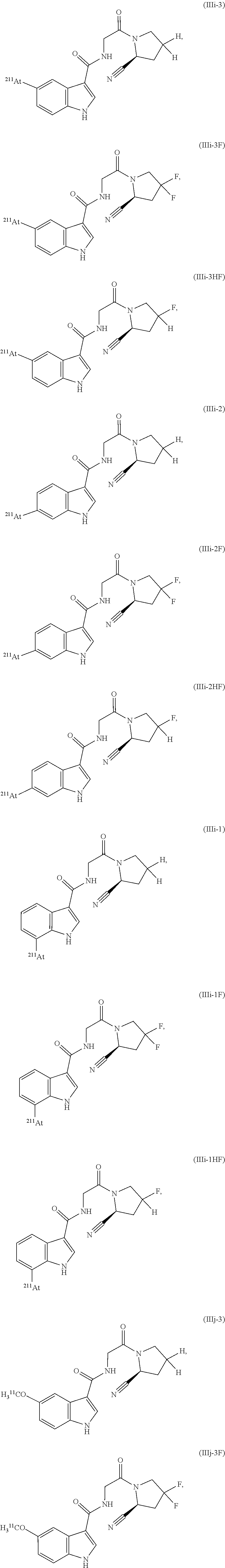

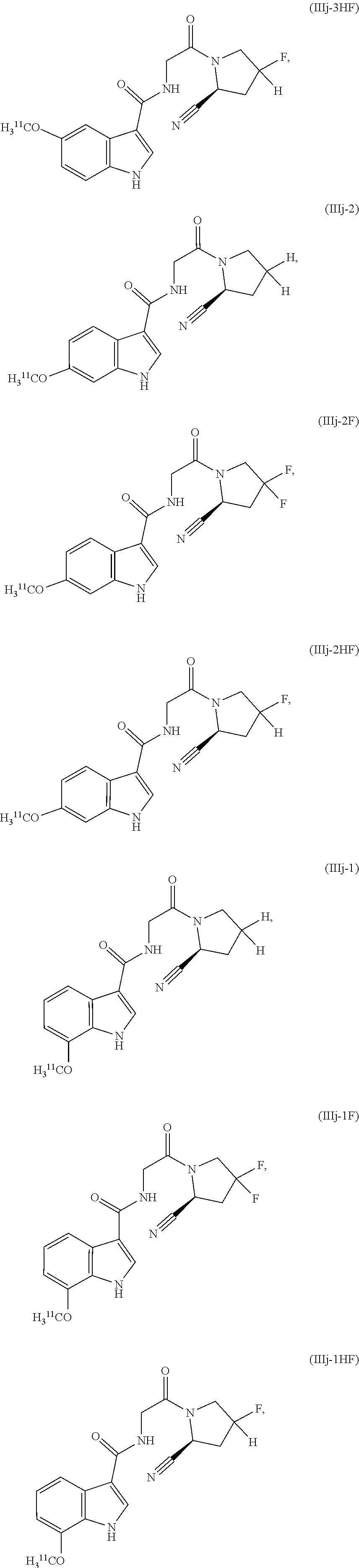

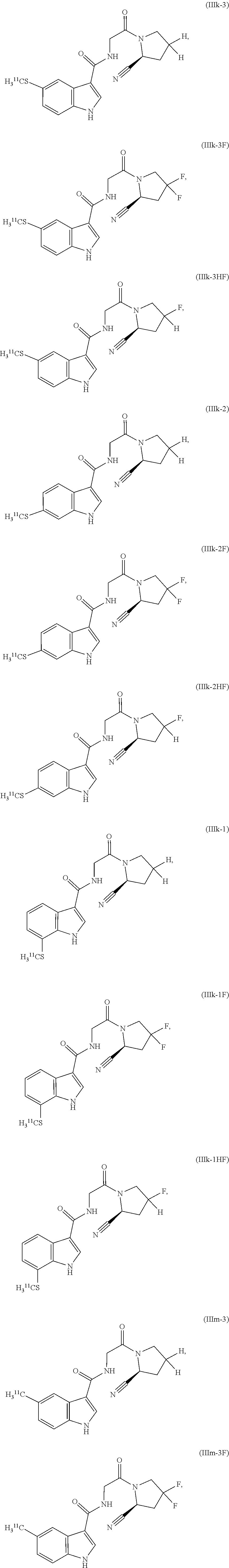

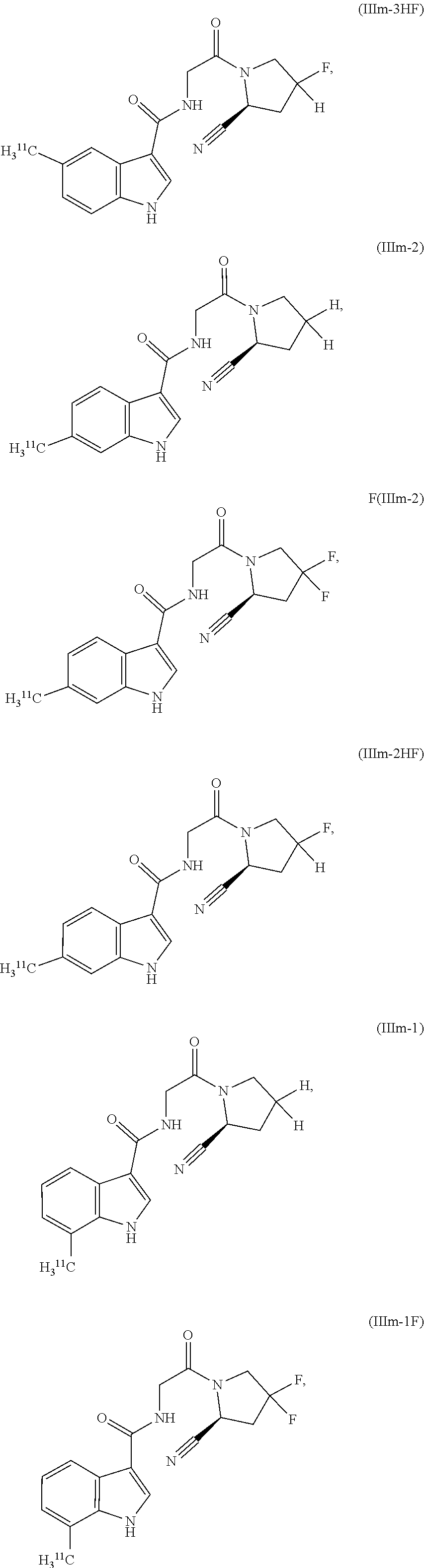

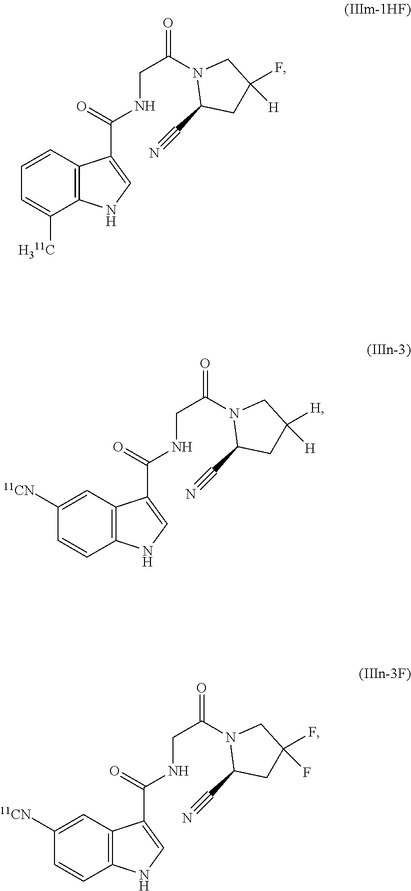

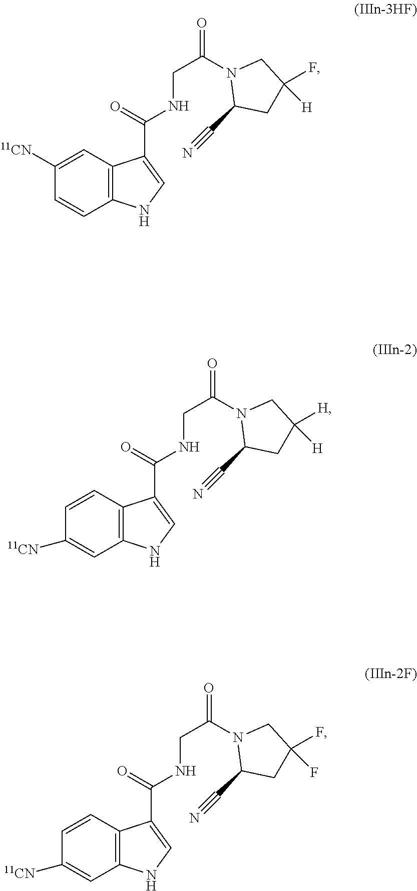

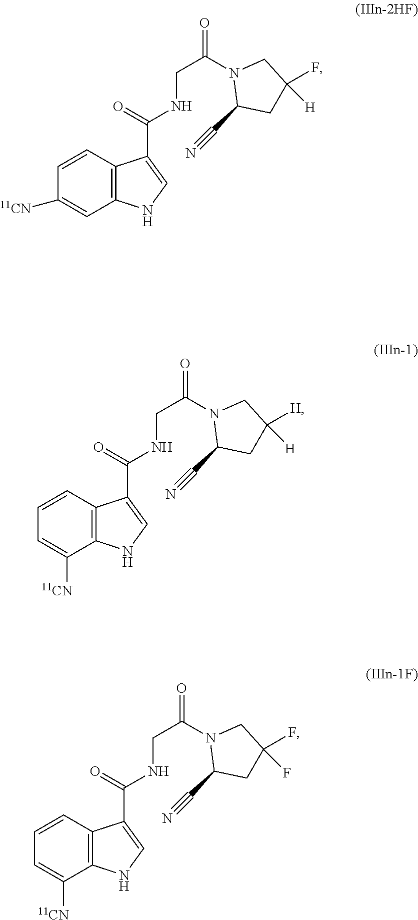

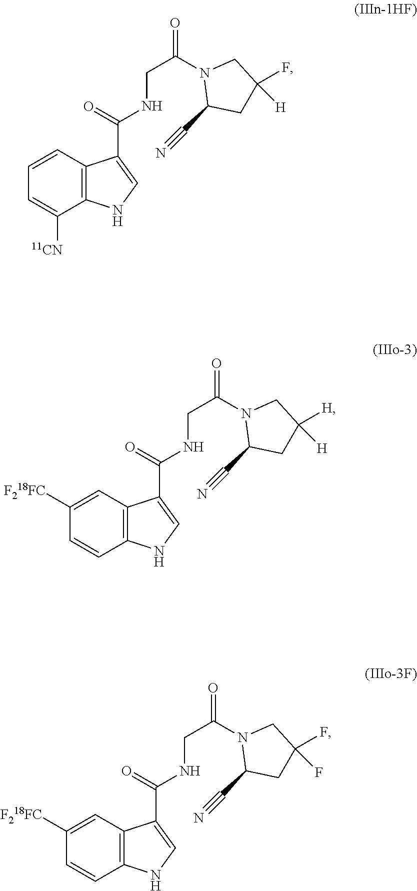

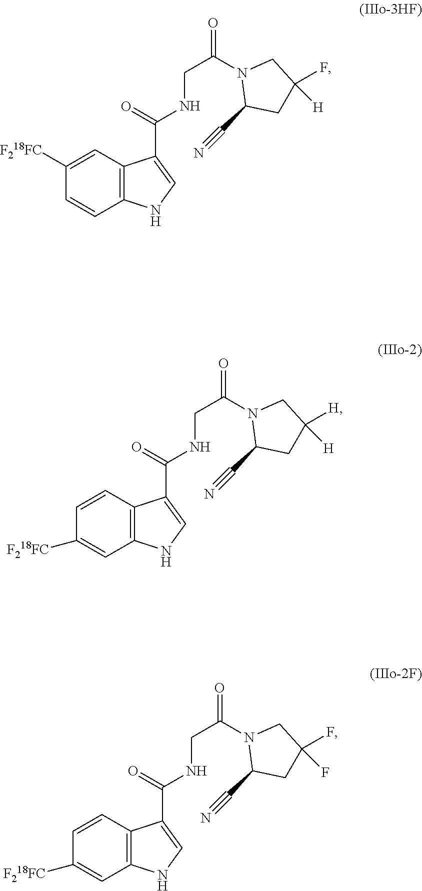

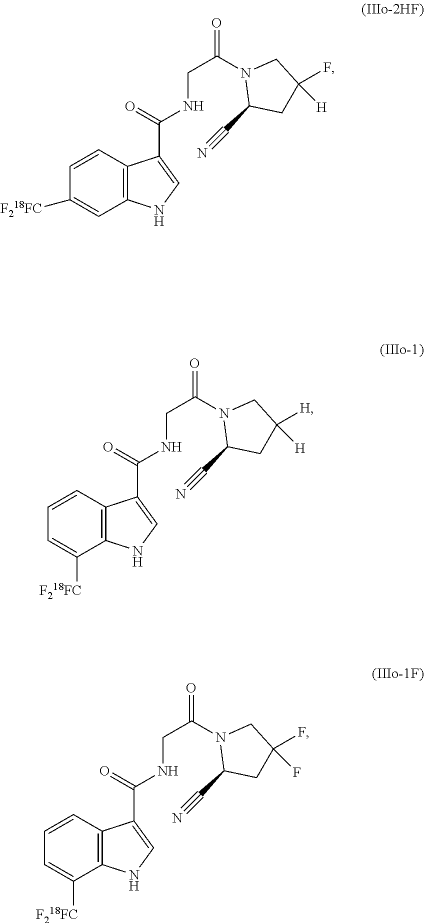

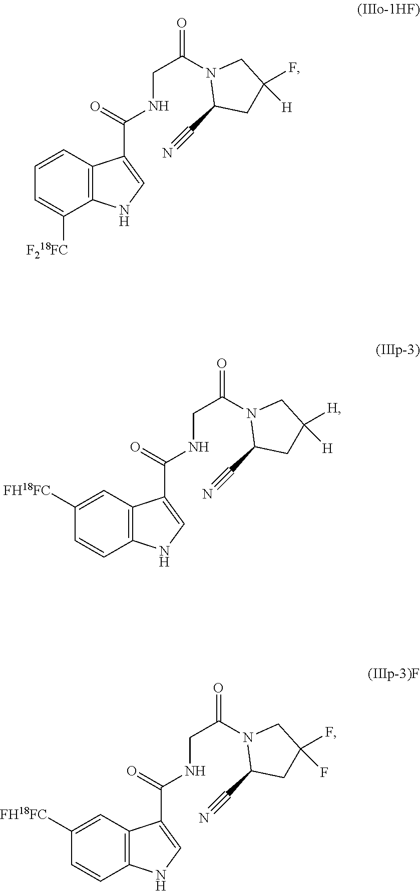

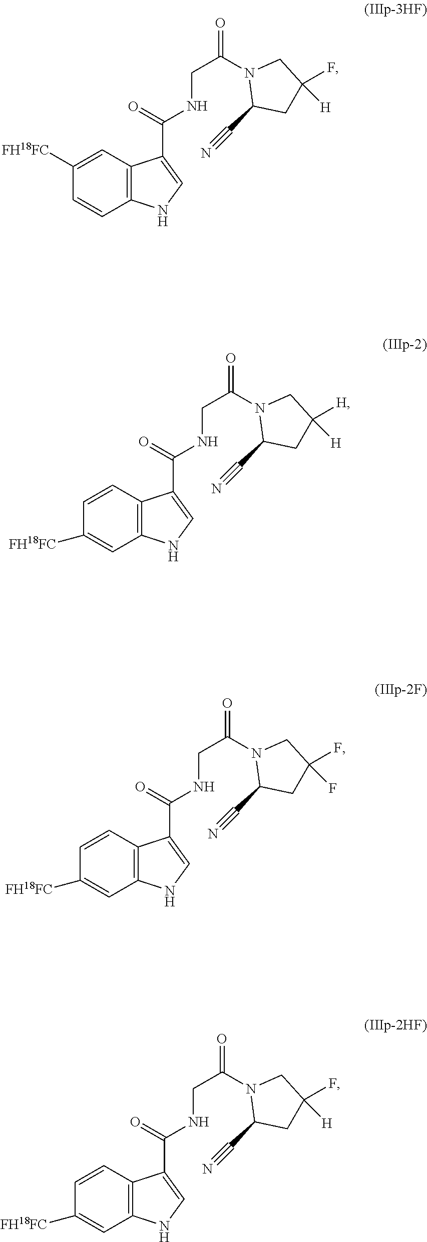

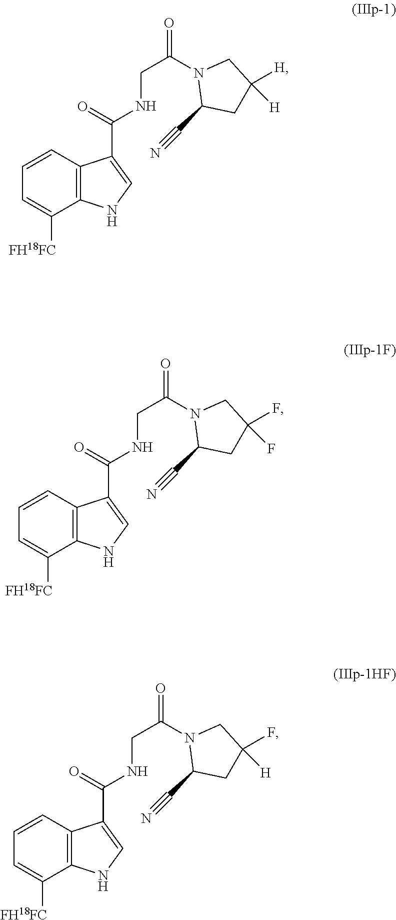

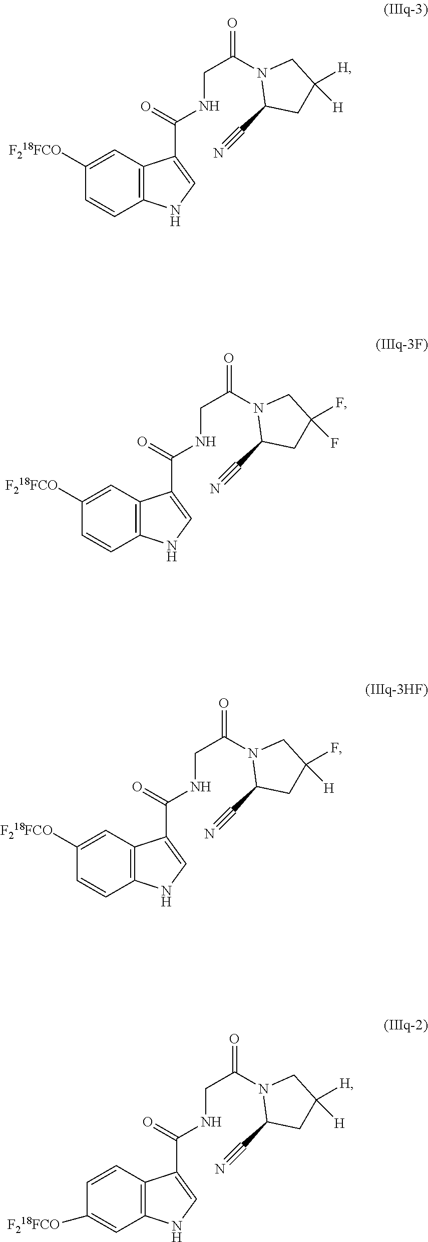

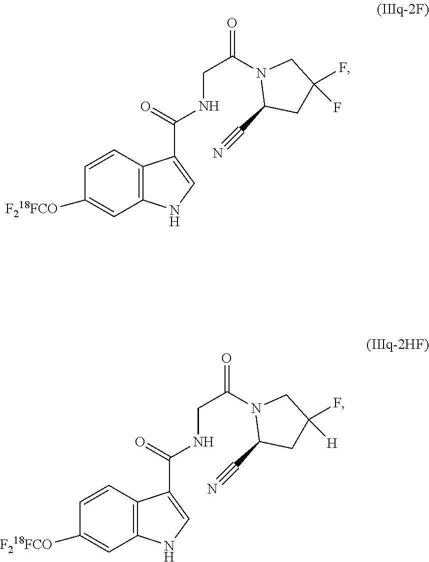

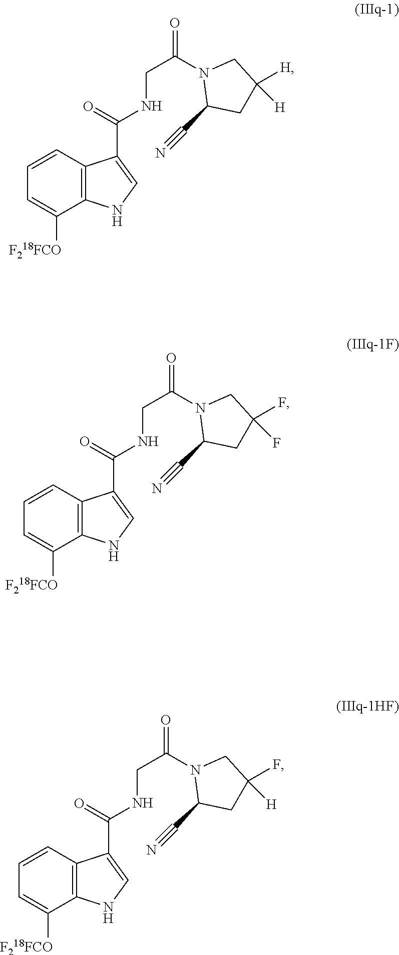

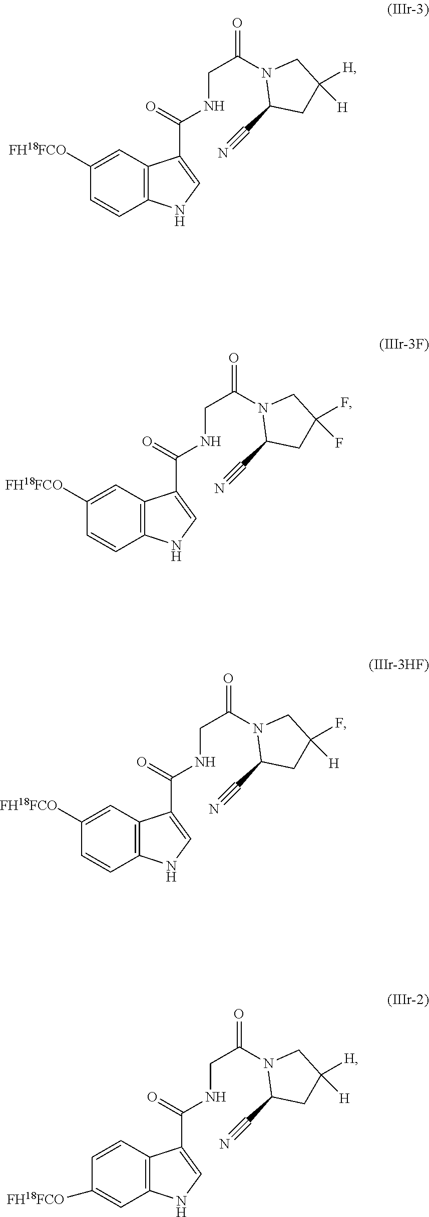

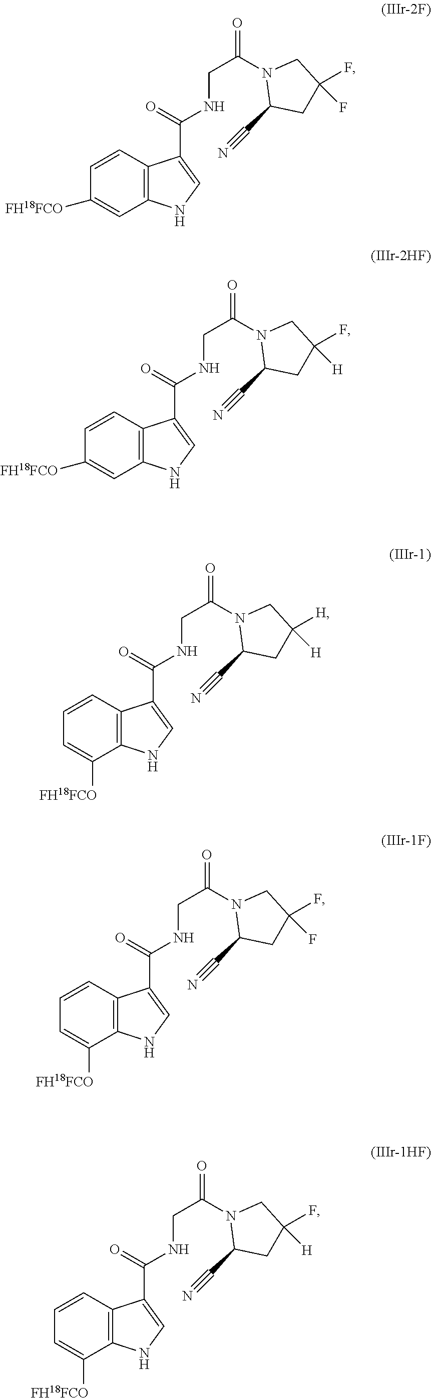

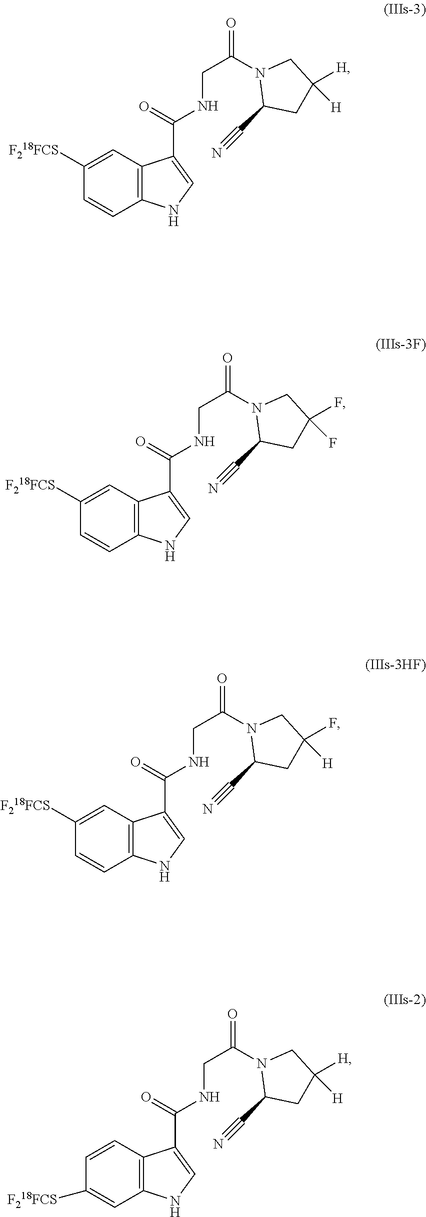

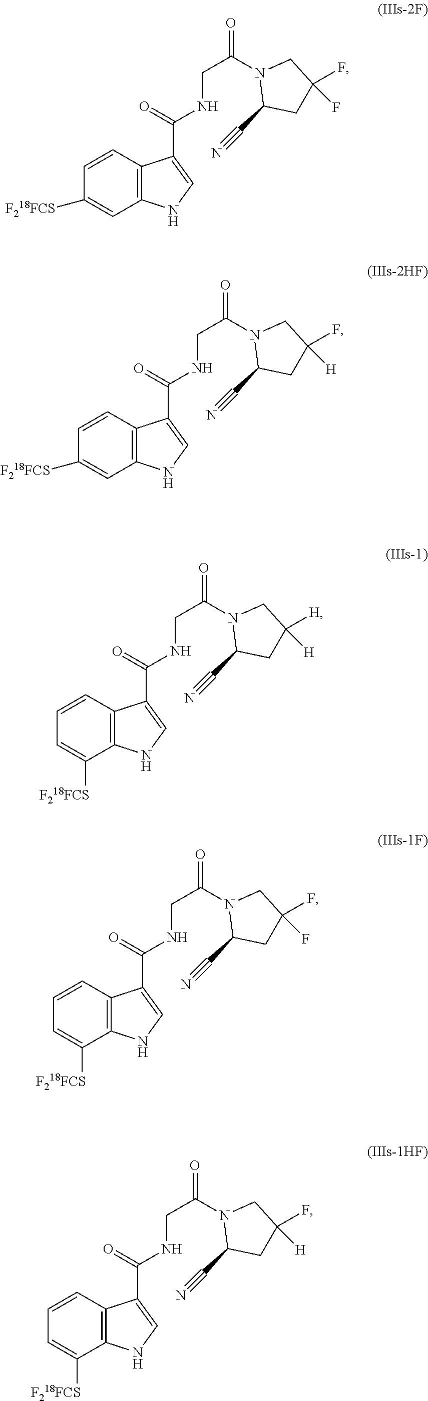

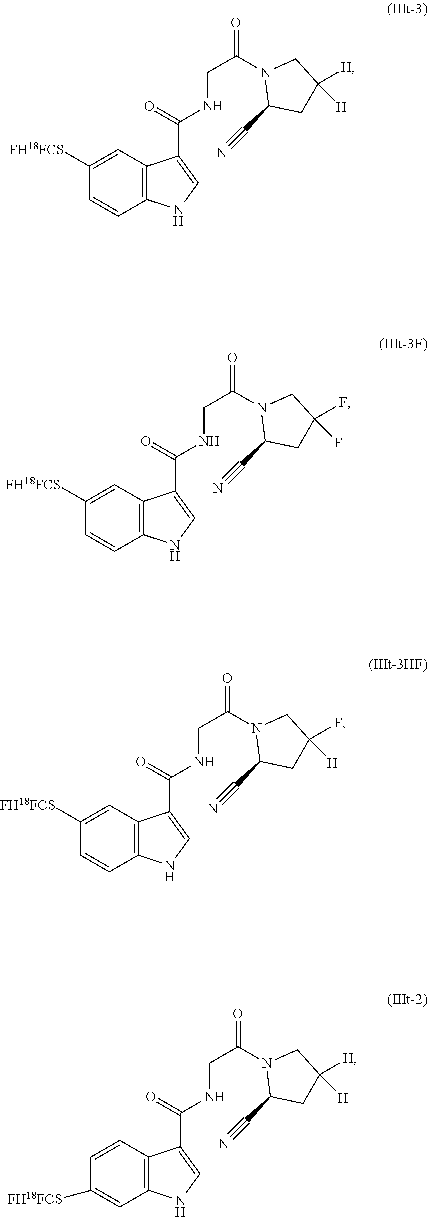

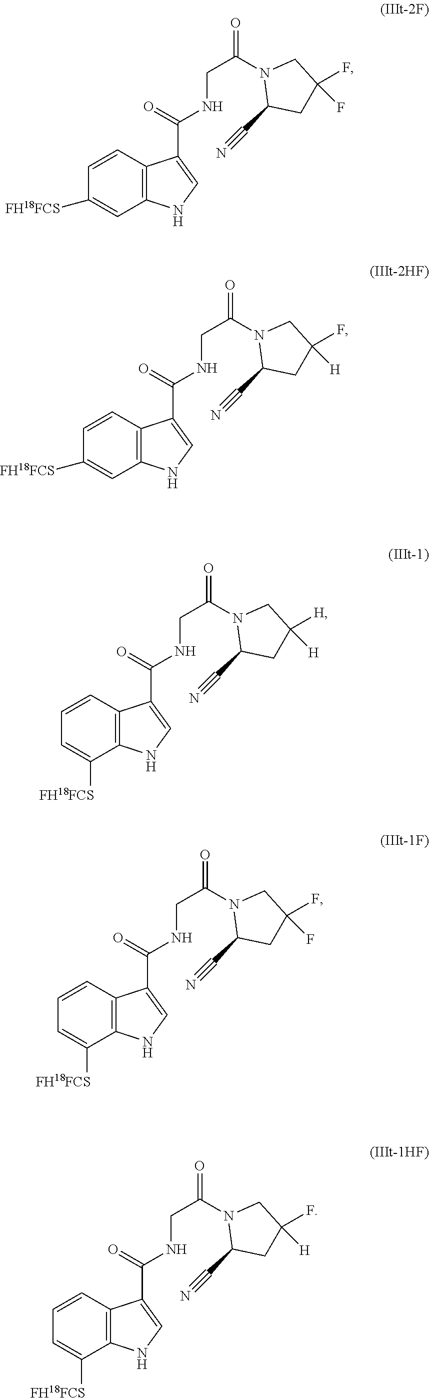

The disclosure further provides methods of preparing radiolabeled FAPIs having structures according to formula (I), formula (II), or formula (III). In general, the radiolabeled FAPIs can be prepared from compounds having a structure according to formula (Ia), formula (IIa), or formula (IIIa):

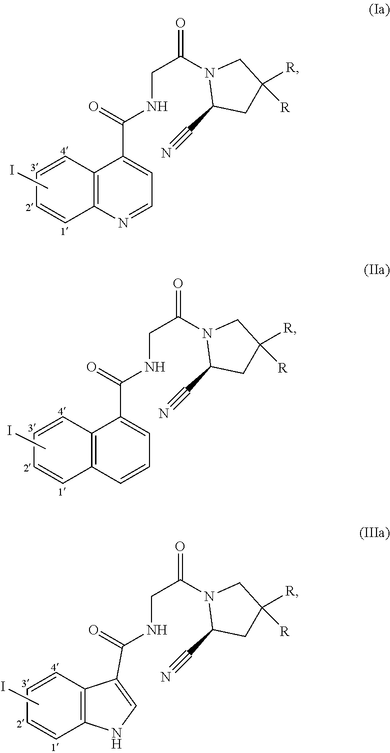

wherein the R groups are the same as the R groups in the FAPIs having a structure according to formula (I), formula (II), or formula (III), through an intermediate having a structure selected from the group of a compound of formula (V), (VI), (VII), (VIII), (X), and (XI):

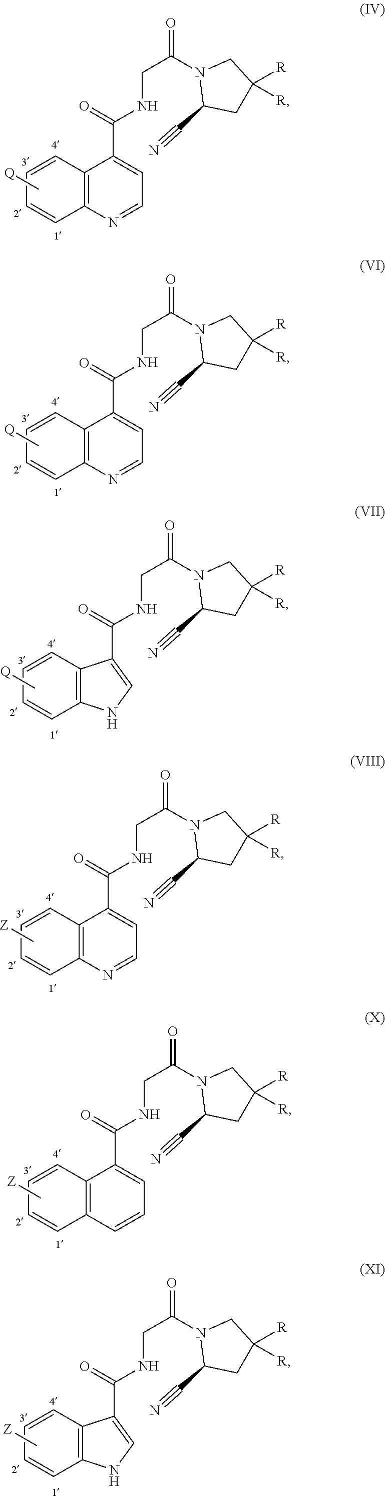

wherein the R groups are the same as the R groups in the FAPIs having a structure according to formula (I), formula (II), or formula (III), Q is selected from trialkyl stannyl, trialkyl germyl, or trialkyl silyl; and each Z has a structure according to a formula selected from the group of:

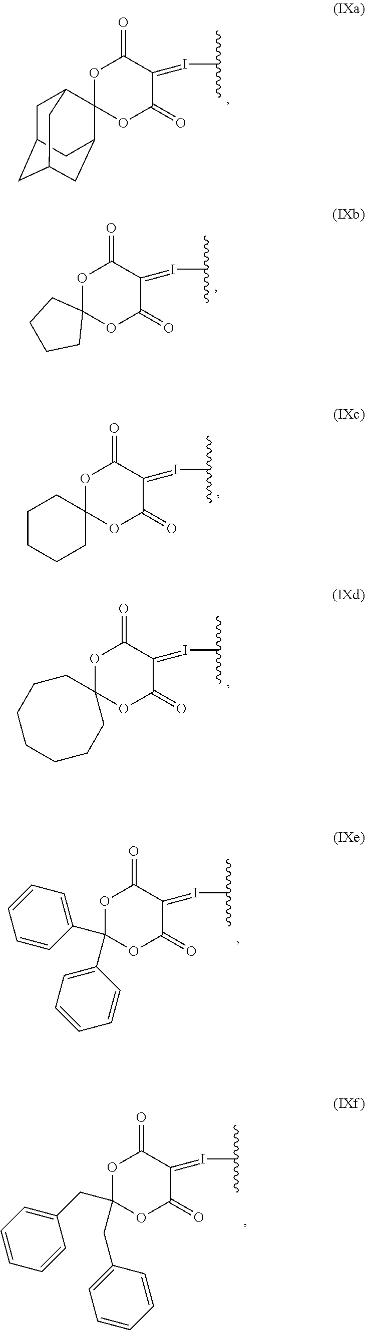 [SMILES: *I=C1C(=O)OC2(OC1=O)C1CC3CC(C1)CC2C3 ; *I=C1C(=O)OC2(CCCC2)OC1=O ; *I=C1C(=O)OC2(CCCCC2)OC1=O ; *I=C1C(=O)OC2(CCCCCCC2)OC1=O ; *I=C1C(=O)OC(c2ccccc2)(c2ccccc2)OC1=O ; *I=C1C(=O)OC(Cc2ccccc2)(Cc2ccccc2)OC1=O]

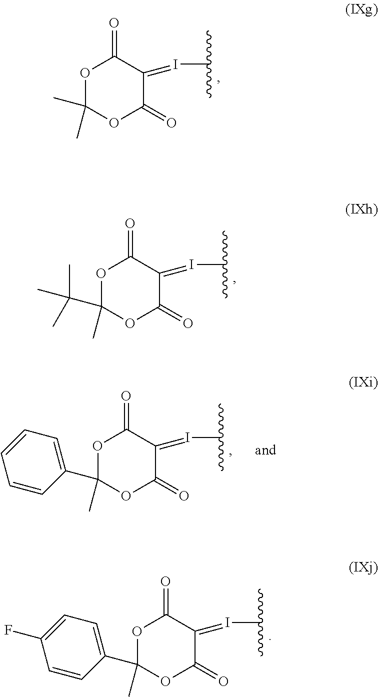 [SMILES: *I=C1C(=O)OC(C)(C)OC1=O ; *I=C1C(=O)OC(C)(C(C)(C)C)OC1=O ; *I=C1C(=O)OC(C)(c2ccccc2)OC1=O ; *I=C1C(=O)OC(C)(c2ccc(F)cc2)OC1=O]

Methods of Preparing Radiolabeled FAPIs Having Structures According to Formula (I), Formula (II), or Formula (III) Through an Intermediate Selected from a Compound Having a Structure Selected from the Group of Formula (V), Formula (VI), and Formula (VII)

In embodiments, the methods of the disclosure can include preparing a radiolabeled compound having a structure according to formula (I), the method comprising admixing a compound having a structure according to formula (IV) with a salt, MX:

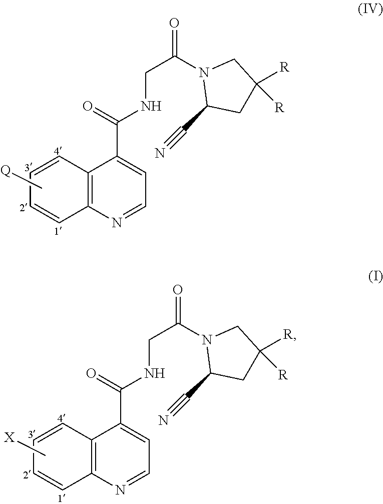

wherein M is selected from the group of K, Li, Na, Rb, Cs, tetraethylammonium ((ethyl)₄N), and tetrabutylammonium ((butyl)₄N), and a combination thereof, in the salt, MX, and in formula (I), X is selected from the group of ¹⁸F, ²¹¹At, ¹²³I, ¹²⁴I, ¹²⁵I, ¹³¹I, ⁷⁶Br, ⁷⁷Br, H₃¹¹CO, CF₂¹⁸F, CHF¹⁸F, OCF₂¹⁸F, OCHF¹⁸F, SCF₂¹⁸F, SCHF¹⁸F, ¹¹CN, ¹¹CH₃, and H₃¹¹CS; and X is the same in the salt and in formula (I); Q is selected from trialkyl stannyl, trialkyl germyl, and trialkyl silyl; in formula (IV) and formula (I), Q and X are located at carbon 1′, 2′, or 3′, and the location of Q in formula (IV) and the location of X in formula (I) are the same; and each R is independently selected from H, F, ²H, CH₃, and cyclopentyl, and the R in formula (I) are the same as the R in formula (IV).

In general, M is any monovalent cation capable of forming a salt with the anion, X. In embodiments, M is selected from the group of K, Li, Na, Rb, Cs, tetraethylammonium ((ethyl)₄N), and tetrabutylammonium ((butyl)₄N). In embodiments, M is selected from the group of K, Li, and Na. In embodiments, M is K.

In general, the compound of having a structure according to formula (IV) can be identical to any compound of having a structure of formula (I) disclosed herein, except that the X group of the compound of formula (I) is replaced with a Q group as defined herein. In embodiments, Q is selected from trialkyl stannyl, trialkyl germyl, and trialkyl silyl. In embodiments, Q is trialkyl stannyl. In embodiments, Q is trialkyl germyl. In embodiments, Q is trialkyl silyl. In embodiments, the alkyl of the trialkyl stannyl, trialkyl germyl and trialkyl silyl are methyl or butyl. In embodiments, the alkyl of the trialkyl stannyl, trialkyl germyl and trialkyl silyl are methyl. In embodiments, the alkyl of the trialkyl stannyl, trialkyl germyl and trialkyl silyl are butyl. In embodiments, Q is trimethyl stannyl. In embodiments, Q is tributyl stannyl.

The methods of the disclosure convert the Q group of the compound having a structure according to formula (IV) to an X group, without otherwise altering the structure of the compound of formula (IV). Thus, the Q group of the compound having a structure according to formula (IV) and the X group of the compound having a structure according to formula (I) are located at the same position on the quinoline ring and the R groups of the compound having a structure according to formula (I) are the same as the R groups of the compound having a structure according to formula (IV). In embodiments, Q and X are each located at carbon 1′. In embodiments, Q and X are each located at carbon 2′. In embodiments, Q and X are each located at carbon 3′.

In general, the admixing of the compound having a structure according to formula (IV) and the salt, MX, is done under conditions sufficient to provide a compound of having a structure according to formula (I). In general, the admixing can take place in a solution comprising a solvent for the compound having a structure according to formula (IV), a solvent for the salt, or a solvent for the compound having a structure according to formula (IV) and the salt. In embodiments, the solvent can comprise any solvent suitable for solvating the compound having a structure according to formula (IV), the salt, or both. In embodiments, the solvent comprises pyridine, dimethylformamide (DMF), dimethylacetamide (DMA), or a combination thereof. In embodiments, the solvent comprises pyridine. In embodiments, the solvent comprises pyridine and DMF. In embodiments, the solvent comprises pyridine and DMA. The concentrations of the compound having a structure according to formula (IV) and the salt can be any concentration. The concentrations are typically chosen such that the compound having a structure according to formula (IV) and/or the salt is fully soluble, without forming saturated solutions.

In embodiments, the admixing can take place in the presence of a catalyst. Suitable catalysts include, but are not limited to, copper(II) triflate (Cu(OTf)₂), tetrakisacetonitrile copper(I) triflate (CH₃CN)₄CuOTf), and copper(I) trifluoromethanesulfonate toluene complex (CuOTf-toluene). In embodiments, the catalyst comprises copper(II) triflate. In embodiments, the catalyst comprises tetrakisacetonitrile copper(I) triflate. In embodiments, the catalyst comprises copper(I) trifluoromethanesulfonate toluene complex.

In general, the admixing can take place at any suitable temperature for any suitable time. In embodiments, the admixing can take place at a temperature in a range of about 20° C. to about 140° C., for example, about 20° C. to about 120° C., about 20° C. to about 100° C., about 20° C. to about 80°, about 20° to about 60° C., about 20° C. to about 40° C., about 20° C. to about 30° C., about 22° C. to about 26° C., about 40° C. to about 140° C., about 60° C. to about 140° C., about 80° C. to about 140° C., or about 85° C. to about 140° C. In general, as the temperature increases, the rate of reaction increases, and the time required for admixing decreases. As the temperature increases, e.g., above about 140° C., the likelihood of decomposition of the reactants, decomposition of the products, and/or evaporation of the solvents increases. In embodiments, the admixing can take place for a time in a range of about 5 minutes to about 72 hours, for example, about 5 minutes to about 60 hours, about 5 minutes to about 48 hours, about 5 minutes to about 36 hours, about 5 minutes to about 24 hours, about 5 minutes to about 12 hours, about 5 minutes to about 8 hours, about 5 minutes to about 6 hours, about 5 minutes to about 4 hours, about 5 minutes to about 2 hours, about 5 minutes to about 1 hour, about 5 minutes to about 45 minutes, or about 5 minutes to about 30 minutes. As the admixing time decreases, the likelihood of the reaction not going to completion increases. Without intending to be bound by theory, it is believed that as the reaction time increases, for example, beyond about 24 hours, 48 hours, or 72 hours, while some reaction product may continue to be formed the amount provided after 24 hours, 48 hours, or 72 hours will have little effect on the total yield, while the likelihood of side reactions and decomposition of the reagents and/or products increases.

The method of preparing a radiolabeled FAPI having a structure according to formula (I) can further include preparing the compound having a structure according to formula (IV), by admixing a compound having a structure according to formula (Ia) with one of hexaalkyldistannane, hexaalkyldigermane, or hexaalkyldisilane:

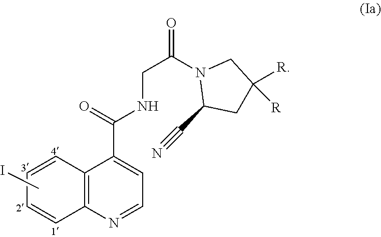

The methods of the disclosure convert the I of the compound having a structure according to formula (Ia) to a Q group, without otherwise altering the structure of the compound of formula (Ia). Thus, the Q group of the compound having a structure according to formula (IV) and the I of the compound having a structure according to formula (Ia) are located at the same position on the quinoline ring and the R groups of the compound having a structure according to formula (Ia) are the same as the R groups of the compound having a structure according to formula (IV).

In embodiments, the compound of formula (Ia) is admixed with a hexaalkyldistannane. In embodiments, the compound of formula (Ia) is admixed with a hexaalkyldigermane. In embodiments, the compound of formula (Ia) is admixed with a hexaalkyldisilane. In embodiments, the alkyl of the hexaalkyldistannane, hexaalkyldigermane, or hexaalkyldisilane is methyl or butyl. In embodiments, the hexaalkyldistannane is hexamethyldistannane. In embodiments, the hexaalkyldistannane is hexabutyldistannane.

In general, the admixing of the compound having a structure according to formula (Ia) and one of hexaalkyldistannane, hexalkyldigermane, or hexaalkyldisilane, is done under conditions sufficient to provide a compound of having a structure according to formula (IV). In general, the admixing can take place in a solution comprising a solvent for the compound having a structure according to formula (Ia), a solvent for the hexaalkyldistannane, hexalkyldigermane, or hexaalkyldisilane, or a solvent for the compound having a structure according to formula (Ia) and the with one of hexaalkyldistannane, hexalkyldigermane, or hexaalkyldisilane. In embodiments, the solvent can comprise any solvent suitable for solvating the compound having a structure according to formula (Ia), the hexaalkyldistannane, hexalkyldigermane, or hexaalkyldisilane, or both. In embodiments, the solvent comprises toluene. The concentrations of the compound having a structure according to formula (Ia) and the hexaalkyldistannane, hexalkyldigermane, or hexaalkyldisilane can be any concentration. The concentrations are typically chosen such that the compound having a structure according to formula (Ia) and/or the hexaalkyldistannane, hexalkyldigermane, or hexaalkyldisilane is fully soluble, without forming saturated solutions.

In embodiments, the admixing can take place in the presence of an alkali metal halide and/or a catalyst. Suitable alkali metal halides include lithium chloride. Suitable catalysts include, but are not limited to, palladium(0) catalysts. In embodiments, the catalyst comprises tetrakis(triphenylphosphine)palladium(0) (Pd(PPh₃)₄).

In general, the admixing can take place at any suitable temperature for any suitable time. In embodiments, the admixing can take place at a temperature in a range of about 20° C. to about 140° C., for example, about 20° C. to about 120° C., about 20° C. to about 100° C., about 20° C. to about 80°, about 20° to about 60° C., about 20° C. to about 40° C., about 20° C. to about 30° C., about 22° C. to about 26° C., about 40° C. to about 140° C., about 60° C. to about 140° C., about 80° C. to about 140° C., or about 85° C. to about 140° C. In general, as the temperature increases, the rate of reaction increases, and the time required for admixing decreases. As the temperature increases, e.g., above about 140° C., the likelihood of decomposition of the reactants, decomposition of the products, and/or evaporation of the solvents increases. In embodiments, the admixing can take place for a time in a range of about 5 minutes to about 72 hours, for example, about 5 minutes to about 60 hours, about 5 minutes to about 48 hours, about 5 minutes to about 36 hours, about 5 minutes to about 24 hours, about 5 minutes to about 12 hours, about 5 minutes to about 8 hours, about 5 minutes to about 6 hours, about 5 minutes to about 4 hours, about 5 minutes to about 2 hours, about 5 minutes to about 1 hour, about 5 minutes to about 45 minutes, or about 5 minutes to about 30 minutes. As the admixing time decreases, the likelihood of the reaction not going to completion increases. Without intending to be bound by theory, it is believed that as the reaction time increases, for example, beyond about 24 hours, 48 hours, or 72 hours, while some reaction product may continue to be formed the amount provided after 24 hours, 48 hours, or 72 hours will have little effect on the total yield, while the likelihood of side reactions and decomposition of the reagents and/or products increases.

In some embodiments, X is located at the 3′ carbon and X is I. In some embodiments, X is located at the 3′ carbon and X is ¹⁸F. In some embodiments, X is located at the 3′ carbon and X is ²¹¹At. In some embodiments, X is located at the 3′ carbon and X is ¹²³I. In some embodiments, X is located at the 3′ carbon and X is ¹²⁴I. In some embodiments, X is located at the 3′ carbon and X is ¹²⁵I. In some embodiments, X is located at the 3′ carbon and X is ¹³¹I. In some embodiments, X is located at the 3′ carbon and X is ⁷⁶Br. In some embodiments, X is located at the 3′ carbon and X is ⁷⁷Br. In some embodiments, X is located at the 3′ carbon and X is H₃¹¹CO. In some embodiments, X is located at the 3′ carbon and X is CF₂¹⁸F. In some embodiments, X is located at the 3′ carbon and X is CHF¹⁸F. In some embodiments, X is located at the 3′ carbon and X is OCF₂¹⁸F. In some embodiments, X is located at the 3′ carbon and X is OCHF¹⁸F. In some embodiments, X is located at the 3′ carbon and X is SCF₂¹⁸F. In some embodiments, X is located at the 3′ carbon and X is SCHF¹⁸F. In some embodiments, X is located at the 3′ carbon and X is ¹¹CN. In some embodiments, X is located at the 3′ carbon and X is ¹¹CH₃. In some embodiments, X is located at the 3′ carbon and X is H₃¹¹CS.

In some embodiments, X is located at the 2′ carbon and X is I. In some embodiments, X is located at the 2′ carbon and X is ¹⁸F. In some embodiments, X is located at the 2′ carbon and X is ²¹¹At. In some embodiments, X is located at the 2′ carbon and X is ¹²³I. In some embodiments, X is located at the 2′ carbon and X is ¹²⁴I. In some embodiments, X is located at the 2′ carbon and X is ¹²⁵I. In some embodiments, X is located at the 2′ carbon and X is ¹³¹I. In some embodiments, X is located at the 2′ carbon and X is ⁷⁶Br. In some embodiments, X is located at the 2′ carbon and X is ⁷⁷Br. In some embodiments, X is located at the 2′ carbon and X is H₃¹¹CO. In some embodiments, X is located at the 2′ carbon and X is CF₂¹⁸F. In some embodiments, X is located at the 2′ carbon and X is CHF¹⁸F. In some embodiments, X is located at the 2′ carbon and X is OCF₂¹⁸F. In some embodiments, X is located at the 2′ carbon and X is OCHF¹⁸F. In some embodiments, X is located at the 2′ carbon and X is SCF₂¹⁸F. In some embodiments, X is located at the 2′ carbon and X is SCHF¹⁸F. In some embodiments, X is located at the 2′ carbon and X is ¹¹CN. In some embodiments, X is located at the 2′ carbon and X is ¹¹CH₃. In some embodiments, X is located at the 2′ carbon and X is H₃¹¹CS.

In some embodiments, X is located at the 1′ carbon and X is 1. In some embodiments, X is located at the 1′ carbon and X is ¹⁸F. In some embodiments, X is located at the 1′ carbon and X is ²¹¹At. In some embodiments, X is located at the 1′ carbon and X is ¹²³I. In some embodiments, X is located at the 1′ carbon and X is ¹²⁴I. In some embodiments, X is located at the 1′ carbon and X is ¹²⁵I. In some embodiments, X is located at the 1′ carbon and X is ¹³¹I. In some embodiments, X is located at the 1′ carbon and X is ⁷⁶Br. In some embodiments, X is located at the 1′ carbon and X is ⁷⁷Br. In some embodiments, X is located at the 1′ carbon and X is H₃¹¹CO. In some embodiments, X is located at the 1′ carbon and X is CF₂¹⁸F. In some embodiments, X is located at the 1′ carbon and X is CHF¹⁸F. In some embodiments, X is located at the 1′ carbon and X is OCF₂¹⁸F. In some embodiments, X is located at the 1′ carbon and X is OCHF¹⁸F. In some embodiments, X is located at the 1′ carbon and X is SCF₂¹⁸F. In some embodiments, X is located at the 1′ carbon and X is SCHF¹⁸F. In some embodiments, X is located at the 1′ carbon and X is ¹¹CN. In some embodiments, X is located at the 1′ carbon and X is ¹¹CH₃. In some embodiments, X is located at the 1′ carbon and X is H₃¹¹CS.

In general, each R is independently selected from H, F, ²H, CH₃, and cyclopentyl. In embodiments, each R is independently selected from H and F. In embodiments, at least one R is H. In embodiments, both R are H. In embodiments, at least one R is F. In embodiments, both R are F. In embodiments, at least one R is ²H. In embodiments, both R are 2H. In embodiments, at least one R is CH₃. In embodiments, both R are CH₃. In embodiments, at least one R is cyclopentyl. In embodiments, both R are cyclopentyl. In embodiments, X is located at carbon 3′ and both R are H. In embodiments, X is located at carbon 3′ and both R are F. In embodiments, X is located at carbon 2′ and both R are H. In embodiments, X is located at carbon 2′ and both R are F. In embodiments, X is located at carbon 1′ and both R are H. In embodiments, X is located at carbon 1′ and both R are F.

In embodiments, Q and X are located at carbon 3′ and all R are H. In embodiments, Q and X are located at carbon 3′ and all R are F. In embodiments, Q and X are located at carbon 2′ and all R are H. In embodiments, Q and X are located at carbon 2′ and all R are F. In embodiments, Q and X are located at carbon 1′ and all R are H. In embodiments, Q and X are located at carbon 1′ and all R are F.

In embodiments, the methods of the disclosure can include preparing a compound having a structure according to formula (II), the method comprising admixing a compound having a structure according to formula (VI) with a salt, MX:

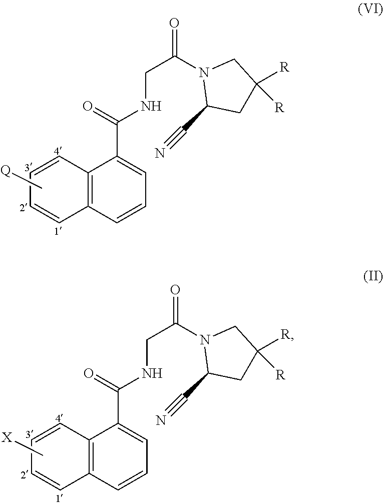

wherein M is selected from the group of K, Li, Na, Rb, Cs, tetraethylammonium ((ethyl)₄N), and tetrabutylammonium ((butyl)₄N), and a combination thereof, in the salt, MX, and in formula (II), X is selected from the group of ¹⁸F, ²¹¹At, ¹²³I, ¹²⁴I, ¹²⁵I, ¹³¹I, ⁷⁶Br, ⁷⁷Br, H₃¹¹CO, CF₂¹⁸F, CHF¹⁸F, OCF₂¹⁸F, OCHF¹⁸F, SCF₂¹⁸F, SCHF¹⁸F, ¹¹CN, ¹¹CH₃, and H₃¹¹CS; and X is the same in the salt and in formula (II); Q is selected from trialkyl stannyl, trialkyl germyl, and trialkyl silyl; in formula (VI) and formula (II), Q and X are located at carbon 1′, 2′, or 3′, and the location of Q in formula (VI) and the location of X in formula (II) are the same; and each R is independently selected from H, F, ²H, CH₃, and cyclopentyl, and the R in formula (II) are the same as the R in formula (VI).

In general, M is any monovalent cation capable of forming a salt with the anion, X. In embodiments, M is selected from the group of K, Li, Na, Rb, Cs, tetraethylammonium ((ethyl)₄N), and tetrabutylammonium ((butyl)₄N). In embodiments, M is selected from the group of K, Li, and Na. In embodiments, M is K.

In general, the compound of having a structure according to formula (VI) can be identical to any compound of having a structure of formula (II) disclosed herein, except that the X group of the compound of formula (II) is replaced with a Q group as defined herein. In embodiments, Q is selected from trialkyl stannyl, trialkyl germyl, and trialkyl silyl. In embodiments, Q is trialkyl stannyl. In embodiments, Q is trialkyl germyl. In embodiments, Q is trialkyl silyl. In embodiments, the alkyl of the trialkyl stannyl, trialkyl germyl and trialkyl silyl are methyl or butyl. In embodiments, the alkyl of the trialkyl stannyl, trialkyl germyl and trialkyl silyl are methyl. In embodiments, the alkyl of the trialkyl stannyl, trialkyl germyl and trialkyl silyl are butyl. In embodiments, Q is trimethyl stannyl. In embodiments, Q is tributyl stannyl.

The methods of the disclosure convert the Q group of the compound having a structure according to formula (VI) to an X group, without otherwise altering the structure of the compound of formula (VI). Thus, the Q group of the compound having a structure according to formula (VI) and the X group of the compound having a structure according to formula (II) are located at the same position on the naphthalene ring and the R groups of the compound having a structure according to formula (II) are the same as the R groups of the compound having a structure according to formula (VI). In embodiments, Q and X are each located at carbon 1′. In embodiments, Q and X are each located at carbon 2′. In embodiments, Q and X are each located at carbon 3′.

In general, the admixing of the compound having a structure according to formula (VI) and the salt, MX, is done under conditions sufficient to provide a compound of having a structure according to formula (II). In general, the admixing can take place in a solution comprising a solvent for the compound having a structure according to formula (VI), a solvent for the salt, or a solvent for the compound having a structure according to formula (VI) and the salt. In embodiments, the solvent can comprise any solvent suitable for solvating the compound having a structure according to formula (VI), the salt, or both. In embodiments, the solvent comprises pyridine, dimethylformamide (DMF), dimethylacetamide (DMA), or a combination thereof. In embodiments, the solvent comprises pyridine. In embodiments, the solvent comprises pyridine and DMF. In embodiments, the solvent comprises pyridine and DMA. The concentrations of the compound having a structure according to formula (VI) and the salt can be any concentration. The concentrations are typically chosen such that the compound having a structure according to formula (VI) and/or the salt is fully soluble, without forming saturated solutions.

In embodiments, the admixing can take place in the presence of a catalyst. Suitable catalysts include, but are not limited to, copper(II) triflate (Cu(OTf)₂), tetrakisacetonitrile copper(I) triflate (CH₃CN)₄CuOTf), and copper(I) trifluoromethanesulfonate toluene complex (CuOTf-toluene). In embodiments, the catalyst comprises copper(II) triflate. In embodiments, the catalyst comprises tetrakisacetonitrile copper(I) triflate. In embodiments, the catalyst comprises copper(I) trifluoromethanesulfonate toluene complex.

In general, the admixing can take place at any suitable temperature for any suitable time. In embodiments, the admixing can take place at a temperature in a range of about 20° C. to about 140° C., for example, about 20° C. to about 120° C., about 20° C. to about 100° C., about 20° C. to about 80°, about 20° to about 60° C., about 20° C. to about 40° C., about 20° C. to about 30° C., about 22° C. to about 26° C., about 40° C. to about 140° C., about 60° C. to about 140° C., about 80° C. to about 140° C., or about 85° C. to about 140° C. In general, as the temperature increases, the rate of reaction increases, and the time required for admixing decreases. As the temperature increases, e.g., above about 140° C., the likelihood of decomposition of the reactants, decomposition of the products, and/or evaporation of the solvents increases. In embodiments, the admixing can take place for a time in a range of about 5 minutes to about 72 hours, for example, about 5 minutes to about 60 hours, about 5 minutes to about 48 hours, about 5 minutes to about 36 hours, about 5 minutes to about 24 hours, about 5 minutes to about 12 hours, about 5 minutes to about 8 hours, about 5 minutes to about 6 hours, about 5 minutes to about 4 hours, about 5 minutes to about 2 hours, about 5 minutes to about 1 hour, about 5 minutes to about 45 minutes, or about 5 minutes to about 30 minutes. As the admixing time decreases, the likelihood of the reaction not going to completion increases. Without intending to be bound by theory, it is believed that as the reaction time increases, for example, beyond about 24 hours, 48 hours, or 72 hours, while some reaction product may continue to be formed the amount provided after 24 hours, 48 hours, or 72 hours will have little effect on the total yield, while the likelihood of side reactions and decomposition of the reagents and/or products increases.

The method of preparing a radiolabeled FAPI having a structure according to formula (II) can further include preparing the compound having a structure according to formula (VI), by admixing a compound having a structure according to formula (IIa) with one of hexaalkyldistannane, hexaalkyldigermane, or hexaalkyldisilane:

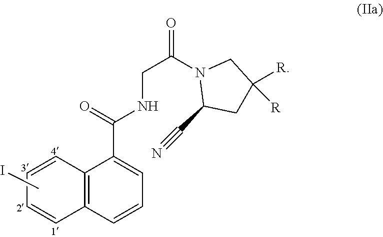

The methods of the disclosure convert the I of the compound having a structure according to formula (IIa) to a Q group, without otherwise altering the structure of the compound of formula (IIa). Thus, the Q group of the compound having a structure according to formula (VI) and the I of the compound having a structure according to formula (IIa) are located at the same position on the naphthalene ring and the R groups of the compound having a structure according to formula (IIa) are the same as the R groups of the compound having a structure according to formula (VI).

In embodiments, the compound of formula (IIa) is admixed with a hexaalkyldistannane. In embodiments, the compound of formula (IIa) is admixed with a hexaalkyldigermane. In embodiments, the compound of formula (IIa) is admixed with a hexaalkyldisilane. In embodiments, the alkyl of the hexaalkyldistannane, hexaalkyldigermane, or hexaalkyldisilane is methyl or butyl. In embodiments, the hexaalkyldistannane is hexamethyldistannane. In embodiments, the hexaalkyldistannane is hexabutyldistannane.

In general, the admixing of the compound having a structure according to formula (IIa) and one of hexaalkyldistannane, hexalkyldigermane, or hexaalkyldisilane, is done under conditions sufficient to provide a compound of having a structure according to formula (VI). In general, the admixing can take place in a solution comprising a solvent for the compound having a structure according to formula (IIa), a solvent for the hexaalkyldistannane, hexalkyldigermane, or hexaalkyldisilane, or a solvent for the compound having a structure according to formula (IIa) and the with one of hexaalkyldistannane, hexalkyldigermane, or hexaalkyldisilane. In embodiments, the solvent can comprise any solvent suitable for solvating the compound having a structure according to formula (IIa), the hexaalkyldistannane, hexalkyldigermane, or hexaalkyldisilane, or both. In embodiments, the solvent comprises toluene. The concentrations of the compound having a structure according to formula (IIa) and the hexaalkyldistannane, hexalkyldigermane, or hexaalkyldisilane can be any concentration. The concentrations are typically chosen such that the compound having a structure according to formula (IIa) and/or the hexaalkyldistannane, hexalkyldigermane, or hexaalkyldisilane is fully soluble, without forming saturated solutions.

In embodiments, the admixing can take place in the presence of an alkali metal halide and/or a catalyst. Suitable alkali metal halides include lithium chloride. Suitable catalysts include, but are not limited to, palladium(0) catalysts. In embodiments, the catalyst comprises tetrakis(triphenylphosphine)palladium(0) (Pd(PPh₃)₄).

In general, the admixing can take place at any suitable temperature for any suitable time. In embodiments, the admixing can take place at a temperature in a range of about 20° C. to about 140° C., for example, about 20° C. to about 120° C., about 20° C. to about 100° C., about 20° C. to about 80°, about 20° to about 60° C., about 20° C. to about 40° C., about 20° C. to about 30° C., about 22° C. to about 26° C., about 40° C. to about 140° C., about 60° C. to about 140° C., about 80° C. to about 140° C., or about 85° C. to about 140° C. In general, as the temperature increases, the rate of reaction increases, and the time required for admixing decreases. As the temperature increases, e.g., above about 140° C., the likelihood of decomposition of the reactants, decomposition of the products, and/or evaporation of the solvents increases. In embodiments, the admixing can take place for a time in a range of about 5 minutes to about 72 hours, for example, about 5 minutes to about 60 hours, about 5 minutes to about 48 hours, about 5 minutes to about 36 hours, about 5 minutes to about 24 hours, about 5 minutes to about 12 hours, about 5 minutes to about 8 hours, about 5 minutes to about 6 hours, about 5 minutes to about 4 hours, about 5 minutes to about 2 hours, about 5 minutes to about 1 hour, about 5 minutes to about 45 minutes, or about 5 minutes to about 30 minutes. As the admixing time decreases, the likelihood of the reaction not going to completion increases. Without intending to be bound by theory, it is believed that as the reaction time increases, for example, beyond about 24 hours, 48 hours, or 72 hours, while some reaction product may continue to be formed the amount provided after 24 hours, 48 hours, or 72 hours will have little effect on the total yield, while the likelihood of side reactions and decomposition of the reagents and/or products increases.

In some embodiments, X is located at the 3′ carbon and X is I. In some embodiments, X is located at the 3′ carbon and X is ¹⁸F. In some embodiments, X is located at the 3′ carbon and X is ²¹¹At. In some embodiments, X is located at the 3′ carbon and X is ¹²³I. In some embodiments, X is located at the 3′ carbon and X is ¹²⁴I. In some embodiments, X is located at the 3′ carbon and X is ¹²⁵I. In some embodiments, X is located at the 3′ carbon and X is ¹³¹I. In some embodiments, X is located at the 3′ carbon and X is ⁷⁶Br. In some embodiments, X is located at the 3′ carbon and X is ⁷⁷Br. In some embodiments, X is located at the 3′ carbon and X is H₃¹¹CO. In some embodiments, X is located at the 3′ carbon and X is CF₂¹⁸F. In some embodiments, X is located at the 3′ carbon and X is CHF¹⁸F. In some embodiments, X is located at the 3′ carbon and X is OCF₂¹⁸F. In some embodiments, X is located at the 3′ carbon and X is OCHF¹⁸F. In some embodiments, X is located at the 3′ carbon and X is SCF₂¹⁸F. In some embodiments, X is located at the 3′ carbon and X is SCHF¹⁸F. In some embodiments, X is located at the 3′ carbon and X is ¹¹CN. In some embodiments, X is located at the 3′ carbon and X is ¹¹CH₃. In some embodiments, X is located at the 3′ carbon and X is H₃¹¹CS.

In some embodiments, X is located at the 2′ carbon and X is I. In some embodiments, X is located at the 2′ carbon and X is ¹⁸F. In some embodiments, X is located at the 2′ carbon and X is ²¹¹At. In some embodiments, X is located at the 2′ carbon and X is ¹²³I. In some embodiments, X is located at the 2′ carbon and X is ¹²⁴I. In some embodiments, X is located at the 2′ carbon and X is ¹²⁵I. In some embodiments, X is located at the 2′ carbon and X is ¹³¹I. In some embodiments, X is located at the 2′ carbon and X is ⁷⁶Br. In some embodiments, X is located at the 2′ carbon and X is ⁷⁷Br. In some embodiments, X is located at the 2′ carbon and X is H₃¹¹CO. In some embodiments, X is located at the 2′ carbon and X is CF₂¹⁸F. In some embodiments, X is located at the 2′ carbon and X is CHF¹⁸F. In some embodiments, X is located at the 2′ carbon and X is OCF₂¹⁸F. In some embodiments, X is located at the 2′ carbon and X is OCHF¹⁸F. In some embodiments, X is located at the 2′ carbon and X is SCF₂¹⁸F. In some embodiments, X is located at the 2′ carbon and X is SCHF¹⁸F. In some embodiments, X is located at the 2′ carbon and X is ¹¹CN. In some embodiments, X is located at the 2′ carbon and X is ¹¹CH₃. In some embodiments, X is located at the 2′ carbon and X is H₃¹¹CS.

In some embodiments, X is located at the 1′ carbon and X is I. In some embodiments, X is located at the 1′ carbon and X is ¹⁸F. In some embodiments, X is located at the 1′ carbon and X is ²¹¹At. In some embodiments, X is located at the 1′ carbon and X is ¹²³I. In some embodiments, X is located at the 1′ carbon and X is ¹²⁴I. In some embodiments, X is located at the 1′ carbon and X is ¹²⁵I. In some embodiments, X is located at the 1′ carbon and X is ¹³¹I. In some embodiments, X is located at the 1′ carbon and X is ⁷⁶Br. In some embodiments, X is located at the 1′ carbon and X is ⁷⁷Br. In some embodiments, X is located at the 1′ carbon and X is H₃¹¹CO. In some embodiments, X is located at the 1′ carbon and X is CF₂¹⁸F. In some embodiments, X is located at the 1′ carbon and X is CHF¹⁸F. In some embodiments, X is located at the 1′ carbon and X is OCF₂¹⁸F. In some embodiments, X is located at the 1′ carbon and X is OCHF¹⁸F. In some embodiments, X is located at the 1′ carbon and X is SCF₂¹⁸F. In some embodiments, X is located at the 1′ carbon and X is SCHF¹⁸F. In some embodiments, X is located at the 1′ carbon and X is ¹¹CN. In some embodiments, X is located at the 1′ carbon and X is ¹¹CH₃. In some embodiments, X is located at the 1′ carbon and X is H₃¹¹CS.

In general, each R is independently selected from H, F, ²H, CH₃, and cyclopentyl. In embodiments, each R is independently selected from H and F. In embodiments, at least one R is H. In embodiments, both R are H. In embodiments, at least one R is F. In embodiments, both R are F. In embodiments, at least one R is ²H. In embodiments, both R are 2H. In embodiments, at least one R is CH₃. In embodiments, both R are CH₃. In embodiments, at least one R is cyclopentyl. In embodiments, both R are cyclopentyl. In embodiments, X is located at carbon 3′ and both R are H. In embodiments, X is located at carbon 3′ and both R are F. In embodiments, X is located at carbon 2′ and both R are H. In embodiments, X is located at carbon 2′ and both R are F. In embodiments, X is located at carbon 1′ and both R are H. In embodiments, X is located at carbon 1′ and both R are F.

In embodiments, Q and X are located at carbon 3′ and all R are H. In embodiments, Q and X are located at carbon 3′ and all R are F. In embodiments, Q and X are located at carbon 2′ and all R are H. In embodiments, Q and X are located at carbon 2′ and all R are F. In embodiments, Q and X are located at carbon 1′ and all R are H. In embodiments, Q and X are located at carbon 1′ and all R are F.

In embodiments, the methods of the disclosure can include preparing a compound having a structure according to formula (III), the method comprising admixing a compound having a structure according to formula (VII) with a salt, MX:

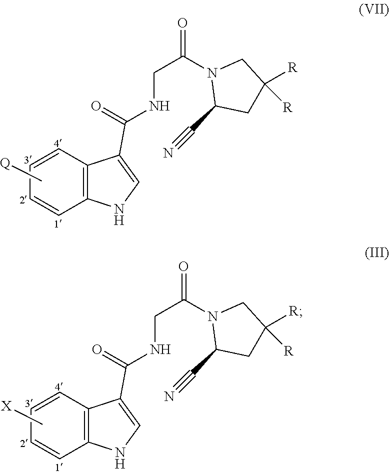

wherein M is selected from the group of K, Li, Na, Rb, Cs, tetraethylammonium ((ethyl)₄N), and tetrabutylammonium ((butyl)₄N), and a combination thereof, in the salt, MX, and in formula (III), X is selected from the group of ¹⁸F, ²¹¹At, ¹²³I, ¹²⁴I, ¹²⁵I, ¹³¹I ⁷⁶Br, ⁷⁷Br, H₃¹¹CO, CF₂¹⁸F, CHF¹⁸F, OCF₂¹⁸F, OCHF¹⁸F, SCF₂¹⁸F, SCHF¹⁸F, ¹¹CN, ¹¹CH₃, and H₃¹¹CS; and X is the same in the salt and in formula (III); Q is selected from trialkyl stannyl, trialkyl germyl, and trialkyl silyl; in formula (VII) and formula (III), Q and X are located at carbon 1′, 2′, or 3′, and the location of Q in formula (VII) and the location of X in formula (III) are the same; and each R is independently selected from H, F, ²H, CH₃, and cyclopentyl, and the R in formula (III) are the same as the R in formula (VII).

In general, M is any monovalent cation capable of forming a salt with the anion, X. In embodiments, M is selected from the group of K, Li, Na, Rb, Cs, tetraethylammonium ((ethyl)₄N), and tetrabutylammonium ((butyl)₄N). In embodiments, M is selected from the group of K, Li, and Na. In embodiments, M is K.

In general, the compound of having a structure according to formula (VII) can be identical to any compound of having a structure of formula (III) disclosed herein, except that the X group of the compound of formula (III) is replaced with a Q group as defined herein. In embodiments, Q is selected from trialkyl stannyl, trialkyl germyl, and trialkyl silyl. In embodiments, Q is trialkyl stannyl. In embodiments, Q is trialkyl germyl. In embodiments, Q is trialkyl silyl. In embodiments, the alkyl of the trialkyl stannyl, trialkyl germyl and trialkyl silyl are methyl or butyl. In embodiments, the alkyl of the trialkyl stannyl, trialkyl germyl and trialkyl silyl are methyl. In embodiments, the alkyl of the trialkyl stannyl, trialkyl germyl and trialkyl silyl are butyl. In embodiments, Q is trimethyl stannyl. In embodiments, Q is tributyl stannyl.

The methods of the disclosure convert the Q group of the compound having a structure according to formula (VII) to an X group, without otherwise altering the structure of the compound of formula (VII). Thus, the Q group of the compound having a structure according to formula (VII) and the X group of the compound having a structure according to formula (III) are located at the same position on the indole ring and the R groups of the compound having a structure according to formula (III) are the same as the R groups of the compound having a structure according to formula (VII). In embodiments, Q and X are each located at carbon 1′. In embodiments, Q and X are each located at carbon 2′. In embodiments, Q and X are each located at carbon 3′.

In general, the admixing of the compound having a structure according to formula (VII) and the salt, MX, is done under conditions sufficient to provide a compound of having a structure according to formula (III). In general, the admixing can take place in a solution comprising a solvent for the compound having a structure according to formula (VII), a solvent for the salt, or a solvent for the compound having a structure according to formula (VII) and the salt. In embodiments, the solvent can comprise any solvent suitable for solvating the compound having a structure according to formula (VII), the salt, or both. In embodiments, the solvent comprises pyridine, dimethylformamide (DMF), dimethylacetamide (DMA), or a combination thereof. In embodiments, the solvent comprises pyridine. In embodiments, the solvent comprises pyridine and DMF. In embodiments, the solvent comprises pyridine and DMA. The concentrations of the compound having a structure according to formula (VII) and the salt can be any concentration. The concentrations are typically chosen such that the compound having a structure according to formula (VII) and/or the salt is fully soluble, without forming saturated solutions.

In embodiments, the admixing can take place in the presence of a catalyst. Suitable catalysts include, but are not limited to, copper(II) triflate (Cu(OTf)₂), tetrakisacetonitrile copper(I) triflate (CH₃CN)₄CuOTf), and copper(I) trifluoromethanesulfonate toluene complex (CuOTf-toluene). In embodiments, the catalyst comprises copper(II) triflate. In embodiments, the catalyst comprises tetrakisacetonitrile copper(I) triflate. In embodiments, the catalyst comprises copper(I) trifluoromethanesulfonate toluene complex.

In general, the admixing can take place at any suitable temperature for any suitable time. In embodiments, the admixing can take place at a temperature in a range of about 20° C. to about 140° C., for example, about 20° C. to about 120° C., about 20° C. to about 100° C., about 20° C. to about 80°, about 20° to about 60° C., about 20° C. to about 40° C., about 20° C. to about 30° C., about 22° C. to about 26° C., about 40° C. to about 140° C., about 60° C. to about 140° C., about 80° C. to about 140° C., or about 85° C. to about 140° C. In general, as the temperature increases, the rate of reaction increases, and the time required for admixing decreases. As the temperature increases, e.g., above about 140° C., the likelihood of decomposition of the reactants, decomposition of the products, and/or evaporation of the solvents increases. In embodiments, the admixing can take place for a time in a range of about 5 minutes to about 72 hours, for example, about 5 minutes to about 60 hours, about 5 minutes to about 48 hours, about 5 minutes to about 36 hours, about 5 minutes to about 24 hours, about 5 minutes to about 12 hours, about 5 minutes to about 8 hours, about 5 minutes to about 6 hours, about 5 minutes to about 4 hours, about 5 minutes to about 2 hours, about 5 minutes to about 1 hour, about 5 minutes to about 45 minutes, or about 5 minutes to about 30 minutes. As the admixing time decreases, the likelihood of the reaction not going to completion increases. Without intending to be bound by theory, it is believed that as the reaction time increases, for example, beyond about 24 hours, 48 hours, or 72 hours, while some reaction product may continue to be formed the amount provided after 24 hours, 48 hours, or 72 hours will have little effect on the total yield, while the likelihood of side reactions and decomposition of the reagents and/or products increases.

The method of preparing a radiolabeled FAPI having a structure according to formula (III) can further include preparing the compound having a structure according to formula (VII), by admixing a compound having a structure according to formula (IIIa) with one of hexaalkyldistannane, hexaalkyldigermane, or hexaalkyldisilane:

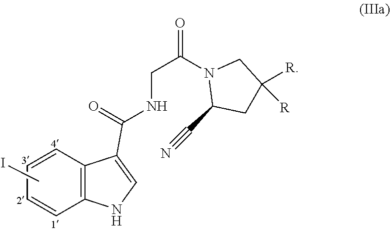

The methods of the disclosure convert the I of the compound having a structure according to formula (IIIa) to a Q group, without otherwise altering the structure of the compound of formula (IIIa). Thus, the Q group of the compound having a structure according to formula (VII) and the I of the compound having a structure according to formula (IIIa) are located at the same position on the indole ring and the R groups of the compound having a structure according to formula (IIIa) are the same as the R groups of the compound having a structure according to formula (VII).

In embodiments, the compound of formula (IIIa) is admixed with a hexaalkyldistannane. In embodiments, the compound of formula (IIIa) is admixed with a hexaalkyldigermane. In embodiments, the compound of formula (IIIa) is admixed with a hexaalkyldisilane. In embodiments, the alkyl of the hexaalkyldistannane, hexaalkyldigermane, or hexaalkyldisilane is methyl or butyl. In embodiments, the hexaalkyldistannane is hexamethyldistannane. In embodiments, the hexaalkyldistannane is hexabutyldistannane.

In general, the admixing of the compound having a structure according to formula (Illa) and one of hexaalkyldistannane, hexalkyldigermane, or hexaalkyldisilane, is done under conditions sufficient to provide a compound of having a structure according to formula (VII). In general, the admixing can take place in a solution comprising a solvent for the compound having a structure according to formula (IIIa), a solvent for the hexaalkyldistannane, hexalkyldigermane, or hexaalkyldisilane, or a solvent for the compound having a structure according to formula (IIIa) and the with one of hexaalkyldistannane, hexalkyldigermane, or hexaalkyldisilane. In embodiments, the solvent can comprise any solvent suitable for solvating the compound having a structure according to formula (IIIa), the hexaalkyldistannane, hexalkyldigermane, or hexaalkyldisilane, or both. In embodiments, the solvent comprises toluene. The concentrations of the compound having a structure according to formula (IIIa) and the hexaalkyldistannane, hexalkyldigermane, or hexaalkyldisilane can be any concentration. The concentrations are typically chosen such that the compound having a structure according to formula (IIIa) and/or the hexaalkyldistannane, hexalkyldigermane, or hexaalkyldisilane is fully soluble, without forming saturated solutions.

In embodiments, the admixing can take place in the presence of an alkali metal halide and/or a catalyst. Suitable alkali metal halides include lithium chloride. Suitable catalysts include, but are not limited to, palladium(0) catalysts. In embodiments, the catalyst comprises tetrakis(triphenylphosphine)palladium(0) (Pd(PPh₃)₄).

In general, the admixing can take place at any suitable temperature for any suitable time. In embodiments, the admixing can take place at a temperature in a range of about 20° C. to about 140° C., for example, about 20° C. to about 120° C., about 20° C. to about 100° C., about 20° C. to about 80°, about 20° to about 60° C., about 20° C. to about 40° C., about 20° C. to about 30° C., about 22° C. to about 26° C., about 40° C. to about 140° C., about 60° C. to about 140° C., about 80° C. to about 140° C., or about 85° C. to about 140° C. In general, as the temperature increases, the rate of reaction increases, and the time required for admixing decreases. As the temperature increases, e.g., above about 140° C., the likelihood of decomposition of the reactants, decomposition of the products, and/or evaporation of the solvents increases. In embodiments, the admixing can take place for a time in a range of about 5 minutes to about 72 hours, for example, about 5 minutes to about 60 hours, about 5 minutes to about 48 hours, about 5 minutes to about 36 hours, about 5 minutes to about 24 hours, about 5 minutes to about 12 hours, about 5 minutes to about 8 hours, about 5 minutes to about 6 hours, about 5 minutes to about 4 hours, about 5 minutes to about 2 hours, about 5 minutes to about 1 hour, about 5 minutes to about 45 minutes, or about 5 minutes to about 30 minutes. As the admixing time decreases, the likelihood of the reaction not going to completion increases. Without intending to be bound by theory, it is believed that as the reaction time increases, for example, beyond about 24 hours, 48 hours, or 72 hours, while some reaction product may continue to be formed the amount provided after 24 hours, 48 hours, or 72 hours will have little effect on the total yield, while the likelihood of side reactions and decomposition of the reagents and/or products increases.

In some embodiments, X is located at the 3′ carbon and X is I. In some embodiments, X is located at the 3′ carbon and X is ¹⁸F. In some embodiments, X is located at the 3′ carbon and X is ²¹¹At. In some embodiments, X is located at the 3′ carbon and X is ¹²³I. In some embodiments, X is located at the 3′ carbon and X is ¹²⁴I. In some embodiments, X is located at the 3′ carbon and X is ¹²⁵I. In some embodiments, X is located at the 3′ carbon and X is ¹³¹I. In some embodiments, X is located at the 3′ carbon and X is ⁷⁶Br. In some embodiments, X is located at the 3′ carbon and X is ⁷⁷Br. In some embodiments, X is located at the 3′ carbon and X is H₃¹¹CO. In some embodiments, X is located at the 3′ carbon and X is CF₂¹⁸F. In some embodiments, X is located at the 3′ carbon and X is CHF¹⁸F. In some embodiments, X is located at the 3′ carbon and X is OCF₂¹⁸F. In some embodiments, X is located at the 3′ carbon and X is OCHF¹⁸F. In some embodiments, X is located at the 3′ carbon and X is SCF₂¹⁸F. In some embodiments, X is located at the 3′ carbon and X is SCHF¹⁸F. In some embodiments, X is located at the 3′ carbon and X is ¹¹CN. In some embodiments, X is located at the 3′ carbon and X is ¹¹CH₃. In some embodiments, X is located at the 3′ carbon and X is H₃¹¹CS.

In some embodiments, X is located at the 2′ carbon and X is I. In some embodiments, X is located at the 2′ carbon and X is ¹⁸F. In some embodiments, X is located at the 2′ carbon and X is ²¹¹At. In some embodiments, X is located at the 2′ carbon and X is ¹²³I. In some embodiments, X is located at the 2′ carbon and X is ¹²⁴I. In some embodiments, X is located at the 2′ carbon and X is ¹²⁵I. In some embodiments, X is located at the 2′ carbon and X is ¹³¹I. In some embodiments, X is located at the 2′ carbon and X is ⁷⁶Br. In some embodiments, X is located at the 2′ carbon and X is ⁷⁷Br. In some embodiments, X is located at the 2′ carbon and X is H₃¹¹CO. In some embodiments, X is located at the 2′ carbon and X is CF₂¹⁸F. In some embodiments, X is located at the 2′ carbon and X is CHF¹⁸F. In some embodiments, X is located at the 2′ carbon and X is OCF₂¹⁸F. In some embodiments, X is located at the 2′ carbon and X is OCHF¹⁸F. In some embodiments, X is located at the 2′ carbon and X is SCF₂¹⁸F. In some embodiments, X is located at the 2′ carbon and X is SCHF¹⁸F. In some embodiments, X is located at the 2′ carbon and X is ¹¹CN. In some embodiments, X is located at the 2′ carbon and X is ¹¹CH₃. In some embodiments, X is located at the 2′ carbon and X is H₃¹¹CS.

In some embodiments, X is located at the 1′ carbon and X is I. In some embodiments, X is located at the 1′ carbon and X is ¹⁸F. In some embodiments, X is located at the 1′ carbon and X is ²¹¹At. In some embodiments, X is located at the 1′ carbon and X is ¹²³I. In some embodiments, X is located at the 1′ carbon and X is ¹²⁴I. In some embodiments, X is located at the 1′ carbon and X is ¹²⁵I. In some embodiments, X is located at the 1′ carbon and X is ¹³¹I. In some embodiments, X is located at the 1′ carbon and X is ⁷⁶Br. In some embodiments, X is located at the 1′ carbon and X is ⁷⁷Br. In some embodiments, X is located at the 1′ carbon and X is H₃¹¹CO. In some embodiments, X is located at the 1′ carbon and X is CF₂¹⁸F. In some embodiments, X is located at the 1′ carbon and X is CHF¹⁸F. In some embodiments, X is located at the 1′ carbon and X is OCF₂¹⁸F. In some embodiments, X is located at the 1′ carbon and X is OCHF¹⁸F. In some embodiments, X is located at the 1′ carbon and X is SCF₂¹⁸F. In some embodiments, X is located at the 1′ carbon and X is SCHF¹⁸F. In some embodiments, X is located at the 1′ carbon and X is ¹¹CN. In some embodiments, X is located at the 1′ carbon and X is ¹¹CH₃. In some embodiments, X is located at the 1′ carbon and X is H₃¹¹CS.

In general, each R is independently selected from H, F, ²H, CH₃, and cyclopentyl. In embodiments, each R is independently selected from H and F. In embodiments, at least one R is H. In embodiments, both R are H. In embodiments, at least one R is F. In embodiments, both R are F. In embodiments, at least one R is ²H. In embodiments, both R are 2H. In embodiments, at least one R is CH₃. In embodiments, both R are CH₃. In embodiments, at least one R is cyclopentyl. In embodiments, both R are cyclopentyl. In embodiments, X is located at carbon 3′ and both R are H. In embodiments, X is located at carbon 3′ and both R are F. In embodiments, X is located at carbon 2′ and both R are H. In embodiments, X is located at carbon 2′ and both R are F. In embodiments, X is located at carbon 1′ and both R are H. In embodiments, X is located at carbon 1′ and both R are F.

In embodiments, Q and X are located at carbon 3′ and all R are H. In embodiments, Q and X are located at carbon 3′ and all R are F. In embodiments, Q and X are located at carbon 2′ and all R are H. In embodiments, Q and X are located at carbon 2′ and all R are F. In embodiments, Q and X are located at carbon 1′ and all R are H. In embodiments, Q and X are located at carbon 1′ and all R are F.

Methods of Preparing Radiolabeled FAPIs Having Structures According to Formula (I), Formula (II), or Formula (III) Through an Intermediate Selected from a Compound Having a Structure Selected from the Group of Formula (VIII), Formula (X), and Formula (XI)

In embodiments, the methods of the disclosure can include preparing a radiolabeled compound having a structure according to formula (I), the method comprising admixing a radiolabeled salt, MX, and a compound having a structure according to formula (VIII):

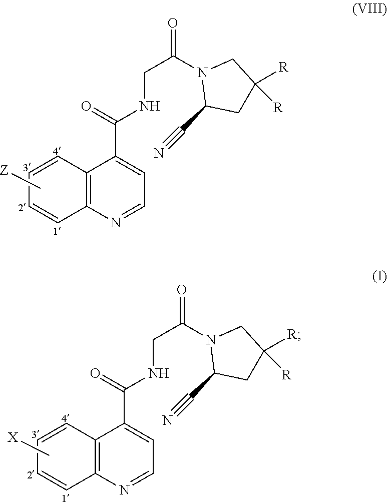

wherein Z has a structure selected from the group of:

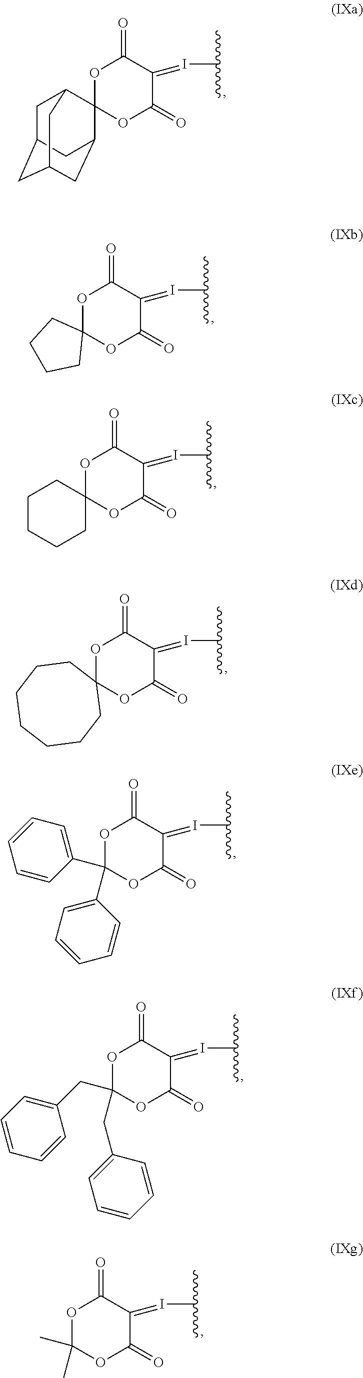

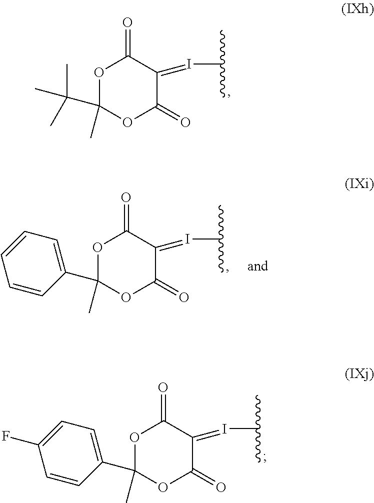 [SMILES: *I=C1C(=O)OC(C)(C(C)(C)C)OC1=O ; *I=C1C(=O)OC(C)(c2ccccc2)OC1=O ; *I=C1C(=O)OC(C)(c2ccc(F)cc2)OC1=O]

and wherein in formula (VIII) and formula (I), Z and X are located at carbon 1′, 2′, or 3′, and the location of Z in formula (VIII) and the location of X in formula (I) are the same; the M is selected from the group of K, Li, Na, Rb, Cs, tetraethylammonium, and tetrabutylammonium; in the salt, MX, and in formula (I), X is selected from the group of ¹⁸F, ²¹¹At, ¹²³I, ¹²⁴I, ¹²⁵I, ¹³¹I, ⁷⁶Br, ⁷⁷Br, and the X in the salt is the same as the X in formula (I); and each R is independently selected from H, F, ²H, CH₃, and cyclopentyl, and the R in formula (I) are the same as the R in formula (VIII).

In general, M is any monovalent cation capable of forming a salt with the anion, X. In embodiments, M is selected from the group of K, Li, Na, Rb, Cs, tetraethylammonium ((ethyl)₄N), and tetrabutylammonium ((butyl)₄N). In embodiments, M is selected from the group of K, Li, and Na. In embodiments, M is K. In embodiments, M is tetraethylammonium.

In general, the compound of having a structure according to formula (VIII) can be identical to any compound of having a structure of formula (I) disclosed herein, except that the X group of the compound of formula (I) is replaced with a Z group as defined herein. In embodiments, Z is

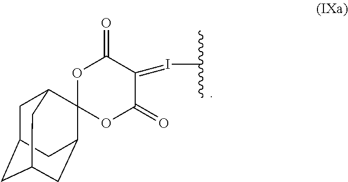 [SMILES: *I=C1C(=O)OC2(OC1=O)C1CC3CC(C1)CC2C3]

In embodiments, Z is

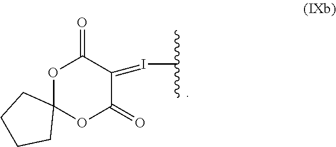 [SMILES: *I=C1C(=O)OC2(CCCC2)OC1=O]

In embodiments, Z is

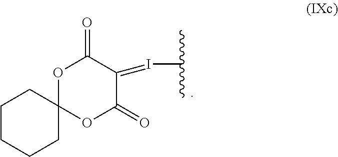 [SMILES: *I=C1C(=O)OC2(CCCCC2)OC1=O]

In embodiments, Z is

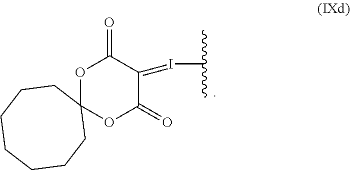 [SMILES: *I=C1C(=O)OC2(CCCCCCC2)OC1=O]

In embodiments, Z is

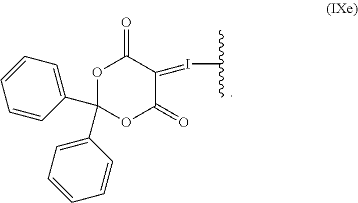 [SMILES: *I=C1C(=O)OC(c2ccccc2)(c2ccccc2)OC1=O]

In embodiments, Z is

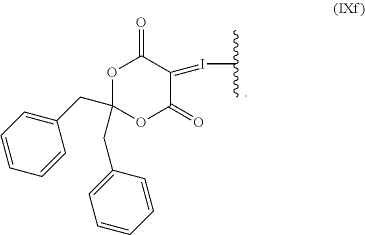 [SMILES: *I=C1C(=O)OC(Cc2ccccc2)(Cc2ccccc2)OC1=O]

In embodiments, Z is

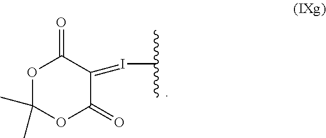 [SMILES: *I=C1C(=O)OC(C)(C)OC1=O]

In embodiments, Z is

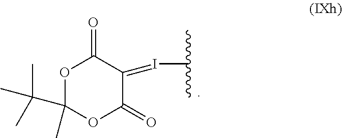 [SMILES: *I=C1C(=O)OC(C)(C(C)(C)C)OC1=O]

In embodiments, Z is

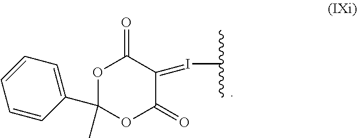 [SMILES: *I=C1C(=O)OC(C)(c2ccccc2)OC1=O]

In embodiments, Z is

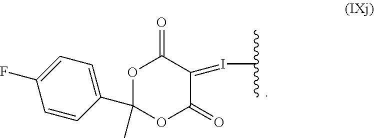 [SMILES: *I=C1C(=O)OC(C)(c2ccc(F)cc2)OC1=O]

The methods of the disclosure convert the Z group of the compound having a structure according to formula (VIII) to an X group, without otherwise altering the structure of the compound of formula (VIII). Thus, the Z group of the compound having a structure according to formula (VIII) and the X group of the compound having a structure according to formula (I) are located at the same position on the quinoline ring and the R groups of the compound having a structure according to formula (I) are the same as the R groups of the compound having a structure according to formula (VIII). In embodiments, Z and X are each located at carbon 1′. In embodiments, Z and X are each located at carbon 2′. In embodiments, Z and X are each located at carbon 3′.

In general, the admixing of the compound having a structure according to formula (VIII) and the radiolabeled salt, MX, is done under conditions sufficient to provide a compound of having a structure according to formula (I). In general, the admixing can take place in a solution comprising a solvent for the compound having a structure according to formula (VIII), a solvent for the salt, or a solvent for the compound having a structure according to formula (VIII) and the salt. In embodiments, the solvent can comprise any solvent suitable for solvating the compound having a structure according to formula (VIII), the salt, or both. In embodiments, the solvent comprises dimethylsulfoxide (DMSO), dimethylformamide (DMF), dimethylacetamide (DMA), or a combination thereof. In embodiments, the solvent comprises DMSO. In embodiments, the solvent comprises pyridine and DMF. In embodiments, the solvent comprises pyridine and DMA. The concentrations of the compound having a structure according to formula (VIII) and the salt can be any concentration. The concentrations are typically chosen such that the compound having a structure according to formula (VIII) and/or the salt is fully soluble, without forming saturated solutions.

In general, the admixing can take place at any suitable temperature for any suitable time. In embodiments, the admixing can take place at a temperature in a range of about 20° C. to about 140° C., for example, about 20° C. to about 120° C., about 20° C. to about 100° C., about 20° C. to about 80°, about 20° to about 60° C., about 20° C. to about 40° C., about 20° C. to about 30° C., about 22° C. to about 26° C., about 40° C. to about 140° C., about 60° C. to about 140° C., about 80° C. to about 140° C., or about 85° C. to about 140° C. In general, as the temperature increases, the rate of reaction increases, and the time required for admixing decreases. As the temperature increases, e.g., above about 140° C., the likelihood of decomposition of the reactants, decomposition of the products, and/or evaporation of the solvents increases. In embodiments, the admixing can take place for a time in a range of about 5 minutes to about 72 hours, for example, about 5 minutes to about 60 hours, about 5 minutes to about 48 hours, about 5 minutes to about 36 hours, about 5 minutes to about 24 hours, about 5 minutes to about 12 hours, about 5 minutes to about 8 hours, about 5 minutes to about 6 hours, about 5 minutes to about 4 hours, about 5 minutes to about 2 hours, about 5 minutes to about 1 hour, about 5 minutes to about 45 minutes, or about 5 minutes to about 30 minutes. As the admixing time decreases, the likelihood of the reaction not going to completion increases. Without intending to be bound by theory, it is believed that as the reaction time increases, for example, beyond about 24 hours, 48 hours, or 72 hours, while some reaction product may continue to be formed the amount provided after 24 hours, 48 hours, or 72 hours will have little effect on the total yield, while the likelihood of side reactions and decomposition of the reagents and/or products increases.

The method of preparing a radiolabeled FAPI having a structure according to formula (I) can further include preparing the compound having a structure according to formula (VIII), by admixing a compound having a structure according to formula (Ia):

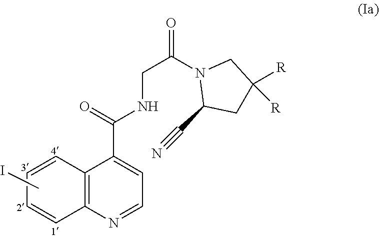

with 1-chloromethyl-4-fluoro-1,4-diazoniabicyclo[2.2.2]octanebis(tetrafluoroborate), and trimethylsilyl acetate to form a reaction product. The method can further include admixing the reaction product of the compound of formula (Ia) with 1-chloromethyl-4-fluoro-1,4-diazoniabicyclo[2.2.2]octanebis(tetrafluoroborate), and trimethylsilyl acetate with a dioxane-dione and sodium carbonate to form the compound having a structure according to formula (VIII).

The methods of the disclosure convert the I of the compound having a structure according to formula (Ia) to a Z group, without otherwise altering the structure of the compound of formula (Ia). Thus, the Z group of the compound having a structure according to formula (VIII) and the I of the compound having a structure according to formula (Ia) are located at the same position on the quinoline ring and the R groups of the compound having a structure according to formula (Ia) are the same as the R groups of the compound having a structure according to formula (VIII).

In embodiments, the compound of formula (Ia) is admixed with 1-chloromethyl-4-fluoro-1,4-diazoniabicyclo[2.2.2]octanebis(tetrafluoroborate) under conditions sufficient to provide a reaction product that can be converted to the compound of having a structure according to formula (VIII). In general, the admixing can take place in a solution comprising a solvent for the compound having a structure according to formula (Ia), a solvent for the 1-chloromethyl-4-fluoro-1,4-diazoniabicyclo[2.2.2]octanebis(tetrafluoroborate), or a solvent for the compound having a structure according to formula (Ia) and the 1-chloromethyl-4-fluoro-1,4-diazoniabicyclo[2.2.2]octanebis(tetrafluoroborate). In embodiments, the solvent can comprise any solvent suitable for solvating the compound having a structure according to formula (Ia), the 1-chloromethyl-4-fluoro-1,4-diazoniabicyclo[2.2.2]octanebis(tetrafluoroborate), or both. In embodiments, the solvent comprises acetonitrile. The concentrations of the compound having a structure according to formula (Ia) can be any concentration. The concentration is typically chosen such that the compound having a structure according to formula (Ia) is fully soluble, without forming saturated solutions.

In embodiments, the compound of formula (Ia) is admixed with 1-chloromethyl-4-fluoro-1,4-diazoniabicyclo[2.2.2]octanebis(tetrafluoroborate) in the presence of trimethyl silyl acetate. In embodiments, the admixing comprises dropwise addition of 1-chloromethyl-4-fluoro-1,4-diazoniabicyclo[2.2.2]octanebis(tetrafluoroborate) to a solution of the compound having a structure according to formula (Ia) and trimethylsilyl acetate.

In general, the admixing of the compound of formula (Ia) is admixed with 1-chloromethyl-4-fluoro-1,4-diazoniabicyclo[2.2.2]octanebis(tetrafluoroborate) can take place at any suitable temperature for any suitable time. In embodiments, the admixing can take place at a temperature in a range of about 20° C. to about 140° C., for example, about 20° C. to about 120° C., about 20° C. to about 100° C., about 20° C. to about 80°, about 20° to about 60° C., about 20° C. to about 40° C., about 20° C. to about 30° C., about 22° C. to about 26° C., about 40° C. to about 140° C., about 60° C. to about 140° C., about 80° C. to about 140° C., or about 85° C. to about 140° C. In general, as the temperature increases, the rate of reaction increases, and the time required for admixing decreases. As the temperature increases, e.g., above about 140° C., the likelihood of decomposition of the reactants, decomposition of the products, and/or evaporation of the solvents increases. In embodiments, the admixing can take place for a time in a range of about 5 minutes to about 72 hours, for example, about 5 minutes to about 60 hours, about 5 minutes to about 48 hours, about 5 minutes to about 36 hours, about 5 minutes to about 24 hours, about 5 minutes to about 12 hours, about 5 minutes to about 8 hours, about 5 minutes to about 6 hours, about 5 minutes to about 4 hours, about 5 minutes to about 2 hours, about 5 minutes to about 1 hour, about 5 minutes to about 45 minutes, about 5 minutes to about 30 minutes, about 15 minutes to about 24 hours, about 1 hour to about 24 hours, about 2 hours to about 23 hours, about 5 hours to about 22 hours, about 8 hours to about 21 hours, about 10 hours to about 20 hours, about 12 hours to about 19 hours, or about 14 hours to about 18 hours. As the admixing time decreases, the likelihood of the reaction not going to completion increases. Without intending to be bound by theory, it is believed that as the reaction time increases, for example, beyond about 24 hours, 48 hours, or 72 hours, while some reaction product may continue to be formed the amount provided after 24 hours, 48 hours, or 72 hours will have little effect on the total yield, while the likelihood of side reactions and decomposition of the reagents and/or products increases.

In embodiments, the admixing of the reaction product of the compound of formula (Ia) with 1-chloromethyl-4-fluoro-1,4-diazoniabicyclo[2.2.2]octanebis(tetrafluoroborate) is admixed with a dioxane-dione under conditions sufficient to provide the compound of having a structure according to formula (VIII). In general, the admixing can take place in a solution comprising a solvent for the reaction product, a solvent for the dioxane-dione, or both. In embodiments, the solvent can comprise any solvent suitable for solvating the reaction product, a solvent for the dioxane-dione, or both. In embodiments, the solvent comprises ethanol. The concentrations of the reactants are typically chosen such that the reactants are fully soluble, without forming saturated solutions.

In embodiments, the dioxane-dione is selected from the group of (1r,3r,5r,7r)-spiro[adamantan-2,2′-[1,3]-dioxane]-4′,6′-dione (SPIAd), 6,10-dioxaspiro[4.5]decane-7,9-dione, 1,5-dioxaspiro[5.5]undecane-2,4-dione, 1,5-dioxaspiro[5.7]tridecane-2,4-dione, 2,2-diphenyl-1,3-dioxane-4,6-dione, 2,2-dibenzyl-1,3-dioxane-4,6-dione, 5,5-dimethylcyclohexane-1,3-dione, 2-(tert-butyl)-2-methyl-1,3-dioxane-4,6-dione, 2-methyl-2-phenyl-1,3-dioxane-4,6-dione, and 2-(4-fluorophenyl)-2-methyl-1,3-dioxane-4,6-dione. In embodiments, the dioxane-dione is (1r,3r,5r,7r)-spiro[adamantan-2,2′-[1,3]-dioxane]-4′,6′-dione (SPIAd). In embodiments, the admixing comprises dropwise addition of the dioxane-dione to the reaction product.

In general, the admixing of the reaction product and the dioxane-dione can take place at any suitable temperature for any suitable time. In embodiments, the admixing can take place at a temperature in a range of about 20° C. to about 140° C., for example, about 20° C. to about 120° C., about 20° C. to about 100° C., about 20° C. to about 80°, about 20° to about 60° C., about 20° C. to about 40° C., about 20° C. to about 30° C., about 22° C. to about 26° C., about 40° C. to about 140° C., about 60° C. to about 140° C., about 80° C. to about 140° C., or about 85° C. to about 140° C. In general, as the temperature increases, the rate of reaction increases, and the time required for admixing decreases. As the temperature increases, e.g., above about 140° C., the likelihood of decomposition of the reactants, decomposition of the products, and/or evaporation of the solvents increases. In embodiments, the admixing can take place for a time in a range of about 5 minutes to about 72 hours, for example, about 5 minutes to about 60 hours, about 5 minutes to about 48 hours, about 5 minutes to about 36 hours, about 5 minutes to about 24 hours, about 5 minutes to about 12 hours, about 5 minutes to about 8 hours, about 5 minutes to about 6 hours, about 5 minutes to about 4 hours, about 5 minutes to about 2 hours, about 5 minutes to about 1 hour, about 5 minutes to about 45 minutes, about 5 minutes to about 30 minutes, about 15 minutes to about 24 hours, about 1 hour to about 8 hours, about 2 hours to about 6 hours, about 3 hours to about 5 hours, about 1 hour to about 24 hours, about 2 hours to about 23 hours, about 5 hours to about 22 hours, about 8 hours to about 21 hours, about 10 hours to about 20 hours, about 12 hours to about 19 hours, or about 14 hours to about 18 hours. As the admixing time decreases, the likelihood of the reaction not going to completion increases. Without intending to be bound by theory, it is believed that as the reaction time increases, for example, beyond about 24 hours, 48 hours, or 72 hours, while some reaction product may continue to be formed the amount provided after 24 hours, 48 hours, or 72 hours will have little effect on the total yield, while the likelihood of side reactions and decomposition of the reagents and/or products increases

In some embodiments, X is located at the 3′ carbon and X is I. In some embodiments, X is located at the 3′ carbon and X is ¹⁸F. In some embodiments, X is located at the 3′ carbon and X is ²¹¹At. In some embodiments, X is located at the 3′ carbon and X is ¹²³I. In some embodiments, X is located at the 3′ carbon and X is ¹²⁴I. In some embodiments, X is located at the 3′ carbon and X is ¹²⁵I. In some embodiments, X is located at the 3′ carbon and X is ¹³¹I. In some embodiments, X is located at the 3′ carbon and X is ⁷⁶Br. In some embodiments, X is located at the 3′ carbon and X is ⁷⁷Br. In some embodiments, X is located at the 3′ carbon and X is H₃¹¹CO. In some embodiments, X is located at the 3′ carbon and X is CF₂¹⁸F. In some embodiments, X is located at the 3′ carbon and X is CHF¹⁸F. In some embodiments, X is located at the 3′ carbon and X is OCF₂¹⁸F. In some embodiments, X is located at the 3′ carbon and X is OCHF¹⁸F. In some embodiments, X is located at the 3′ carbon and X is SCF₂¹⁸F. In some embodiments, X is located at the 3′ carbon and X is SCHF¹⁸F. In some embodiments, X is located at the 3′ carbon and X is ¹¹CN. In some embodiments, X is located at the 3′ carbon and X is ¹¹CH₃. In some embodiments, X is located at the 3′ carbon and X is H₃¹¹CS.

In some embodiments, X is located at the 2′ carbon and X is I. In some embodiments, X is located at the 2′ carbon and X is ¹⁸F. In some embodiments, X is located at the 2′ carbon and X is ²¹¹At. In some embodiments, X is located at the 2′ carbon and X is ¹²³I. In some embodiments, X is located at the 2′ carbon and X is ¹²⁴I. In some embodiments, X is located at the 2′ carbon and X is ¹²⁵I. In some embodiments, X is located at the 2′ carbon and X is ¹³¹I. In some embodiments, X is located at the 2′ carbon and X is ⁷⁶Br. In some embodiments, X is located at the 2′ carbon and X is ⁷⁷Br. In some embodiments, X is located at the 2′ carbon and X is H₃¹¹CO. In some embodiments, X is located at the 2′ carbon and X is CF₂¹⁸F. In some embodiments, X is located at the 2′ carbon and X is CHF¹⁸F. In some embodiments, X is located at the 2′ carbon and X is OCF₂¹⁸F. In some embodiments, X is located at the 2′ carbon and X is OCHF¹⁸F. In some embodiments, X is located at the 2′ carbon and X is SCF₂¹⁸F. In some embodiments, X is located at the 2′ carbon and X is SCHF¹⁸F. In some embodiments, X is located at the 2′ carbon and X is ¹¹CN. In some embodiments, X is located at the 2′ carbon and X is ¹¹CH₃. In some embodiments, X is located at the 2′ carbon and X is H₃¹¹CS.

In some embodiments, X is located at the 1′ carbon and X is I. In some embodiments, X is located at the 1′ carbon and X is ¹⁸F. In some embodiments, X is located at the 1′ carbon and X is ²¹¹At. In some embodiments, X is located at the 1′ carbon and X is ¹²³I. In some embodiments, X is located at the 1′ carbon and X is ¹²⁴I. In some embodiments, X is located at the 1′ carbon and X is ¹²⁵I. In some embodiments, X is located at the 1′ carbon and X is ¹³¹I. In some embodiments, X is located at the 1′ carbon and X is ⁷⁶Br. In some embodiments, X is located at the 1′ carbon and X is ⁷⁷Br. In some embodiments, X is located at the 1′ carbon and X is H₃¹¹CO. In some embodiments, X is located at the 1′ carbon and X is CF₂¹⁸F. In some embodiments, X is located at the 1′ carbon and X is CHF¹⁸F. In some embodiments, X is located at the 1′ carbon and X is OCF₂¹⁸F. In some embodiments, X is located at the 1′ carbon and X is OCHF¹⁸F. In some embodiments, X is located at the 1′ carbon and X is SCF₂¹⁸F. In some embodiments, X is located at the 1′ carbon and X is SCHF¹⁸F. In some embodiments, X is located at the 1′ carbon and X is ¹¹CN. In some embodiments, X is located at the 1′ carbon and X is ¹¹CH₃. In some embodiments, X is located at the 1′ carbon and X is H₃¹¹CS.

In general, each R is independently selected from H, F, ²H, CH₃, and cyclopentyl. In embodiments, each R is independently selected from H and F. In embodiments, at least one R is H. In embodiments, both R are H. In embodiments, at least one R is F. In embodiments, both R are F. In embodiments, at least one R is ²H. In embodiments, both R are 2H. In embodiments, at least one R is CH₃. In embodiments, both R are CH₃. In embodiments, at least one R is cyclopentyl. In embodiments, both R are cyclopentyl. In embodiments, X is located at carbon 3′ and both R are H. In embodiments, X is located at carbon 3′ and both R are F. In embodiments, X is located at carbon 2′ and both R are H. In embodiments, X is located at carbon 2′ and both R are F. In embodiments, X is located at carbon 1′ and both R are H. In embodiments, X is located at carbon 1′ and both R are F.

In embodiments, Q and X are located at carbon 3′ and all R are H. In embodiments, Q and X are located at carbon 3′ and all R are F. In embodiments, Q and X are located at carbon 2′ and all R are H. In embodiments, Q and X are located at carbon 2′ and all R are F. In embodiments, Q and X are located at carbon 1′ and all R are H. In embodiments, Q and X are located at carbon 1′ and all R are F.

In embodiments, the methods of the disclosure can include preparing a radiolabeled compound having a structure according to formula (II), the method comprising admixing a radiolabeled salt, MX, with a compound having a structure according to formula (X):

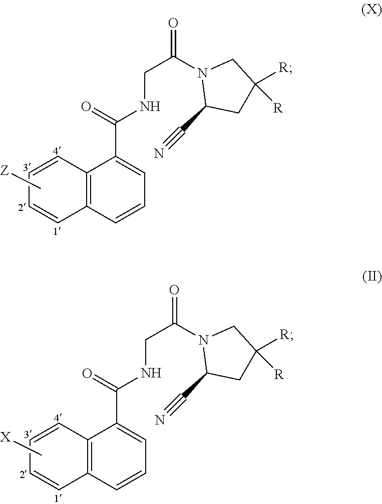

wherein Z has a structure selected from the group of:

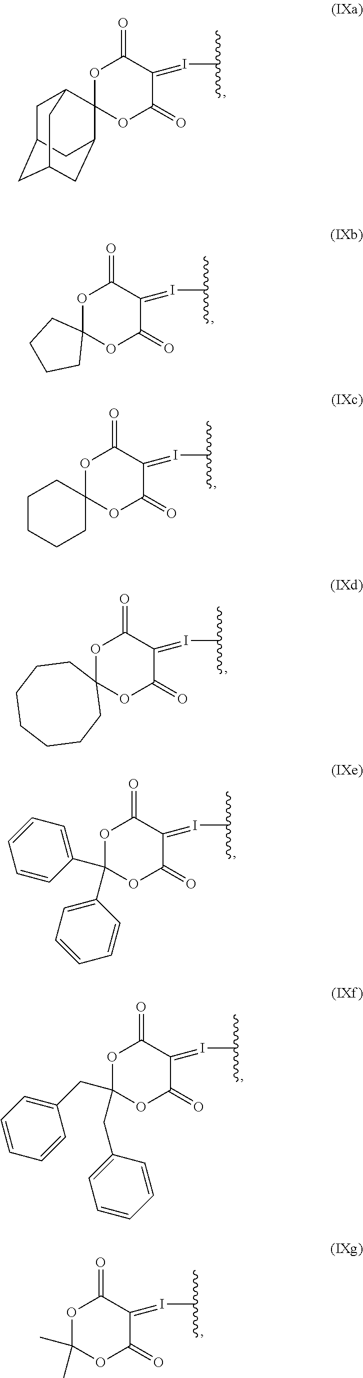

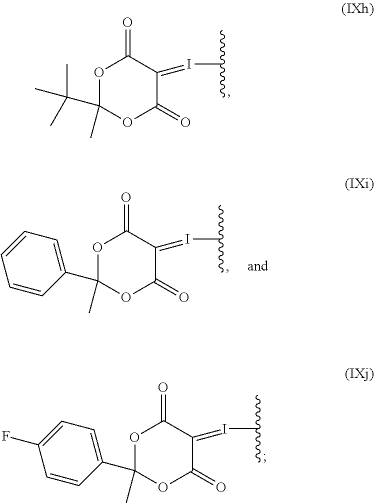 [SMILES: *I=C1C(=O)OC(C)(C(C)(C)C)OC1=O ; *I=C1C(=O)OC(C)(c2ccccc2)OC1=O ; *I=C1C(=O)OC(C)(c2ccc(F)cc2)OC1=O]

and wherein in formula (X) and formula (II), Z and X are located at carbon 1′, 2′, or 3′, and the location of Z in formula (X) and the location of X in formula (II) are the same; M is selected from the group of K, Li, Na, Rb, Cs, tetraethylammonium, and tetrabutylammonium; in the salt, MX, and in formula (II), X is selected from the group of ¹⁸F, ²¹¹At, ¹²³I, ¹²⁴I, ¹²⁵I, ¹³¹I, ⁷⁶Br, ⁷⁷Br, and the X in the salt is the same as the X in formula (II); and each R is independently selected from H, F, ²H, CH₃, and cyclopentyl, and the R in formula (II) are the same as the R in formula (X).

In general, M is any monovalent cation capable of forming a salt with the anion, X. In embodiments, M is selected from the group of K, Li, Na, Rb, Cs, tetraethylammonium ((ethyl)₄N), and tetrabutylammonium ((butyl)₄N). In embodiments, M is selected from the group of K, Li, and Na. In embodiments, M is K. In embodiments, M is tetraethylammonium.

In general, the compound of having a structure according to formula (X) can be identical to any compound of having a structure of formula (II) disclosed herein, except that the X group of the compound of formula (II) is replaced with a Z group as defined herein. In embodiments, Z is

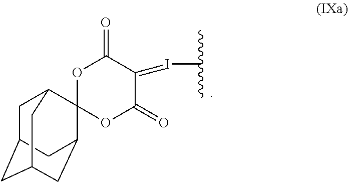 [SMILES: *I=C1C(=O)OC2(OC1=O)C1CC3CC(C1)CC2C3]

In embodiments, Z is

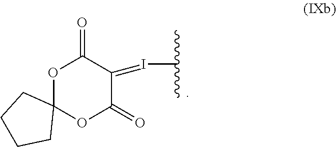 [SMILES: *I=C1C(=O)OC2(CCCC2)OC1=O]

In embodiments, Z is

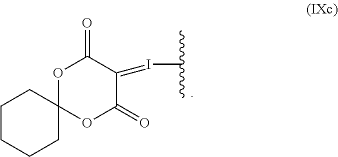 [SMILES: *I=C1C(=O)OC2(CCCCC2)OC1=O]

In embodiments, Z is

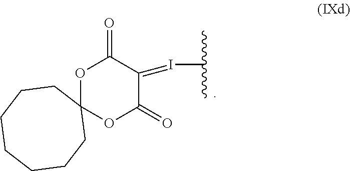 [SMILES: *I=C1C(=O)OC2(CCCCCCC2)OC1=O]

In embodiments, Z is

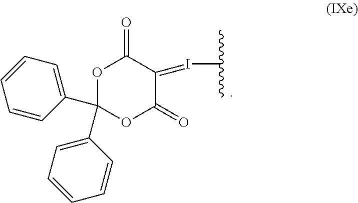 [SMILES: *I=C1C(=O)OC(c2ccccc2)(c2ccccc2)OC1=O]

In embodiments, Z is

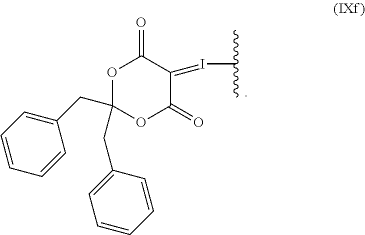 [SMILES: *I=C1C(=O)OC(Cc2ccccc2)(Cc2ccccc2)OC1=O]

In embodiments, Z is

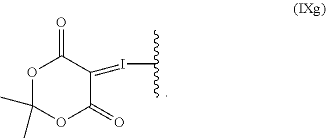 [SMILES: *I=C1C(=O)OC(C)(C)OC1=O]

In embodiments, Z is

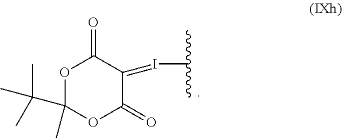 [SMILES: *I=C1C(=O)OC(C)(C(C)(C)C)OC1=O]

In embodiments, Z is

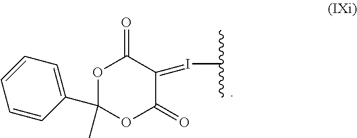 [SMILES: *I=C1C(=O)OC(C)(c2ccccc2)OC1=O]

In embodiments, Z is

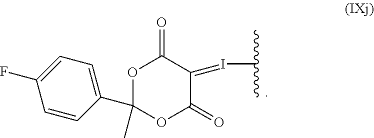 [SMILES: *I=C1C(=O)OC(C)(c2ccc(F)cc2)OC1=O]

The methods of the disclosure convert the Z group of the compound having a structure according to formula (X) to an X group, without otherwise altering the structure of the compound of formula (X). Thus, the Z group of the compound having a structure according to formula (X) and the X group of the compound having a structure according to formula (II) are located at the same position on the naphthalene ring and the R groups of the compound having a structure according to formula (II) are the same as the R groups of the compound having a structure according to formula (X). In embodiments, Z and X are each located at carbon 1′. In embodiments, Z and X are each located at carbon 2′. In embodiments, Z and X are each located at carbon 3′.

In general, the admixing of the compound having a structure according to formula (X) and the radiolabeled salt, MX, is done under conditions sufficient to provide a compound of having a structure according to formula (II). In general, the admixing can take place in a solution comprising a solvent for the compound having a structure according to formula (X), a solvent for the salt, or a solvent for the compound having a structure according to formula (X) and the salt. In embodiments, the solvent can comprise any solvent suitable for solvating the compound having a structure according to formula (X), the salt, or both. In embodiments, the solvent comprises dimethylsulfoxide (DMSO), dimethylformamide (DMF), dimethylacetamide (DMA), or a combination thereof. In embodiments, the solvent comprises DMSO. In embodiments, the solvent comprises pyridine and DMF. In embodiments, the solvent comprises pyridine and DMA. The concentrations of the compound having a structure according to formula (X) and the salt can be any concentration. The concentrations are typically chosen such that the compound having a structure according to formula (X) and/or the salt is fully soluble, without forming saturated solutions.

In general, the admixing can take place at any suitable temperature for any suitable time. In embodiments, the admixing can take place at a temperature in a range of about 20° C. to about 140° C., for example, about 20° C. to about 120° C., about 20° C. to about 100° C., about 20° C. to about 80°, about 20° to about 60° C., about 20° C. to about 40° C., about 20° C. to about 30° C., about 22° C. to about 26° C., about 40° C. to about 140° C., about 60° C. to about 140° C., about 80° C. to about 140° C., or about 85° C. to about 140° C. In general, as the temperature increases, the rate of reaction increases, and the time required for admixing decreases. As the temperature increases, e.g., above about 140° C., the likelihood of decomposition of the reactants, decomposition of the products, and/or evaporation of the solvents increases. In embodiments, the admixing can take place for a time in a range of about 5 minutes to about 72 hours, for example, about 5 minutes to about 60 hours, about 5 minutes to about 48 hours, about 5 minutes to about 36 hours, about 5 minutes to about 24 hours, about 5 minutes to about 12 hours, about 5 minutes to about 8 hours, about 5 minutes to about 6 hours, about 5 minutes to about 4 hours, about 5 minutes to about 2 hours, about 5 minutes to about 1 hour, about 5 minutes to about 45 minutes, or about 5 minutes to about 30 minutes. As the admixing time decreases, the likelihood of the reaction not going to completion increases. Without intending to be bound by theory, it is believed that as the reaction time increases, for example, beyond about 24 hours, 48 hours, or 72 hours, while some reaction product may continue to be formed the amount provided after 24 hours, 48 hours, or 72 hours will have little effect on the total yield, while the likelihood of side reactions and decomposition of the reagents and/or products increases.

The method of preparing a radiolabeled FAPI having a structure according to formula (II) can further include preparing the compound having a structure according to formula (X), by admixing a compound having a structure according to formula (IIa):

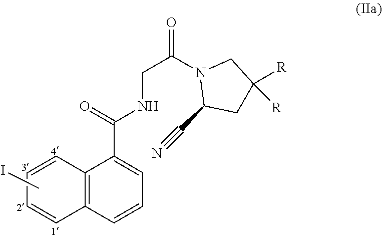

with 1-chloromethyl-4-fluoro-1,4-diazoniabicyclo[2.2.2]octanebis(tetrafluoroborate), and trimethylsilyl acetate to form a reaction product. The method can further include admixing the reaction product of the compound of formula (IIa) with 1-chloromethyl-4-fluoro-1,4-diazoniabicyclo[2.2.2]octanebis(tetrafluoroborate), and trimethylsilyl acetate with a dioxane-dione and sodium carbonate to form the compound having a structure according to formula (X).

The methods of the disclosure convert the I of the compound having a structure according to formula (IIa) to a Z group, without otherwise altering the structure of the compound of formula (IIa). Thus, the Z group of the compound having a structure according to formula (X) and the I of the compound having a structure according to formula (IIa) are located at the same position on the naphthalene ring and the R groups of the compound having a structure according to formula (IIa) are the same as the R groups of the compound having a structure according to formula (X).

In embodiments, the compound of formula (IIa) is admixed with 1-chloromethyl-4-fluoro-1,4-diazoniabicyclo[2.2.2]octanebis(tetrafluoroborate) under conditions sufficient to provide a reaction product that can be converted to the compound of having a structure according to formula (X). In general, the admixing can take place in a solution comprising a solvent for the compound having a structure according to formula (IIa), a solvent for the 1-chloromethyl-4-fluoro-1,4-diazoniabicyclo[2.2.2]octanebis(tetrafluoroborate), or a solvent for the compound having a structure according to formula (IIa) and the 1-chloromethyl-4-fluoro-1,4-diazoniabicyclo[2.2.2]octanebis(tetrafluoroborate). In embodiments, the solvent can comprise any solvent suitable for solvating the compound having a structure according to formula (IIa), the 1-chloromethyl-4-fluoro-1,4-diazoniabicyclo[2.2.2]octanebis(tetrafluoroborate), or both. In embodiments, the solvent comprises acetonitrile. The concentrations of the compound having a structure according to formula (IIa) can be any concentration. The concentration is typically chosen such that the compound having a structure according to formula (IIa) is fully soluble, without forming saturated solutions.

In embodiments, the compound of formula (IIa) is admixed with 1-chloromethyl-4-fluoro-1,4-diazoniabicyclo[2.2.2]octanebis(tetrafluoroborate) in the presence of trimethyl silyl acetate. In embodiments, the admixing comprises dropwise addition of 1-chloromethyl-4-fluoro-1,4-diazoniabicyclo[2.2.2]octanebis(tetrafluoroborate) to a solution of the compound having a structure according to formula (IIa) and trimethylsilyl acetate.

In general, the admixing of the compound of formula (IIa) is admixed with 1-chloromethyl-4-fluoro-1,4-diazoniabicyclo[2.2.2]octanebis(tetrafluoroborate) can take place at any suitable temperature for any suitable time. In embodiments, the admixing can take place at a temperature in a range of about 20° C. to about 140° C., for example, about 20° C. to about 120° C., about 20° C. to about 100° C., about 20° C. to about 80°, about 20° to about 60° C., about 20° C. to about 40° C., about 20° C. to about 30° C., about 22° C. to about 26° C., about 40° C. to about 140° C., about 60° C. to about 140° C., about 80° C. to about 140° C., or about 85° C. to about 140° C. In general, as the temperature increases, the rate of reaction increases, and the time required for admixing decreases. As the temperature increases, e.g., above about 140° C., the likelihood of decomposition of the reactants, decomposition of the products, and/or evaporation of the solvents increases. In embodiments, the admixing can take place for a time in a range of about 5 minutes to about 72 hours, for example, about 5 minutes to about 60 hours, about 5 minutes to about 48 hours, about 5 minutes to about 36 hours, about 5 minutes to about 24 hours, about 5 minutes to about 12 hours, about 5 minutes to about 8 hours, about 5 minutes to about 6 hours, about 5 minutes to about 4 hours, about 5 minutes to about 2 hours, about 5 minutes to about 1 hour, about 5 minutes to about 45 minutes, about 5 minutes to about 30 minutes, about 15 minutes to about 24 hours, about 1 hour to about 24 hours, about 2 hours to about 23 hours, about 5 hours to about 22 hours, about 8 hours to about 21 hours, about 10 hours to about 20 hours, about 12 hours to about 19 hours, or about 14 hours to about 18 hours. As the admixing time decreases, the likelihood of the reaction not going to completion increases. Without intending to be bound by theory, it is believed that as the reaction time increases, for example, beyond about 24 hours, 48 hours, or 72 hours, while some reaction product may continue to be formed the amount provided after 24 hours, 48 hours, or 72 hours will have little effect on the total yield, while the likelihood of side reactions and decomposition of the reagents and/or products increases.

In embodiments, the admixing of the reaction product of the compound of formula (IIa) with 1-chloromethyl-4-fluoro-1,4-diazoniabicyclo[2.2.2]octanebis(tetrafluoroborate) is admixed with a dioxane-dione under conditions sufficient to provide the compound of having a structure according to formula (X). In general, the admixing can take place in a solution comprising a solvent for the reaction product, a solvent for the dioxane-dione, or both. In embodiments, the solvent can comprise any solvent suitable for solvating the reaction product, a solvent for the dioxane-dione, or both. In embodiments, the solvent comprises ethanol. The concentrations of the reactants are typically chosen such that the reactants are fully soluble, without forming saturated solutions.

In embodiments, the dioxane-dione is selected from the group of (1r,3r,5r,7r)-spiro[adamantan-2,2′-[1,3]-dioxane]-4′,6′-dione (SPIAd), 6,10-dioxaspiro[4.5]decane-7,9-dione, 1,5-dioxaspiro[5.5]undecane-2,4-dione, 1,5-dioxaspiro[5.7]tridecane-2,4-dione, 2,2-diphenyl-1,3-dioxane-4,6-dione, 2,2-dibenzyl-1,3-dioxane-4,6-dione, 5,5-dimethylcyclohexane-1,3-dione, 2-(tert-butyl)-2-methyl-1,3-dioxane-4,6-dione, 2-methyl-2-phenyl-1,3-dioxane-4,6-dione, and 2-(4-fluorophenyl)-2-methyl-1,3-dioxane-4,6-dione. In embodiments, the dioxane-dione is (1r,3r,5r,7r)-spiro[adamantan-2,2′-[1,3]-dioxane]-4′,6′-dione (SPIAd). In embodiments, the admixing comprises dropwise addition of the dioxane-dione to the reaction product.

In general, the admixing of the reaction product and the dioxane-dione can take place at any suitable temperature for any suitable time. In embodiments, the admixing can take place at a temperature in a range of about 20° C. to about 140° C., for example, about 20° C. to about 120° C., about 20° C. to about 100° C., about 20° C. to about 80°, about 20° to about 60° C., about 20° C. to about 40° C., about 20° C. to about 30° C., about 22° C. to about 26° C., about 40° C. to about 140° C., about 60° C. to about 140° C., about 80° C. to about 140° C., or about 85° C. to about 140° C. In general, as the temperature increases, the rate of reaction increases, and the time required for admixing decreases. As the temperature increases, e.g., above about 140° C., the likelihood of decomposition of the reactants, decomposition of the products, and/or evaporation of the solvents increases. In embodiments, the admixing can take place for a time in a range of about 5 minutes to about 72 hours, for example, about 5 minutes to about 60 hours, about 5 minutes to about 48 hours, about 5 minutes to about 36 hours, about 5 minutes to about 24 hours, about 5 minutes to about 12 hours, about 5 minutes to about 8 hours, about 5 minutes to about 6 hours, about 5 minutes to about 4 hours, about 5 minutes to about 2 hours, about 5 minutes to about 1 hour, about 5 minutes to about 45 minutes, about 5 minutes to about 30 minutes, about 15 minutes to about 24 hours, about 1 hour to about 8 hours, about 2 hours to about 6 hours, about 3 hours to about 5 hours, about 1 hour to about 24 hours, about 2 hours to about 23 hours, about 5 hours to about 22 hours, about 8 hours to about 21 hours, about 10 hours to about 20 hours, about 12 hours to about 19 hours, or about 14 hours to about 18 hours. As the admixing time decreases, the likelihood of the reaction not going to completion increases. Without intending to be bound by theory, it is believed that as the reaction time increases, for example, beyond about 24 hours, 48 hours, or 72 hours, while some reaction product may continue to be formed the amount provided after 24 hours, 48 hours, or 72 hours will have little effect on the total yield, while the likelihood of side reactions and decomposition of the reagents and/or products increases

In some embodiments, X is located at the 3′ carbon and X is I. In some embodiments, X is located at the 3′ carbon and X is ¹⁸F. In some embodiments, X is located at the 3′ carbon and X is ²¹¹At. In some embodiments, X is located at the 3′ carbon and X is ¹²³I. In some embodiments, X is located at the 3′ carbon and X is ¹²⁴I. In some embodiments, X is located at the 3′ carbon and X is ¹²⁵I. In some embodiments, X is located at the 3′ carbon and X is ¹³¹I. In some embodiments, X is located at the 3′ carbon and X is ⁷⁶Br. In some embodiments, X is located at the 3′ carbon and X is ⁷⁷Br. In some embodiments, X is located at the 3′ carbon and X is H₃¹¹CO. In some embodiments, X is located at the 3′ carbon and X is CF₂¹⁸F. In some embodiments, X is located at the 3′ carbon and X is CHF¹⁸F. In some embodiments, X is located at the 3′ carbon and X is OCF₂¹⁸F. In some embodiments, X is located at the 3′ carbon and X is OCHF¹⁸F. In some embodiments, X is located at the 3′ carbon and X is SCF₂¹⁸F. In some embodiments, X is located at the 3′ carbon and X is SCHF¹⁸F. In some embodiments, X is located at the 3′ carbon and X is ¹¹CN. In some embodiments, X is located at the 3′ carbon and X is ¹¹CH₃. In some embodiments, X is located at the 3′ carbon and X is H₃¹¹CS.

In some embodiments, X is located at the 2′ carbon and X is I. In some embodiments, X is located at the 2′ carbon and X is ¹⁸F. In some embodiments, X is located at the 2′ carbon and X is ²¹¹At. In some embodiments, X is located at the 2′ carbon and X is ¹²³I. In some embodiments, X is located at the 2′ carbon and X is ¹²⁴I. In some embodiments, X is located at the 2′ carbon and X is ¹²⁵I. In some embodiments, X is located at the 2′ carbon and X is ¹³¹I. In some embodiments, X is located at the 2′ carbon and X is ⁷⁶Br. In some embodiments, X is located at the 2′ carbon and X is ⁷⁷Br. In some embodiments, X is located at the 2′ carbon and X is H₃¹¹CO. In some embodiments, X is located at the 2′ carbon and X is CF₂¹⁸F. In some embodiments, X is located at the 2′ carbon and X is CHF¹⁸F. In some embodiments, X is located at the 2′ carbon and X is OCF₂¹⁸F. In some embodiments, X is located at the 2′ carbon and X is OCHF¹⁸F. In some embodiments, X is located at the 2′ carbon and X is SCF₂¹⁸F. In some embodiments, X is located at the 2′ carbon and X is SCHF¹⁸F. In some embodiments, X is located at the 2′ carbon and X is ¹¹CN. In some embodiments, X is located at the 2′ carbon and X is ¹¹CH₃. In some embodiments, X is located at the 2′ carbon and X is H₃¹¹CS.

In some embodiments, X is located at the 1′ carbon and X is I. In some embodiments, X is located at the 1′ carbon and X is ¹⁸F. In some embodiments, X is located at the 1′ carbon and X is ²¹¹At. In some embodiments, X is located at the 1′ carbon and X is ¹²³I. In some embodiments, X is located at the 1′ carbon and X is ¹²⁴I. In some embodiments, X is located at the 1′ carbon and X is ¹²⁵I. In some embodiments, X is located at the 1′ carbon and X is ¹³¹I. In some embodiments, X is located at the 1′ carbon and X is ⁷⁶Br. In some embodiments, X is located at the 1′ carbon and X is ⁷⁷Br. In some embodiments, X is located at the 1′ carbon and X is H₃¹¹CO. In some embodiments, X is located at the 1′ carbon and X is CF₂¹⁸F. In some embodiments, X is located at the 1′ carbon and X is CHF¹⁸F. In some embodiments, X is located at the 1′ carbon and X is OCF₂¹⁸F. In some embodiments, X is located at the 1′ carbon and X is OCHF¹⁸F. In some embodiments, X is located at the 1′ carbon and X is SCF₂¹⁸F. In some embodiments, X is located at the 1′ carbon and X is SCHF¹⁸F. In some embodiments, X is located at the 1′ carbon and X is ¹¹CN. In some embodiments, X is located at the 1′ carbon and X is ¹¹CH₃. In some embodiments, X is located at the 1′ carbon and X is H₃¹¹CS.

In general, each R is independently selected from H, F, ²H, CH₃, and cyclopentyl. In embodiments, each R is independently selected from H and F. In embodiments, at least one R is H. In embodiments, both R are H. In embodiments, at least one R is F. In embodiments, both R are F. In embodiments, at least one R is ²H. In embodiments, both R are 2H. In embodiments, at least one R is CH₃. In embodiments, both R are CH₃. In embodiments, at least one R is cyclopentyl. In embodiments, both R are cyclopentyl. In embodiments, X is located at carbon 3′ and both R are H. In embodiments, X is located at carbon 3′ and both R are F. In embodiments, X is located at carbon 2′ and both R are H. In embodiments, X is located at carbon 2′ and both R are F. In embodiments, X is located at carbon 1′ and both R are H. In embodiments, X is located at carbon 1′ and both R are F.

In embodiments, Q and X are located at carbon 3′ and all R are H. In embodiments, Q and X are located at carbon 3′ and all R are F. In embodiments, Q and X are located at carbon 2′ and all R are H. In embodiments, Q and X are located at carbon 2′ and all R are F. In embodiments, Q and X are located at carbon 1′ and all R are H. In embodiments, Q and X are located at carbon 1′ and all R are F.

In embodiments, the methods of the disclosure can include preparing a radiolabeled compound having a structure according to formula (III), the method comprising admixing a radiolabeled salt, MX, with a compound having a structure according to formula (XI):

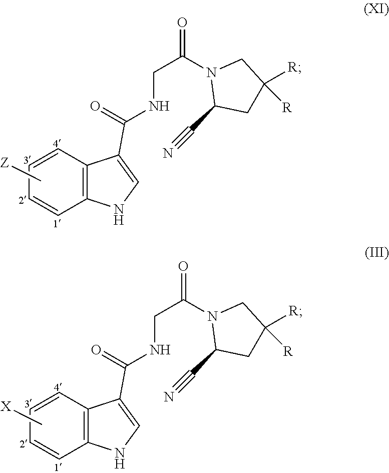

wherein Z has a structure selected from the group of:

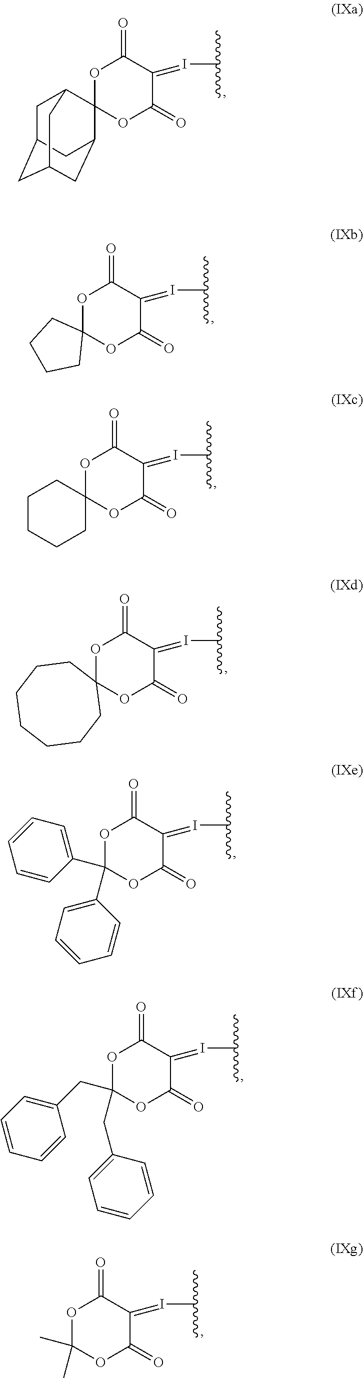

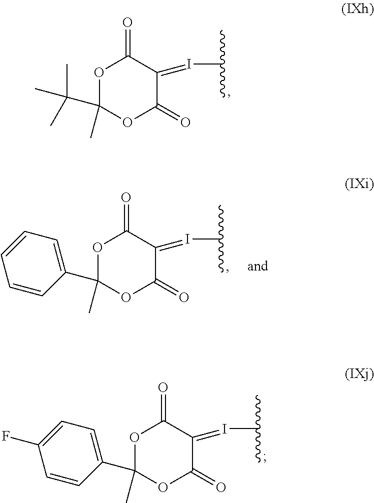 [SMILES: *I=C1C(=O)OC(C)(C(C)(C)C)OC1=O ; *I=C1C(=O)OC(C)(c2ccccc2)OC1=O ; *I=C1C(=O)OC(C)(c2ccc(F)cc2)OC1=O]

and wherein in formula (XI) and formula (III), Z and X are located at carbon 1′, 2′, or 3′, and the location of Z in formula (XI) and the location of X in formula (III) are the same; M is selected from the group of K, Li, Na, Rb, Cs, tetraethylammonium, and tetrabutylammonium; in the salt, MX, and in formula (III); X is selected from the group of ¹⁸F, ²¹¹At, ¹²³I, ¹²⁴I, ¹²⁵I, ¹³¹I, ⁷⁶Br, ⁷⁷Br, and X in the salt is the same as X in formula (III); and each R is independently selected from H, F, ²H, CH₃, and cyclopentyl, and the R in formula (III) are the same as the R in formula (XI).

In general, M is any monovalent cation capable of forming a salt with the anion, X. In embodiments, M is selected from the group of K, Li, Na, Rb, Cs, tetraethylammonium ((ethyl)₄N), and tetrabutylammonium ((butyl)₄N). In embodiments, M is selected from the group of K, Li, and Na. In embodiments, M is K. In embodiments, M is tetraethylammonium.

In general, the compound of having a structure according to formula (XI) can be identical to any compound of having a structure of formula (III) disclosed herein, except that the X group of the compound of formula (III) is replaced with a Z group as defined herein. In embodiments, Z is

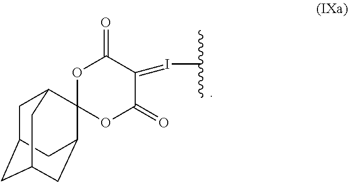 [SMILES: *I=C1C(=O)OC2(OC1=O)C1CC3CC(C1)CC2C3]

In embodiments, Z is

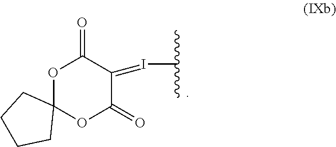 [SMILES: *I=C1C(=O)OC2(CCCC2)OC1=O]

In embodiments, Z is

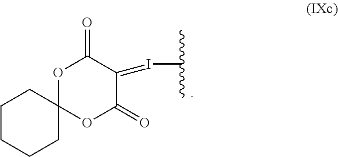 [SMILES: *I=C1C(=O)OC2(CCCCC2)OC1=O]

In embodiments, Z is

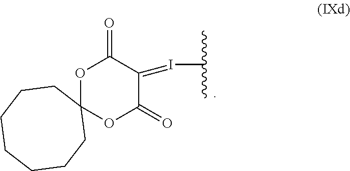 [SMILES: *I=C1C(=O)OC2(CCCCCCC2)OC1=O]

In embodiments, Z is

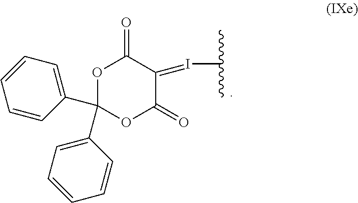 [SMILES: *I=C1C(=O)OC(c2ccccc2)(c2ccccc2)OC1=O]

In embodiments, Z is

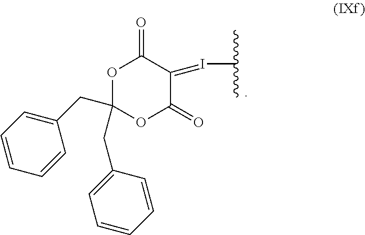 [SMILES: *I=C1C(=O)OC(Cc2ccccc2)(Cc2ccccc2)OC1=O]

In embodiments, Z is

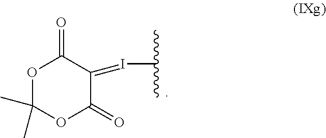 [SMILES: *I=C1C(=O)OC(C)(C)OC1=O]

In embodiments, Z is

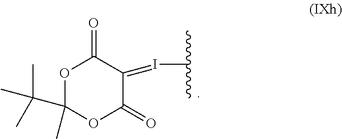 [SMILES: *I=C1C(=O)OC(C)(C(C)(C)C)OC1=O]

In embodiments, Z is

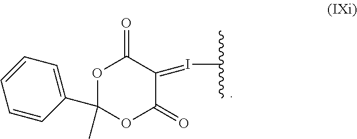 [SMILES: *I=C1C(=O)OC(C)(c2ccccc2)OC1=O]

In embodiments, Z is

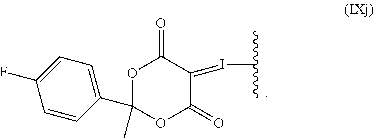 [SMILES: *I=C1C(=O)OC(C)(c2ccc(F)cc2)OC1=O]

The methods of the disclosure convert the Z group of the compound having a structure according to formula (XI) to an X group, without otherwise altering the structure of the compound of formula (XI). Thus, the Z group of the compound having a structure according to formula (XI) and the X group of the compound having a structure according to formula (III) are located at the same position on the indole ring and the R groups of the compound having a structure according to formula (III) are the same as the R groups of the compound having a structure according to formula (XI). In embodiments, Z and X are each located at carbon 1′. In embodiments, Z and X are each located at carbon 2′. In embodiments, Z and X are each located at carbon 3′.

In general, the admixing of the compound having a structure according to formula (XI) and the radiolabeled salt, MX, is done under conditions sufficient to provide a compound of having a structure according to formula (III). In general, the admixing can take place in a solution comprising a solvent for the compound having a structure according to formula (XI), a solvent for the salt, or a solvent for the compound having a structure according to formula (XI) and the salt. In embodiments, the solvent can comprise any solvent suitable for solvating the compound having a structure according to formula (XI), the salt, or both. In embodiments, the solvent comprises dimethylsulfoxide (DMSO), dimethylformamide (DMF), dimethylacetamide (DMA), or a combination thereof. In embodiments, the solvent comprises DMSO. In embodiments, the solvent comprises pyridine and DMF. In embodiments, the solvent comprises pyridine and DMA. The concentrations of the compound having a structure according to formula (XI) and the salt can be any concentration. The concentrations are typically chosen such that the compound having a structure according to formula (XI) and/or the salt is fully soluble, without forming saturated solutions.

In general, the admixing can take place at any suitable temperature for any suitable time. In embodiments, the admixing can take place at a temperature in a range of about 20° C. to about 140° C., for example, about 20° C. to about 120° C., about 20° C. to about 100° C., about 20° C. to about 80°, about 20° to about 60° C., about 20° C. to about 40° C., about 20° C. to about 30° C., about 22° C. to about 26° C., about 40° C. to about 140° C., about 60° C. to about 140° C., about 80° C. to about 140° C., or about 85° C. to about 140° C. In general, as the temperature increases, the rate of reaction increases, and the time required for admixing decreases. As the temperature increases, e.g., above about 140° C., the likelihood of decomposition of the reactants, decomposition of the products, and/or evaporation of the solvents increases. In embodiments, the admixing can take place for a time in a range of about 5 minutes to about 72 hours, for example, about 5 minutes to about 60 hours, about 5 minutes to about 48 hours, about 5 minutes to about 36 hours, about 5 minutes to about 24 hours, about 5 minutes to about 12 hours, about 5 minutes to about 8 hours, about 5 minutes to about 6 hours, about 5 minutes to about 4 hours, about 5 minutes to about 2 hours, about 5 minutes to about 1 hour, about 5 minutes to about 45 minutes, or about 5 minutes to about 30 minutes. As the admixing time decreases, the likelihood of the reaction not going to completion increases. Without intending to be bound by theory, it is believed that as the reaction time increases, for example, beyond about 24 hours, 48 hours, or 72 hours, while some reaction product may continue to be formed the amount provided after 24 hours, 48 hours, or 72 hours will have little effect on the total yield, while the likelihood of side reactions and decomposition of the reagents and/or products increases.

The method of preparing a radiolabeled FAPI having a structure according to formula (ill) can further include preparing the compound having a structure according to formula (XI), by admixing a compound having a structure according to formula (IIIa):

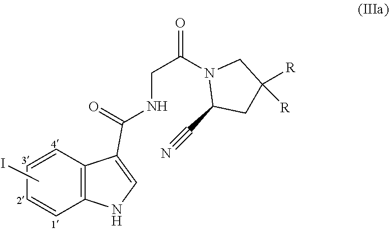

with 1-chloromethyl-4-fluoro-1,4-diazoniabicyclo[2.2.2]octanebis(tetrafluoroborate), and trimethylsilyl acetate to form a reaction product. The method can further include admixing the reaction product of the compound of formula (IIIa) with 1-chloromethyl-4-fluoro-1,4-diazoniabicyclo[2.2.2]octanebis(tetrafluoroborate), and trimethylsilyl acetate with a dioxane-dione and sodium carbonate to form the compound having a structure according to formula (XI).

The methods of the disclosure convert the I of the compound having a structure according to formula (IIIa) to a Z group, without otherwise altering the structure of the compound of formula (IIIa). Thus, the Z group of the compound having a structure according to formula (XI) and the I of the compound having a structure according to formula (IIIa) are located at the same position on the indole ring and the R groups of the compound having a structure according to formula (IIIa) are the same as the R groups of the compound having a structure according to formula (XI).

In embodiments, the compound of formula (IIIa) is admixed with 1-chloromethyl-4-fluoro-1,4-diazoniabicyclo[2.2.2]octanebis(tetrafluoroborate) under conditions sufficient to provide a reaction product that can be converted to the compound of having a structure according to formula (XI). In general, the admixing can take place in a solution comprising a solvent for the compound having a structure according to formula (IIIa), a solvent for the 1-chloromethyl-4-fluoro-1,4-diazoniabicyclo[2.2.2]octanebis(tetrafluoroborate), or a solvent for the compound having a structure according to formula (IIIa) and the 1-chloromethyl-4-fluoro-1,4-diazoniabicyclo[2.2.2]octanebis(tetrafluoroborate). In embodiments, the solvent can comprise any solvent suitable for solvating the compound having a structure according to formula (IlIa), the 1-chloromethyl-4-fluoro-1,4-diazoniabicyclo[2.2.2]octanebis(tetrafluoroborate), or both. In embodiments, the solvent comprises acetonitrile. The concentrations of the compound having a structure according to formula (IIIa) can be any concentration. The concentration is typically chosen such that the compound having a structure according to formula (IIIa) is fully soluble, without forming saturated solutions.

In embodiments, the compound of formula (IIIa) is admixed with 1-chloromethyl-4-fluoro-1,4-diazoniabicyclo[2.2.2]octanebis(tetrafluoroborate) in the presence of trimethyl silyl acetate. In embodiments, the admixing comprises dropwise addition of 1-chloromethyl-4-fluoro-1,4-diazoniabicyclo[2.2.2]octanebis(tetrafluoroborate) to a solution of the compound having a structure according to formula (IIIa) and trimethylsilyl acetate.

In general, the admixing of the compound of formula (IIIa) is admixed with 1-chloromethyl-4-fluoro-1,4-diazoniabicyclo[2.2.2]octanebis(tetrafluoroborate) can take place at any suitable temperature for any suitable time. In embodiments, the admixing can take place at a temperature in a range of about 20° C. to about 140° C., for example, about 20° C. to about 120° C., about 20° C. to about 100° C., about 20° C. to about 80°, about 20° to about 60° C., about 20° C. to about 40° C., about 20° C. to about 30° C., about 22° C. to about 26° C., about 40° C. to about 140° C., about 60° C. to about 140° C., about 80° C. to about 140° C., or about 85° C. to about 140° C. In general, as the temperature increases, the rate of reaction increases, and the time required for admixing decreases. As the temperature increases, e.g., above about 140° C., the likelihood of decomposition of the reactants, decomposition of the products, and/or evaporation of the solvents increases. In embodiments, the admixing can take place for a time in a range of about 5 minutes to about 72 hours, for example, about 5 minutes to about 60 hours, about 5 minutes to about 48 hours, about 5 minutes to about 36 hours, about 5 minutes to about 24 hours, about 5 minutes to about 12 hours, about 5 minutes to about 8 hours, about 5 minutes to about 6 hours, about 5 minutes to about 4 hours, about 5 minutes to about 2 hours, about 5 minutes to about 1 hour, about 5 minutes to about 45 minutes, about 5 minutes to about 30 minutes, about 15 minutes to about 24 hours, about 1 hour to about 24 hours, about 2 hours to about 23 hours, about 5 hours to about 22 hours, about 8 hours to about 21 hours, about 10 hours to about 20 hours, about 12 hours to about 19 hours, or about 14 hours to about 18 hours. As the admixing time decreases, the likelihood of the reaction not going to completion increases. Without intending to be bound by theory, it is believed that as the reaction time increases, for example, beyond about 24 hours, 48 hours, or 72 hours, while some reaction product may continue to be formed the amount provided after 24 hours, 48 hours, or 72 hours will have little effect on the total yield, while the likelihood of side reactions and decomposition of the reagents and/or products increases.

In embodiments, the admixing of the reaction product of the compound of formula (IIIa) with 1-chloromethyl-4-fluoro-1,4-diazoniabicyclo[2.2.2]octanebis(tetrafluoroborate) is admixed with a dioxane-dione under conditions sufficient to provide the compound of having a structure according to formula (XI). In general, the admixing can take place in a solution comprising a solvent for the reaction product, a solvent for the dioxane-dione, or both. In embodiments, the solvent can comprise any solvent suitable for solvating the reaction product, a solvent for the dioxane-dione, or both. In embodiments, the solvent comprises ethanol. The concentrations of the reactants are typically chosen such that the reactants are fully soluble, without forming saturated solutions.

In embodiments, the dioxane-dione is selected from the group of (1r,3r,5r,7r)-spiro[adamantan-2,2′-[1,3]-dioxane]-4′,6′-dione (SPIAd), 6,10-dioxaspiro[4.5]decane-7,9-dione, 1,5-dioxaspiro[5.5]undecane-2,4-dione, 1,5-dioxaspiro[5.7]tridecane-2,4-dione, 2,2-diphenyl-1,3-dioxane-4,6-dione, 2,2-dibenzyl-1,3-dioxane-4,6-dione, 5,5-dimethylcyclohexane-1,3-dione, 2-(tert-butyl)-2-methyl-1,3-dioxane-4,6-dione, 2-methyl-2-phenyl-1,3-dioxane-4,6-dione, and 2-(4-fluorophenyl)-2-methyl-1,3-dioxane-4,6-dione. In embodiments, the dioxane-dione is (1r,3r,5r,7r)-spiro[adamantan-2,2′-[1,3]-dioxane]-4′,6′-dione (SPIAd). In embodiments, the admixing comprises dropwise addition of the dioxane-dione to the reaction product.

In general, the admixing of the reaction product and the dioxane-dione can take place at any suitable temperature for any suitable time. In embodiments, the admixing can take place at a temperature in a range of about 20° C. to about 140° C., for example, about 20° C. to about 120° C., about 20° C. to about 100° C., about 20° C. to about 80°, about 20° to about 60° C., about 20° C. to about 40° C., about 20° C. to about 30° C., about 22° C. to about 26° C., about 40° C. to about 140° C., about 60° C. to about 140° C., about 80° C. to about 140° C., or about 85° C. to about 140° C. In general, as the temperature increases, the rate of reaction increases, and the time required for admixing decreases. As the temperature increases, e.g., above about 140° C., the likelihood of decomposition of the reactants, decomposition of the products, and/or evaporation of the solvents increases. In embodiments, the admixing can take place for a time in a range of about 5 minutes to about 72 hours, for example, about 5 minutes to about 60 hours, about 5 minutes to about 48 hours, about 5 minutes to about 36 hours, about 5 minutes to about 24 hours, about 5 minutes to about 12 hours, about 5 minutes to about 8 hours, about 5 minutes to about 6 hours, about 5 minutes to about 4 hours, about 5 minutes to about 2 hours, about 5 minutes to about 1 hour, about 5 minutes to about 45 minutes, about 5 minutes to about 30 minutes, about 15 minutes to about 24 hours, about 1 hour to about 8 hours, about 2 hours to about 6 hours, about 3 hours to about 5 hours, about 1 hour to about 24 hours, about 2 hours to about 23 hours, about 5 hours to about 22 hours, about 8 hours to about 21 hours, about 10 hours to about 20 hours, about 12 hours to about 19 hours, or about 14 hours to about 18 hours. As the admixing time decreases, the likelihood of the reaction not going to completion increases. Without intending to be bound by theory, it is believed that as the reaction time increases, for example, beyond about 24 hours, 48 hours, or 72 hours, while some reaction product may continue to be formed the amount provided after 24 hours, 48 hours, or 72 hours will have little effect on the total yield, while the likelihood of side reactions and decomposition of the reagents and/or products increases

In some embodiments, X is located at the 3′ carbon and X is I. In some embodiments, X is located at the 3′ carbon and X is ¹⁸F. In some embodiments, X is located at the 3′ carbon and X is ²¹¹At. In some embodiments, X is located at the 3′ carbon and X is ¹²³I. In some embodiments, X is located at the 3′ carbon and X is ¹²⁴I. In some embodiments, X is located at the 3′ carbon and X is ¹²⁵I. In some embodiments, X is located at the 3′ carbon and X is ¹³¹I. In some embodiments, X is located at the 3′ carbon and X is ⁷⁶Br. In some embodiments, X is located at the 3′ carbon and X is ⁷⁷Br. In some embodiments, X is located at the 3′ carbon and X is H₃¹¹CO. In some embodiments, X is located at the 3′ carbon and X is CF₂¹⁸F. In some embodiments, X is located at the 3′ carbon and X is CHF¹⁸F. In some embodiments, X is located at the 3′ carbon and X is OCF₂¹⁸F. In some embodiments, X is located at the 3′ carbon and X is OCHF¹⁸F. In some embodiments, X is located at the 3′ carbon and X is SCF₂¹⁸F. In some embodiments, X is located at the 3′ carbon and X is SCHF¹⁸F. In some embodiments, X is located at the 3′ carbon and X is ¹¹CN. In some embodiments, X is located at the 3′ carbon and X is ¹¹CH₃. In some embodiments, X is located at the 3′ carbon and X is H₃¹¹CS.

In some embodiments, X is located at the 2′ carbon and X is I. In some embodiments, X is located at the 2′ carbon and X is ¹⁸F. In some embodiments, X is located at the 2′ carbon and X is ²¹¹At. In some embodiments, X is located at the 2′ carbon and X is ¹²³I. In some embodiments, X is located at the 2′ carbon and X is ¹²⁴I. In some embodiments, X is located at the 2′ carbon and X is ¹²⁵I. In some embodiments, X is located at the 2′ carbon and X is ¹³¹I. In some embodiments, X is located at the 2′ carbon and X is ⁷⁶Br. In some embodiments, X is located at the 2′ carbon and X is ⁷⁷Br. In some embodiments, X is located at the 2′ carbon and X is H₃¹¹CO. In some embodiments, X is located at the 2′ carbon and X is CF₂¹⁸F. In some embodiments, X is located at the 2′ carbon and X is CHF¹⁸F. In some embodiments, X is located at the 2′ carbon and X is OCF₂¹⁸F. In some embodiments, X is located at the 2′ carbon and X is OCHF¹⁸F. In some embodiments, X is located at the 2′ carbon and X is SCF₂¹⁸F. In some embodiments, X is located at the 2′ carbon and X is SCHF¹⁸F. In some embodiments, X is located at the 2′ carbon and X is ¹¹CN. In some embodiments, X is located at the 2′ carbon and X is ¹¹CH₃. In some embodiments, X is located at the 2′ carbon and X is H₃¹¹CS.

In some embodiments, X is located at the 1′ carbon and X is I. In some embodiments, X is located at the 1′ carbon and X is ¹⁸F. In some embodiments, X is located at the 1′ carbon and X is ²¹¹At. In some embodiments, X is located at the 1′ carbon and X is ¹²³I. In some embodiments, X is located at the 1′ carbon and X is ¹²⁴I. In some embodiments, X is located at the 1′ carbon and X is ¹²⁵I. In some embodiments, X is located at the 1′ carbon and X is ¹³¹I. In some embodiments, X is located at the 1′ carbon and X is ⁷⁶Br. In some embodiments, X is located at the 1′ carbon and X is ⁷⁷Br. In some embodiments, X is located at the 1′ carbon and X is H₃¹¹CO. In some embodiments, X is located at the 1′ carbon and X is CF₂¹⁸F. In some embodiments, X is located at the 1′ carbon and X is CHF¹⁸F. In some embodiments, X is located at the 1′ carbon and X is OCF₂¹⁸F. In some embodiments, X is located at the 1′ carbon and X is OCHF¹⁸F. In some embodiments, X is located at the 1′ carbon and X is SCF₂¹⁸F. In some embodiments, X is located at the 1′ carbon and X is SCHF¹⁸F. In some embodiments, X is located at the 1′ carbon and X is ¹¹CN. In some embodiments, X is located at the 1′ carbon and X is ¹¹CH₃. In some embodiments, X is located at the 1′ carbon and X is H₃¹¹CS.

In general, each R is independently selected from H, F, ²H, CH₃, and cyclopentyl. In embodiments, each R is independently selected from H and F. In embodiments, at least one R is H. In embodiments, both R are H. In embodiments, at least one R is F. In embodiments, both R are F. In embodiments, at least one R is ²H. In embodiments, both R are 2H. In embodiments, at least one R is CH₃. In embodiments, both R are CH₃. In embodiments, at least one R is cyclopentyl. In embodiments, both R are cyclopentyl. In embodiments, X is located at carbon 3′ and both R are H. In embodiments, X is located at carbon 3′ and both R are F. In embodiments, X is located at carbon 2′ and both R are H. In embodiments, X is located at carbon 2′ and both R are F. In embodiments, X is located at carbon 1′ and both R are H. In embodiments, X is located at carbon 1′ and both R are F.

In embodiments, Q and X are located at carbon 3′ and all R are H. In embodiments, Q and X are located at carbon 3′ and all R are F. In embodiments, Q and X are located at carbon 2′ and all R are H. In embodiments, Q and X are located at carbon 2′ and all R are F. In embodiments, Q and X are located at carbon 1′ and all R are H. In embodiments, Q and X are located at carbon 1′ and all R are F.

The disclosure further provides methods of preparing compounds having a structure according to formulae (Ia), (IIa) or (Illa). Compounds having a structure according to formula (Ia-3) can be prepared by admixing 6-iodoquinoline-4-carboxylic acid with (S)-1-glycylpyrrolidine-2-carbonitrile HCl salt in DMF at a temperature in a range of about −5° C. to about 30° C., for example about 0° C. to about 25° C. in the presence of 2-(1H-Benzotriazole-1-yl)-1,1,3,3-tetramethylaminium tetrafluoroborate (TBTU) and Hunig's base. In embodiments, the method further comprises preparing the 6-iodoquinoline-4-carboxylic acid by refluxing 6-iodoquinoline-2,4-carboxylic acid in the presence of N-methyl-2-pyrrolidone (NMP), for a suitable time to allow the reaction to proceed, for example, about 1 hour to about 24 hours, about 6 hours to about 22 hours, about 12 hours to about 20 hours, or about 14 hours to about 18 hours. In embodiments, the method further comprises preparing the 6-iodoquinoline-2,4-carboxylic acid by admixing 5-iodoindoline-2,3-dione and NaOH in water, followed by addition of sodium pyruvate and allowing the resulting reaction mixture to stir for 48 hours at reflux. Alternative compounds according to formula (Ia) (e.g., Ia-2 or Ia-1) and compounds according to formulae (IIa) and (IIIa) can be prepared in the same way using appropriate quinoline, naphthalene, and indole derivatives.

In embodiments, the compounds having a structure according to formulae (IV), (VI), (VII), can be converted to compounds having a structure according to formulae (VIII), (X), and (XI), respectively. The compounds having a structure according to formulae (VIII), (X), and (XI) can be prepared from the compounds having a structure according to formulae (IV), (VI), and (VII) in the same way as described above for preparing the compounds having a structure according to formulae (VIII), (X), and (XI) from the compounds having a structure according to formulae (I), (II), and (III).

The disclosure further provides a method of treating, detecting, or imaging cancer comprising administering to a patient in need thereof a fibroblast activation protein inhibitor according to the disclosure. In embodiments, the methods of the disclosure comprise treating cancer comprising administering to a patient in need thereof a fibroblast activation protein inhibitor according to the disclosure. In embodiments, the methods of the disclosure comprise detecting cancer comprising administering to a patient in need thereof a fibroblast activation protein inhibitor according to the disclosure. In refinements of the foregoing embodiment, the method further comprises detecting the fibroblast activation protein inhibitor in the patient. In embodiments, the methods of the disclosure comprise imaging cancer comprising administering to a patient in need thereof a fibroblast activation protein inhibitor according to the disclosure. In refinements of the foregoing embodiment, the method further comprises detecting the fibroblast activation protein inhibitor in the patient. The disclosure further provides use of a fibroblast activation protein inhibitor according to the disclosure in the treatment, detection, or imaging of cancer. In embodiments, the methods provide use of a fibroblast activation protein inhibitor according to the disclosure in the treatment of cancer. In embodiments, the methods provide use of a fibroblast activation protein inhibitor according to the disclosure in the detection of cancer. In embodiments, the methods provide use of a fibroblast activation protein inhibitor according to the disclosure in the imaging of cancer. The disclosure further provides the use of a fibroblast activation protein inhibitor according to the disclosure in the manufacture of a medicament for the treatment, detection, or imaging of cancer. In embodiments, the medicament is for the treatment of cancer. In embodiments, the medicament is for the detection of cancer. In embodiments, the medicament is for the imaging of cancer. In refinements of any of the foregoing embodiments, the cancer comprises an epithelial tumor, sarcoma, mesothelioma, or a combination thereof.

Fibroblast activation protein (FAP) is generally expressed in human development, growth, and wound healing. Thus, the fibroblast activation protein inhibitors of the disclosure can have clinical uses in detecting growth and/or wound healing and/or the lack of growth or wound healing. For example, in a diabetic wound, FAP may not be appropriately expressed, and the wound may not be healing. A FAPI compound of the disclosure can be used as a diagnostic agent to indicate the lack of expression of FAP as the cause of the lack of wound healing. As another example, FAP may be expressed in cardiac remodeling after a myocardial infarction and a FAPI compound of the disclosure can be used as a diagnostic agent to confirm the expression of FAP and the growth during remodeling. As another example, the FAPI compounds of the disclosure can be used to as a diagnostic agent for the expression, or lack thereof, of FAP in an amputee limb, to determine the healing status of the amputee limb.

Thus, disclosure further provides a method of detecting fibroblast activation protein expression in human development, human growth, human wound healing, or a combination thereof, the method comprising administering to a patient a fibroblast activation protein inhibitor of the disclosure. In embodiments, the method further comprises detecting the fibroblast activation protein inhibitor in the patient. In embodiments, the methods provide the use of a fibroblast activation protein inhibitor according to the disclosure in the detection of the expression of fibroblast activation protein or the lack of expression of fibroblast activation protein in a human. In embodiments, the methods provide the use of a fibroblast activation protein inhibitor of the disclosure in the manufacture of a medicament for the detection of the expression of fibroblast activation protein. The disclosure further provides methods of detecting a cardiovascular pathology comprising, administering to a patient a fibroblast activation protein inhibitor according to the disclosure. In embodiments, the methods provide the use of a fibroblast activation protein inhibitor of the disclosure in the detection of a cardiovascular pathology in a human. In embodiments, the methods provide the use of a fibroblast activation protein inhibitor of the disclosure in the manufacture of a medicament for the detection of a cardiovascular pathology. In refinements of the foregoing embodiments, the cardiovascular pathology comprises a myocardial infarction or cardiac remodeling after infarction. In alternative refinements of the foregoing embodiments, the cardiovascular pathology comprises aortic remodeling. In embodiments, the methods provide the use of a fibroblast activation protein inhibitor of the disclosure in the manufacture of a medicament for the detection of aortic remodeling. In embodiments, the methods provide the use of a fibroblast activation protein inhibitor of the disclosure in the detection of a pulmonary pathology in a human. In refinements of the foregoing embodiments, the pulmonary pathology may be the result of an auto immune or rheumatologic response, primary pulmonary pathology such as interstitial lung disease, or post treatment effects from chemotherapy, radiation therapy, or a combination thereof. In embodiments, the methods provide the use of a fibroblast activation protein inhibitor of the disclosure in the manufacture of a medicament for the detection of a pulmonary pathology.

In embodiments wherein the compounds of the disclosure are used for detecting and/or imaging, in the compounds having a structure according to formula (I), (II), or (III), X can be 18F, ¹²³I, ¹²⁵I, or ¹³¹I.

As demonstrated in the Examples below, amounts of expected radiation absorbed by human organs from the compounds of the disclosure are typical amounts relative to known tracer compounds. for example, the data in Table 1 demonstrates that a compound of the disclosure including the ¹⁸F radioisotope has estimated radiation absorption amounts typical of known ¹⁸F tracer molecules.

“Comprising” as used herein means that various components, ingredients, or steps that can be conjointly employed in practicing the present disclosure. Accordingly, the term “comprising” encompasses the more restrictive terms “consisting essentially of” and “consisting of.” The present compositions can comprise, consist essentially of, or consist of any of the required and optional elements disclosed herein. The disclosure illustratively disclosed herein suitably may be practiced in the absence of any element or step which is not specifically disclosed herein.

The term “about” is used according to its ordinary meaning, for example, to mean approximately or around. In one embodiment, the term “about” means±10% of a stated value or range of values. In another embodiment, the term “about” means±5% of a stated value or range of values. A value or range described in combination with the term “about” expressly includes the specific value and/or range as well (e.g., for a value described as “about 40,” “40” is also expressly contemplated).

All ranges set forth herein include all possible subsets of ranges and any combinations of such subset ranges. By default, ranges are inclusive of the stated endpoints, unless stated otherwise. Where a range of values is provided, it is understood that each intervening value between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also contemplated to be part of the disclosure

The compositions and methods in accordance with the disclosure can be better understood in light of the following examples, which are merely intended to illustrate the compositions and methods and are not meant to limit the scope thereof in any way.

Specifically contemplated embodiments of the disclosure are herein described in the following numbered paragraphs. These embodiments are intended to be illustrative in nature and not intended to be limiting.
A1. A fibroblast activation protein inhibitor having a structure according to formula (I), formula (II), or formula (III):

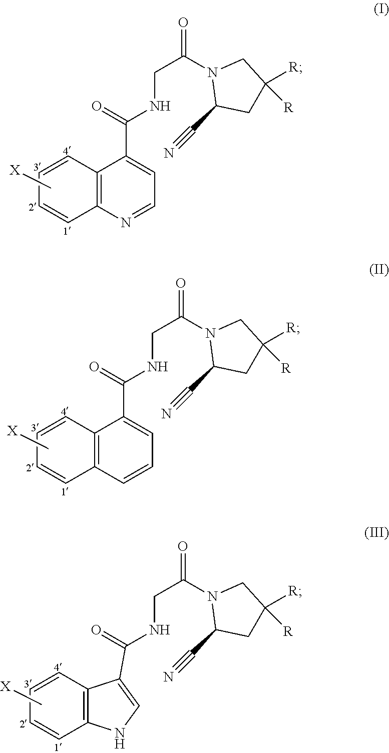

wherein
X is located at carbon 1′, 2′, or 3′ and is selected from the group of I, ¹⁸F, ²¹¹At, ¹²³I, ¹²⁴I, ¹²⁵I, ¹³¹I, ⁷⁶Br, ⁷⁷Br, H₃¹¹CO, CF₂¹⁸F, CHF¹⁸F, OCF₂¹⁸F, OCHF¹⁸F, SCF₂¹⁸F, SCHF¹⁸F, ¹¹CN, ¹¹CH₃, and H₃¹¹CS;
each R is independently selected from H, F, ²H, CH₃, and cyclopentyl;
with the proviso that in formula (I), when X is H₃¹¹CO and both R are H or both R are F, then X is not located at carbon 3′.
A2. The fibroblast activation protein inhibitor of paragraph A1, wherein X is located at the 3′ carbon.
A3. The fibroblast activation protein inhibitor of paragraph A1, wherein X is located at the 2′ carbon.
A4. The fibroblast activation protein inhibitor of paragraph A1, wherein X is located at the 1′ carbon.
A5. The fibroblast activation protein inhibitor of any one of the preceding paragraphs, wherein X is 1.
A6. The fibroblast activation protein inhibitor of any one of the preceding paragraphs, wherein X is ¹⁸F.
A7. The fibroblast activation protein inhibitor of any one of the preceding paragraphs, wherein X is ²¹¹At.
A8. The fibroblast activation protein inhibitor of any one of the preceding paragraphs, wherein X is ¹²³I.
A9. The fibroblast activation protein inhibitor of any one of the preceding paragraphs, wherein X is ¹²⁴I.
A10. The fibroblast activation protein inhibitor of any one of the preceding paragraphs, wherein X is ¹²⁵I.
A11. The fibroblast activation protein inhibitor of any one of the preceding paragraphs, wherein X is ¹³¹I.
A12. The fibroblast activation protein inhibitor of any one of the preceding paragraphs, wherein X is ⁷⁶Br.
A13. The fibroblast activation protein inhibitor of any one of the preceding paragraphs, wherein X is ⁷⁷Br.
A14. The fibroblast activation protein inhibitor of any one of the preceding paragraphs, wherein X is H₃¹¹CO.
A15. The fibroblast activation protein inhibitor of any one of the preceding paragraphs, wherein X is CF₂¹⁸F.
A16. The fibroblast activation protein inhibitor of any one of the preceding paragraphs, wherein X is CHF¹⁸F.
A17. The fibroblast activation protein inhibitor of any one of the preceding paragraphs, wherein X is OCF₂¹⁸F.
A18. The fibroblast activation protein inhibitor of any one of the preceding paragraphs, wherein X is OCHF¹⁸F.
A19. The fibroblast activation protein inhibitor of any one of the preceding paragraphs, wherein X is SCF₂¹⁸F.
A20. The fibroblast activation protein inhibitor of any one of the preceding paragraphs, wherein X is SCHF¹⁸F.
A21. The fibroblast activation protein inhibitor of any one of the preceding paragraphs, wherein X is ¹¹CN.
A22. The fibroblast activation protein inhibitor of any one of the preceding paragraphs, wherein X is ¹¹CH₃.
A23. The fibroblast activation protein inhibitor of any one of the preceding paragraphs, wherein X is H₃¹¹CS.
A24. The fibroblast activation protein inhibitor of any one of the preceding paragraphs, wherein at least one R is H.
A25. The fibroblast activation protein inhibitor of any one of the preceding paragraphs, wherein both R are H.
A26. The fibroblast activation protein inhibitor of any one of paragraphs A1 to A24, wherein at least one R is F.
A27. The fibroblast activation protein inhibitor of any one of paragraphs A1 to A23, wherein both R are F.
A28. The fibroblast activation protein inhibitor of any one of paragraphs A1 to A24 or 26, wherein at least one R is ²H.
A29. The fibroblast activation protein inhibitor of any one of paragraphs A1 to A23, wherein both R are ²H.
A30. The fibroblast activation protein inhibitor of any one of paragraphs A1 to A24, A26, or A28, wherein at least one R is CH₃.
A31. The fibroblast activation protein inhibitor of any one of paragraphs A1 to A23, wherein both R are CH₃.
A32. The fibroblast activation protein inhibitor of any one of paragraphs A1 to A24, A26, A28, or A31, wherein at least one R is cyclopentyl.
A33. The fibroblast activation protein inhibitor of any one of paragraphs A1 to A23, wherein both R are cyclopentyl.
A34. The fibroblast activation protein inhibitor of any one of the preceding paragraphs, having a structure according to formula (I).
A35. The fibroblast activation protein inhibitor of paragraph A34, wherein X is located at carbon 3′ and both R are H.
A36. The fibroblast activation protein inhibitor of paragraph A34, wherein X is located at carbon 3′ and both R are F.
A37. The fibroblast activation protein inhibitor of paragraph A34, wherein X is located at carbon 2′ and both R are H.
A38. The fibroblast activation protein inhibitor of paragraph A34, wherein X is located at carbon 2′ and both R are F.
A39. The fibroblast activation protein inhibitor of paragraph A34, wherein X is located at carbon 1′ and both R are H.
A40. The fibroblast activation protein inhibitor of paragraph A34, wherein X is located at carbon 1′ and both R are F.
A41. The fibroblast activation protein inhibitor of any one of paragraphs A1 to A33, having a structure according to formula (II).
A42. The fibroblast activation protein inhibitor of paragraph A41, wherein X is located at carbon 3′ and both R are H.
A43. The fibroblast activation protein inhibitor of paragraph A41, wherein X is located at carbon 3′ and both R are F.
A44. The fibroblast activation protein inhibitor of paragraph A41, wherein X is located at carbon 2′ and both R are H.
A45. The fibroblast activation protein inhibitor of paragraph A41, wherein X is located at carbon 2′ and both R are F.
A46. The fibroblast activation protein inhibitor of paragraph A41, wherein X is located at carbon 1′ and both R are H.
A47. The fibroblast activation protein inhibitor of paragraph A41, wherein X is located at carbon 1′ and both R are F.
A48. The fibroblast activation protein inhibitor of any one of paragraphs A1 to A33, having a structure according to formula (III).
A49. The fibroblast activation protein inhibitor of paragraph A48, wherein X is located at carbon 3′ and both R are H.
A50. The fibroblast activation protein inhibitor of paragraph A48, wherein X is located at carbon 3′ and both R are F.
A51. The fibroblast activation protein inhibitor of paragraph A48, wherein X is located at carbon 2′ and both R are H.
A52. The fibroblast activation protein inhibitor of paragraph A48, wherein X is located at carbon 2′ and both R are F.
A53. The fibroblast activation protein inhibitor of paragraph A48, wherein X is located at carbon 1′ and both R are H.
A54. The fibroblast activation protein inhibitor of paragraph A48, wherein X is located at carbon 1′ and both R are F.
A55. The fibroblast activation protein inhibitor of paragraph A1 having a structure according to one of the following formulae:

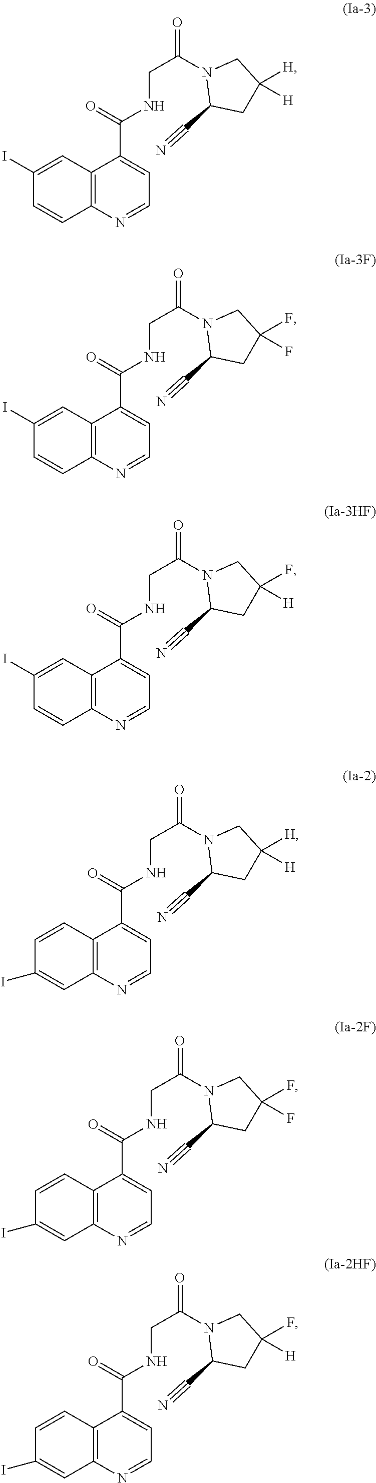

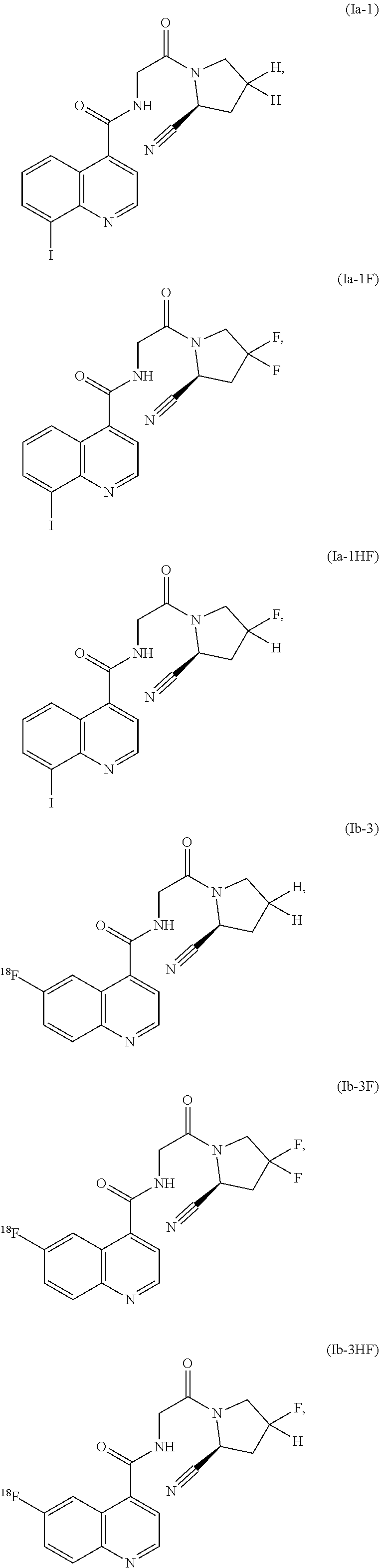

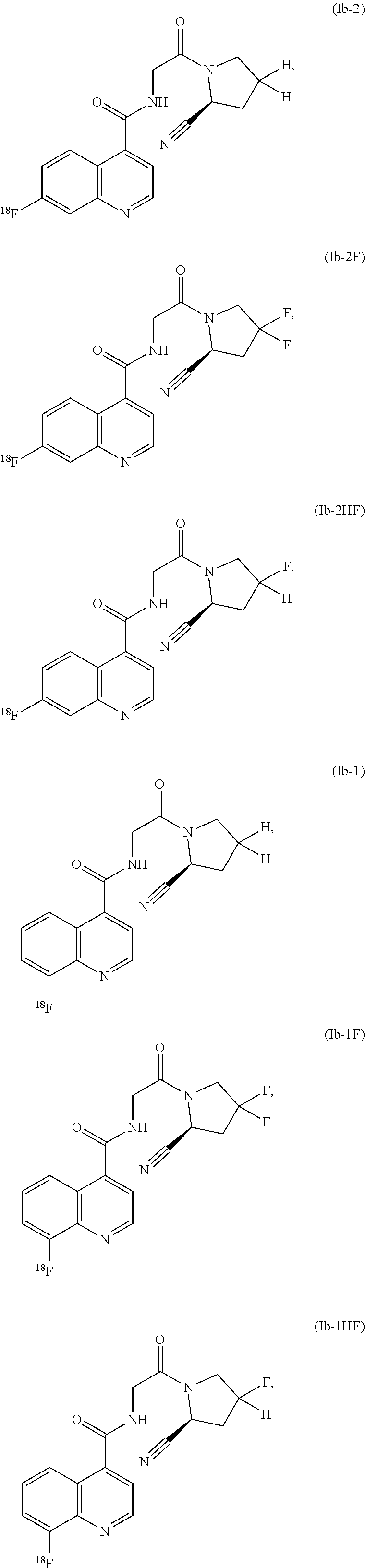

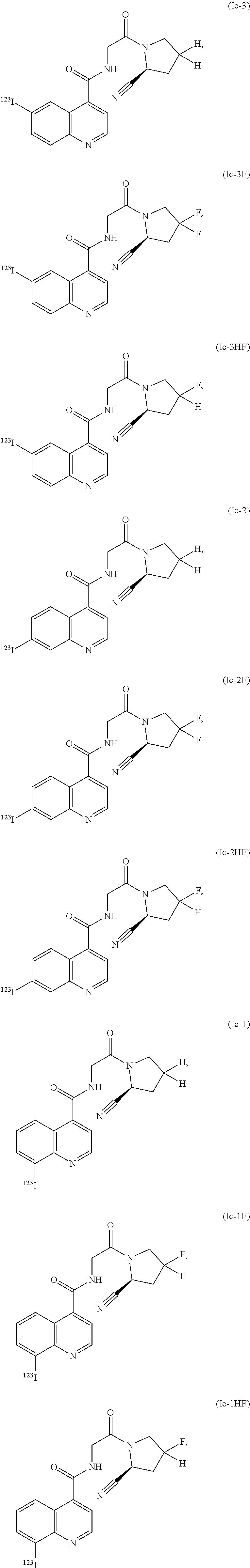

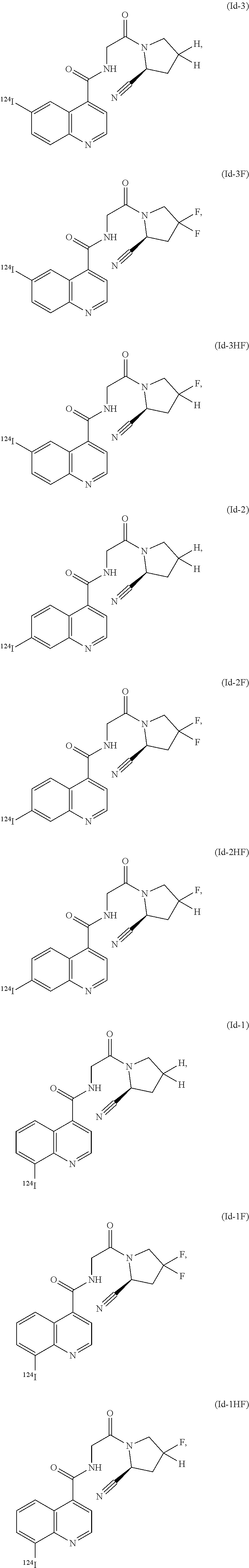

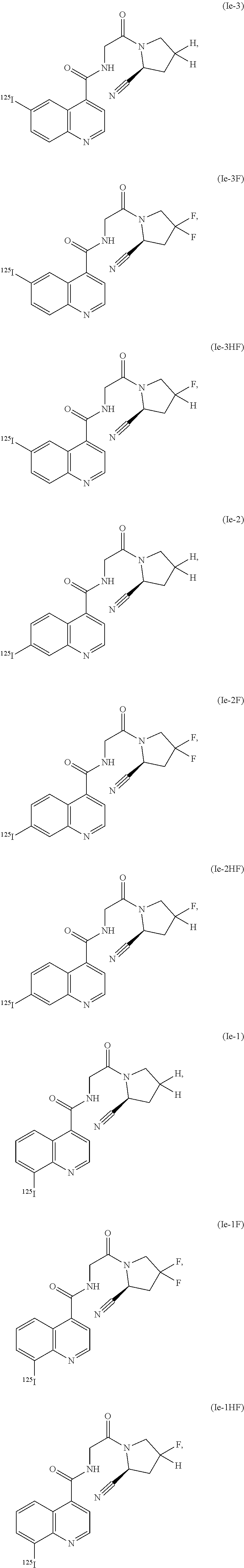

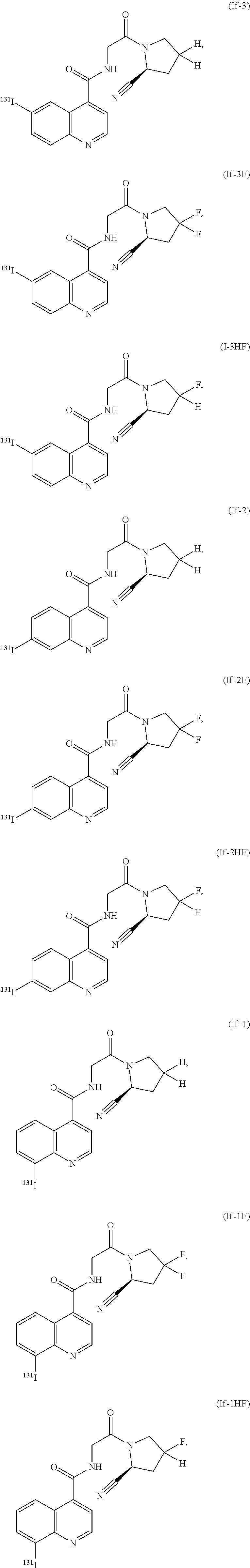

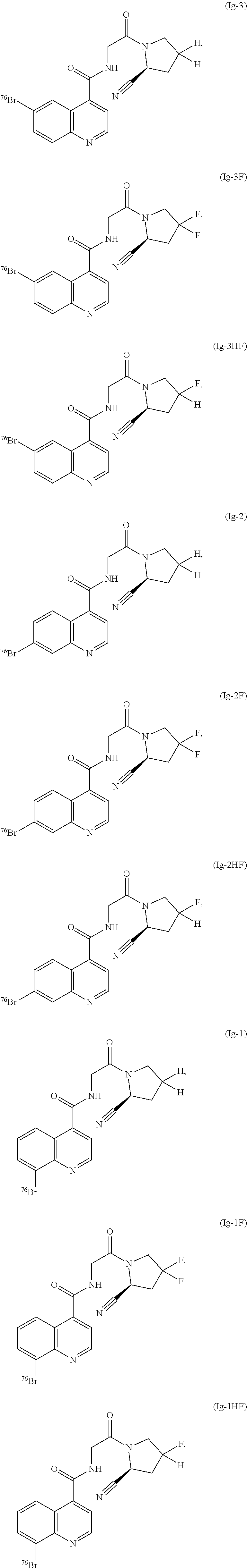

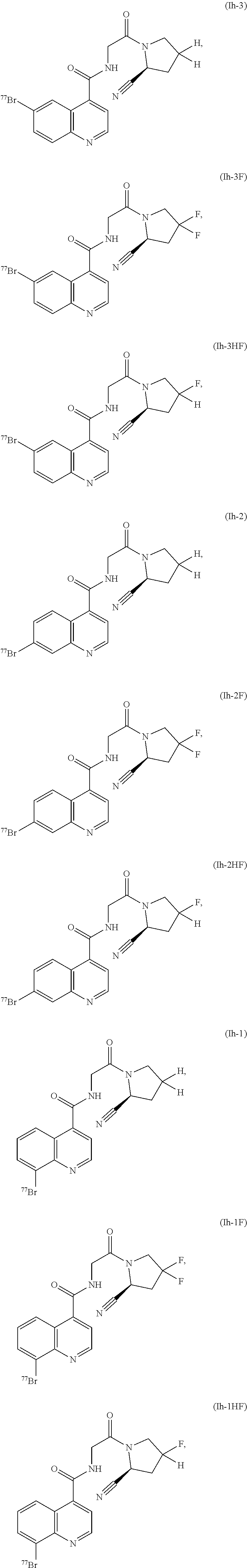

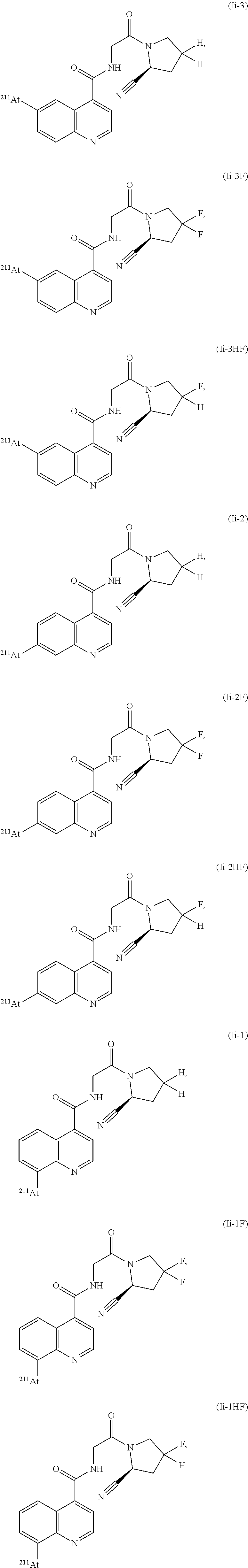

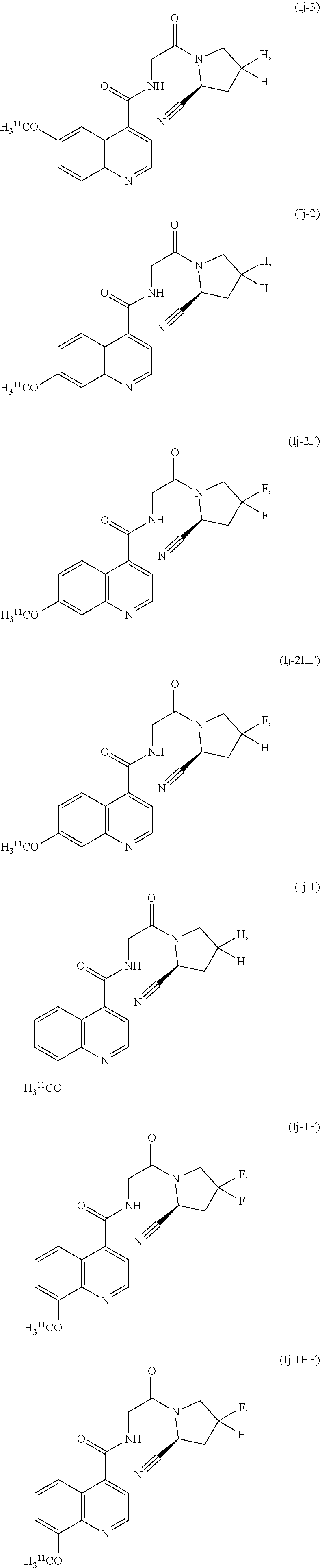

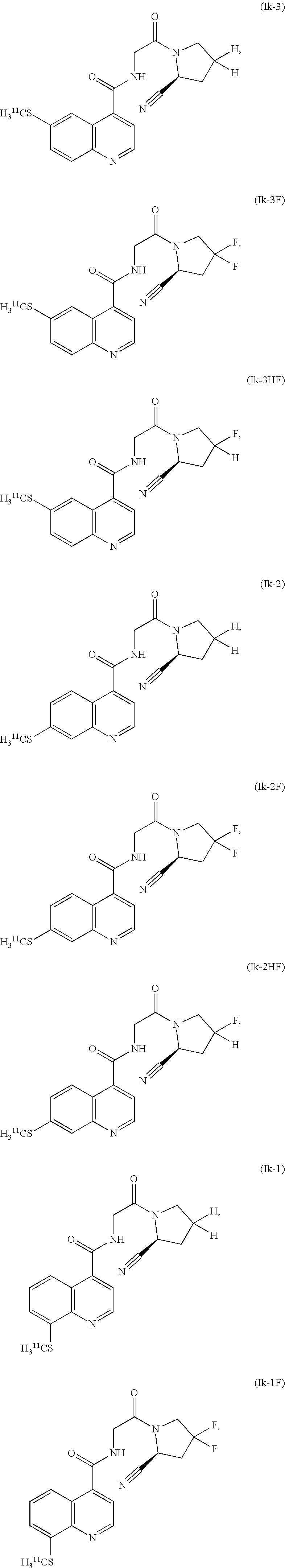

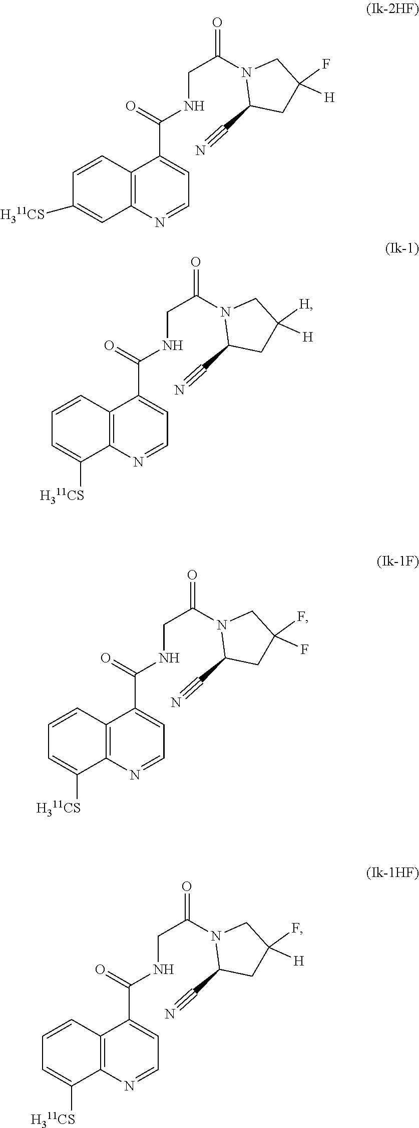

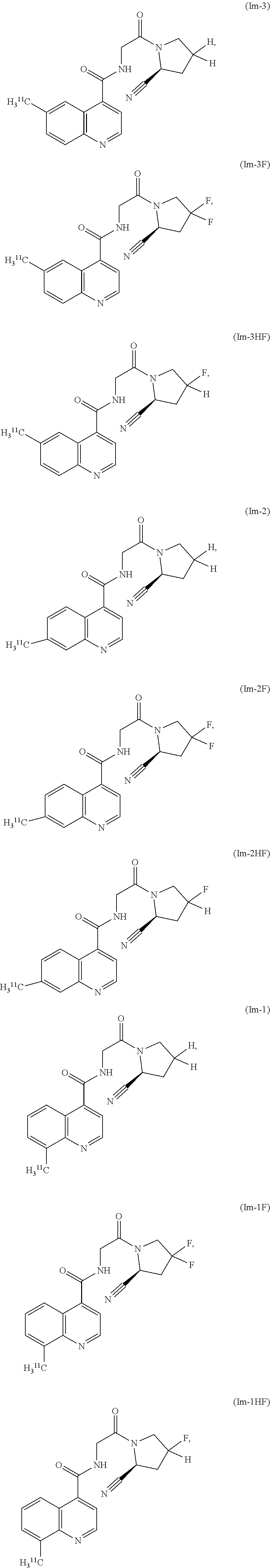

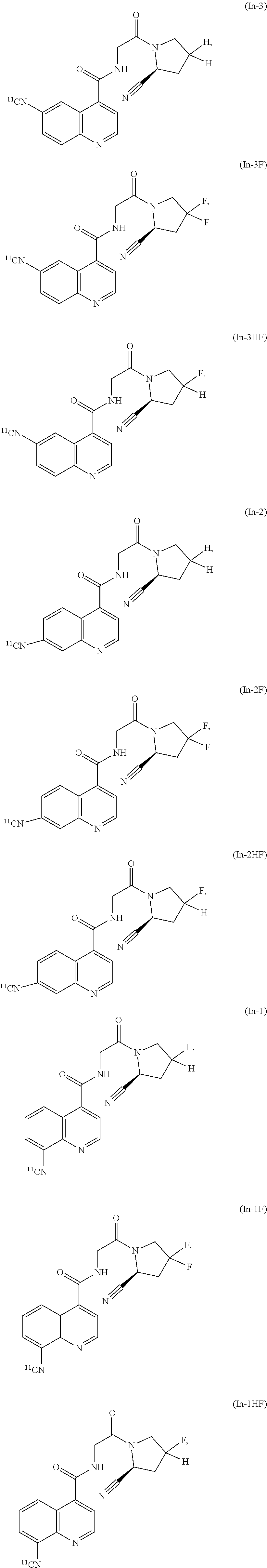

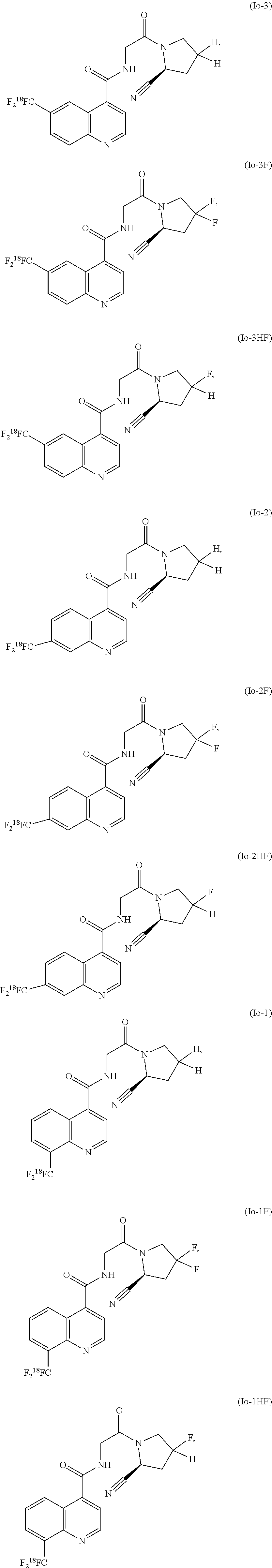

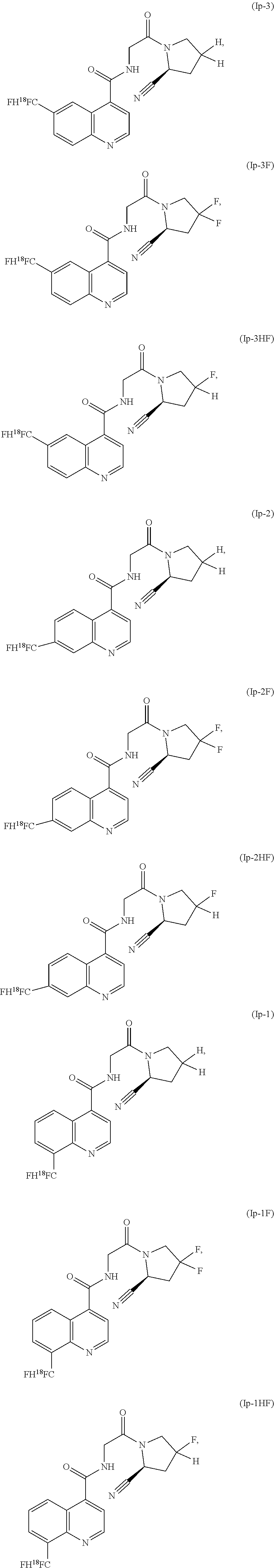

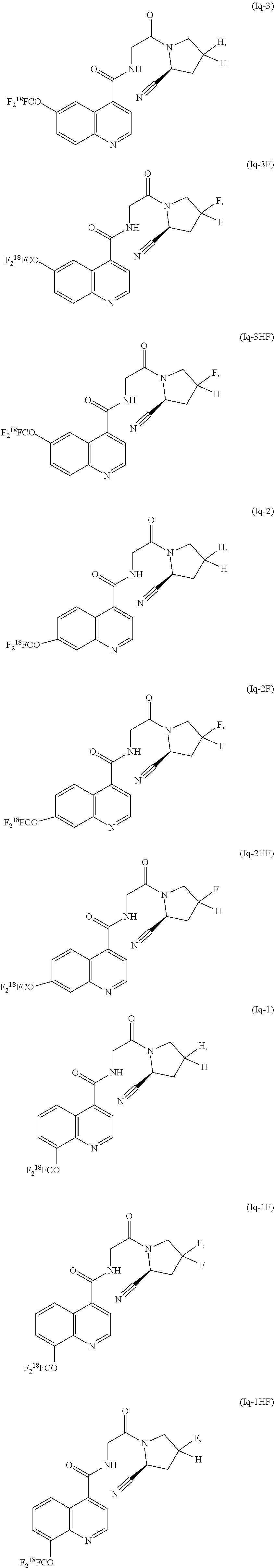

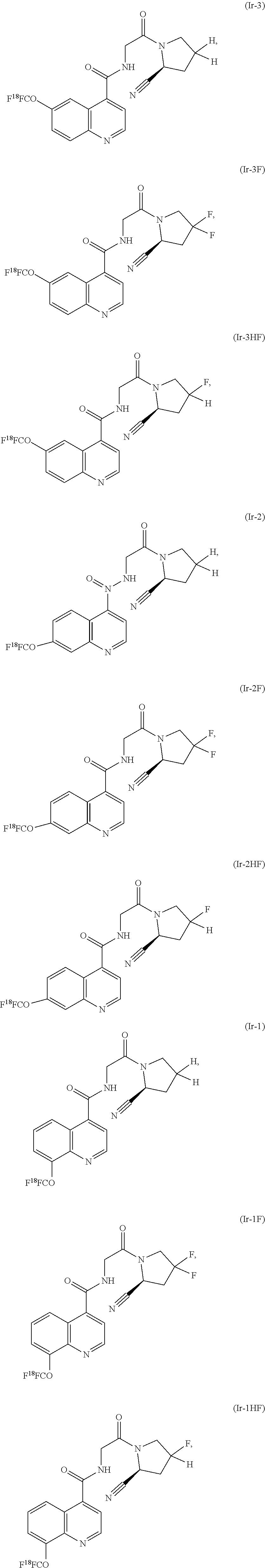

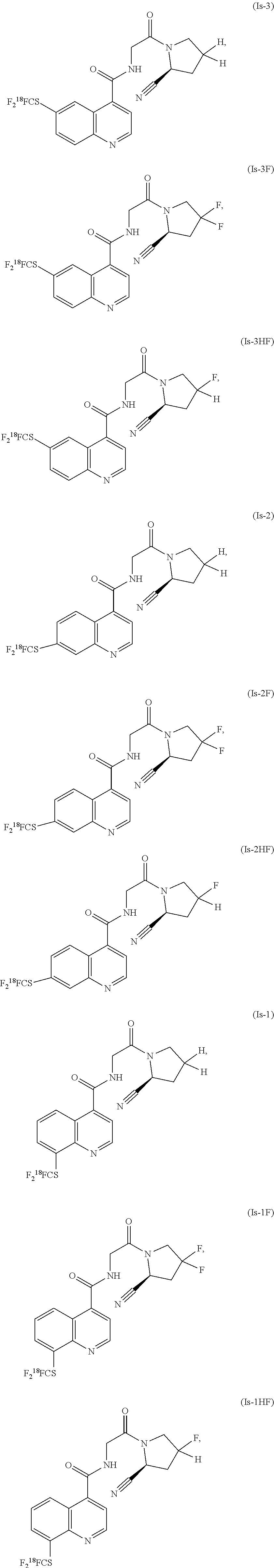

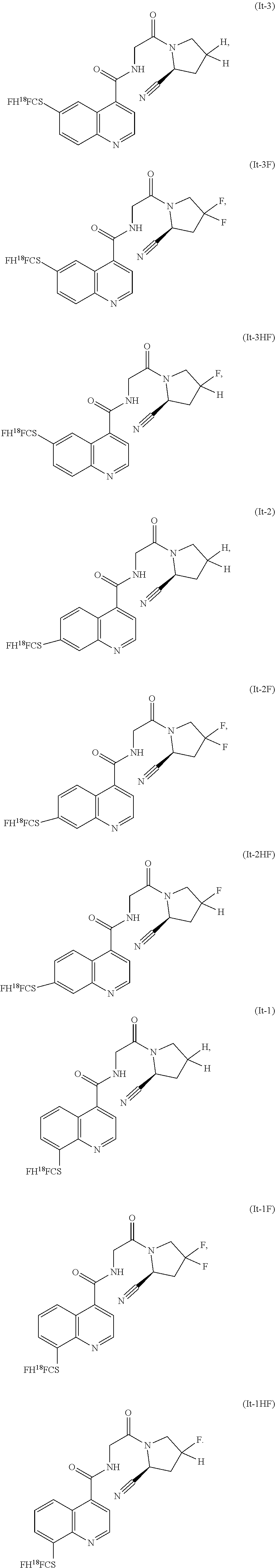

A56. The fibroblast activation protein inhibitor of paragraph A1 having a structure according to one of the following formulae:

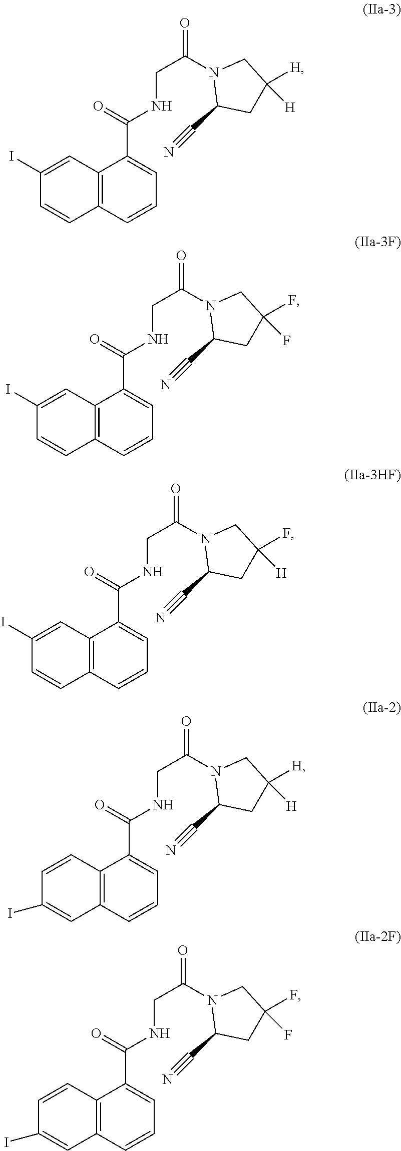

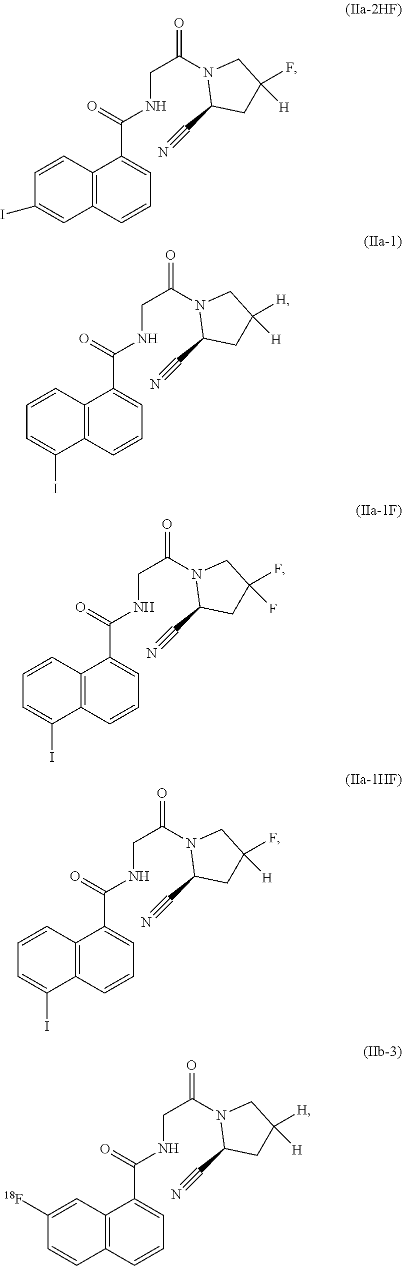

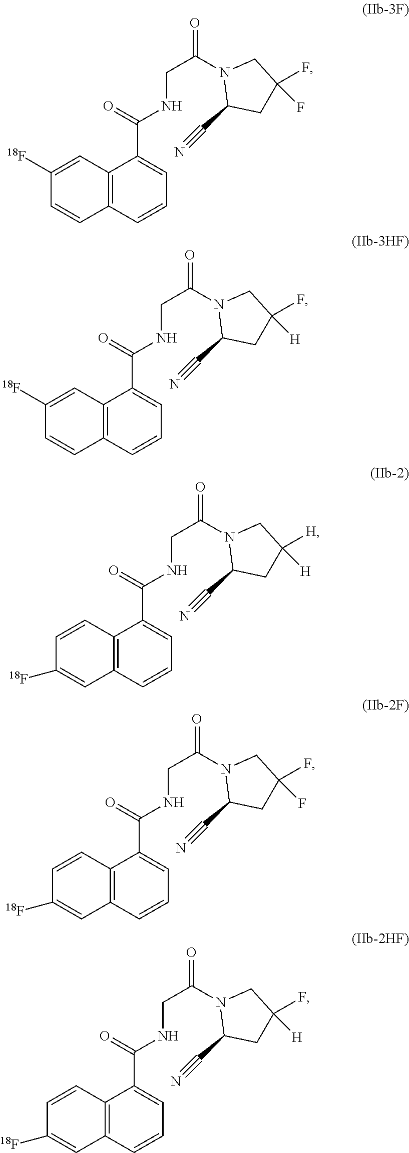

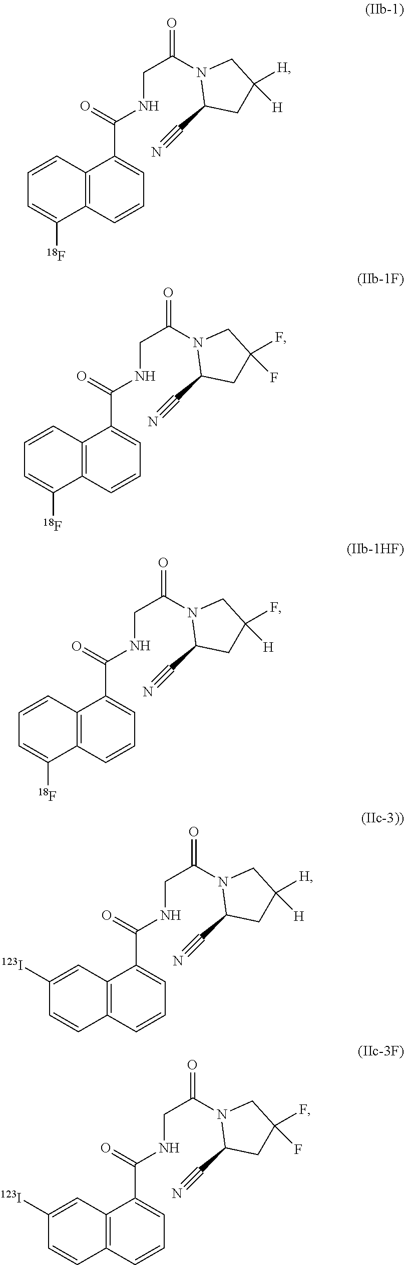

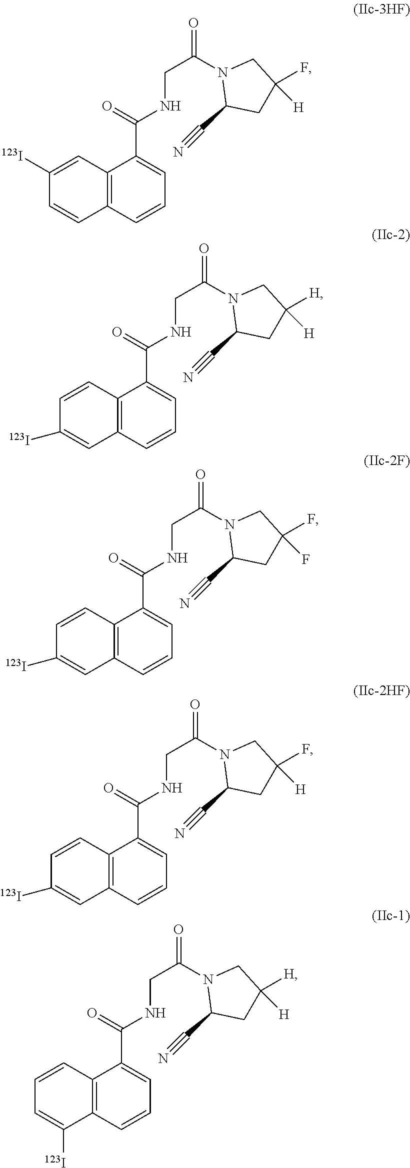

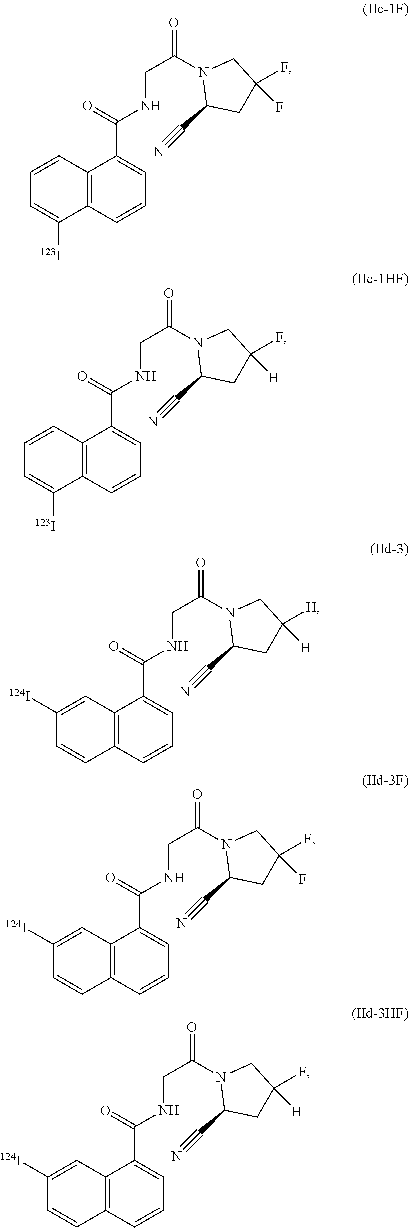

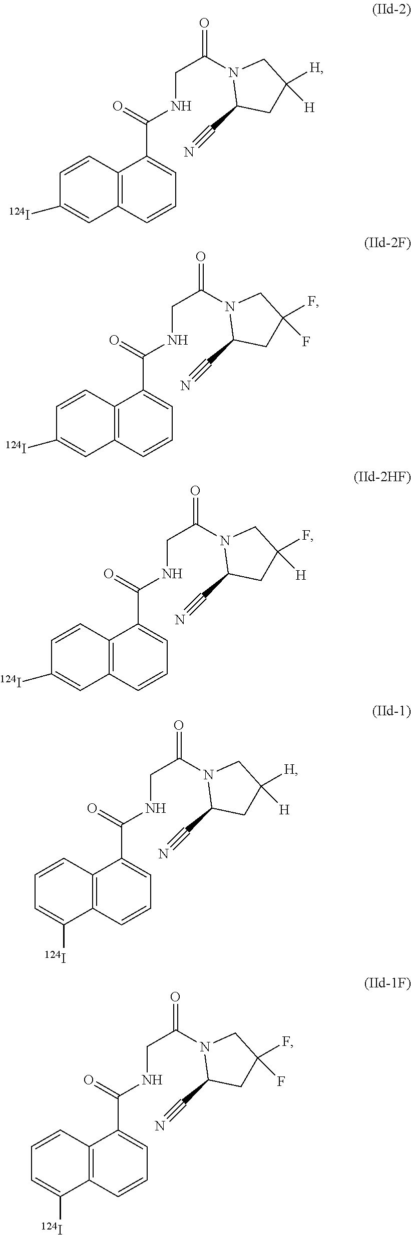

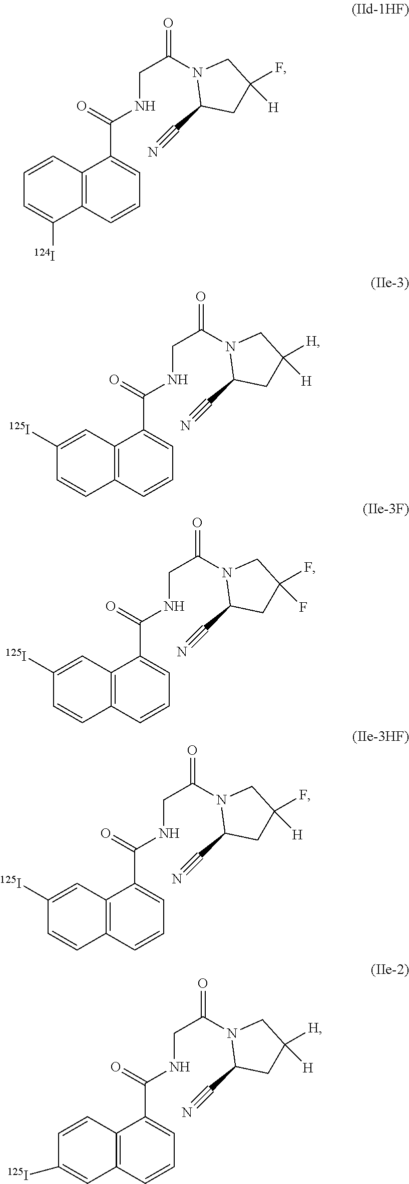

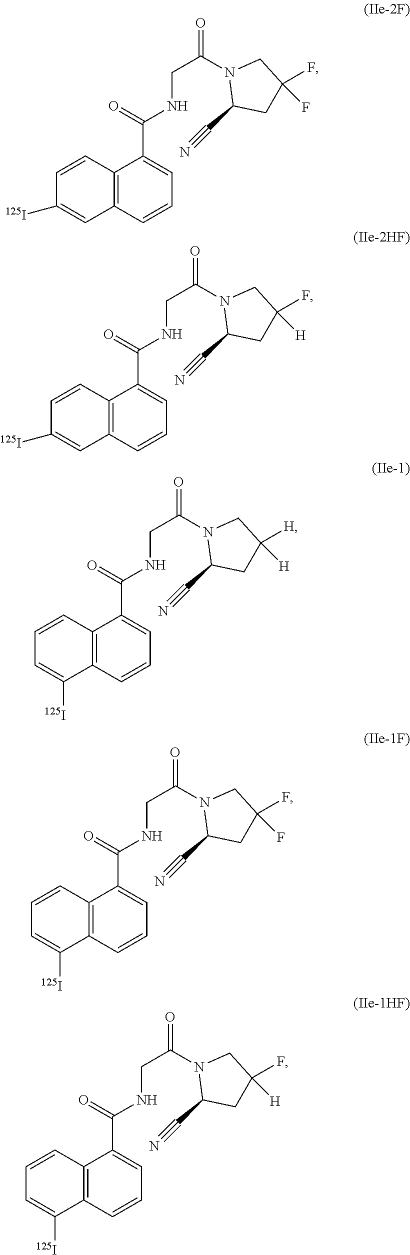

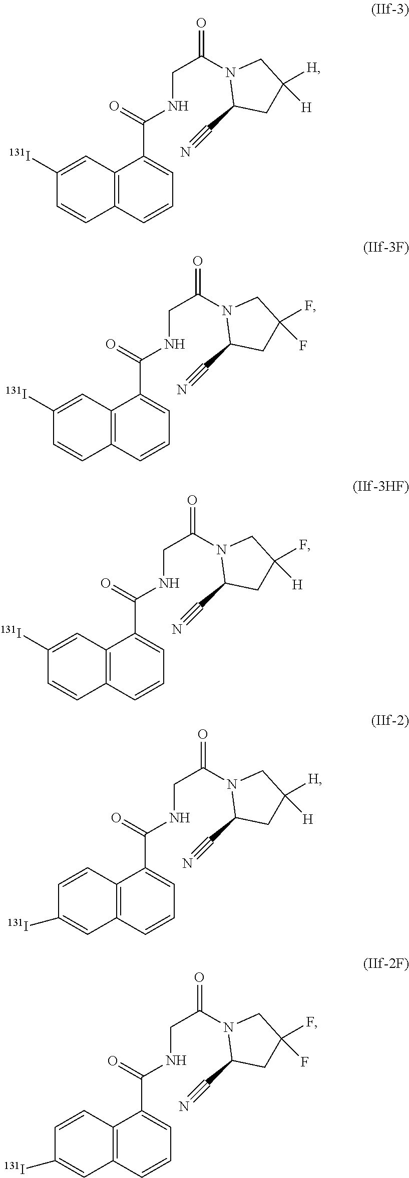

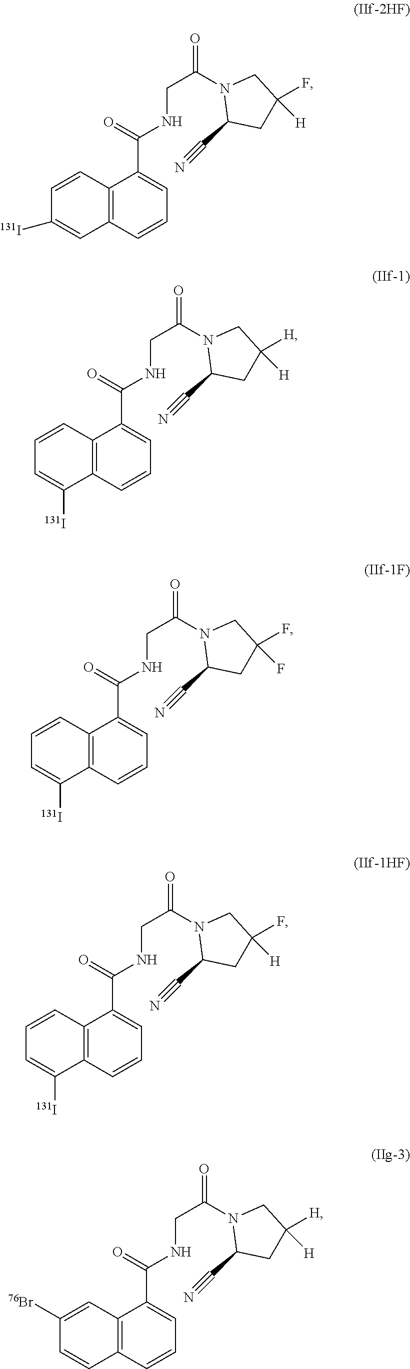

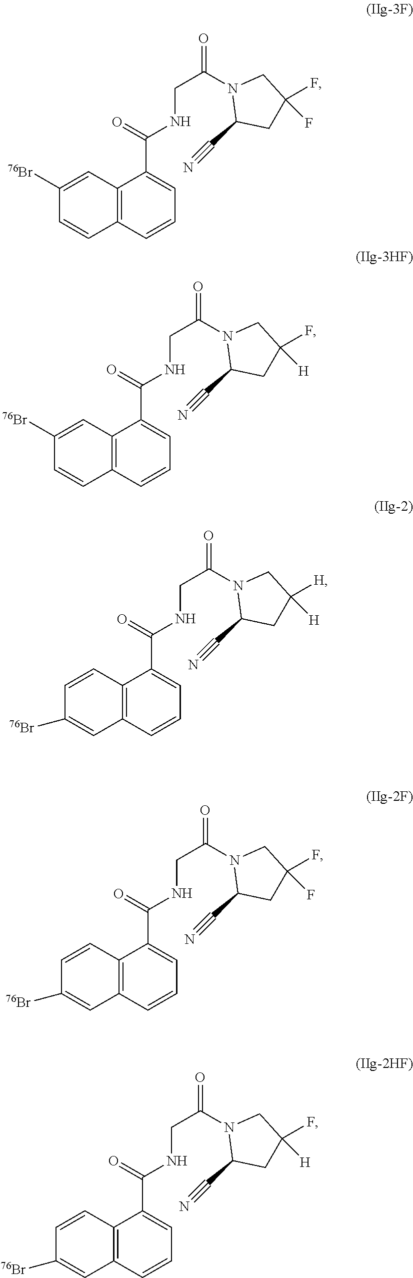

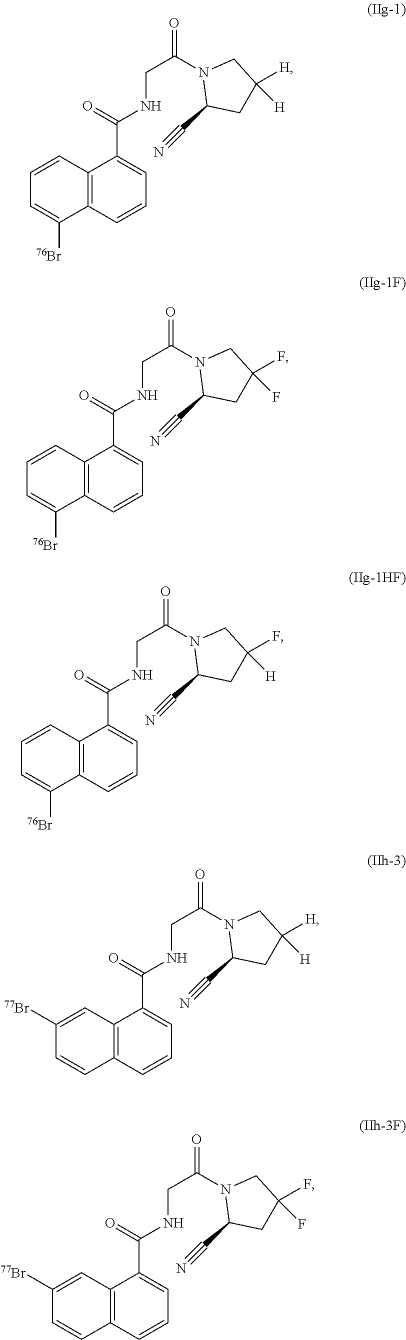

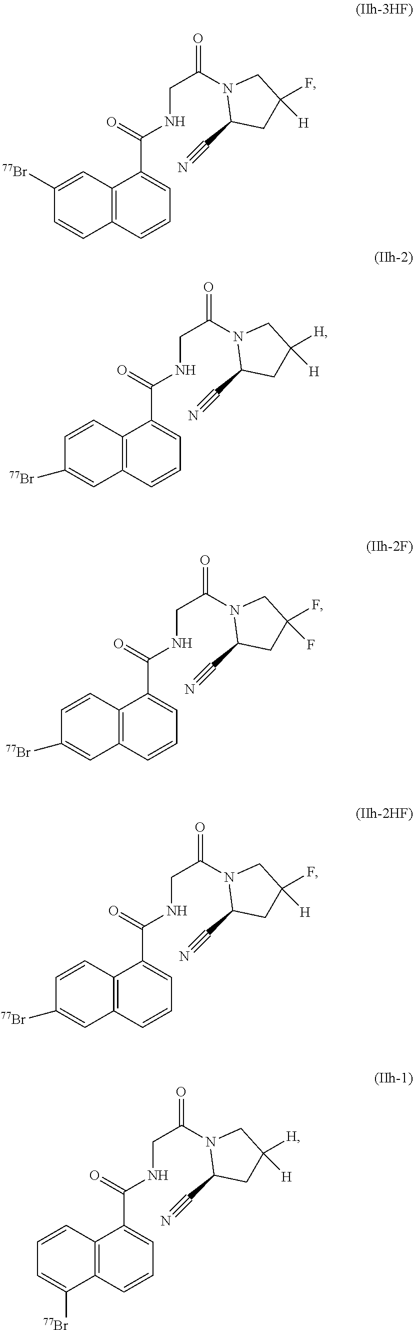

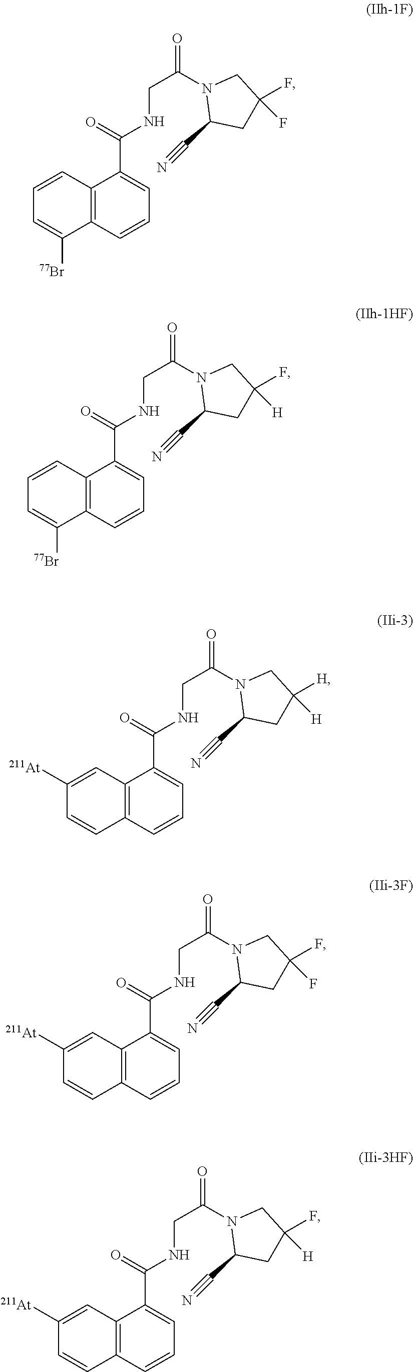

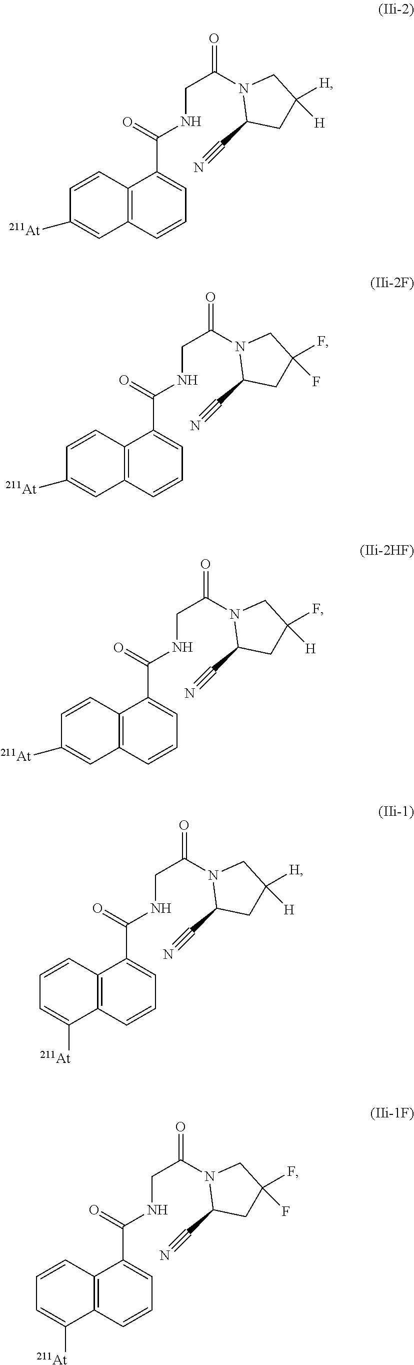

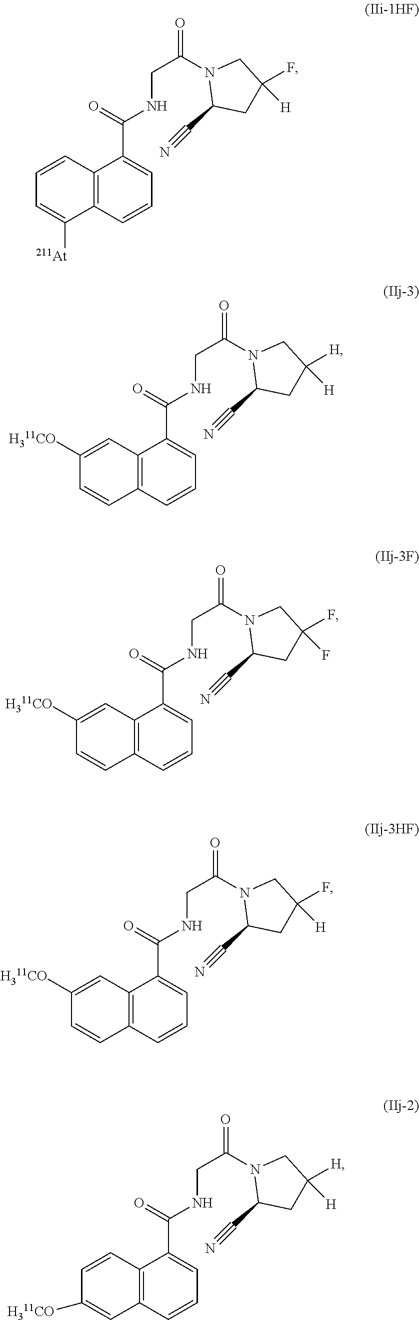

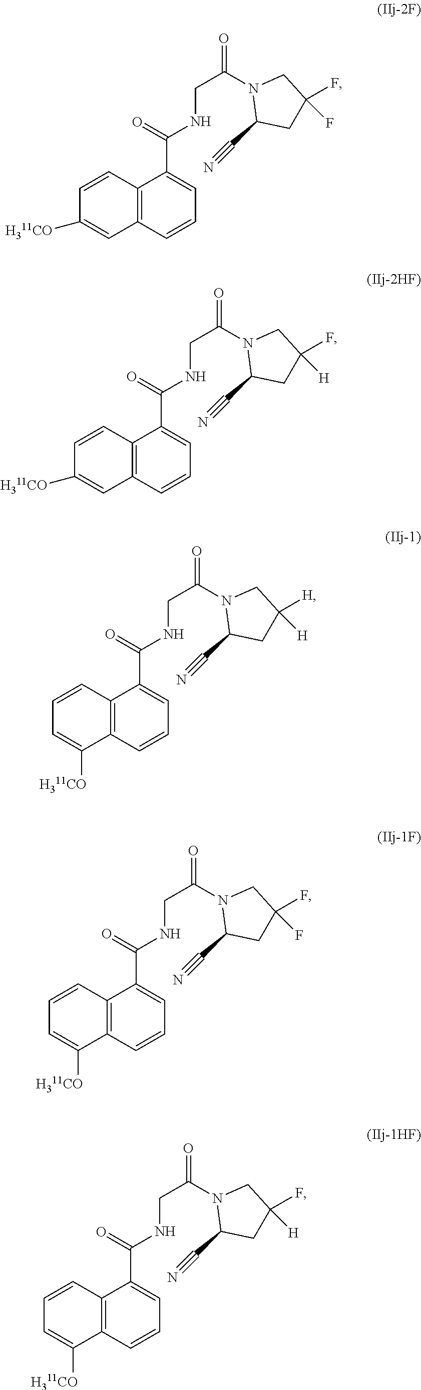

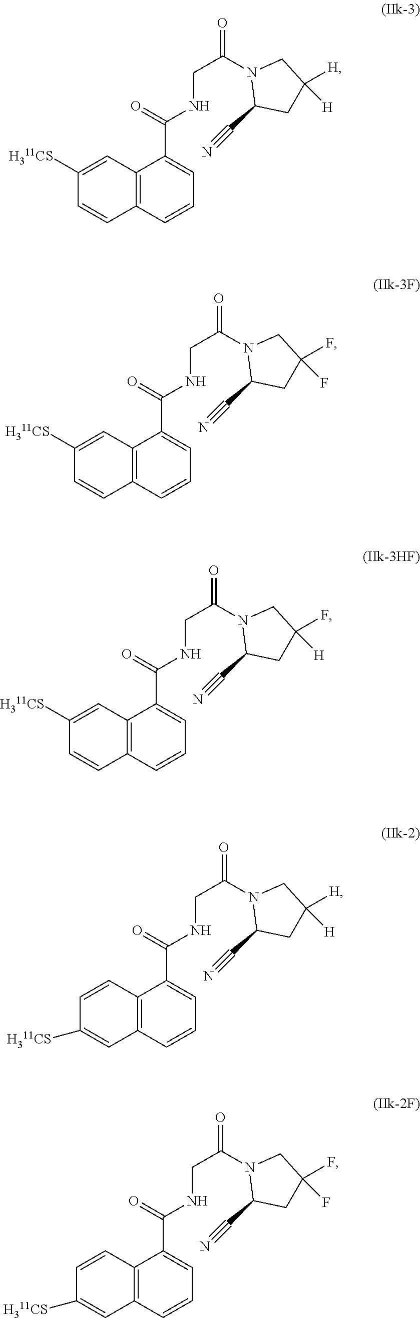

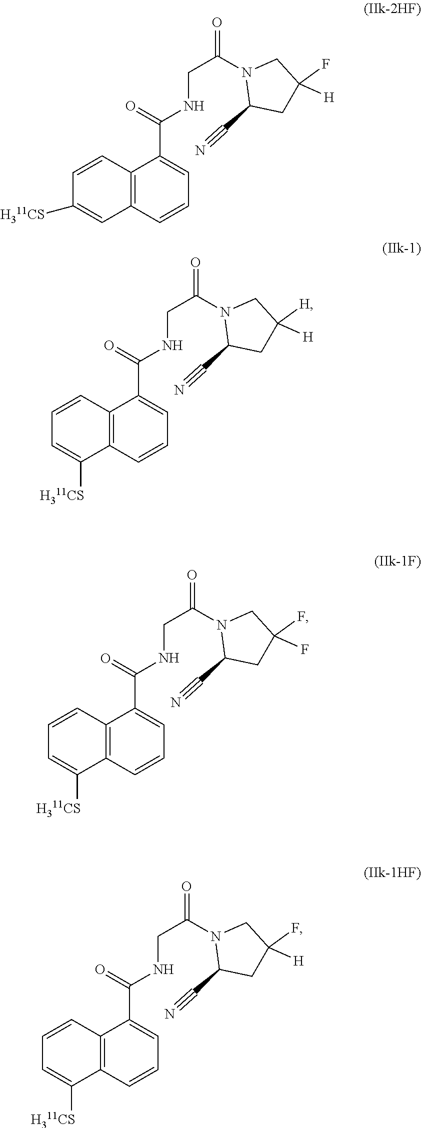

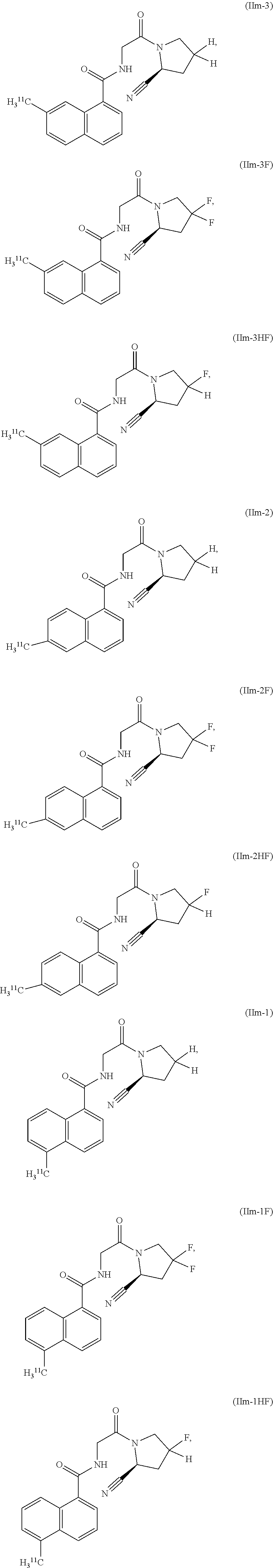

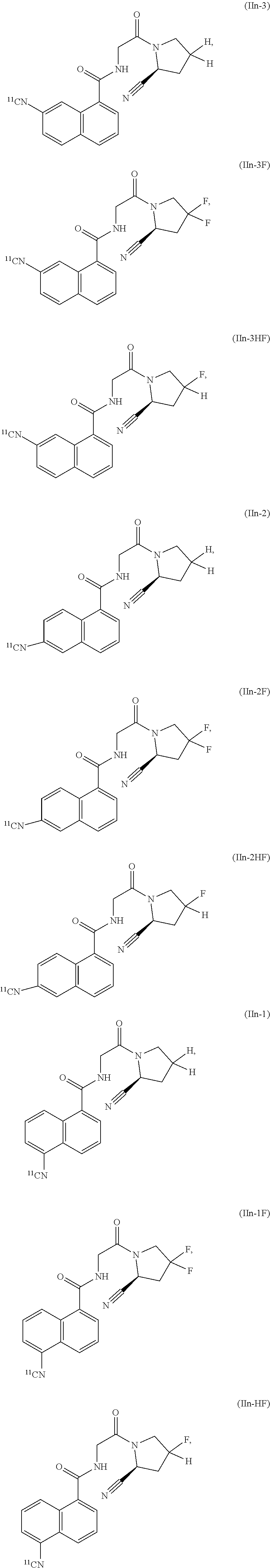

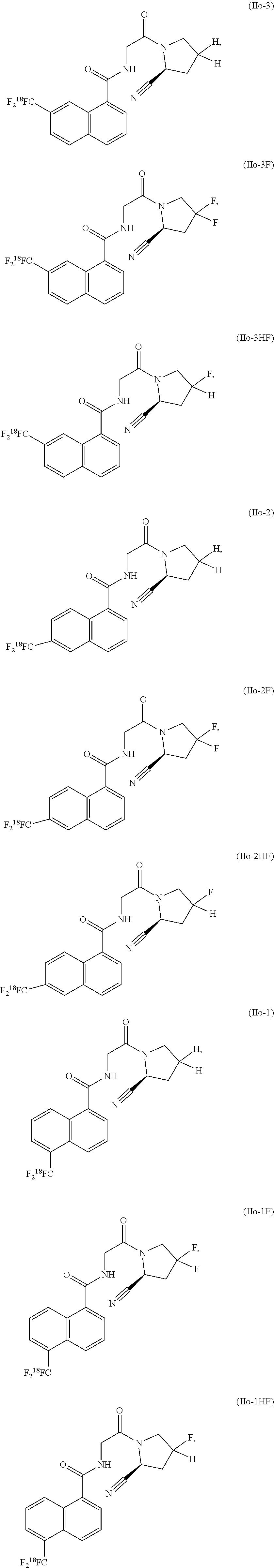

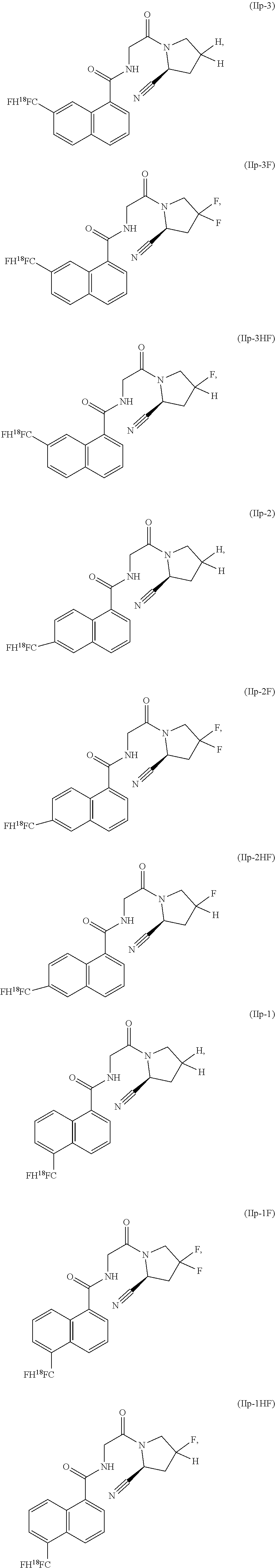

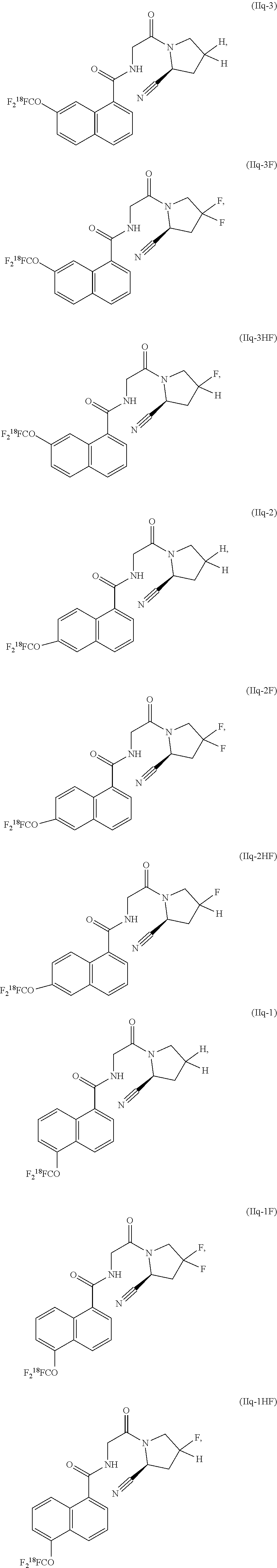

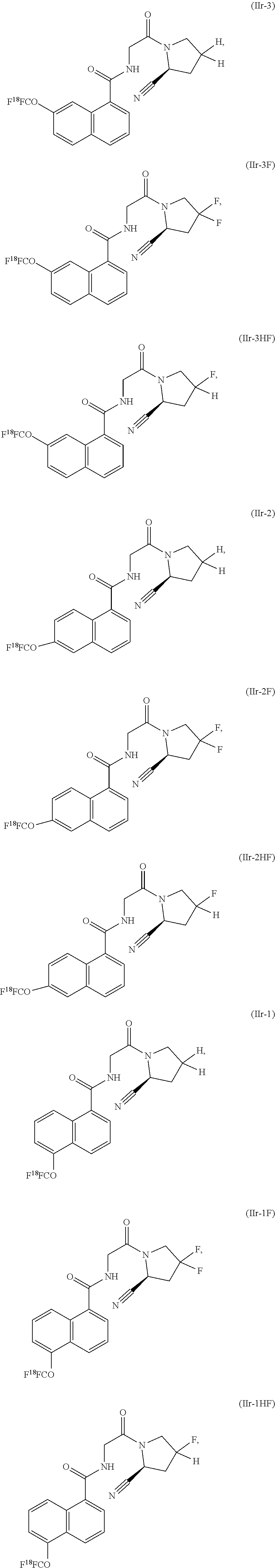

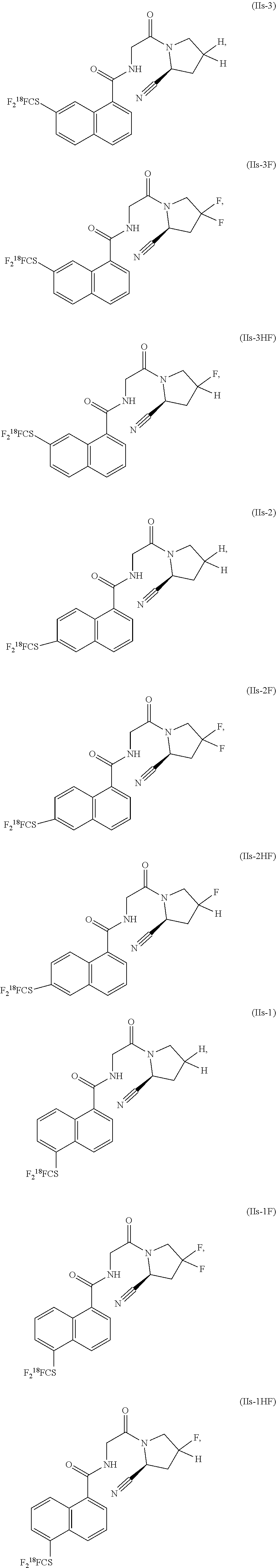

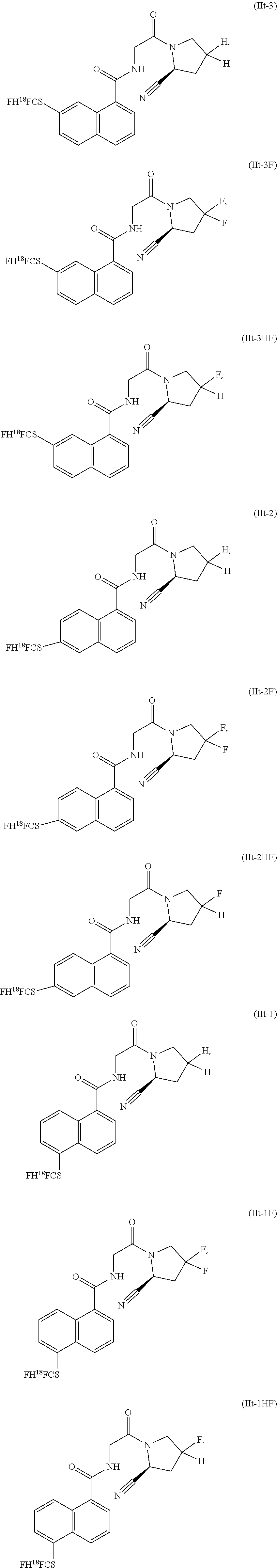

A57. The fibroblast activation protein inhibitor of paragraph A1 having a structure according to one of the following formulae:

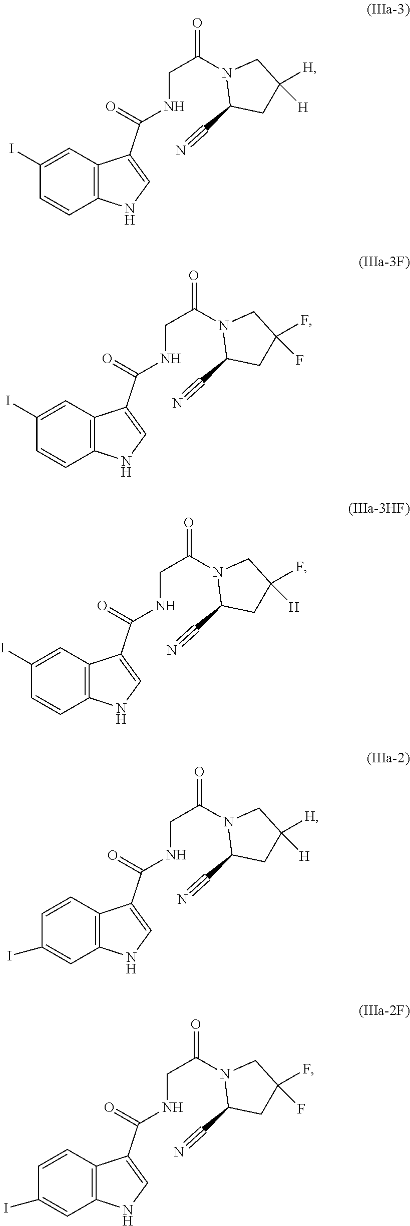

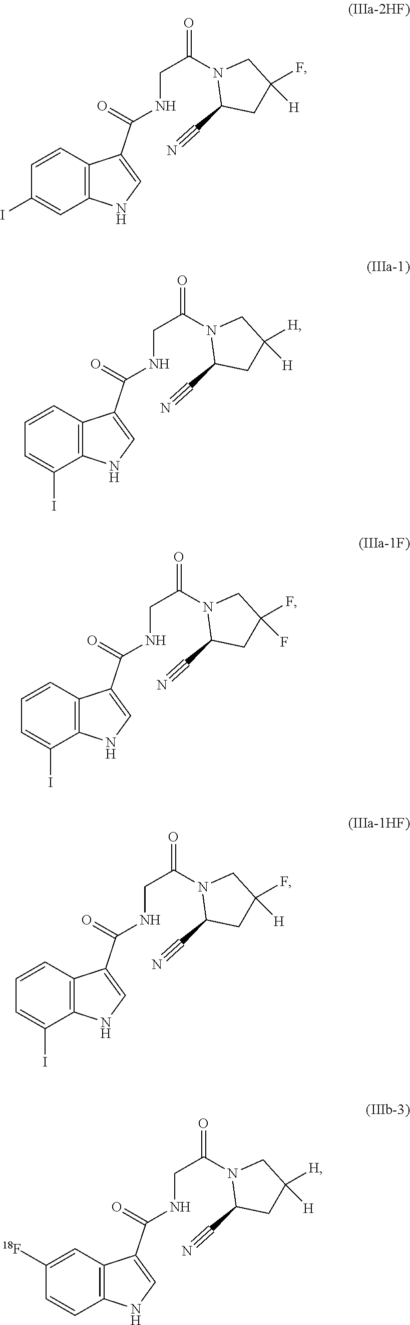

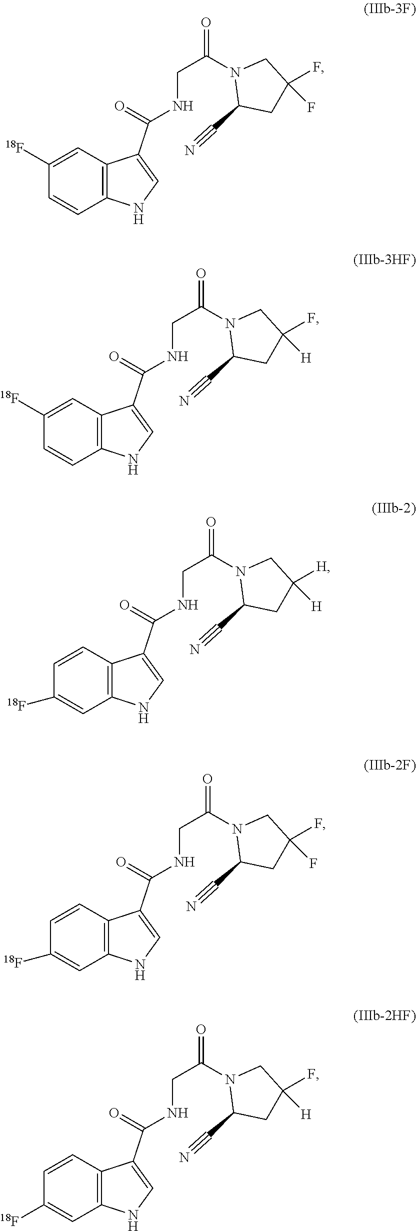

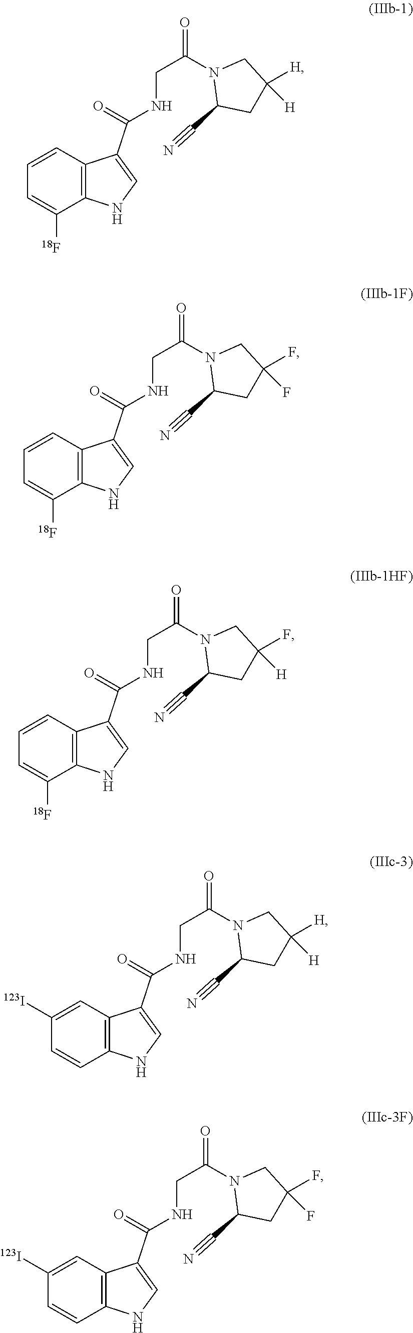

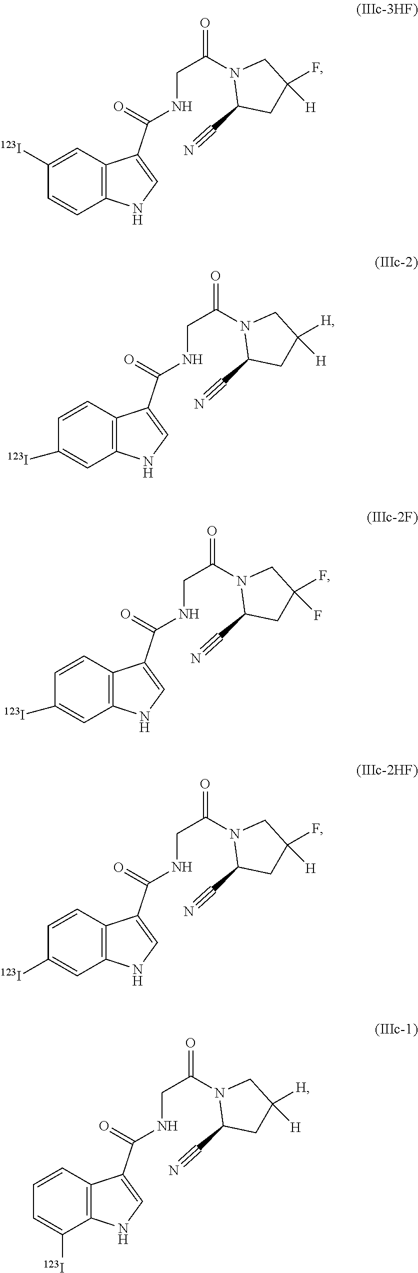

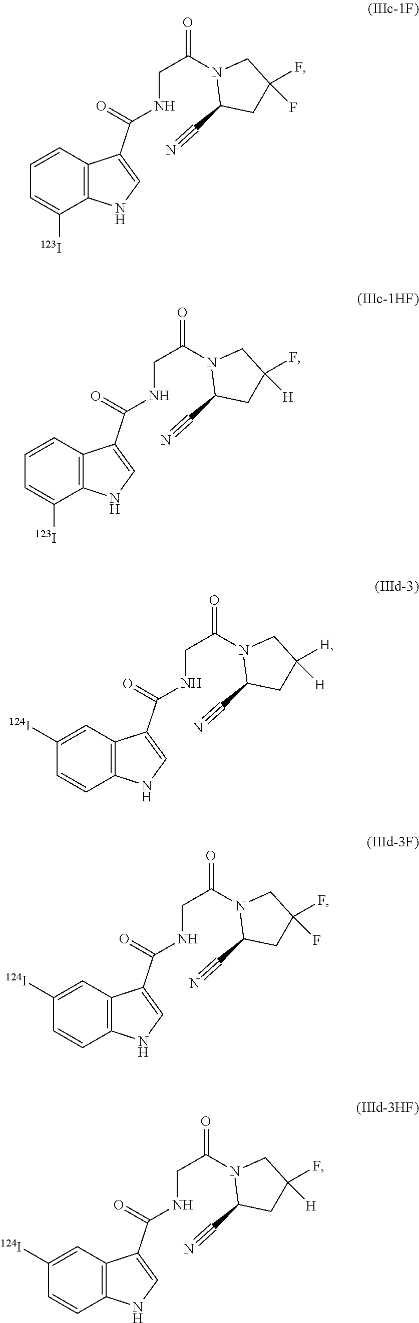

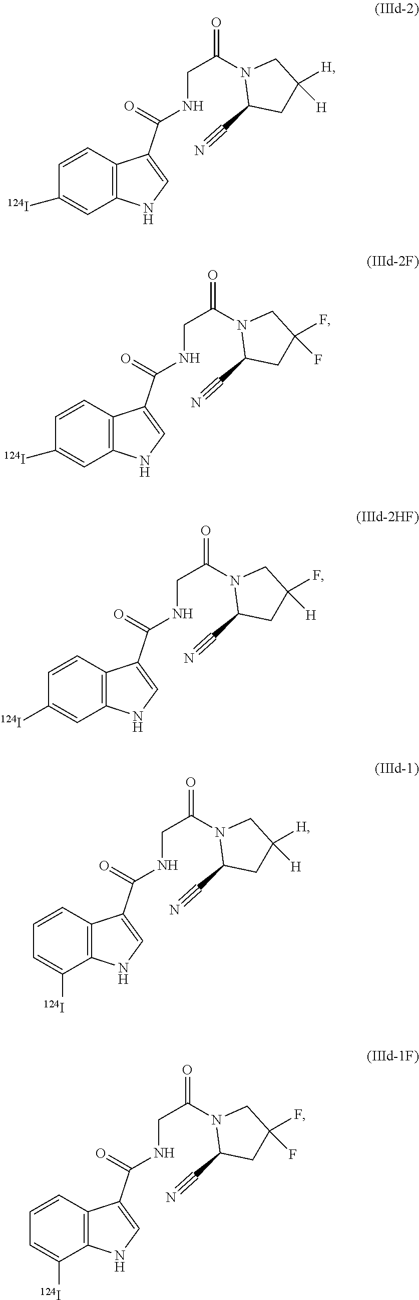

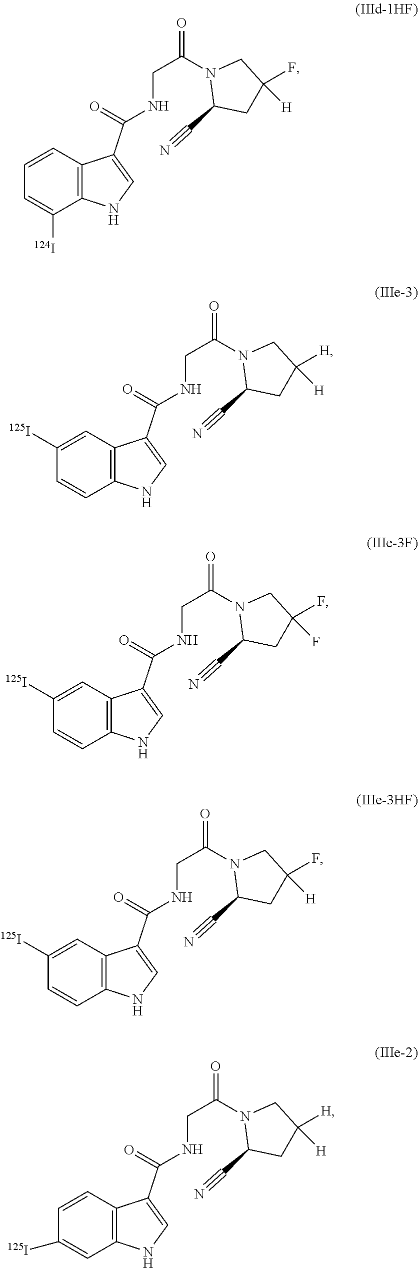

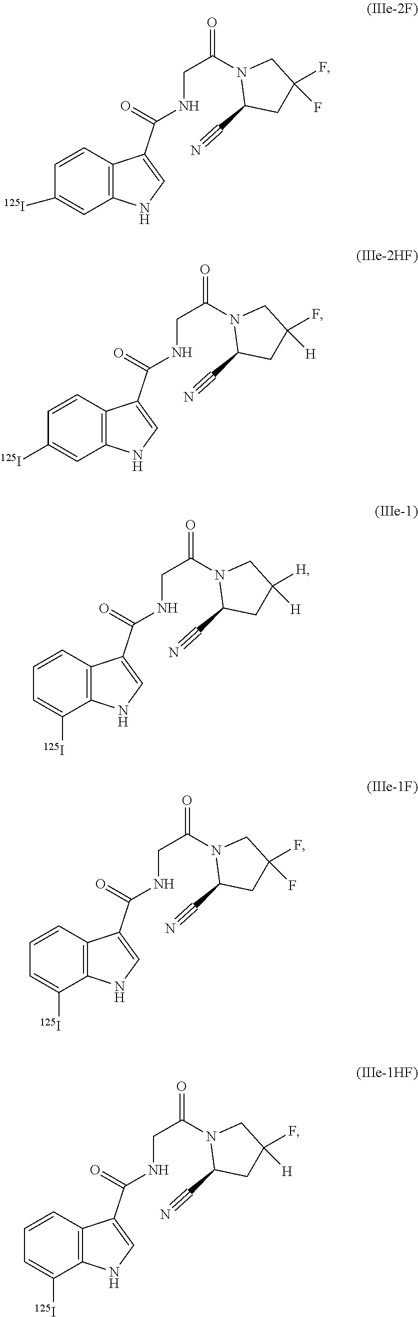

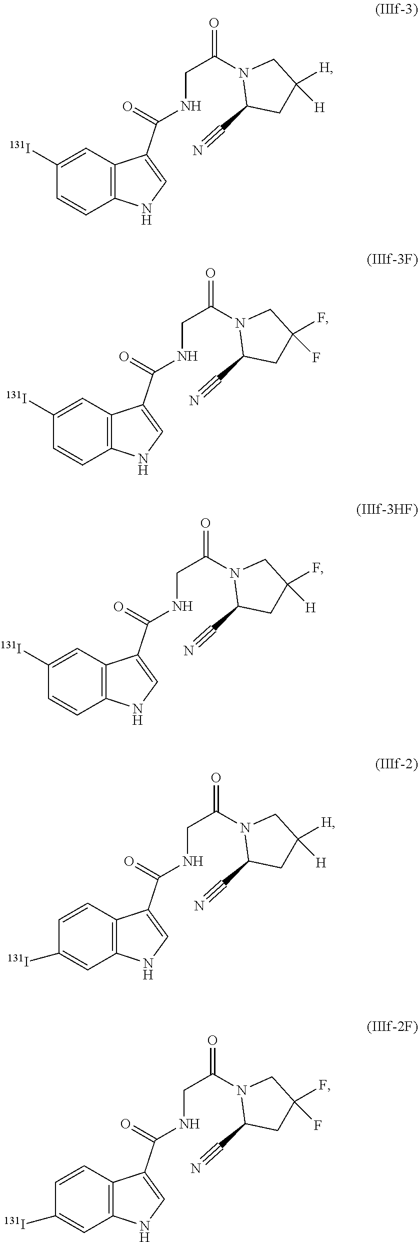

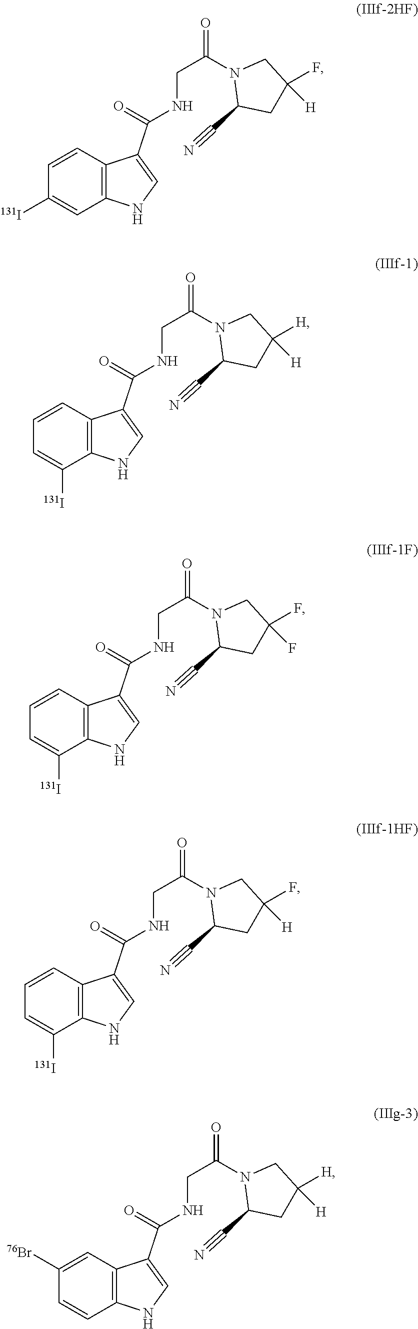

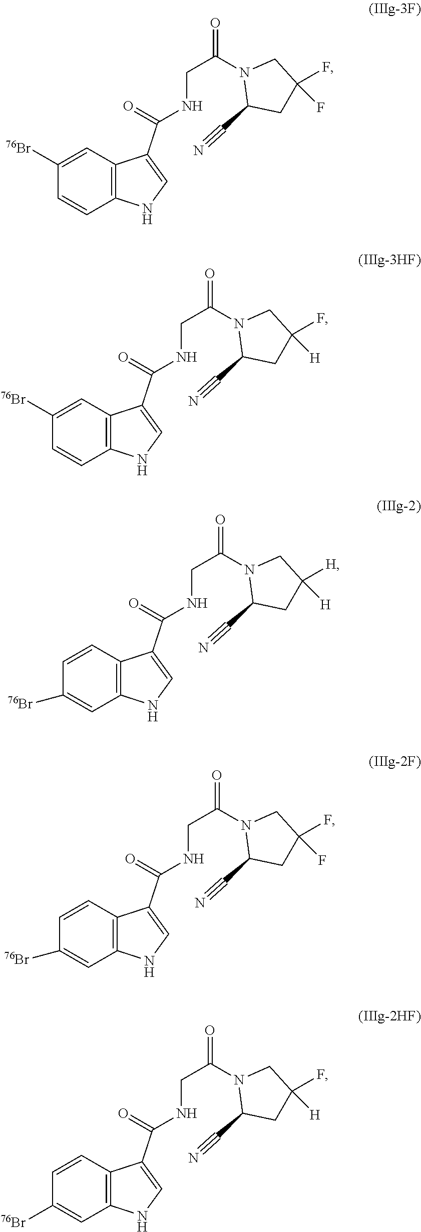

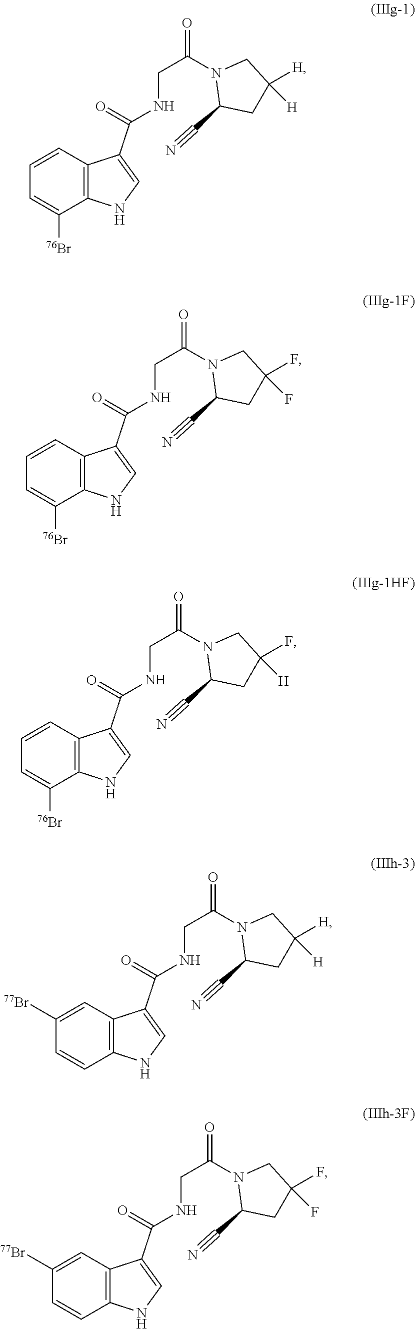

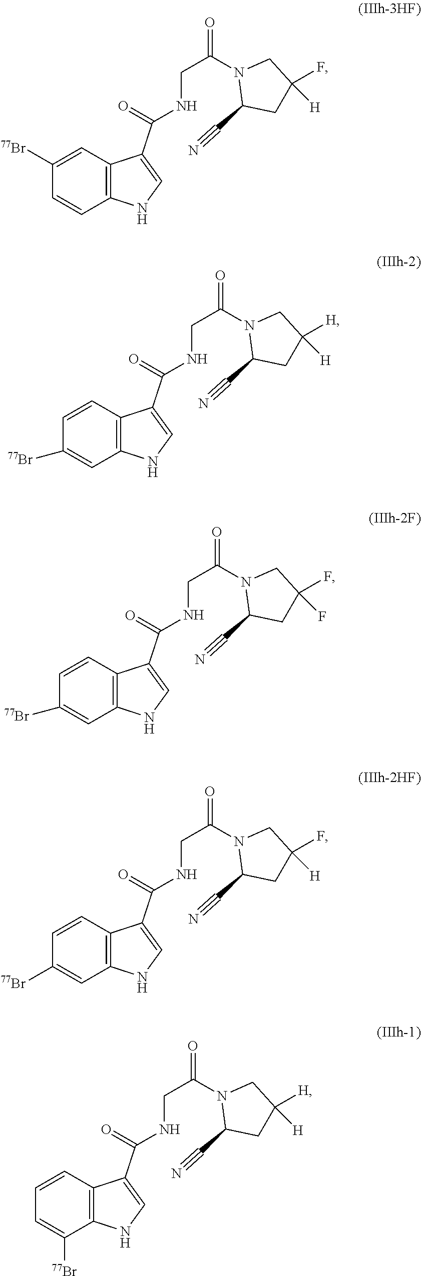

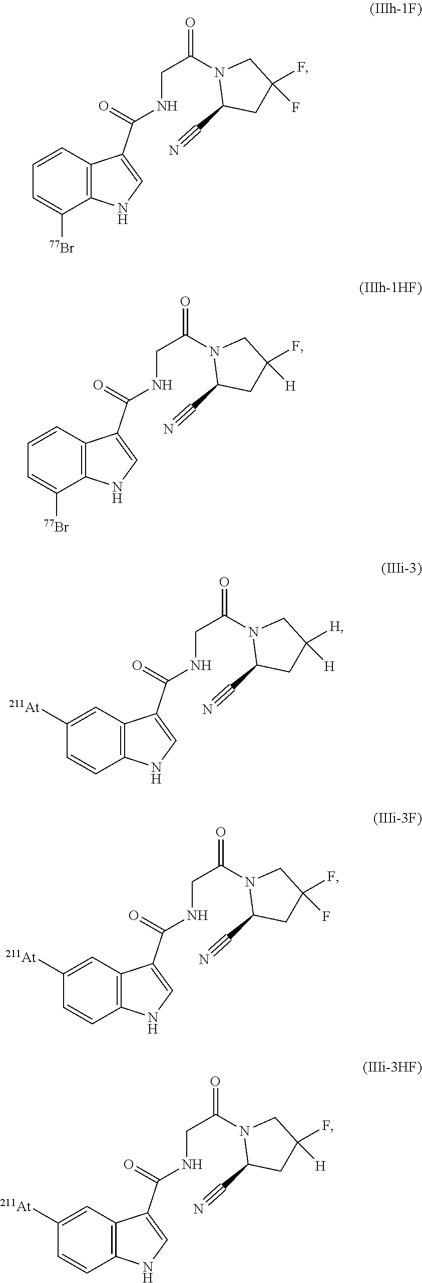

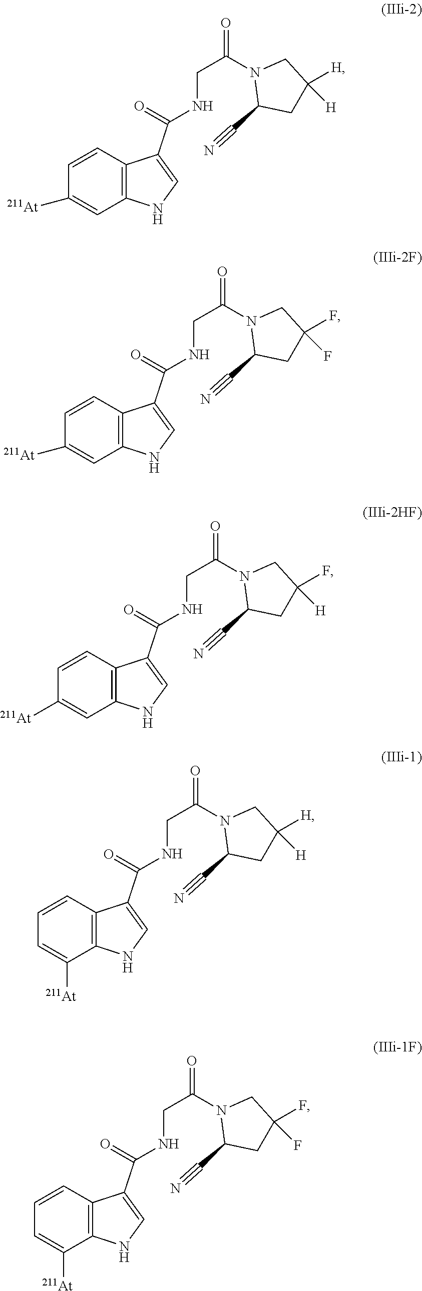

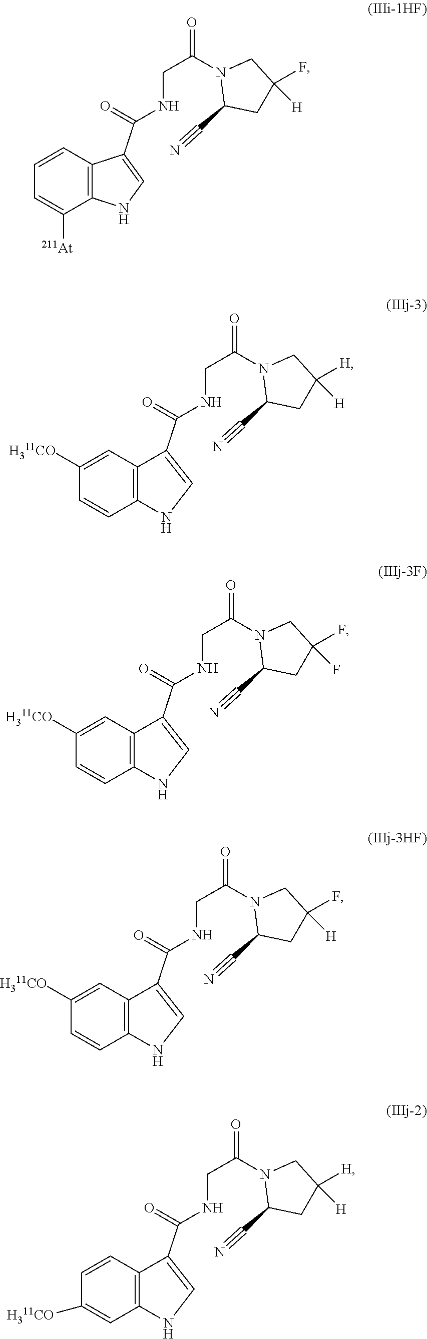

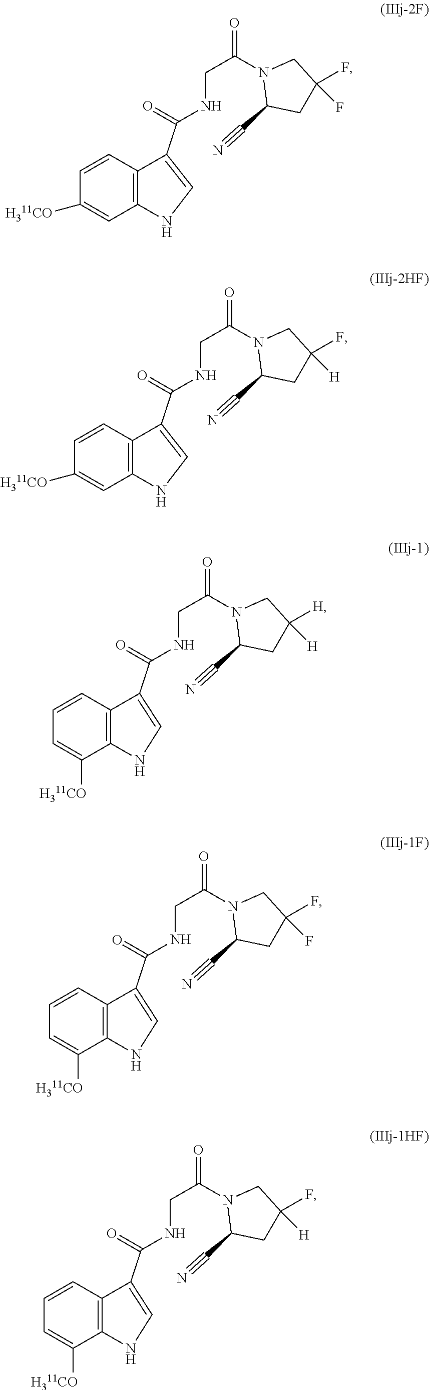

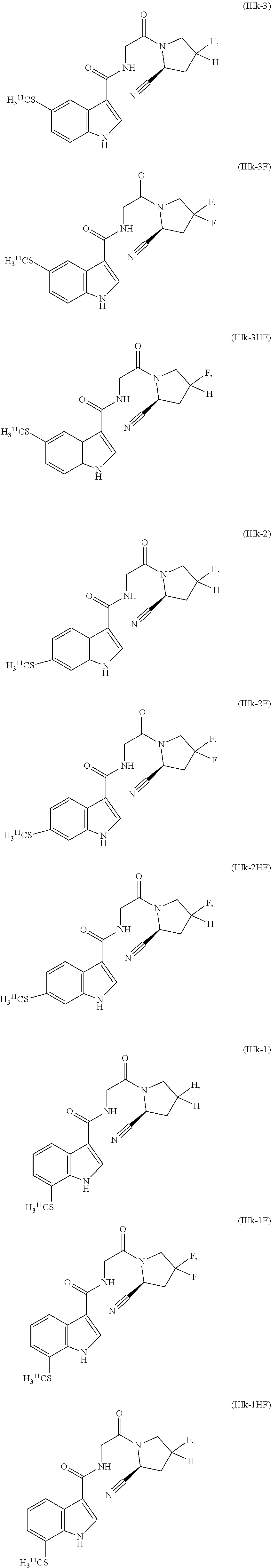

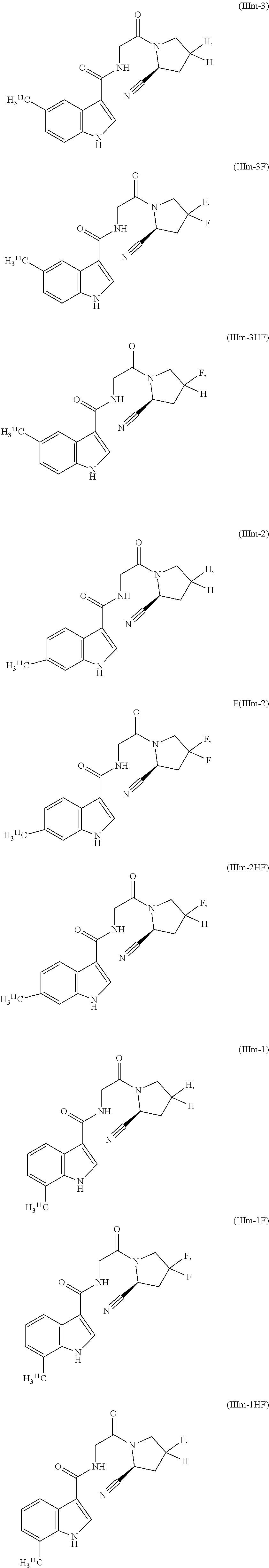

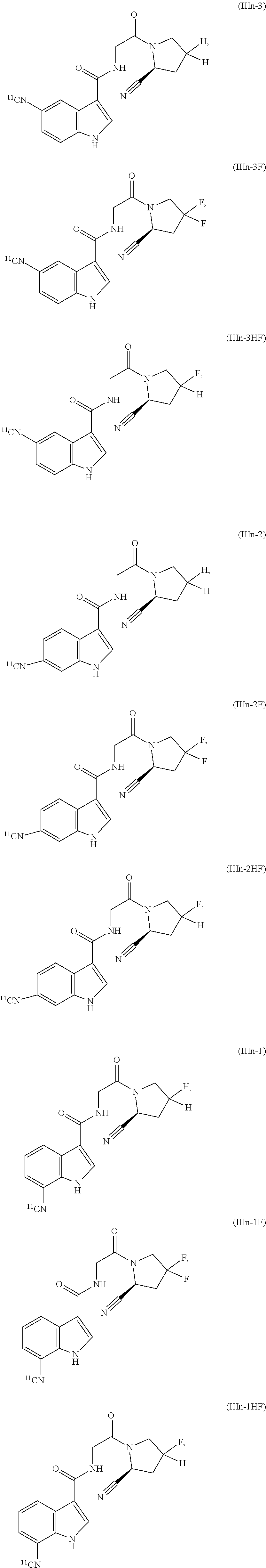

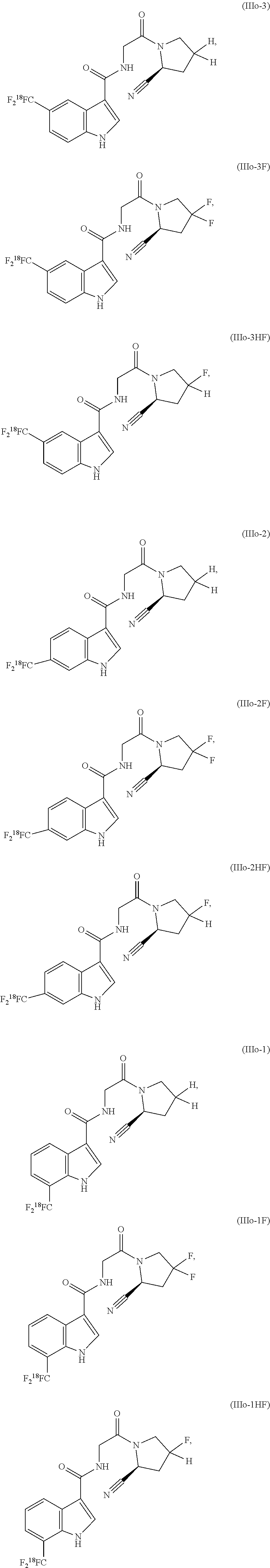

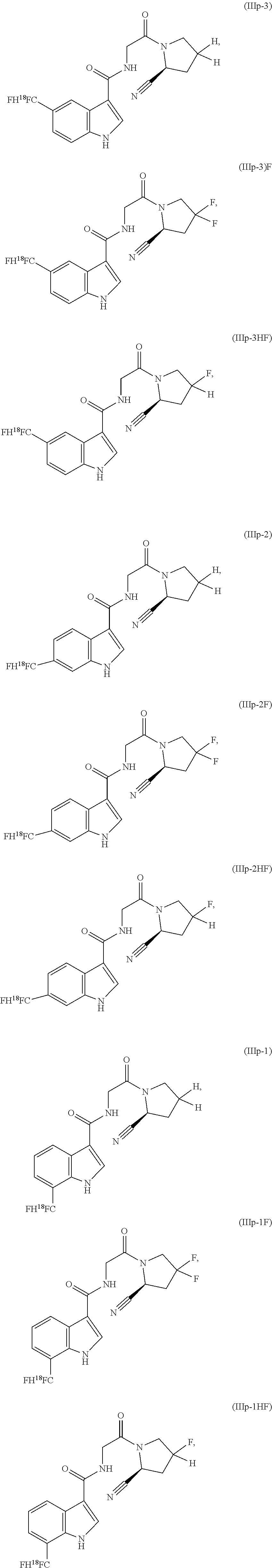

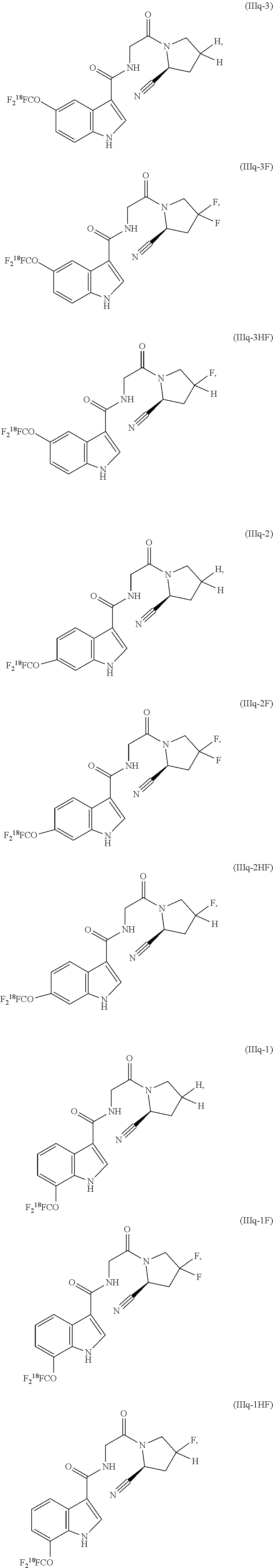

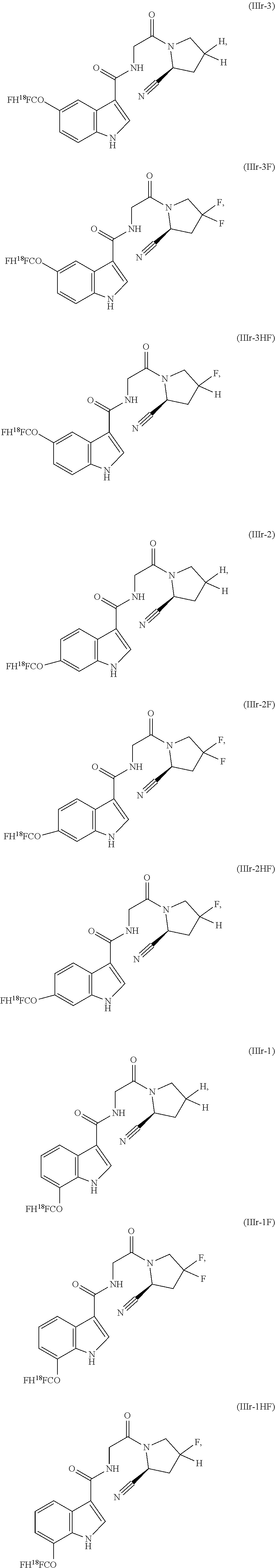

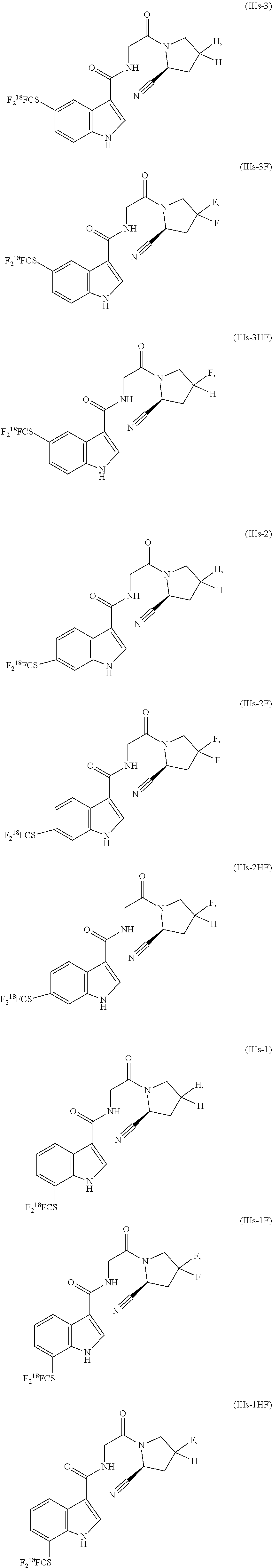

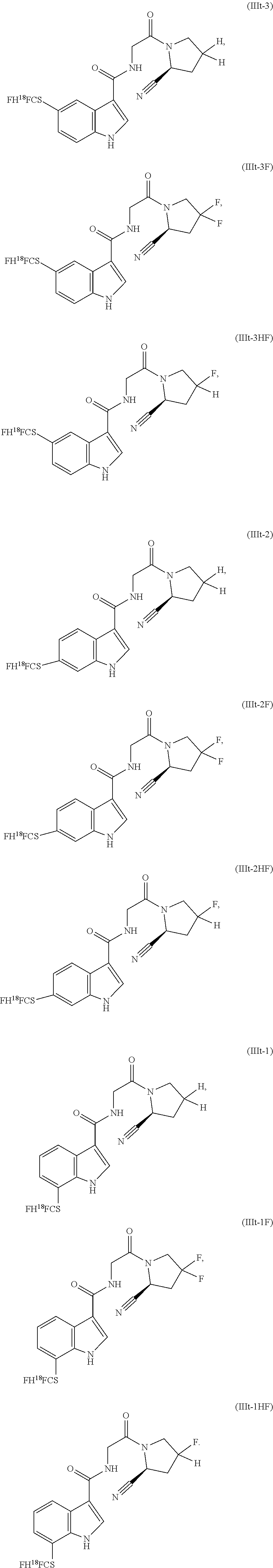

A58. A method of preparing a fibroblast activation protein inhibitor comprising:
admixing a compound having a structure according to formula (IV) with a salt, MX, to form a compound having a structure according to formula (I):

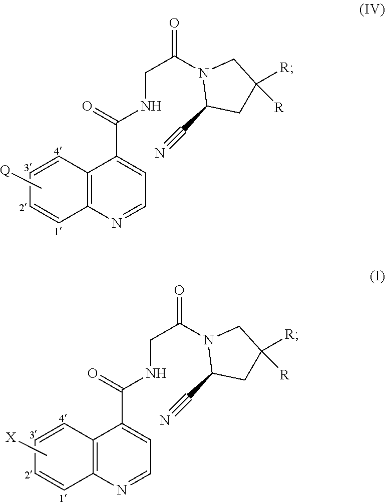

wherein
M is selected from the group of K, Li, Na, Rb, Cs, tetraethylammonium, and tetrabutylammonium;
in the salt, KX, and in formula (I), X is selected from the group of ¹²³I, ¹²⁴I, ¹²⁵I, ¹³¹I, ²¹¹At, ⁷⁶Br, ⁷⁷Br, ¹⁸F, H₃¹¹CO, CF₂¹⁸F, CHF¹⁸F, OCF₂¹⁸F, OCHF¹⁸F, SCF₂¹⁸F, SCHF¹⁸F, ¹¹CN, ¹¹CH₃, and H₃¹¹CS; and X is the same in the salt and in formula (I);
Q is selected from trialkyl stannyl, trialkyl germyl, and trialkyl silyl;
in formula (IV) and formula (I), Q and X are located at carbon 1′, 2′, or 3′, and the location of Q in formula (IV) and the location of X in formula (I) are the same; and
each R is independently selected from H, F, ²H, CH₃, and cyclopentyl, and the R in formula (I) are the same as the R in formula (IV).
A59. The method of paragraph A58, wherein the admixing is performed in the presence of Cu(OTf)₂, (CH₃CN)₄CuOTf, or CuOTf-toluene.
A60. The method of paragraph A58 or A59, wherein the admixing is performed in the presence of pyridine and DMF or DMA.
A61. The method of any one of paragraph A58 or paragraph A60, wherein the admixing is performed at a temperature in a range of about 20° C. to about 140° C. ° C. for about 5 minutes to about 72 hrs.
A62. The method of any one of paragraphs A58 to A61, further comprising preparing the compound having a structure according to formula (IV), by admixing a compound having a structure according to formula (Ia) with one of hexaalkyldistannane, hexaalkyldigermane, or hexaalkyldisilane:

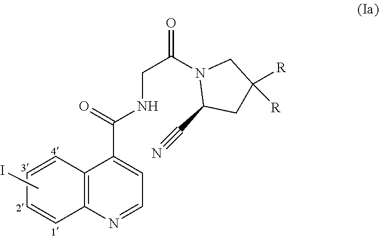

wherein the R in formula (Ia) are the same R as in the compound according to formula (IV) and the I is located at the same carbon as the Q in the compound according to formula (IV).
A63. The method of paragraph A62, wherein the admixing of the compound having a structure according to formula (Ia) with one of hexaalkyldistannane, hexalkyldigermane, or hexaalkyldisilane is performed in the presence of LiCl, toluene, and Pd(PPh₃)₄.
A64. The method of any one of paragraphs A62 or A63, wherein the admixing wherein the compound having a structure according to formula (Ia) with one of hexaalkyldistannane, hexalkyldigermane, or hexaalkyldisilane is performed at a temperature in a range of about 20° C. to about 140° C. ° C. for a time in a range of 5 minutes to 72 hrs.
A65. The method of any one of paragraphs A62 to A64, wherein the alkyl of the hexaalkyldistannane, hexaalkyldigermane, or hexaalkyldisilane is methyl or butyl.
A66. The method of any one of paragraphs A62 to A65, wherein the hexaalkyldistannane is hexamethyldistannane.
A67. The method of any one of paragraphs A62 to A65, wherein the hexaalkyldistannane is hexabutyldistannane.
A68. The method of any one of paragraphs A58 to A67, wherein Q and X are located at the 3′ carbon.
A69. The method of any one of paragraphs A58 to A67, wherein Q and X are located at the 2′ carbon.
A70. The method of any one of paragraphs A58 to A67, wherein Q and X are located at the 1′ carbon.
A71. The method of any one of paragraphs A58 to A70, wherein X is ¹⁸F.
A72. The method of any one of paragraphs A58 to A70, wherein X is ²¹¹At.
A73. The method of any one of paragraphs A58 to A70, wherein X is ¹²³I.
A74. The method of any one of paragraphs A58 to A70, wherein X is ¹²⁴I.
A75. The method of any one of paragraphs A58 to A70, wherein X is ¹²⁵I.
A76. The method of any one of paragraphs A58 to A70, wherein X is ¹³¹I.
A77. The method of any one of paragraphs A58 to A70, wherein X is ⁷⁶Br.
A78. The method of any one of paragraphs A58 to A70, wherein X is ⁷⁷Br.
A79. The method of any one of paragraphs A58 to A70, wherein X is H₃¹¹CO.
A80. The method of any one of paragraphs A58 to A70, wherein X is CF₂¹⁸F.
A81. The method of any one of paragraphs A58 to A70, wherein X is CHF¹⁸F.
A82. The method of any one of paragraphs A58 to A70, wherein X is OCF₂¹⁸F.
A83. The method of any one of paragraphs A58 to A70, wherein X is OCHF¹⁸F.
A84. The method of any one of paragraphs A58 to A70, wherein X is SCF₂¹⁸F.
A85. The method of any one of paragraphs A58 to A70, wherein X is SCHF¹⁸F.
A86. The method of any one of paragraphs A58 to A70, wherein X is ¹¹CN.
A87. The method of any one of paragraphs A58 to A70, wherein X is ¹¹CH₃.
A88. The method of any one of paragraphs A58 to A70, wherein X is H₃¹¹CS
A89. The method of any one of paragraphs A58 to A88, wherein in the compounds having a structure according to formulae (IV), (I), and (Ia), at least one R is H.
A90. The method of any one of paragraphs A58 to A88, wherein both R are H.
A91. The method of any one of paragraphs A58 to A89, wherein at least one R is F.
A92. The method of any one of paragraphs A58 to A88, wherein both R are F.
A93. The method of any one of paragraphs A58 to A89 or A91, wherein at least one R is ²H.
A94. The method of any one of paragraphs A58 to A88, wherein both R are ²H.
A95. The method of any one of paragraphs A58 to A89, A91, or A93, wherein at least one R is CH₃.
A96. The method of any one of paragraphs A58 to A88, wherein both R are CH₃.
A97. The method of any one of paragraphs A58 to A89, A91, A93, or A95, wherein at least one R is cyclopentyl.
A98. The method of any one of paragraphs A58 to A88, wherein both R are cyclopentyl.
A99. The method of any one of paragraphs A58 to A67, wherein Q and X are located at carbon 3′ and all R are H.
A100. The method of any one of paragraphs A58 to A67, wherein Q and X are located at carbon 3′ and all R are F.
A101. The method of any one of paragraphs A58 to A67, wherein Q and X are located at carbon 2′ and all R are H.
A102. The method of any one of paragraphs A58 to A67, wherein Q and X are located at carbon 2′ and all R are F.
A103. The method of any one of paragraphs A58 to A67, wherein Q and X are located at carbon 1′ and all R are H.
A104. The method of any one of paragraphs A58 to A67, wherein Q and X are located at carbon 1′ and all R are F.
A105. The method of any one of paragraphs A58 to A104, wherein Q is trialkyl stannyl.
A106. The method of any one of paragraphs A58 to A104, wherein Q is trialkyl germyl.
A107. The method of any one of paragraphs A58 to A104, wherein Q is trialkyl silyl.
A108. The method of any one of paragraphs A58 to A107, wherein the alkyl of the trialkyl tin, trialkyl germanium, or trialkyl silyl is methyl or butyl.
A109. The method of paragraph A108, wherein the alkyl of the trialkyl stannyl, trialkyl germyl, or trialkyl silyl is methyl.
A110. The method of claim A108, wherein the alkyl of the trialkyl stannyl, trialkyl germyl, or trialkyl silyl is butyl.
A111. A method of preparing a fibroblast activation protein inhibitor comprising: admixing a compound having a structure according to formula (VI) with a salt, MX, to form a compound having a structure according to formula (II):

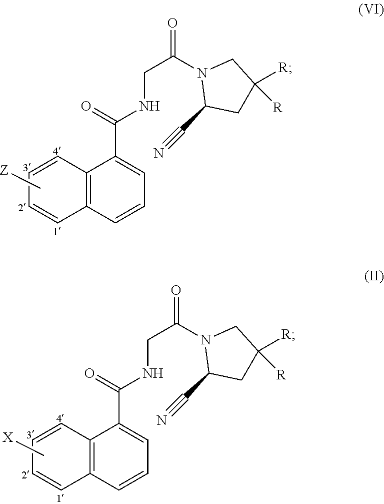

wherein
M is selected from the group of K, Li, Na, Rb, Cs, tetraethylammonium, and tetrabutylammonium;
in the salt, KX, and in formula (II), X is selected from the group of ¹²³I, ¹²⁴I, ¹²⁵I, ¹³¹I, ²¹¹At, ⁷⁶Br, ⁷⁷Br, ¹⁸F, H₃¹¹CO, CF₂¹⁸F, CHF¹⁸F, OCF₂¹⁸F, OCHF¹⁸F, SCF₂¹⁸F, SCHF¹⁸F, ¹¹CN, ¹¹CH₃, and H₃¹¹CS, and X is the same in the salt and in formula (II);
Q is selected from trialkyl stannyl, trialkyl germyl, or trialkyl silyl;
in formula (VI) and formula (II), Q and X are located at carbon 1′, 2′, or 3′, and the location of Q in formula (VI) and the location of X in formula (II) are the same; and
each R is independently selected from H, F, ²H, CH₃, and cyclopentyl, and the R in formula (II) are the same as the R in formula (VI).
A112. The method of paragraph A111, wherein the admixing is performed in the presence of Cu(OTf)₂, (CH₃CN)₄CuOTf, or CuOTf-toluene.
A113. The method of paragraph A111 or A112, wherein the admixing is performed in the presence of pyridine and DMF or DMA.
A114. The method of any one of paragraphs A111 to 1A13, wherein the admixing is performed at a temperature in a range of 20° C. to 140° C. for a time in a range of 5 minutes to 72 hrs.
A115. The method of any one of claims 111 to 114, further comprising preparing the compound having a structure according to formula (VI), by admixing a compound having a structure according to formula (IIa) with one of hexaalkyldistannane, hexaalkyldigermane, or hexaalkyldisilane:

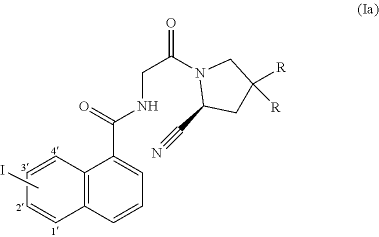

wherein the R in formula (IIa) are the same R as in the compound according to formula (VI) and the I is located at the same carbon as the Q in the compound according to formula (VI).
A116. The method of paragraph A115, wherein the admixing of the compound having a structure according to formula (IIa) with one of hexaalkyldistannane, hexalkyldigermane, or hexaalkyldisilane is performed in the presence of LiCl, toluene, and Pd(PPh₃)₄.
A117. The method of any one of paragraphs A115 or A116, wherein the admixing wherein the compound having a structure according to formula (IIa) with one of hexaalkyldistannane, hexalkyldigermane, or hexaalkyldisilane is performed at a temperature in a range of 20° C. to 140° C. for a time in a range of about 5 minutes to about 72 hrs.
A118. The method of any one of paragraphs A115 to A117, wherein the alkyl of the hexaalkyldistannane, hexaalkyldigermane, or hexaalkyldisilane is methyl or butyl.
A119. The method of any one of paragraphs A115 to A116, wherein the hexaalkyldistannane is hexamethyldistannane.
A120. The method of any one of paragraphs A115 to A116, wherein the hexaalkyldistannane is hexabutyldistannane.
A121. The method of any one of paragraphs A111 to A118, wherein Q and X are located at the 3′ carbon.
A122. The method of any one of paragraphs A111 to A118, wherein Q and X are located at the 2′ carbon.
A123. The method of any one of paragraphs A111 to A118, wherein Q and X are located at the 1′ carbon.
A124. The method of any one of paragraphs A111 to A123, wherein X is ¹⁸F.
A125. The method of any one of paragraphs A111 to A123, wherein X is ²¹¹At.
A126. The method of any one of paragraphs A111 to A123, wherein X is ¹²³I.
A127. The method of any one of paragraphs A111 to A123, wherein X is ¹²⁴I.
A128. The method of any one of paragraphs A111 to A123, wherein X is ¹²⁵I.
A128. The method of any one of paragraphs A111 to A123, wherein X is ¹³¹I.
A130. The method of any one of paragraphs A111 to A123, wherein X is ⁷⁶Br.
A131. The method of any one of paragraphs A111 to A123, wherein X is ⁷⁷Br.
A132. The method of any one of paragraphs A111 to A123, wherein X is H₃¹¹CO.
A133. The method of any one of paragraphs A111 to A123, wherein X is CF₂¹⁸F.
A134. The method of any one of paragraphs A111 to A123, wherein X is CHF¹⁸F.
A135. The method of any one of paragraphs A111 to A123, wherein X is OCF₂¹⁸F.
A136. The method of any one of paragraphs A111 to A123, wherein X is OCHF¹⁸F.
A137. The method of any one of paragraphs A111 to A123, wherein X is SCF₂¹⁸F.
A138. The method of any one of paragraphs A111 to A123, wherein X is SCHF¹⁸F.
A139. The method of any one of paragraphs A111 to A123, wherein X is ¹¹CN.
A140. The method of any one of paragraphs A111 to A123, wherein X is ¹¹CH₃.
A141. The method of any one of paragraphs A111 to A123, wherein X is H₃¹¹CS.
A142. The method of any one of claims A111 to A132, wherein in the compounds having a structure according to formulae (IV), (1), and (Ia), at least one R is H.
A143. The method of any one of paragraphs A111 to A132, wherein both R are H.
A144. The method of any one of paragraphs A111 to A133, wherein at least one R is F.
A145. The method of any one of paragraphs A111 to A132, wherein both R are F.
A146. The method of any one of paragraphs A111 to A133 or A135, wherein at least one R is ²H.
A147. The method of any one of paragraphs A111 to A132, wherein both R are ²H.
A148. The method of any one of paragraphs A111 to A133, A135, or A137, wherein at least one R is CH₃.
A149. The method of any one of paragraphs A111 to A132, wherein both R are CH₃.
A150. The method of any one of paragraphs A111 to A133, A135, A137, or A139, wherein at least one R is cyclopentyl.
A151. The method of any one of paragraphs A111 to A132, wherein both R are cyclopentyl.
A152. The method of any one of paragraphs A111 to A120, wherein Q and X are located at carbon 3′ and all R are H.
A153. The method of any one of paragraphs A111 to A120, wherein Q and X are located at carbon 3′ and all R are F.
A154. The method of any one of paragraphs A111 to A120, wherein Q and X are located at carbon 2′ and all R are H.
A155. The method of any one of paragraphs A111 to A120, wherein Q and X are located at carbon 2′ and all R are F.
A156. The method of any one of paragraphs A111 to A120, wherein Q and X are located at carbon 1′ and all R are H.
A157. The method of any one of paragraphs A111 to A120, wherein Q and X are located at carbon 1′ and all R are F.
A158. The method of any one of paragraphs A111 to A157, wherein Q is trialkyl tin.
A159. The method of any one of paragraphs A111 to A157, wherein Q is trialkyl germanium.
A160. The method of any one of paragraphs A111 to A157, wherein Q is trialkyl silyl.
A161. The method of any one of paragraphs A111 to A157, wherein the alkyl of the trialkyl stannyl, trialkyl germyl, or trialkyl silyl is methyl or butyl.
A162. The method of paragraph A161, wherein the alkyl of the trialkyl stannyl, trialkyl germyl, or trialkyl silyl is methyl.
A163. The method of paragraph A161, wherein the alkyl of the trialkyl stannyl, trialkyl germyl, or trialkyl silyl is butyl.
A164. A method of preparing a fibroblast activation protein inhibitor comprising: admixing a compound having a structure according to formula (VII) with a salt, MX, to form a compound having a structure according to formula (III):

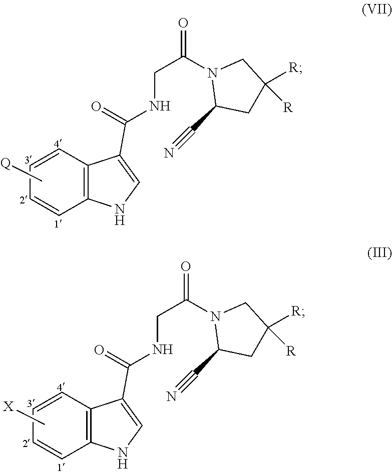

wherein
M is selected from the group of K, Li, Na, Rb, Cs, tetraethylammonium, and tetrabutylammonium;
in the salt, MX, and in formula (III), X is selected from the group of ¹²³I, ¹²⁴I, ¹²⁵I, ¹³¹I, ²¹¹At, ⁷⁶Br, ⁷⁷Br, ¹⁸F, H₃¹¹CO, CF₂¹⁸F, CHF¹⁸F, OCF₂¹⁸F, OCHF¹⁸F, SCF₂¹⁸F, SCHF¹⁸F, ¹¹CN, ¹¹CH₃, and H₃¹¹CS, and X is the same in the salt and in formula (III);
Q is selected from trialkyl stannyl, trialkyl germyl, or trialkyl silyl;
in formula (VII) and formula (III), Q and X are located at carbon 1′, 2′, or 3′, and the location of Q in formula (VII) and the location of X in formula (III) are the same; and
each R is independently selected from H, F, ²H, CH₃, and cyclopentyl, and the R in formula (III) are the same as the R in formula (VII).
A165. The method of paragraph A164, wherein the admixing is performed in the presence of Cu(OTf)₂, (CH₃CN)₄CuOTf, or CuOTf-toluene.
A166. The method of paragraphs A164 or A165, wherein the admixing is performed in the presence of pyridine and DMF or DMA.
A167. The method of any one of paragraphs A165 to A166, wherein the admixing is performed at a temperature in a range of about 20° C. to about 140° C. for a time in a range of about 5 minutes to about 72 hrs.
A168. The method of any one of paragraphs A164 to A167, further comprising preparing the compound having a structure according to formula (VII), by admixing a compound having a structure according to formula (Illa) with one of hexaalkyldistannane, hexaalkyldigermane, or hexaalkyldisilane:

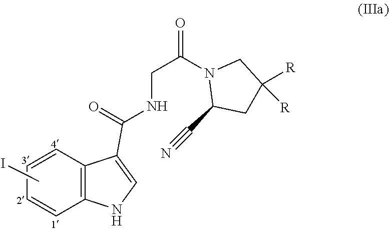

wherein the R in formula (IIIa) are the same R as in the compound according to formula (VII) and the I is located at the same carbon as the Q in the compound according to formula (VII).
A169. The method of paragraph A168, wherein the admixing of the compound having a structure according to formula (IIIa) with one of hexaalkyldistannane, hexalkyldigermane, or hexaalkyldisilane is performed in the presence of LiCl, toluene, and Pd(PPh₃)₄.
A170. The method of any one of paragraphs A168 or A169, wherein the admixing wherein the compound having a structure according to formula (IIIa) with one of hexaalkyldistannane, hexalkyldigermane, or hexaalkyldisilane is performed at a temperature in a range of 20° C. to about 140° C. for a time in a range of about 5 minutes to about 72 hrs.
A171. The method of any one of paragraphs A168 to A170, wherein the alkyl of the hexaalkyldistannane, hexaalkyldigermane, or hexaalkyldisilane is methyl or butyl.
A172. The method of any one of paragraphs A168 to A171, wherein the hexaalkyldistannane is hexamethyldistannane.
A173. The method of any one of paragraphs A168 to A171, wherein the hexaalkyldistannane is hexabutyldistannane.
A174. The method of any one of paragraphs A164 to A173, wherein Q and X are located at the 3′ carbon.
A175. The method of any one of paragraphs A164 to A173, wherein Q and X are located at the 2′ carbon.
A176. The method of any one of paragraphs A164 to A173, wherein Q and X are located at the 1′ carbon.
A177. The method of any one of paragraphs A164 to A176, wherein X is ¹⁸F.
A178. The method of any one of paragraphs A164 to A176, wherein X is ²¹¹At.
A179. The method of any one of paragraphs A164 to A176, wherein X is ¹²³I.
A180. The method of any one of paragraphs A164 to A176, wherein X is ¹²⁴I.
A181. The method of any one of paragraphs A164 to A176, wherein X is ¹²⁵I.
A182. The method of any one of paragraphs A164 to A176, wherein X is ¹³¹I.
A183. The method of any one of paragraphs A164 to A176, wherein X is ⁷⁶Br.
A184. The method of any one of paragraphs A164 to A176, wherein X is ⁷⁷Br.
A185. The method of any one of paragraphs A164 to A176, wherein X is H₃¹¹CO.
A186. The method of any one of paragraphs A164 to A176, wherein X is CF₂¹⁸F.
A187. The method of any one of paragraphs A164 to A176, wherein X is CHF¹⁸F.
A188. The method of any one of paragraphs A164 to A176, wherein X is OCF₂¹⁸F.
A189. The method of any one of paragraphs A164 to A176, wherein X is OCHF¹⁸F.
A190. The method of any one of paragraphs A164 to A176, wherein X is SCF₂¹⁸F.
A191. The method of any one of paragraphs A164 to A176, wherein X is SCHF¹⁸F.
A192. The method of any one of paragraphs A164 to A176, wherein X is ¹¹CN.
A193. The method of any one of paragraphs A164 to A176, wherein X is ¹¹CH₃.
A194. The method of any one of paragraphs A164 to A176, wherein X is H₃¹¹CS.
A195. The method of any one of paragraphs A164 to A194, wherein in the compounds having a structure according to formulae (IV), (I), and (Ia), at least one R is H.
A196. The method of any one of paragraphs A164 to A194, wherein both R are H.
A197. The method of any one of paragraphs A164 to A195, wherein at least one R is F.
A198. The method of any one of paragraphs A164 to A194, wherein both R are F.
A199. The method of any one of paragraphs A164 to A195 or A197, wherein at least one R is ²H.
A200. The method of any one of paragraphs A164 to A194, wherein both R are ²H.
A201. The method of any one of paragraphs A164 to A195, A197, or A199, wherein at least one R is CH₃.
A202. The method of any one of paragraphs A164 to A194, wherein both R are CH₃.
A203. The method of any one of paragraphs A164 to A195, A197, A199, or A201, wherein at least one R is cyclopentyl.
A204. The method of any one of paragraphs A164 to A194, wherein both R are cyclopentyl.
A205. The method of any one of paragraphs A164 to A173, wherein Q and X are located at carbon 3′ and all R are H.
A206. The method of any one of paragraphs A164 to A173, wherein Q and X are located at carbon 3′ and all R are F.
A207. The method of any one of paragraphs A164 to A173, wherein Q and X are located at carbon 2′ and all R are H.
A208. The method of any one of paragraphs A164 to A173, wherein Q and X are located at carbon 2′ and all R are F.
A209. The method of any one of paragraphs A164 to A173, wherein Q and X are located at carbon 1′ and all R are H.
A210. The method of any one of paragraphs A164 to A173, wherein Q and X are located at carbon 1′ and all R are F.
A211. The method of any one of paragraphs A164 to A210, wherein Q is trialkyl stannyl.
A212. The method of any one of paragraphs A164 to A210, wherein Q is trialkyl germyl.
A213. The method of any one of paragraphs A164 to A210, wherein Q is trialkyl silyl.
A214. The method of any one of paragraphs A164 to A210, wherein the alkyl of the trialkyl stannyl, trialkyl germyl, or trialkyl silyl is methyl or butyl.
A215. The method of paragraph A214, wherein the alkyl of the trialkyl stannyl, trialkyl germyl, or trialkyl silyl is methyl.
A216. The method of paragraphs A214, wherein the alkyl of the trialkyl stannyl, trialkyl germyl, or trialkyl silyl is butyl.
A217. A method of preparing a fibroblast activation protein inhibitor comprising: admixing a compound having a structure according to formula (VIII) with a radiolabeled salt, MX, to form a compound having a structure according to formula (I):

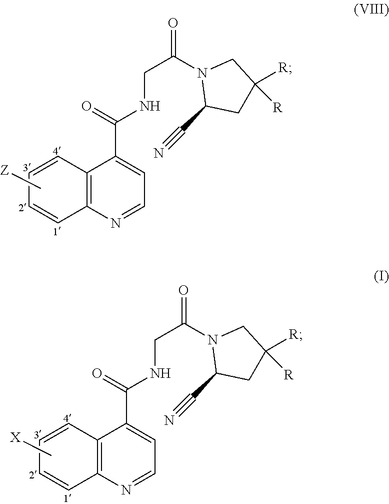

wherein
Z has a structure according selected from the group of:

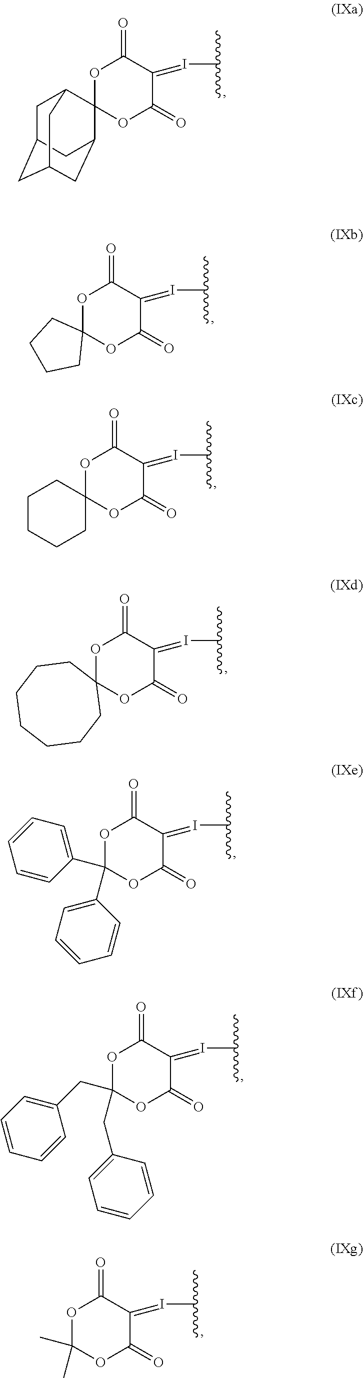

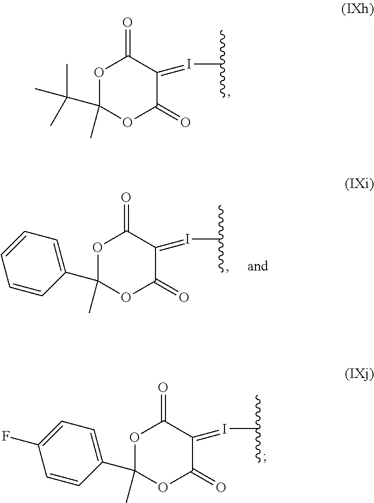 [SMILES: *I=C1C(=O)OC(C)(C(C)(C)C)OC1=O ; *I=C1C(=O)OC(C)(c2ccccc2)OC1=O ; *I=C1C(=O)OC(C)(c2ccc(F)cc2)OC1=O]

in formula (VIII) and formula (I), Z and X are located at carbon 1′, 2′, or 3′, and the location of Z in formula (VIII) and the location of X in formula (I) are the same;
M is selected from the group of K, Li, Na, Rb, Cs, tetraethylammonium, and tetrabutylammonium;
in the salt, MX, and in formula (I), X is selected from the group of ¹⁸F, ²¹¹At, ¹²³I, ¹²⁴I, ¹²⁵I, ¹³¹I, ⁷⁶Br, ⁷⁷Br; and
each R is independently selected from H, F, ²H, CH₃, and cyclopentyl, and the R in formula (I) are the same as the R in formula (VIII).
A218. The method of paragraph A217, wherein the admixing is performed in the presence of dimethylsulfoxide (DMSO), dimethylformamide (DMF), dimethylacetamide (DMA), or a combination thereof.
A219. The method of paragraphs A217 or claim A218, wherein the admixing is performed at a temperature in a range of 85° C. to 140° C. for a time in a range of about 5 minutes to about 12 hrs.
A220. The method of any one of paragraphs A217 to A219, further comprising preparing the compound having a structure according to formula (VIII), by admixing a compound having a structure according to formula (Ia):

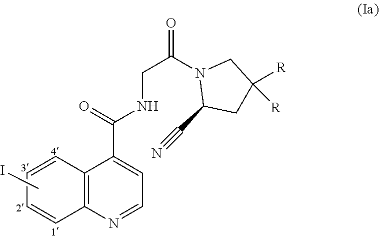

with 1-chloromethyl-4-fluoro-1,4-diazoniabicyclo[2.2.2]octanebis(tetrafluoroborate), and trimethylsilyl acetate to form a reaction product;
wherein the R in formula (Ia) are the same R as in the compound according to formula (VIII) and the I is located at the same carbon as the Z in the compound according to formula (VIII).
A221. The method of paragraph A220, wherein the admixing is performed in the presence of acetonitrile
A222. The method of paragraphs A220 or A221, wherein the admixing comprises dropwise addition of 1-chloromethyl-4-fluoro-1,4-diazoniabicyclo[2.2.2]octanebis(tetrafluoroborate) to a solution of the compound having a structure according to formula (Ia) and trimethylsilyl acetate at about 23° C. and stirring for about 16 hrs.
A223. The method of any one of paragraphs A220 to A222, further comprising admixing (a) the reaction product of the compound having a structure according to formula (Ia) with 1-chloromethyl-4-fluoro-1,4-diazoniabicyclo[2.2.2]octanebis(tetrafluoroborate), and trimethylsilyl acetate with (b) a dioxane-dione and sodium carbonate to form the compound having a structure according to formula (VIII),
wherein the dioxane-dione is selected from the group of (1r,3r,5r,7r)-spiro[adamantan-2,2′-[1,3]-dioxane]-4′,6′-dione (SPIAd), 6,10-dioxaspiro[4.5]decane-7,9-dione, 1,5-dioxaspiro[5.5]undecane-2,4-dione, 1,5-dioxaspiro[5.7]tridecane-2,4-dione, 2,2-diphenyl-1,3-dioxane-4,6-dione, 2,2-dibenzyl-1,3-dioxane-4,6-dione, 5,5-dimethylcyclohexane-1,3-dione, 2-(tert-butyl)-2-methyl-1,3-dioxane-4,6-dione, 2-methyl-2-phenyl-1,3-dioxane-4,6-dione, and 2-(4-fluorophenyl)-2-methyl-1,3-dioxane-4,6-dione.
A224. The method of paragraph A223, wherein the admixing is performed in the presence of ethanol.
A225. The method of paragraphs A223 or A224, wherein the admixing comprises adding the dioxane-dione and sodium carbonate to the reaction product of the compound having a structure according to formula (Ia) with 1-chloromethyl-4-fluoro-1,4-diazoniabicyclo[2.2.2]octanebis(tetrafluoroborate), methyl cyanide, and trimethylsilyl acetate dropwise at about 23° C. and stirring for about 4 hrs.
A226. A method of preparing a fibroblast activation protein inhibitor comprising: admixing a compound having a structure according to formula (X) with a radiolabeled salt, MX, to form a compound having a structure according to formula (II):

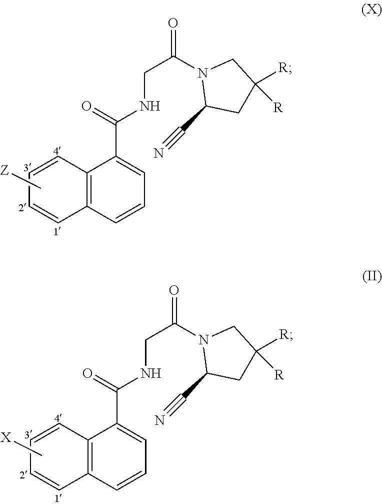

wherein
Z has a structure selected from the group of:

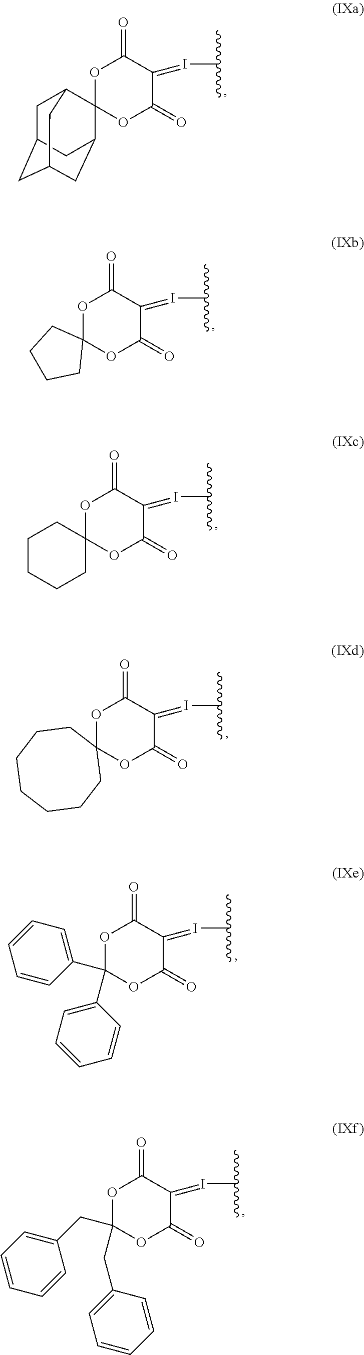 [SMILES: *I=C1C(=O)OC2(OC1=O)C1CC3CC(C1)CC2C3 ; *I=C1C(=O)OC2(CCCC2)OC1=O ; *I=C1C(=O)OC2(CCCCC2)OC1=O ; *I=C1C(=O)OC2(CCCCCCC2)OC1=O ; *I=C1C(=O)OC(c2ccccc2)(c2ccccc2)OC1=O ; *I=C1C(=O)OC(Cc2ccccc2)(Cc2ccccc2)OC1=O]

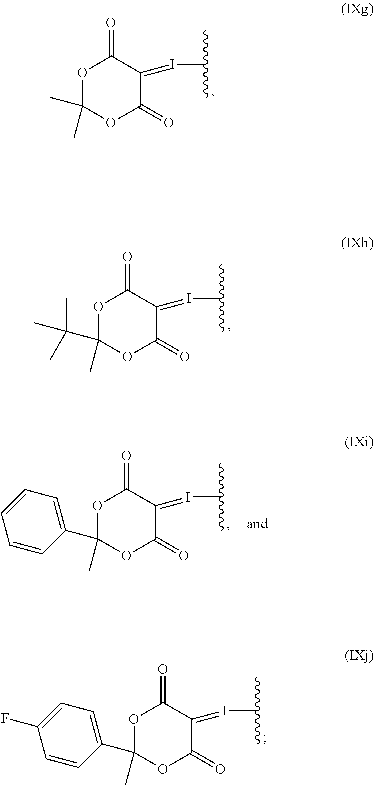 [SMILES: *I=C1C(=O)OC(C)(C)OC1=O ; *I=C1C(=O)OC(C)(C(C)(C)C)OC1=O ; *I=C1C(=O)OC(C)(c2ccccc2)OC1=O ; *I=C1C(=O)OC(C)(c2ccc(F)cc2)OC1=O]

in formula (X) and formula (II), Z and X are located at carbon 1′, 2′, or 3′, and the location of Z in formula (X) and the location of X in formula (II) are the same;
M is selected from the group of K, Li, Na, Rb, Cs, tetraethylammonium, and tetrabutylammonium;
in the salt, MX, and in formula (II), X is selected from the group of ¹⁸F, ²¹¹At, ¹²³I, ¹²⁴I, ¹²⁵I, ¹³¹I, ⁷⁶Br, ⁷⁷Br; and
each R is independently selected from H, F, ²H, CH₃, and cyclopentyl, and the R in formula (II) are the same as the R in formula (X).
A227. The method of paragraph A226, wherein the admixing is performed in the presence of dimethylsulfoxide (DMSO), dimethylformamide (DMF), dimethylacetamide (DMA), or a combination thereof.
A228. The method of paragraphs A226 or A227, wherein the admixing is performed at a temperature in a range of 85° C. to 140° C. for a time in a range of about 5 minutes to about 12 hrs.
A229. The method of any one of paragraphs A226 to A228, further comprising preparing the compound having a structure according to formula (X), by admixing a compound having a structure according to formula (IIa):

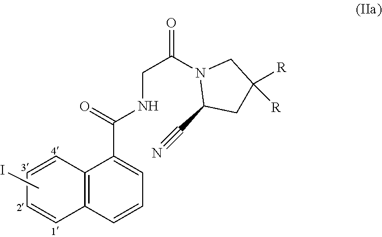

with 1-chloromethyl-4-fluoro-1,4-diazoniabicyclo[2.2.2]octanebis(tetrafluoroborate), and trimethylsilyl acetate to form a reaction product;
wherein the R in formula (IIa) are the same R as in the compound according to formula (X) and the I is located at the same carbon as the Z in the compound according to formula (X).
A230. The method of paragraph A229, wherein the admixing is performed in the presence of acetonitrile.
A231. The method of paragraphs A229 or A230, wherein the admixing comprises dropwise addition of 1-chloromethyl-4-fluoro-1,4-diazoniabicyclo[2.2.2]octanebis(tetrafluoroborate) to a solution of the compound having a structure according to formula (IIa) and trimethylsilyl acetate at about 23° C. and stirring for about 16 hrs.
A232. The method of any one of paragraphs A229 to A230, further comprising
admixing (a) the reaction product of the compound having a structure according to formula (IIa) with 1-chloromethyl-4-fluoro-1,4-diazoniabicyclo[2.2.2]octanebis(tetrafluoroborate), and trimethylsilyl acetate with (b) a dioxane-dione and sodium carbonate to form the compound having a structure according to formula (X),
wherein the dioxane-dione is selected from the group of (1r,3r,5r,7r)-spiro[adamantan-2,2′-[1,3]-dioxane]-4′,6′-dione (SPIAd), 6,10-dioxaspiro[4.5]decane-7,9-dione, 1,5-dioxaspiro[5.5]undecane-2,4-dione, 1,5-dioxaspiro[5.7]tridecane-2,4-dione, 2,2-diphenyl-1,3-dioxane-4,6-dione, 2,2-dibenzyl-1,3-dioxane-4,6-dione, 5,5-dimethylcyclohexane-1,3-dione, 2-(tert-butyl)-2-methyl-1,3-dioxane-4,6-dione, 2-methyl-2-phenyl-1,3-dioxane-4,6-dione, and 2-(4-fluorophenyl)-2-methyl-1,3-dioxane-4,6-dione.
A233. The method of paragraph A222, wherein the admixing is performed in the presence of ethanol.
A234. The method of paragraphs A222 or A233, wherein the admixing comprises adding the SPIAd and sodium carbonate to the reaction product of the compound having a structure according to formula (IIa) with 1-chloromethyl-4-fluoro-1,4-diazoniabicyclo[2.2.2]octanebis(tetrafluoroborate), methyl cyanide, and trimethylsilyl acetate dropwise at about 23° C. and stirring for about 4 hrs.
A235. A method of preparing a fibroblast activation protein inhibitor comprising: admixing a compound having a structure according to formula (XI) with a radiolabeled salt, MX, to form a compound having a structure according to formula (III):

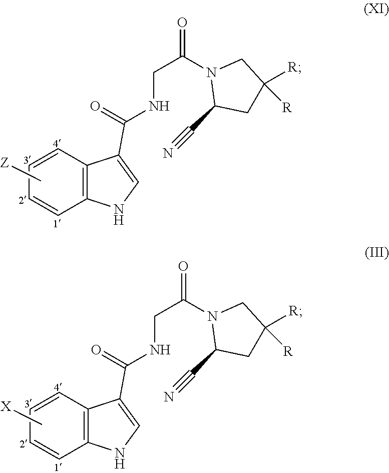

wherein
Z has a structure selected from the group of:

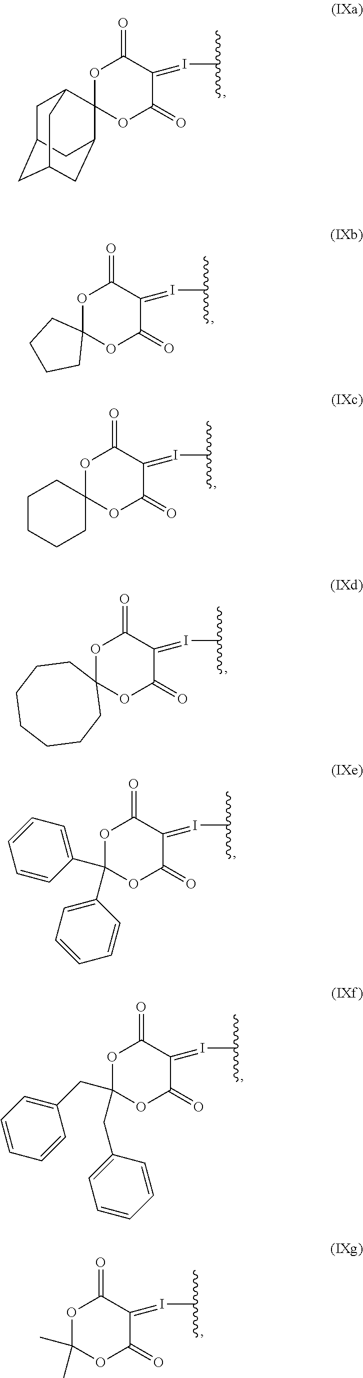

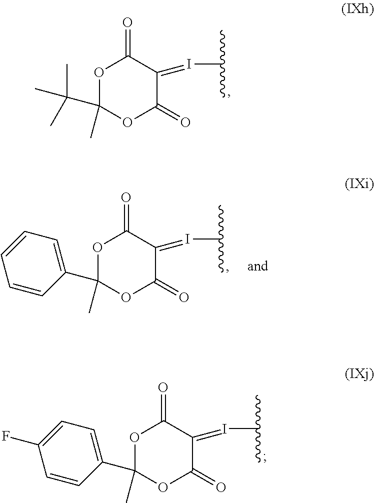 [SMILES: *I=C1C(=O)OC(C)(C(C)(C)C)OC1=O ; *I=C1C(=O)OC(C)(c2ccccc2)OC1=O ; *I=C1C(=O)OC(C)(c2ccc(F)cc2)OC1=O]

in formula (XI) and formula (III), Z and X are located at carbon 1′, 2′, or 3′, and the location of Z in formula (XI) and the location of X in formula (III) are the same;
M is selected from the group of K, Li, Na, Rb, Cs, tetraethylammonium, and tetrabutylammonium;
in the salt, MX, and in formula (III), X is selected from the group of ¹⁸F, ²¹¹At, ¹²³I, ¹²⁴I, ¹²⁵I, ¹³¹I, ⁷⁶Br, ⁷⁷Br; and
each R is independently selected from H, F, ²H, CH₃, and cyclopentyl, and the R in formula (III) are the same as the R in formula (XI).
A236. The method of paragraph A235, wherein the admixing is performed in the presence of dimethylsulfoxide (DMSO), dimethylformamide (DMF), dimethylacetamide (DMA), or a combination thereof.
A237. The method of paragraphs A235 or A236, wherein the admixing is performed at a temperature in a range of 85° C. to 140° C. for a time in a range of about 5 minutes to about 12 hrs.
A238. The method of any one of paragraphs A235 to A237, further comprising preparing the compound having a structure according to formula (XI), by admixing a compound having a structure according to formula (IIIa):

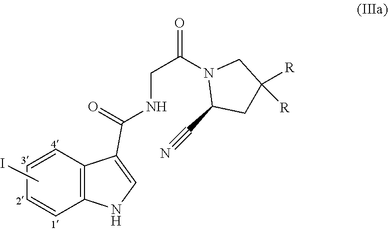

with 1-chloromethyl-4-fluoro-1,4-diazoniabicyclo[2.2.2]octanebis(tetrafluoroborate), and trimethylsilyl acetate to form a reaction product;
wherein the R in formula (IIIa) are the same R as in the compound according to formula (XI) and the I is located at the same carbon as the Z in the compound according to formula (XI).
A239. The method of paragraph A238, wherein the admixing is performed in the presence of acetonitrile.
A240. The method of paragraphs A238 or A239, wherein the admixing comprises dropwise addition of 1-chloromethyl-4-fluoro-1,4-diazoniabicyclo[2.2.2]octanebis(tetrafluoroborate) to a solution of the compound having a structure according to formula (IIa) and trimethylsilyl acetate at about 23° C. and stirring for about 16 hrs.
A241. The method of any one of paragraphs A238 to A240, further comprising
admixing (a) the reaction product of the compound having a structure according to formula (IIIa) with 1-chloromethyl-4-fluoro-1,4-diazoniabicyclo[2.2.2]octanebis(tetrafluoroborate), and trimethylsilyl acetate with (b) a dioxane-dione and sodium carbonate to form the compound having a structure according to formula (XI),
wherein the dioxane-dione is selected from the group of (1r,3r,5r,7r)-spiro[adamantan-2,2′-[1,3]-dioxane]-4′,6′-dione (SPIAd), 6,10-dioxaspiro[4.5]decane-7,9-dione, 1,5-dioxaspiro[5.5]undecane-2,4-dione, 1,5-dioxaspiro[5.7]tridecane-2,4-dione, 2,2-diphenyl-1,3-dioxane-4,6-dione, 2,2-dibenzyl-1,3-dioxane-4,6-dione, 5,5-dimethylcyclohexane-1,3-dione, 2-(tert-butyl)-2-methyl-1,3-dioxane-4,6-dione, 2-methyl-2-phenyl-1,3-dioxane-4,6-dione, and 2-(4-fluorophenyl)-2-methyl-1,3-dioxane-4,6-dione.
A242. The method of paragraph A241, wherein the admixing is performed in the presence of ethanol.
A243. The method of paragraphs A241 or A242, wherein the admixing comprises adding the SPIAd and sodium carbonate to the reaction product of the compound having a structure according to formula (IIIa) with 1-chloromethyl-4-fluoro-1,4-diazoniabicyclo[2.2.2]octanebis(tetrafluoroborate), methyl cyanide, and trimethylsilyl acetate dropwise at about 23° C. and stirring for about 4 hrs.
A244. A method treating, detecting, or imaging cancer comprising, administering to a patient a fibroblast activation protein inhibitor according to any one of paragraphs A1 to A57.
A245. Use of a fibroblast activation protein inhibitor according to any one of paragraphs A1 to A57 in the treatment, detection, or imaging of cancer.
A246. Use in the manufacture of a medicament for the treatment, detection, or imaging of cancer, of a fibroblast activation protein inhibitor according to any one of paragraphs A1 to A57.
A247. The method or use of any one of paragraphs A244 to A246, wherein the cancer comprises an epithelial tumor, sarcoma, or mesothelioma.
A248. A method of detecting fibroblast activation protein expression in human development, growth, wound healing, or a combination thereof comprising administering to a patient a fibroblast activation protein inhibitor according to any one of paragraphs A1 to A57.
A249. Use of a fibroblast activation protein inhibitor according to any one of paragraphs A1 to A57 in the detection of the expression of fibroblast activation protein or the lack of expression of fibroblast activation protein in a human.
A250. Use of a fibroblast activation protein inhibitor according to any one of paragraphs A1 to A57 in the manufacture of a medicament for the detection of the expression of fibroblast activation protein.
A251. A method of detecting a cardiovascular pathology comprising, administering to a patient a fibroblast activation protein inhibitor according to any one of paragraphs A1 to A57.
A252. Use of a fibroblast activation protein inhibitor according to any one of paragraphs A1 to A57 in the detection of a cardiovascular pathology in a human.
A253. Use of a fibroblast activation protein inhibitor according to any one of paragraphs A1 to A57 in the manufacture of a medicament for the detection of a cardiovascular pathology.
A254. The method of any one of paragraphs A251 to A253, wherein the cardiovascular pathology comprises a myocardial infarction, cardiac remodeling after infarction, or aortic remodeling.
A255. Use of a fibroblast activation protein inhibitor according to any one of paragraphs A1 to A57 in the detection of a pulmonary pathology in a human.
A256. Use of a fibroblast activation protein inhibitor according to any one of paragraphs A1 to A57 in the manufacture of a medicament for the detection of a pulmonary pathology.
A257. The method of paragraphs A255 or A256, wherein the pulmonary pathology is the result of an auto immune or rheumatologic response, primary pulmonary pathology such as interstitial lung disease, or post treatment effects from chemotherapy, radiation therapy, or a combination thereof.

### EXAMPLES

### Example 1: Preparation of (S)—N-(2-(2-cyanopyrrolidin-1-yl)-2-oxoethyl)-6-iodoquinoline-4-carboxamide (Ia-3)

(S)—N-(2-(2-cyanopyrrolidin-1-yl)-2-oxoethyl)-6-iodoquinoline-4-carboxamide (Ia-3) was prepared according to the following scheme:

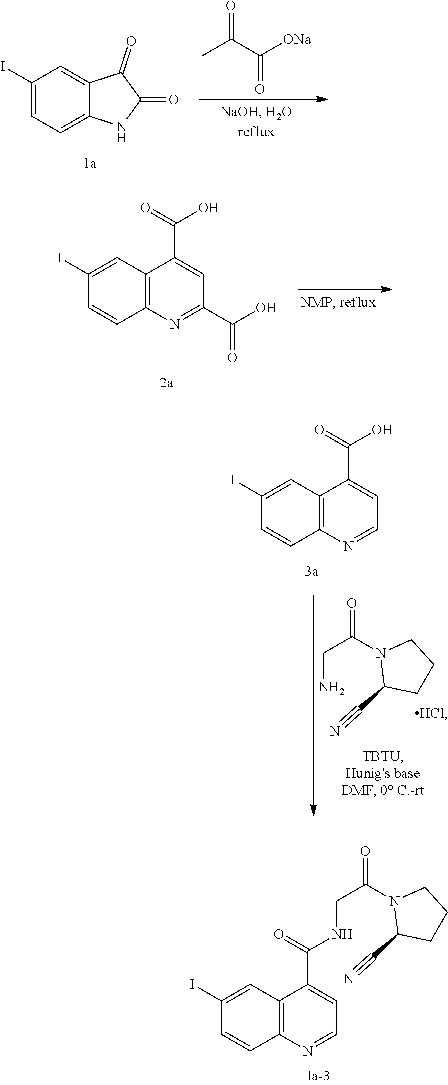

To a round bottom flask, 5-iodoindoline-2,3-dione (1 mmol) (1a) and water (10 mL) were added at room temperature. NaOH (240 mg), 6 mmol) was added portion wise, followed by addition of sodium pyruvate (176 mg, 1.6 mmol). The reaction mixture was heated to reflux and allowed to stir for 48 hours. Upon cooling to room temperature, the pH of the reaction mixture was adjusted to a pH of 2 using 1 M HCl, resulting in a precipitate. The precipitate was filtered, washed with water, dichloromethane, and hexanes, and dried in vacuo to afford 6-iodoquinoline-2,4-dicarboxylic acid (2a) as a light orange solid (2.5 g, 100% yield).

To a round bottom flask, compound 2a (1 mmol) and N-methyl-2-pyrrolidone (NMP) (15 mL) were added. The reaction mixture was heated to reflux and stirred for 16 hours. After cooling to room temperature, the reaction mixture was filtered over celite. The resulting solution was diluted with water (30 mL) and extracted with ethyl acetate three times (15 mL). The combined organic layers were washed with water and brine, dried over sodium sulfate, and concentrated in vacuo. The resulting residue was triturated with a mixture of dichloromethane and hexanes, and filtered. The obtained solid was dried in vacuo to give afford 6-iodoquinoline-4-carboxylic acid (3a) as a brown solid (175 mg, 20% yield).

To a round bottom flask was added compound 3a (1.05 mmol) and dimethylformamide (DMF) (10 mL) at 0° C. along with Hunig's base (523 μL, 3 mmol) and (S)-1-glycylpyrrolidine-2-carbonitrile HCL (1.00 mmol). 2-(1H-benzotrazole-1-yl)-1,1,3,3-tetramethylaminium tetrafluoroborate (TBTU) (1.05 mmol) was added slowly portion wise and the reaction mixture was allowed to stir for 30 min at 0° C. TBTU (1.05 mmol) was added portion wise and the solution was warmed to room temperature. After stirring for 48 h the reaction mixture was diluted with dichloromethane (10 mL), washed with water, brine, and sat. aq. LiCl, and dried over sodium sulfate. The combined organic layers were dried in vacuo and the resulting residue was purified by column chromatography (1-10% MeOH/DCM) to give (S)—N-(2-(2-cyanopyrrolidin-1-yl)-2-oxoethyl)-6-iodoquinoline-4-carboxamide (Ia-3) as a brown foam (24.5 mg, 71% yield). ¹H NMR (400 MHz, DMSO-d6) δ 9.12 (t, J=5.8 Hz, 1H), 9.00 (d, J=4.4 Hz, 1H), 8.74 (d, J=2.0 Hz, 1H), 8.07 (dd, J=8.8, 2.0 Hz, 1H), 7.85 (d, J=8.8 Hz, 1H), 7.59 (d, J=4.4 Hz, 1H), 4.84 (dd, J=7.4, 3.4 Hz, 1H), 4.20 (qd, J=16.9, 5.89 Hz, 2H), 3.76-3.69 (m, 1H), 3.58-3.47 (m, 1H), 3.42-3.34 (m, 1H), 2.22-2.05 (m, 3H); ¹³C NMR (100 MHz, DMSO-d6) δ 167.7, 167.1, 151.3, 147.2, 141.1, 138.7, 134.7, 131.7, 126.3, 120.3, 119.8, 94.7, 46.8, 45.8, 42.0, 29.9, 25.3.

Accordingly, Example 1 demonstrates preparation of a compound of the disclosure.

### Example 2: Preparation of N-(2-((S)-2-cyanopyrrolidin-1-yl)-2-oxoethyl)-6-(((1r,3r,5r,7r)-4′,6′-dioxospiroadamantane-2,2′-[1,3]dioxan]-5′-ylidene)-λ3-iodaneyl)quinoline-4-carboxamide (VIII-3)

N-(2-((S)-2-cyanopyrrolidin-1-yl)-2-oxoethyl)-6-(((1r,3r,5r,7r)-4′,6′-dioxospiro[adamantane-2,2′-[1,3]dioxan]-5′-ylidene)-λ3-iodaneyl)quinoline-4-carboxamide (VIII-3) was prepared by the following scheme:

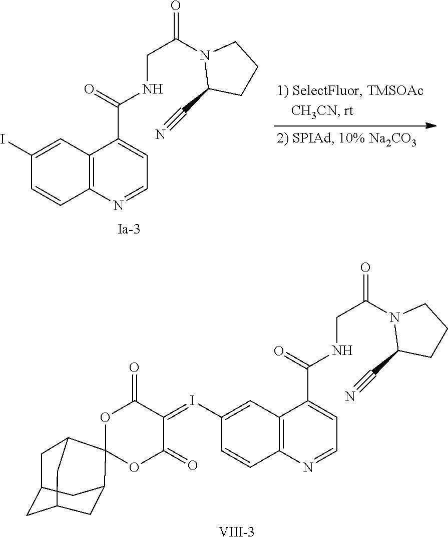

To a round bottom flask containing compound Ia-3 as prepared in Example 1 (66.1 mg, 0.152 mmol) in acetonitrile (1.52 mL) at room temperature was added trimethylsilyl acetate (TMSOAc) (68.4 μL, 0.456 mmol) slowly dropwise followed by addition of 1-chloromethyl-4-fluoro-1,4-diazoniabicyclo[2.2.2]octanebis(tetrafluoroborate) (SelectFluor) (135 mg, 0.380 mmol). The reaction mixture was allowed to stir 16 h and then was concentrated in vacuo. The residue was then suspended in ethyl acetate (1.52 mL) and 0.5 M sodium acetate (1.53 mL) was added. The organic layer was separated, and the aqueous layer was extracted two times with ethyl acetate. The combined organic layers were dried over sodium sulfate and concentrated in vacuo. Ethanol (1.52 mL) was then added to suspend the obtained residue and a separately prepared (1r,3r,5r,7r)-spiro[adamantan-2,2′-[1,3]-dioxane]-4′,6′-dione (SpIAd)/10% aq. sodium carbonate solution was added slowly dropwise at room temperature. After stirring for 4 h, the reaction mixture was extracted three times with dichloromethane (1.52 mL), and dried over sodium sulfate to give N-(2-((S)-2-cyanopyrrolidin-1-yl)-2-oxoethyl)-6-(((1r,3r,5r,7r)-4′,6′-dioxospiro[adamantane-2,2′-[1,3]dioxan]-5′-ylidene)-λ3-iodaneyl)quinoline-4-carboxamide (VIII-3) as a light yellow solid (4.5 mg, 5% yield).

Thus, Example 2 demonstrates preparation of an intermediate compound for use in the preparation of the fibroblast activation protein inhibitors of the disclosure.

### Example 3: Preparation of (S)—N-(2-(2-cyanopyrrolidin-1-yl)-2-oxoethyl)-6-(trimethylstannyl)quinoline-4-carboxamide

(S)—N-(2-(2-cyanopyrrolidin-1-yl)-2-oxoethyl)-6-(trimethylstannyl)quinoline-4-carboxamide (VI-3) can be prepared according to the following scheme:

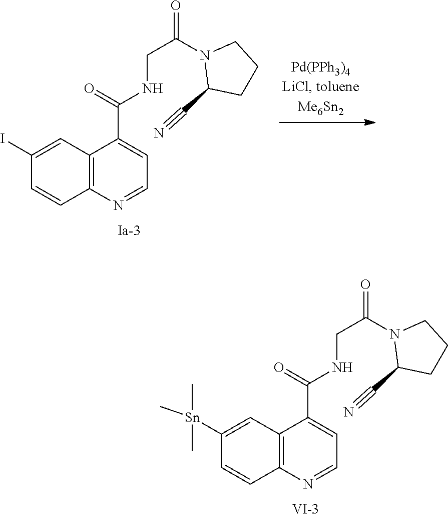

In an inert atmosphere, compound Ia-3 as prepared in Example 1 (1 mmol) in toluene at room temperature is admixed with Pd(PPh₃)₄ in toluene. Lithium chloride is added to the reaction mixture followed by hexamethyldistannane to give (S)—N-(2-(2-cyanopyrrolidin-1-yl)-2-oxoethyl)-6-(trimethylstannyl)quinoline-4-carboxamide (VI-3).

Specifically, to a flame dried round bottom flask was added Pd(PPh)₃ (77.4 mg, 0.067 mmol) and LiCl (67.8 mg, 1.60 mmol). The round bottom flask was then placed under vacuum and backfilled with argon 3×. A solution of Ia-3 as prepared in Example 1 in toluene (3.3 mL) was then added followed by dropwise addition of hexamethylditin. The reaction mixture was then heated to 100° C. and stirred for 16 hours. Upon cooling to room temperature, 2 M aqueous KF (1.7 mL) was added and the mixture was allowed to stir for 30 mins. The contents of the round bottom flask were then poured over celite, washed with brine, dried over Na₂SO₄ and concentrated in vacuo. After purification using flash column chromatography (1-10% methanol/dichloromethane) the desired product was obtained as a white foam. ¹H NMR (400 MHz, DMSO-d6) 9.04 (t, J=5.4 Hz, 1H), 8.93 (d, J=3.6 Hz, 1H), 8.60-8.46 (m, 1H), 8.04-7.83 (m, 2H), 7.54-7.49 (m, 1H), 4.79 (dd, J=8.0, 4.0 Hz, 1H), 4.27-4.12 (m, 2H), 3.77-3.68 (m, 1H), 3.54 (q, J=8.0 Hz, 1H), 2.25-1.96 (m, 4H), 0.43-0.24 (m, 9H); ¹³C NMR (100 MHz, DMSO-d6) δ 167.78, 167.75, 150.6, 148.4, 142.7, 142.2, 136.7, 134.2, 128.6, 124.3, 119.7, 119.4, 46.8, 45.7, 42.0, 30.0, 25.3, −8.67; HRMS (ESI⁺-TOF) (C₂₀H₂₅N₄O₂Sn) calcd 472.0921 (M+H), found 473.0994.

Thus, Example 3 demonstrates a preparation of an intermediate compound for use in the preparation of the fibroblast activation protein inhibitors of the disclosure.

### Example 4: Preparation of (S)—N-(2-(2-cyanopyrrolidin-1-yl)-2-oxoethyl)-6-[18F]fluoroquinoline-4-carboxamide (1b-3)

(S)—N-(2-(2-cyanopyrrolidin-1-yl)-2-oxoethyl)-6-[18F]fluoroquinoline-4-carboxamide (1b-3) can be prepared according to the following scheme:

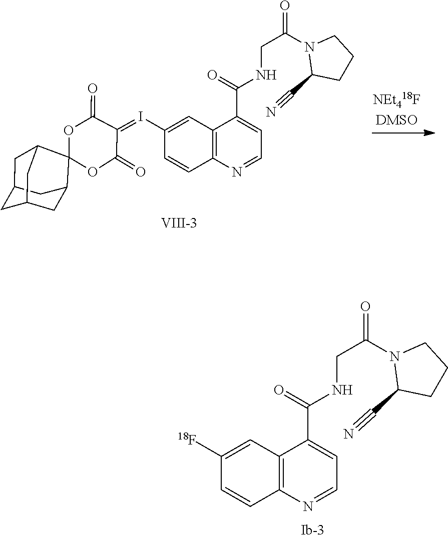

To a round bottom flask containing compound VIII-3 as prepared in Example 2 and dimethyl sulfoxide (DMSO) is added tetraethyl ammonium [18F]fluoride to provide (S)—N-(2-(2-cyanopyrrolidin-1-yl)-2-oxoethyl)-6-[18F]fluoroquinoline-4-carboxamide (1b-3).

Accordingly, Example 4 demonstrates a preparation of a compound of the disclosure.

### Example 5: Preparation of (S)—N-(2-(2-cyanopyrrolidin-1-yl)-2-oxoethyl)-6-[18F]fluoroquinoline-4-carboxamide (1b-3)

(S)—N-(2-(2-cyanopyrrolidin-1-yl)-2-oxoethyl)-6-[18F]fluoroquinoline-4-carboxamide (1b-3) can be prepared according to the following scheme:

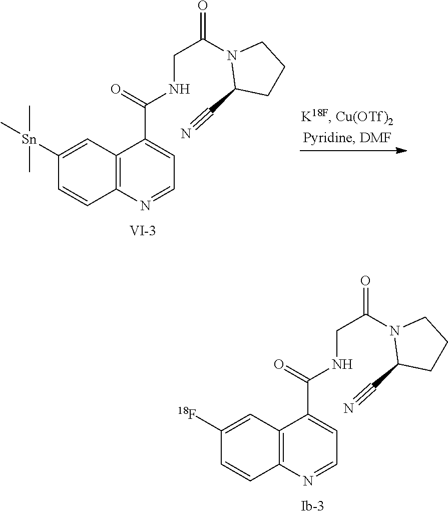

(S)—N-(2-(2-cyanopyrrolidin-1-yl)-2-oxoethyl)-6-(trimethylstannyl)quinoline-4-carboxamide (VI-3) as prepared in Example 3 is admixed with potassium [18F]fluoride in the presence of copper(II) triflate (Cu(OTf)₂), pyridine, and dimethylformamide (DMF) to provide (S)—N-(2-(2-cyanopyrrolidin-1-yl)-2-oxoethyl)-6-[18F]fluoroquinoline-4-carboxamide.

Accordingly, Example 5 demonstrates a preparation of a compound of the disclosure.

### Example 6: Preparation of (S)—N-(2-(2-cyanopyrrolidin-1-yl)-2-oxoethyl)-6-fluroquinoline-4-carboxamide

(S)—N-(2-(2-cyanopyrrolidin-1-yl)-2-oxoethyl)-6-fluroquinoline-4-carboxamide was prepared according to the following scheme:

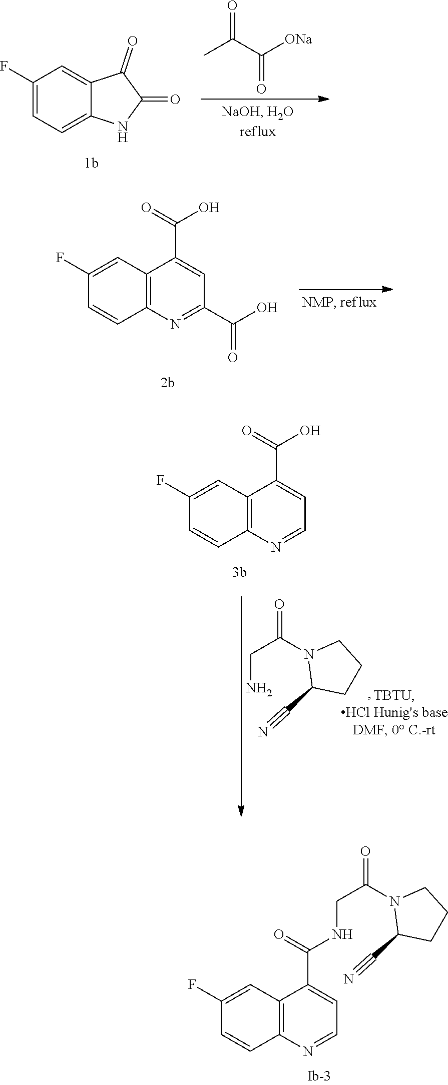

To a round bottom flask, 5-fluorindoline-2,3-dione (1 mmol) (1b) and water (10 mL) were added at room temperature. NaOH (240 mg), 6 mmol) was added portion wise, followed by addition of sodium pyruvate (176 mg, 1.6 mmol). The reaction mixture was heated to reflux and allowed to stir for 48 hours. Upon cooling to room temperature, the pH of the reaction mixture was adjusted to a pH of 2 using 1 M HCl, resulting in a precipitate. The precipitate was filtered, washed with water, dichloromethane, and hexanes, and dried in vacuo to afford 6-fluroquinoline-2,4-dicarboxylic acid (2b) as a light tan solid (2.58 g, 91% yield).

To a round bottom flask, compound 2b (1 mmol) and N-methyl-2-pyrrolidone (NMP) (15 mL) were added. The reaction mixture was heated to reflux and stirred for 16 hours. After cooling to room temperature, the reaction mixture was filtered over celite. The resulting solution was diluted with water (30 mL) and extracted with ethyl acetate three times (15 mL). The combined organic layers were washed with water and brine, dried over sodium sulfate, and concentrated in vacuo. The resulting residue was triturated with a mixture of dichloromethane and hexanes, and filtered. The obtained solid was dried in vacuo to give afford 6-fluroquinoline-4-carboxylic acid (3b) as a brown solid (181 mg, 46% yield).

To a round bottom flask was added compound 3b (1.05 mmol) and dimethylformamide (DMF) (10 mL) at 0° C. along with Hunig's base (523 μL, 3 mmol) and (S)-1-glycylpyrrolidine-2-carbonitrile HCL (1.00 mmol). 2-(1H-benzotrazole-1-yl)-1,1,3,3-tetramethylaminium tetrafluoroborate (TBTU) (1.05 mmol) was added slowly portion wise and the reaction mixture was allowed to stir for 30 min at 0° C. TBTU (1.05 mmol) was added portion wise and the solution was warmed to room temperature. After stirring for 48 h the reaction mixture was diluted with dichloromethane (10 mL), washed with water, brine, and sat. aq. LiCl, and dried over sodium sulfate. The combined organic layers were dried in vacuo and the resulting residue was purified by column chromatography (1-10% MeOH/DCM) to give (S)—N-(2-(2-cyanopyrrolidin-1-yl)-2-oxoethyl)-6-fluroquinoline-4-carboxamide as a brown foam (11.9 mg, 46% yield). ¹H NMR (400 MHz, DMSO-d6) 9.12 (t, J=6.0 Hz, 1H), 8.98 (d, J=4.0 Hz, 1H), 8.20-8.13 (m, 2H), 7.76 (dt, J=8.0, 4.0 Hz, 1H), 7.63 (d, J=4.0 Hz, 1H), 4.86 (dd, J=7.26, 3.66 Hz, 1H), 4.28-4.12 (m, 2H), 3.77-3.69 (m, 1H), 3.58-3.49 (m, 1H), 2.22-2.00 (m, 4H); ¹³C NMR (100 MHz, DMSO-d6) δ 167.9, 167.3, 159.6, 150.2, 145.7, 142.0, 132.7, 120.6, 120.4, 120.3, 119.8, 109.8, 46.8, 45.8, 42.0, 29.9, 25.3; ¹⁹F NMR (376 MHz, DMSO-d6) −111.6−(−) 111.8 (m, 1F).

Accordingly, Example 6 demonstrates preparation of (S)—N-(2-(2-cyanopyrrolidin-1-yl)-2-oxoethyl)-6-fluroquinoline-4-carboxamide.

### Example 7: Preparation of (S)—N-(2-(2-cyanopyrrolidin-1-yl)-2-oxoethyl)-6-iodoquinoline-4-carboxamide (Ia-3)

(S)—N-(2-(2-cyanopyrrolidin-1-yl)-2-oxoethyl)-6-iodoquinoline-4-carboxamide (Ia-3) was prepared according to the following scheme:

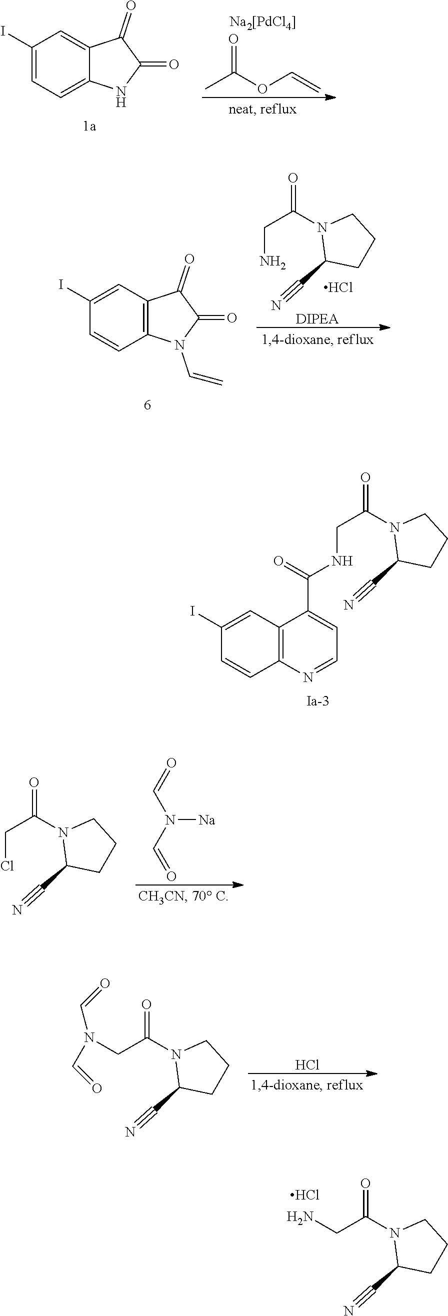

5-iodo-1-vinylindoline-2,3-dione (6) was prepared as follows. To a round bottom flast at room temperature was added 5-iodoindoline-2,3-dione (2.5 g, 9.16 mmol) followed by vinyl acetate (23 mL). Na₂[PdCl₄] was then added batchwise, and the reaction was heated to reflux and allowed to stir for 24 hours. The reaction mixture was cooled to room temperature, concentrated in vacuo and purified by column chromatograph (10-40% ethyl acetate/hexantes). The desired product, 6, was obtained as a red solid (1.25 g, 45% yield).

(S)-1-glycylpyrrolidine-2-carbonitrile HCl was prepared as follows. To a round bottom flask containing (S)-1-(2-chloroacetyl)pyrrolidine-2-carbonitrile (2.5 g, 14.4 mmol) at room temperature was added sodium diformylamide (1.7 g, 17.38 mmol) batchwise. The reaction mixture was then heated to 70° C. and stirred for 96 hours. After cooling to room temperature and filtering, the resulting solution was concentrated in vacuo. Crude (S)—N-(2-(2-cyanopyrrolidin-1-yl)-2-oxoethyl)-N-formylformamide (3.03 g, 14.48 mmol) was added to a new round bottom flask followed by addition of 6 N HCl in 1,4-dioxane (25 mL). The reaction was heated to reflux and stirred for 24 hours. After cooling to room temperature, the mixture was filtered, and the resulting solution was concentrated in vacuo. The residue was then triturated with methanol, filtered, and dried under vacuum to give the desired HCl salt, (S)-1-glycylpyrrolidine-2-carbonitrile HCL (246.7 mg, 19% yield) as a white solid.

(S)—N-(2-(2-cyanopyrrolidin-1-yl)-2-oxoethyl)-6-iodoquinoline-4-carboxamide was prepared as follows. To a round bottom flask containing 6 (400 mg, 1.34 mmol) and 1,4-dioxane (13.4 mL) at room temperature was added N,N-diisopropylethylamine (DIPEA) (700 microliter, 4.02 mmol) dropwise. (S)-1-glycylpyrrolidine-2-carbonitrile HCl (132 mg, 1.47 mmol) was then added portionwise. The reaction mixture was then heated to reflux and allowed to stir for 48 hours. After cooling to room temperature, the mixture was diluted with ethyl acetate (13 mL), washed with water, brine, and dried over sodium sulfate and concentrated in vacuo. The residue was purified by column chromatography (1-10% methanol/dichloromethane) to give (Ia-3) as a tan solid (54.1 mg, 9%).

Accordingly, Example 7 demonstrates preparation of a compound of the disclosure.

### Example 8: Preparation of (S)—N-(2-(2-cyanopyrrolidin-1-yl)-2-oxoethyl)-6-iodoquinoline-4-carboxamide (Ia-3)

(S)—N-(2-(2-cyanopyrrolidin-1-yl)-2-oxoethyl)-6-iodoquinoline-4-carboxamide (Ia-3) was prepared according to the following scheme:

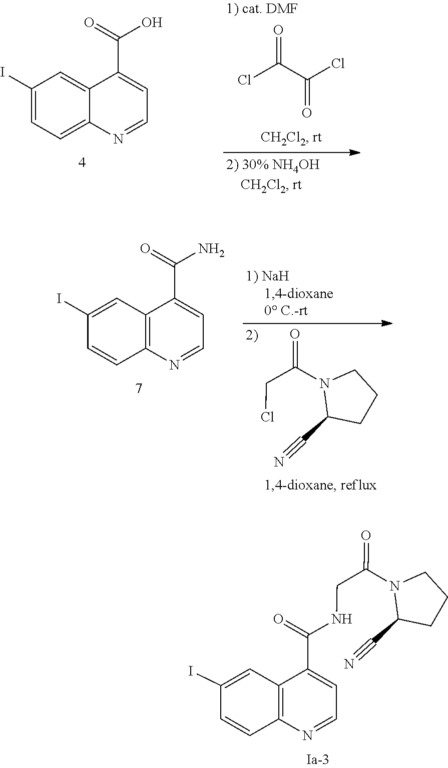 [SMILES: O=C(O)c1ccnc2ccc(I)cc12 ; O=C(Cl)C(=O)Cl ; NC(=O)c1ccnc2ccc(I)cc12 ; N#C[C@@H]1CCCN1C(=O)CCl ; N#C[C@@H]1CCCN1C(=O)CNC(=O)c1ccnc2ccc(I)cc12]

To a suspension of 6-iodoquinoline-4-carboxylic acid, 4, (500 mg, 1.67 mmol) in dichloromethane (16.7 mL) at 0° C. was added oxalyl chloride (315 microliter, 3.67 mmol) dropwise followed by catalytic DMF (3 drops from a glass pasteur pipet). The reaction was warmed up to room temperature and allowed to stir for 2 hours. After concentration in vacuo, dichloromethane (9.28 mL) was added at room temperature followed by dropwise addition of 30% NH₄OH (3.34 mL). The reaction mixture was stirred for 16 hours, diluted with water (9.3 mL), filtered, washed with hexanes, and dried in vacuo. The obtained crude 6-iodoquinoline-4-carboxamide, 7, (469.1 mg, 1.57 mmol) was dissolved in 1,4-dioxane (800 microliter). It was then added dropwise to a flame dried round bottom flask containing 60% by weight suspension of NaH (81.6 mg, 2.04 mmol) in 1,4-dioxane (800 microliter) at 0° C. (S)-1-(2-chloroacetyl)pyrrolidine-2-carbonitrile (542 mg, 3.14 mmol) was then added to the reaction mixture. The reaction mixture was then heated to reflux and allowed to stir for 48 hours. Upon cooling to room temperature, saturated NH₄Cl was added slowly dropwise to quench the reaction. It was then extracted three times with ethyl acetate (1.6 mL), dried over Na₂SO₄, and concentrated in vacuo. The resulting residue was purified by column chromatography (1-10% methanol/dichloromethane) to give (Ia-3) (50.6 mg, 7% yield) as a light yellow solid.

Accordingly, Example 8 demonstrates preparation of a compound of the disclosure.

### Example 9: Automated radiosynthesis of (S)—N-(2-(2-cyanopyrrolidin-1-yl)-2-oxoethyl)-6-fluroquinoline-4-carboxamide (Ib-3)

(S)—N-(2-(2-cyanopyrrolidin-1-yl)-2-oxoethyl)-6-fluroquinoline-4-carboxamide (lb-3) was prepared according to the following scheme:

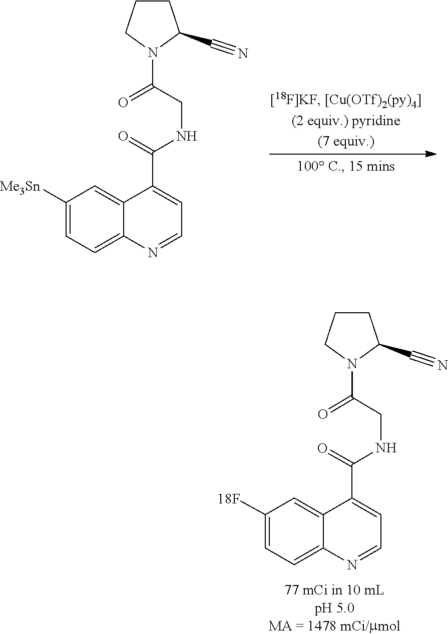

Synthesis of (S)—N-(2-(2-cyanopyrrolidin-1-yl)-2-oxoethyl)-6-fluroquinoline-4-carboxamide (Ib-3) was automated in a TRACERLab FX_{FN} module. Briefly, cyclotron produced ¹⁸F was trapped on a QMA Sep-Pak and eluted into the reactor using KOTf (10 mg) and minimal K₂CO₃ (50 microgram) in H₂O (0.5 mL). Azeotropic drying was then carried out using acetonitrile (1 mL). To the dried [¹⁸F]KF was added a solution of the stannane precursor, (S)—N-(2-(2-cyanopyrrolidin-1-yl)-2-oxoethyl)-6-(trimethylstannyl)quinoline-4-carboxamide (VI-3), (5.0 mg, 0.01 mmol) in DMA (830 microliter) followed by addition of a solution of [Cu(OTf)₂(py)₄](0.2 M stock solution in DMA, 100 μL, 0.02 mmol) and pyridine (1 M stock solution in DMA, 70 μL, 0.14 mmol). The reaction mixture was then heated to 100° C. and allowed to stir for 15 mins. Upon cooling to 50° C., 2 mL of buffer (20% acetonitrile, 10 mM NH₄HCO₃, pH 10) was added and after stirring for an additional 1 min was transferred to an HPLC loop for injection and purification by semi-preparative chromatography (Gemini 5 μm NXC18 110 Å, 250×10, 4 mL/min). The product peak (retention time ~20 min) was collected and diluted into 50 mL of MQ H₂O followed by trapping on a C18 extraction disk. The trapped product was washed with 10 mL of sterile water, eluted with 500 μL of EtOH and then rinsed with 4.0 mL of saline into the collection vial containing 5.5 mL of saline. The resulting 10 mL solution was then passed through a sterile filter into a sterile 10 mL dose vial. The identity and purity (S)—N-(2-(2-cyanopyrrolidin-1-yl)-2-oxoethyl)-6-fluroquinoline-4-carboxamide (Ib-3) was then confirmed using HPLC (Luna C18(2), 150×4.6, 5p, Buffer: 20% acetonitrile, 10 mM NH₄OAc, pH 5.0, 2 mL/min at 40° C.).

Accordingly, Example 9 demonstrates the automated radiosynthesis of a compound of the disclosure.

### Example 10: Ib-3 Uptake and Dosimetry Studies

Uptake and dosimetry studies of (S)—N-(2-(2-cyanopyrrolidin-1-yl)-2-oxoethyl)-6-fluroquinoline-4-carboxamide (Ib-3) were performed in Sprague-Dawley rats (n=4, 2 males, 2 females) at 10, 20, 60, and 120 min. Rats were anesthetized with isoflurane, and (S)—N-(2-(2-cyanopyrrolidin-1-yl)-2-oxoethyl)-6-fluroquinoline-4-carboxamide (Ib-3) was administered via tail vein injections. At the appropriate time points, animals were euthanized, and their tissues procured for measuring radioactivity. Radioactivity was measured in a well counter and expressed as decay-corrected percentage injected dose per gram of tissue. Radiation dosimetry was calculated from the distribution data and was used to determine estimates of human dosimetry with OLINDA/EXM 2.0 software, or equivalent.

Human estimated dosimetry is given in Table 1, below, in mSv/MBq injected for each organ for males, females, and gender averaged:

TABLE 1

| Males | | Females | | Gender Averaged | |
|---|---|---|---|---|---|
| Target Organ | mSv/MBq | Target Organ | mSv/MBq | Target Organ | mSv/MBq |
| Adrenals | 9.10E−03 | Adrenals | 1.02E−02 | Adrenals | 9.65E−03 |
| Brain | 2.79E−03 | Brain | 3.83E−03 | Brain | 3.31E−03 |
| | | Breasts | 1.23E−02 | Breasts | 1.23E−02 |
| Esophagus | 1.13E−02 | Esophagus | 1.29E−02 | Esophagus | 1.21E−02 |
| Eyes | 5.39E−03 | Eyes | 6.79E−03 | Eyes | 6.09E−03 |
| Gallbladder Wall | 1.33E−02 | Gallbladder Wall | 1.64E−02 | Gallbladder Wall | 1.49E−02 |
| Left colon | 1.80E−02 | Left colon | 2.18E−02 | Left colon | 1.99E−02 |
| Small Intestine | 2.42E−02 | Small Intestine | 2.75E−02 | Small Intestine | 2.59E−02 |
| Stomach Wall | 1.40E−02 | Stomach Wall | 1.69E−02 | Stomach Wall | 1.55E−02 |
| Right colon | 1.65E−02 | Right colon | 1.99E−02 | Right colon | 1.82E−02 |
| Rectum | 1.62E−02 | Rectum | 1.94E−02 | Rectum | 1.78E−02 |
| Heart Wall | 1.06E−02 | Heart Wall | 1.46E−02 | Heart Wall | 1.26E−02 |
| Kidneys | 1.56E−02 | Kidneys | 1.48E−02 | Kidneys | 1.52E−02 |
| Liver | 7.83E−03 | Liver | 9.11E−03 | Liver | 8.47E−03 |
| Lungs | 7.17E−03 | Lungs | 9.36E−03 | Lungs | 8.27E−03 |
| | | Ovaries | 1.40E−02 | Ovaries | 1.40E−02 |
| Pancreas | 1.00E−02 | Pancreas | 1.19E−02 | Pancreas | 1.10E−02 |
| Prostate | 1.66E−02 | | | Prostate | 1.66E−02 |
| Salivary Glands | 1.17E−02 | Salivary Glands | 1.34E−02 | Salivary Glands | 1.26E−02 |
| Red Marrow | 1.27E−02 | Red Marrow | 1.58E−02 | Red Marrow | 1.43E−02 |
| Osteogenic Cells | 1.78E−02 | Osteogenic Cells | 1.90E−02 | Osteogenic Cells | 1.84E−02 |
| Spleen | 8.20E−03 | Spleen | 1.00E−02 | Spleen | 9.10E−03 |
| Testes | 7.86E−03 | | | Testes | 7.86E−03 |
| Thymus | 1.18E−02 | Thymus | 1.50E−02 | Thymus | 1.34E−02 |
| Thyroid | 1.20E−02 | Thyroid | 1.38E−02 | Thyroid | 1.29E−02 |
| Urinary Bladder | 4.87E−02 | Urinary Bladder | 3.42E−02 | Urinary Bladder | 4.15E−02 |
| Wall | | Wall | | Wall | |
| | | Uterus | 1.49E−02 | Uterus | 1.49E−02 |
| Total Body | 1.25E−02 | Total Body | 1.49E−02 | Total Body | 1.37E−02 |
| Effective Dose | 1.10E−02 | Effective Dose | 1.39E−02 | Effective Dose | 1.25E−02 |

The data in Table 1 demonstrates that a compound of the disclosure including the ¹⁸F radioisotope has estimated radiation absorption amounts typical of known ¹⁸F tracer molecules. See, for example, Jackson et al. EJNMMI Radiopharm Chem 2020; 5:24 and Zanotti-Fregonara et al. J. Nucl Med. 2021; 62:158-159 and the references cited therein.

Because modifications and changes varied to fit particular operating requirements and environments will be apparent to those skilled in the art, the disclosure is not considered limited to the examples chosen for purposes of illustration and covers all changes and modifications which do not constitute departures from the true spirit and scope of this disclosure.

Accordingly, the foregoing description is given for clearness of understanding only and no unnecessary limitations should be understood therefrom, as modifications within the scope of the disclosure may be apparent to those having ordinary skill in the art.

Throughout the specification, where the compositions and methods are described as including components, steps, or materials, it is contemplated that the compositions and methods can also comprise, consist essentially of, or consist of, any combination of the recited components, steps, or materials, unless described otherwise.

What is claimed is:

1. A fibroblast activation protein inhibitor having a structure according to formula (I), formula (II), or formula (III): 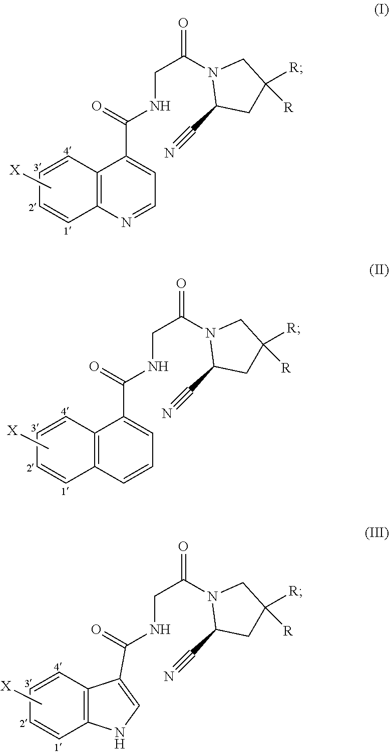
wherein
X is located at carbon 1′, 2′, or 3′ and is selected from the group of I, ¹⁸F, ²¹¹At, ¹²³I, ¹²⁴I, ¹²⁵I, ¹³¹I, ⁷⁶Br, ⁷⁷Br, H₃¹¹CO, CF₂¹⁸F, CHF¹⁸F, OCF₂¹⁸F, OCHF¹⁸F, SCF₂¹⁸F, SCHF¹⁸F, ¹¹CN, ¹¹CH₃, and H₃¹¹CS;
each R is independently selected from H, F, ²H, CH₃, and cyclopentyl;
with the proviso that in formula (I), when X is H₃¹¹CO and both R are H or both R are F, then X is not located at carbon 3′.

2. The fibroblast activation protein inhibitor of claim 1, wherein X is located at the 3′ carbon.

3. The fibroblast activation protein inhibitor of claim 1, wherein X is located at the 2′ carbon.

4. The fibroblast activation protein inhibitor of claim 1, wherein X is located at the 1′ carbon.

5. The fibroblast activation protein inhibitor of any one of the preceding claims, wherein X is selected from the group of ¹⁸F, ²¹¹At, ¹²³I, ¹²⁴I, ¹²⁵I, ¹³¹I, ⁷⁶Br, ⁷⁷Br, and H₃¹¹CO.

6. The fibroblast activation protein inhibitor of any one of the preceding claims, wherein X is ¹⁸F.

7. The fibroblast activation protein inhibitor of any one of the preceding claims, wherein at least one R is H or ²H.

8. The fibroblast activation protein inhibitor of any one of the preceding claims, wherein both R are H or ²H.

9. The fibroblast activation protein inhibitor of any one of claims 1 to 7, wherein at least one R is F.

10. The fibroblast activation protein inhibitor of any one of claims 1 to 7, wherein both R are F.

11. The fibroblast activation protein inhibitor of any one of claims 1 to 7, 8, or 9, wherein at least one R is CH₃ or cyclopentyl.

12. The fibroblast activation protein inhibitor of any one of claims 1 to 7, wherein both R are CH₃ or cyclopentyl.

13. The fibroblast activation protein inhibitor of any one of the preceding claims, having a structure according to formula (I).

14. The fibroblast activation protein inhibitor of claim 13, wherein X is located at carbon 3′ and both R are H or F.

15. The fibroblast activation protein inhibitor of claim 134, wherein X is located at carbon 2′ and both R are H or F.

16. The fibroblast activation protein inhibitor of claim 13, wherein X is located at carbon 1′ and both R are H or F.

17. The fibroblast activation protein inhibitor of any one of claims 1 to 12, having a structure according to formula (II).

18. The fibroblast activation protein inhibitor of claim 17, wherein X is located at carbon 3′ and both R are H or F.

19. The fibroblast activation protein inhibitor of claim 17, wherein X is located at carbon 2′ and both R are H or F.

20. The fibroblast activation protein inhibitor of claim 17, wherein X is located at carbon 1′ and both R are H or F.

21. The fibroblast activation protein inhibitor of any one of claims 1 to 12, having a structure according to formula (III).

22. The fibroblast activation protein inhibitor of claim 21, wherein X is located at carbon 3′ and both R are H or F.

23. The fibroblast activation protein inhibitor of claim 21, wherein X is located at carbon 2′ and both R are H or F.

24. The fibroblast activation protein inhibitor of claim 21, wherein X is located at carbon 1′ and both R are H or F.

25. The fibroblast activation protein inhibitor of claim 1 having a structure according to one of the following formulae:

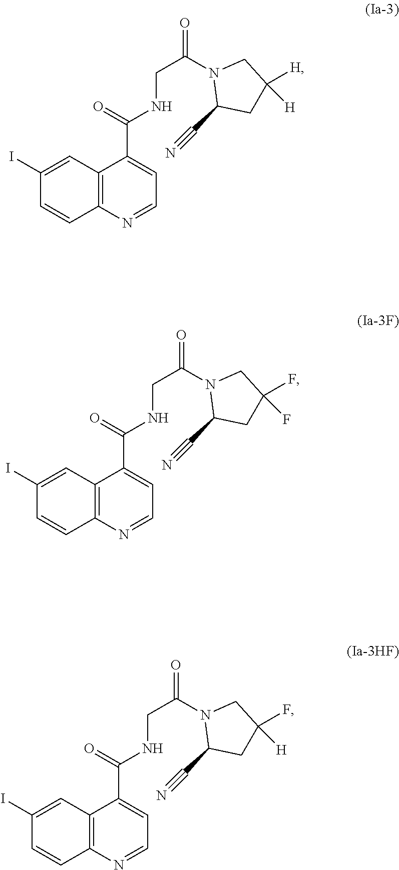

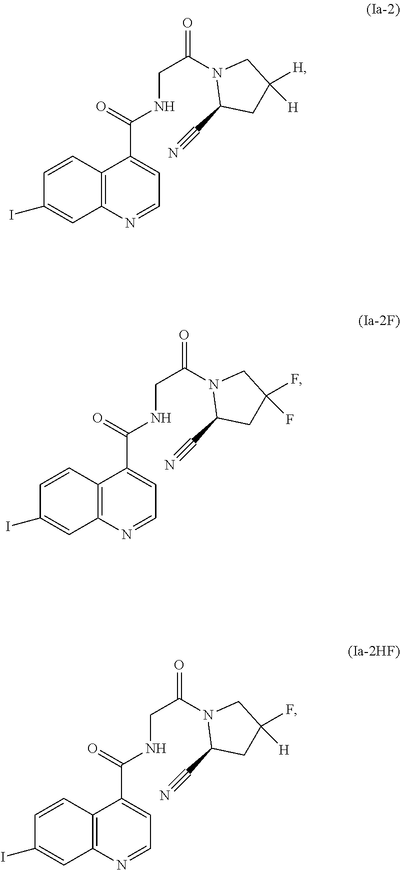

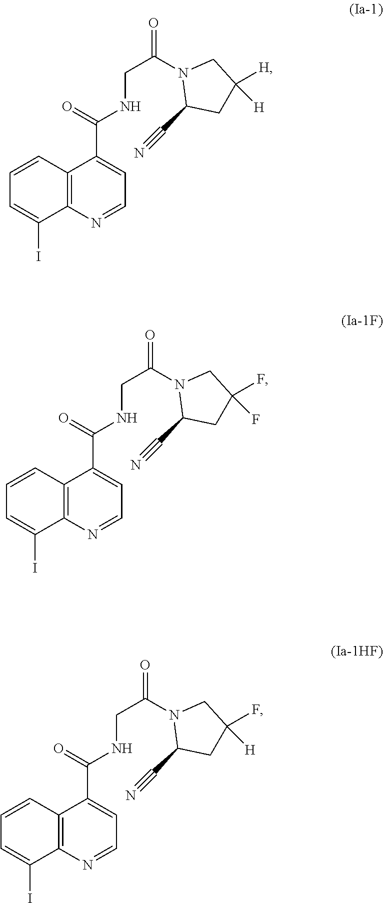

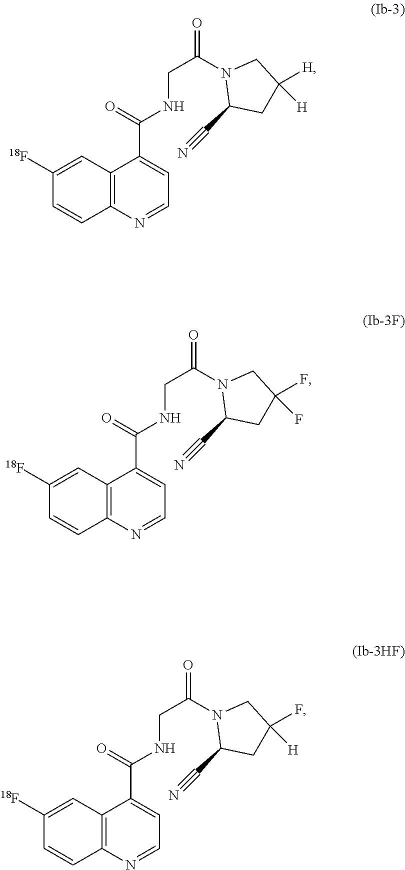

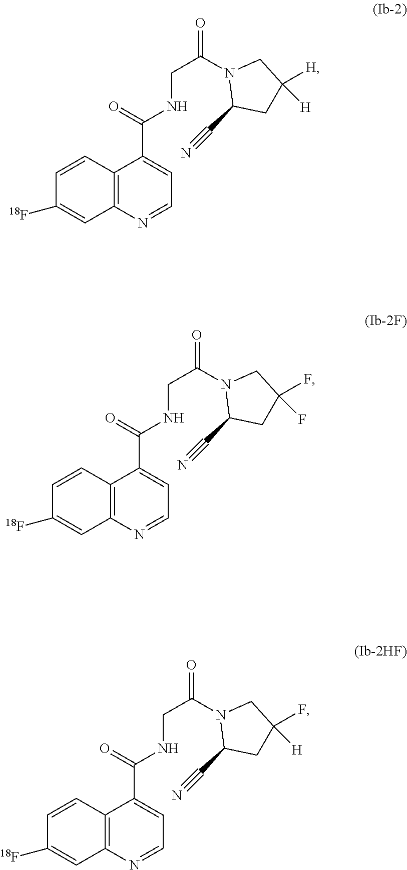

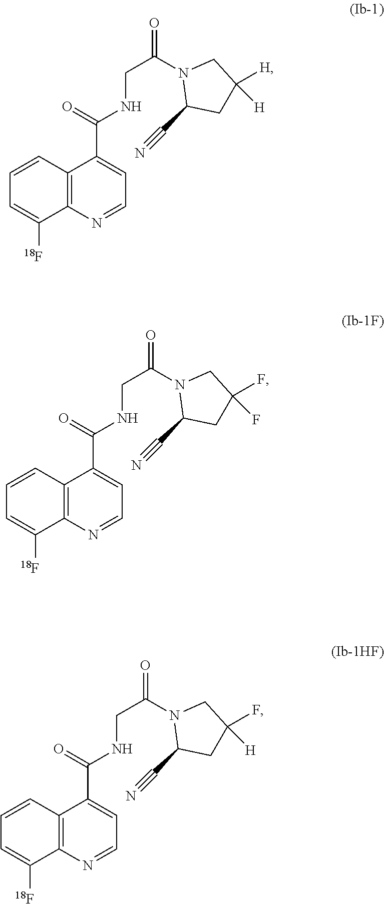

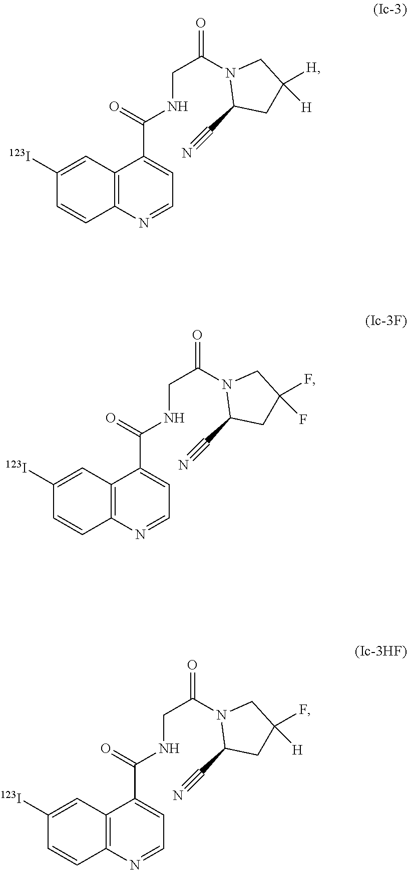

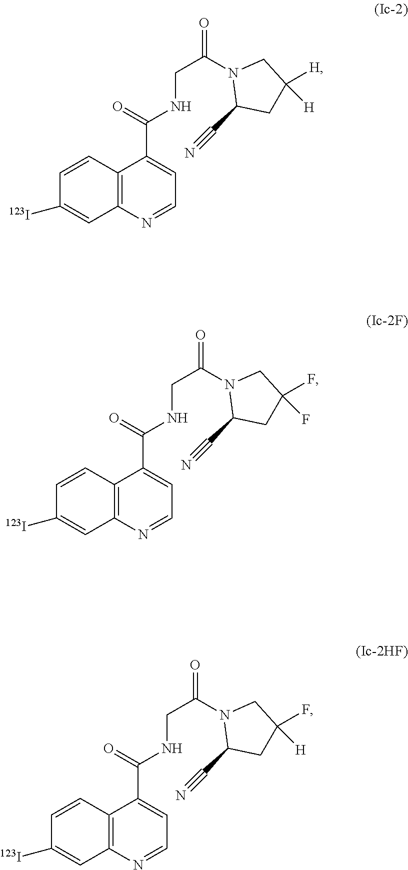

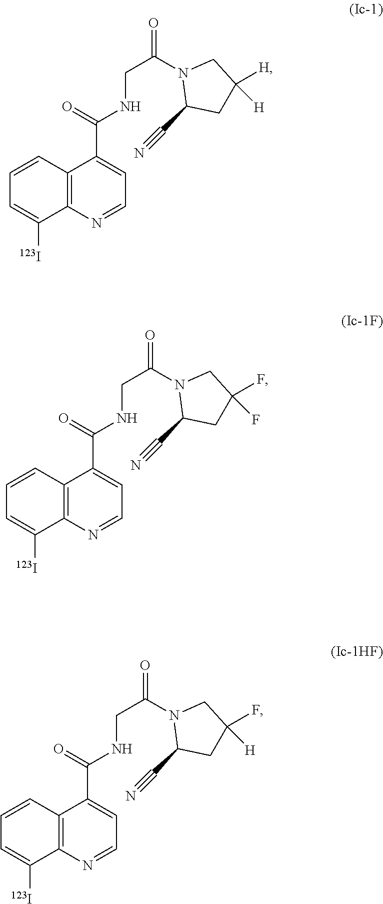

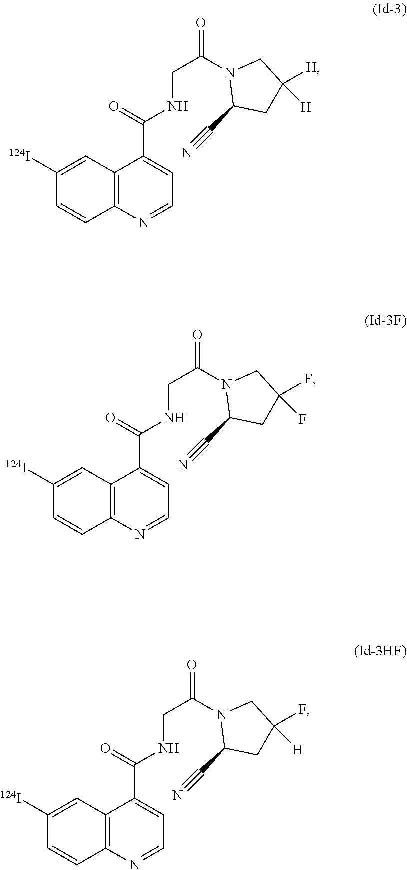

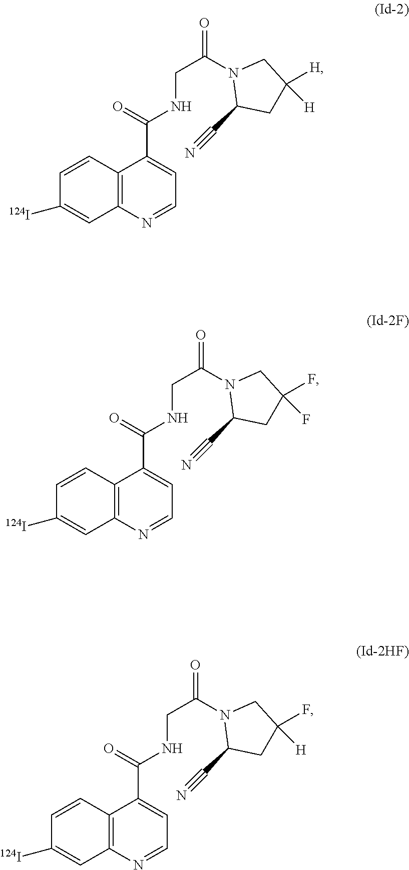

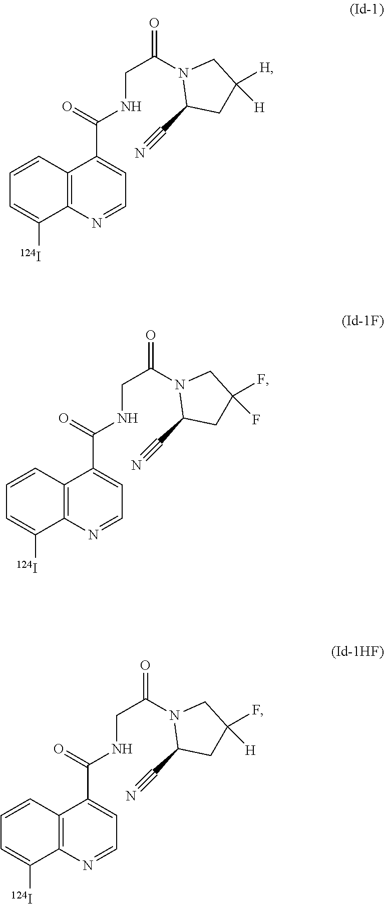

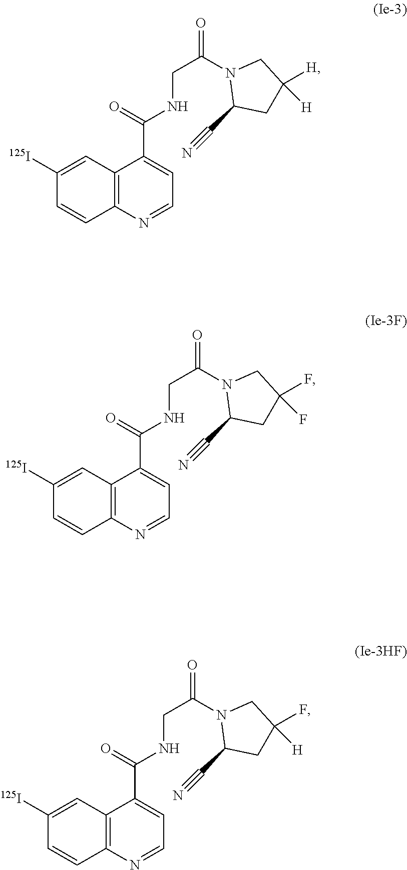

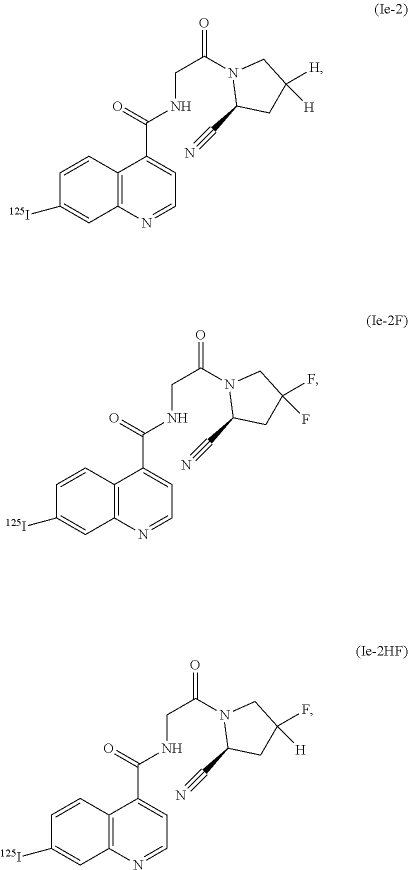

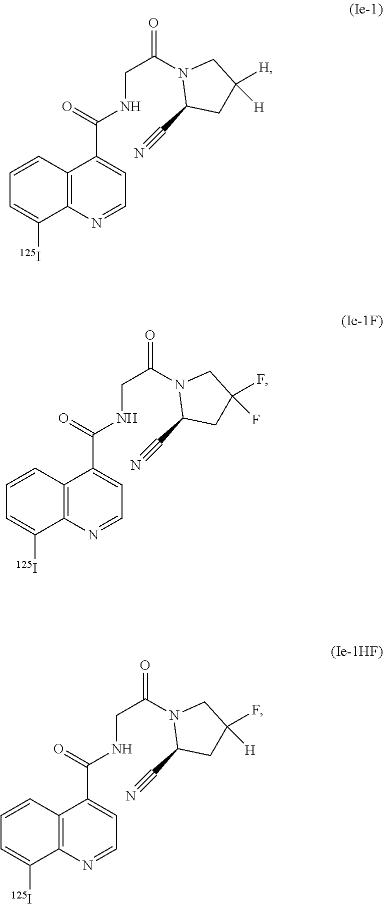

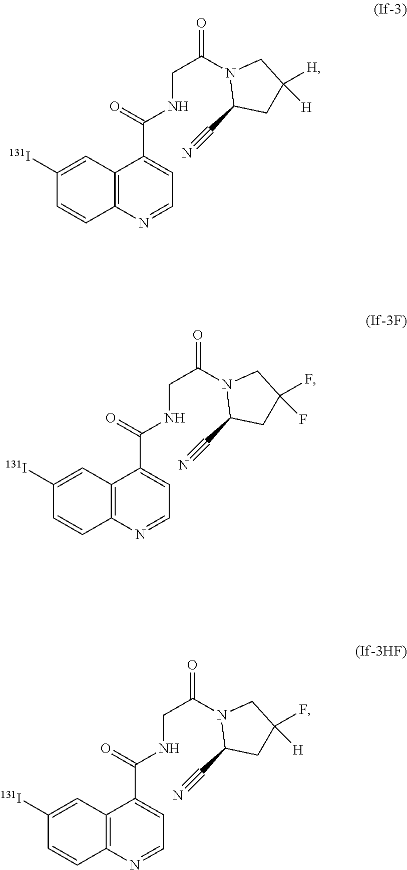

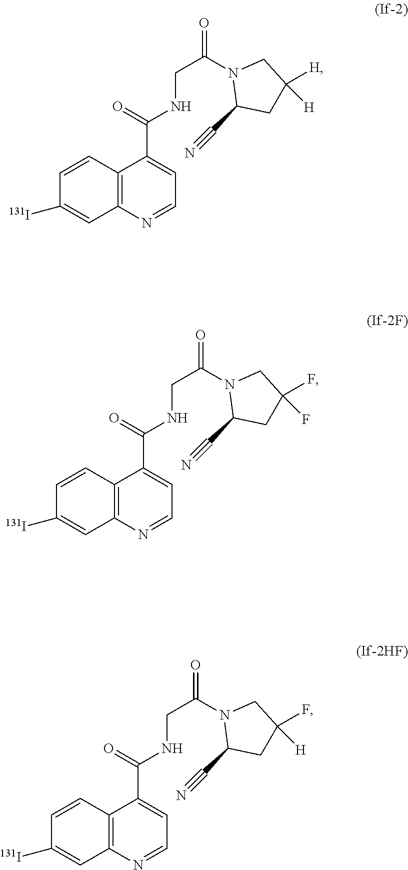

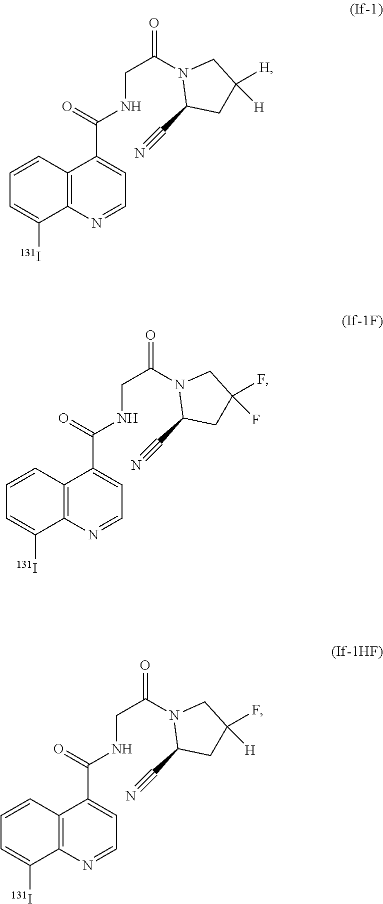

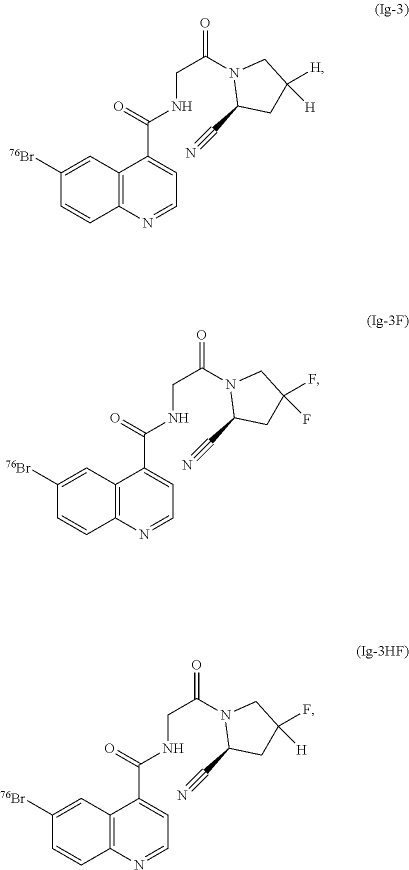

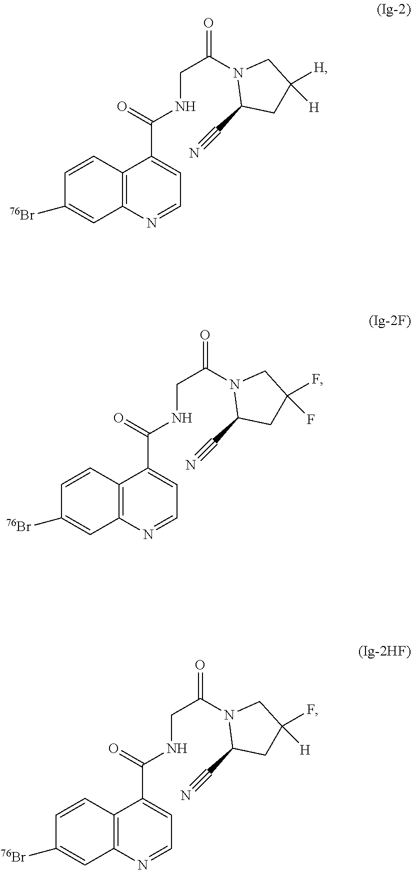

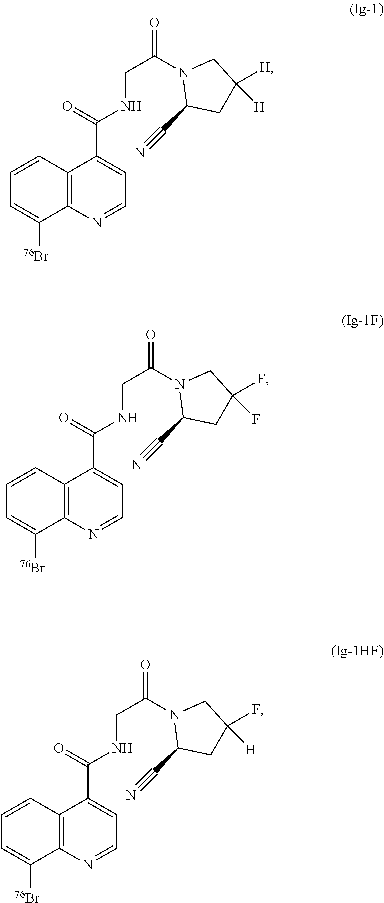

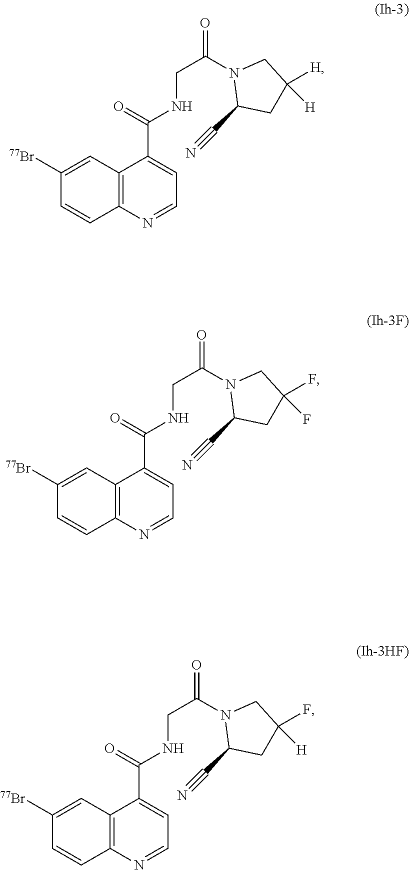

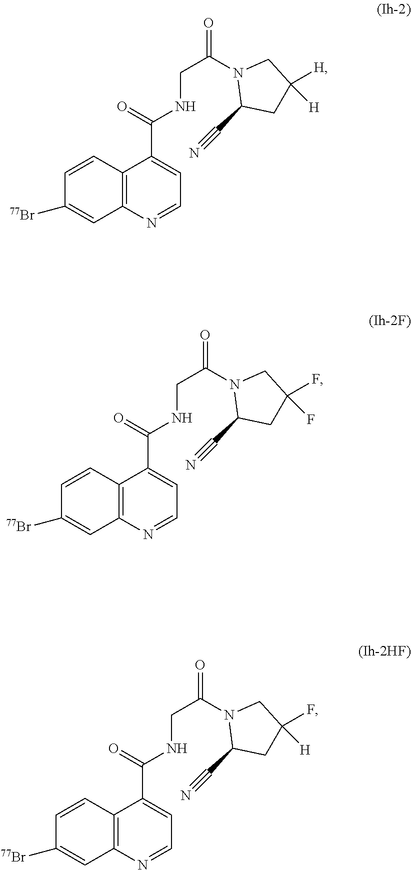

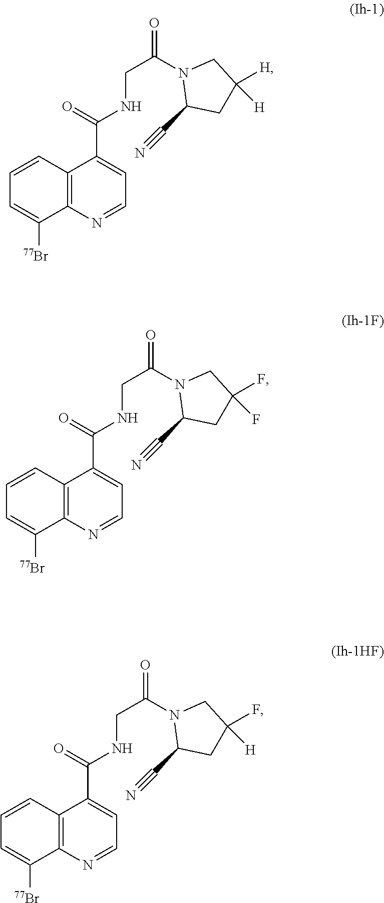

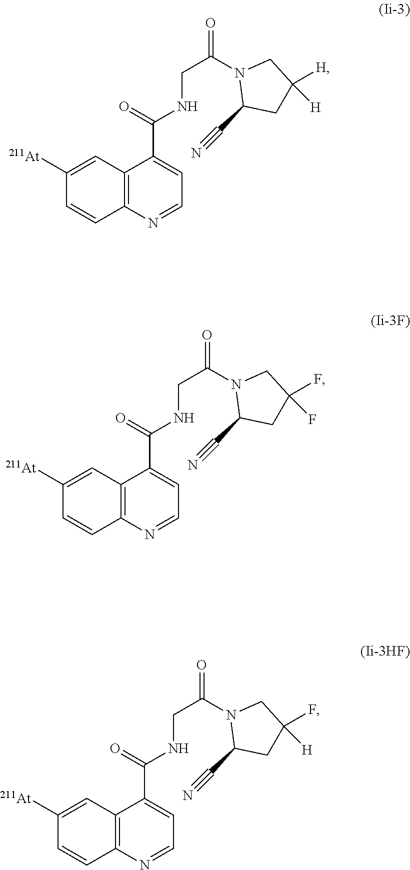

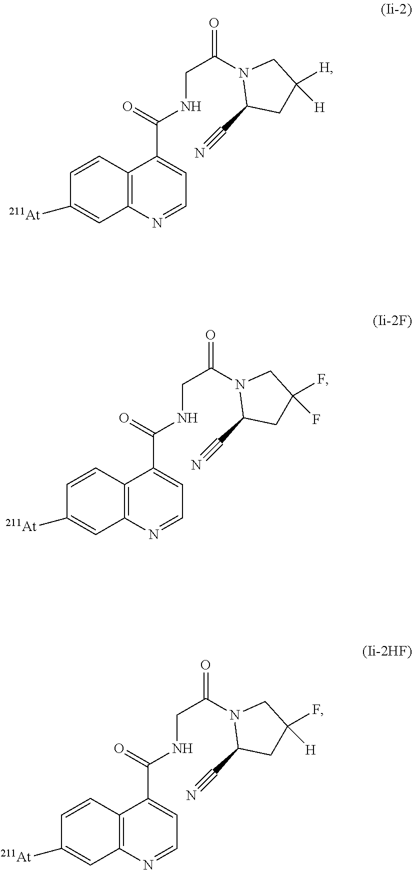

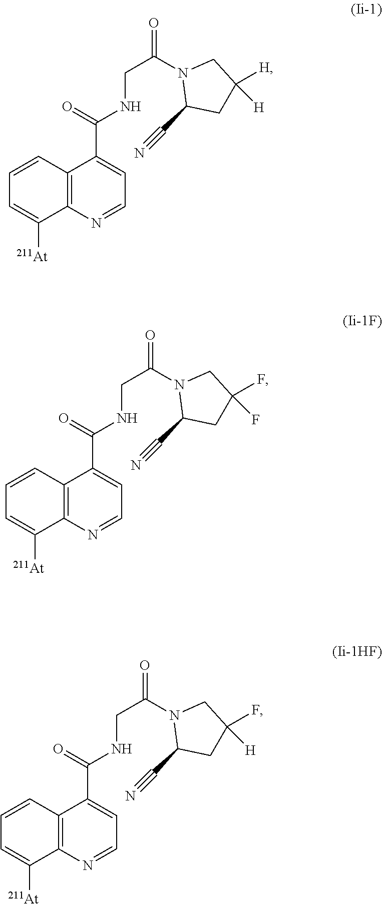

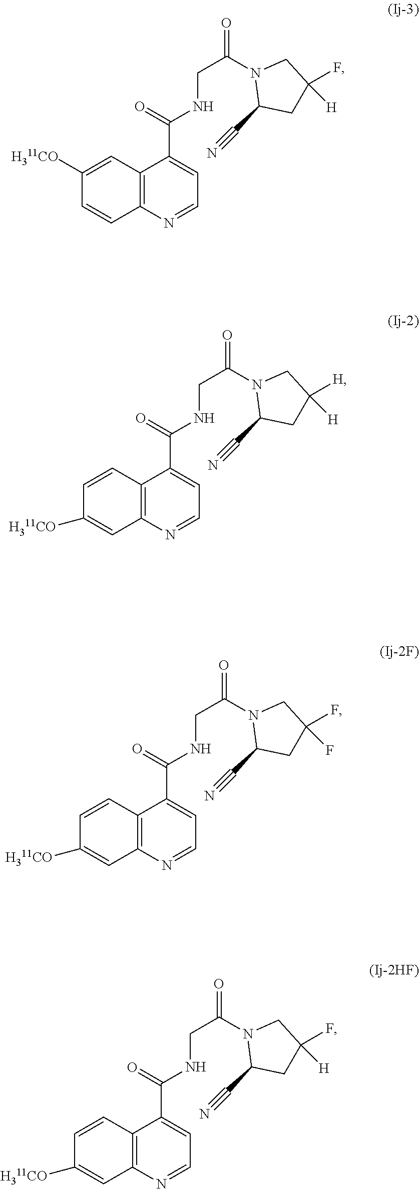

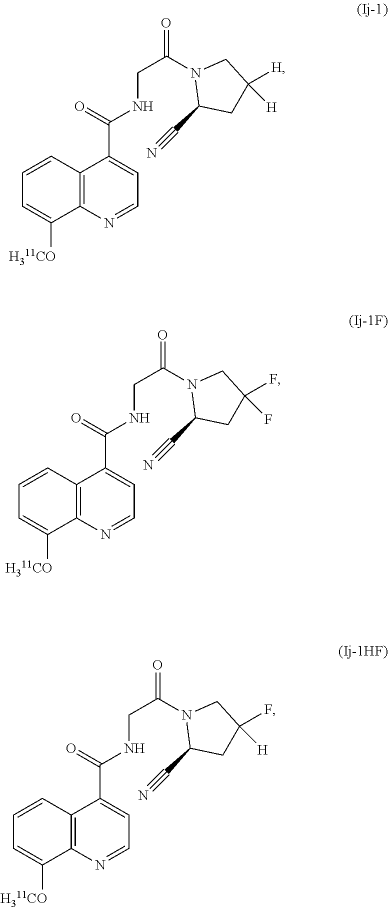

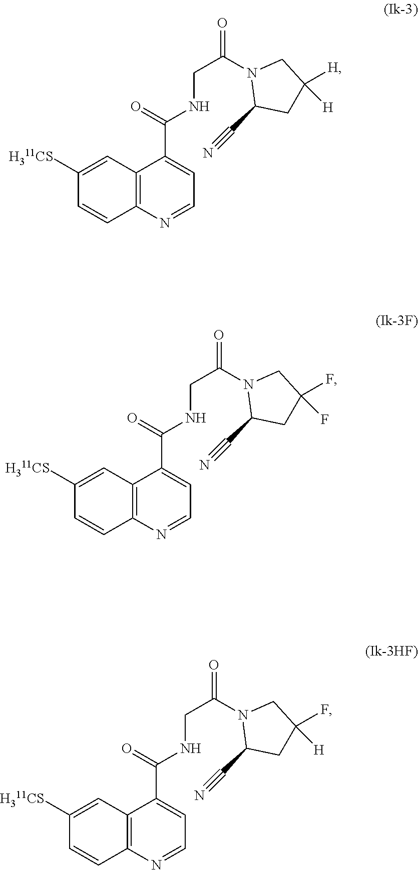

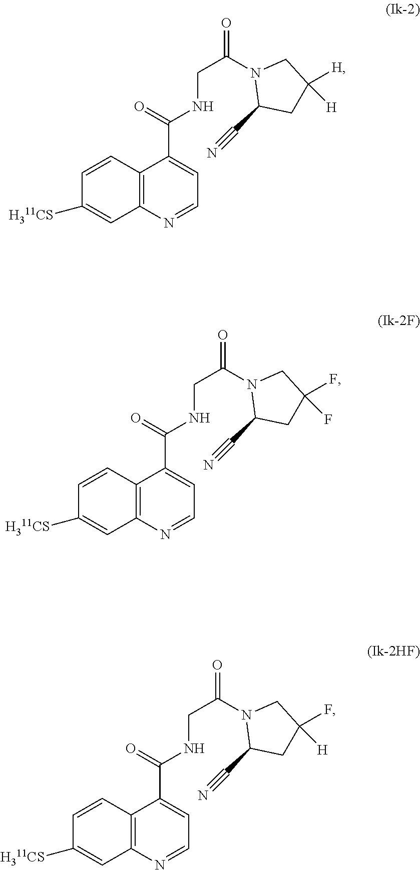

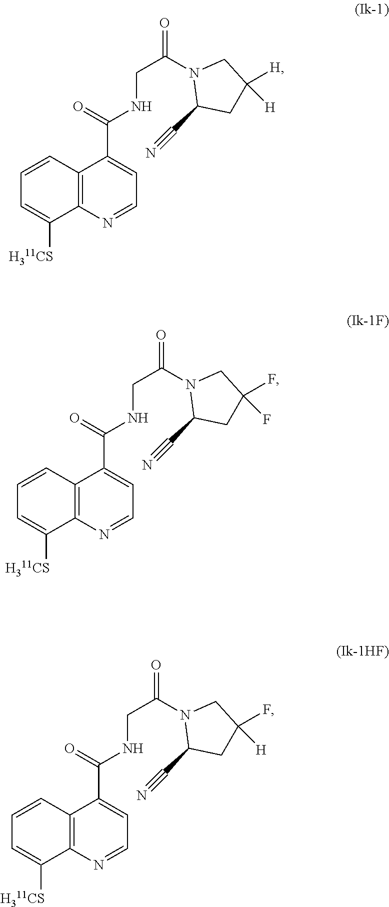

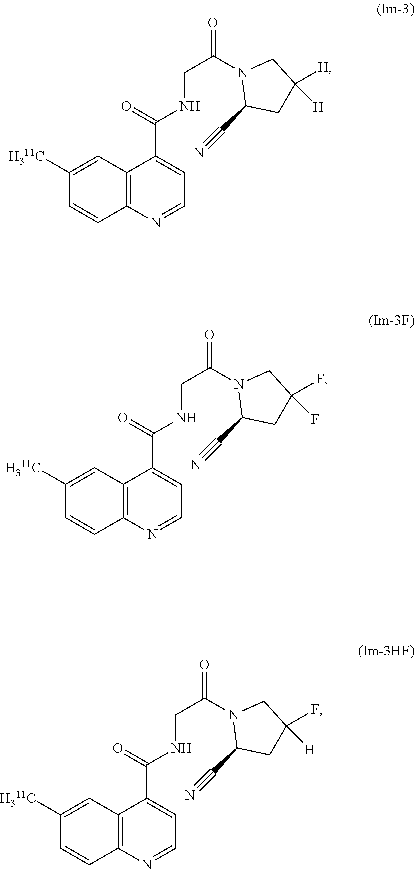

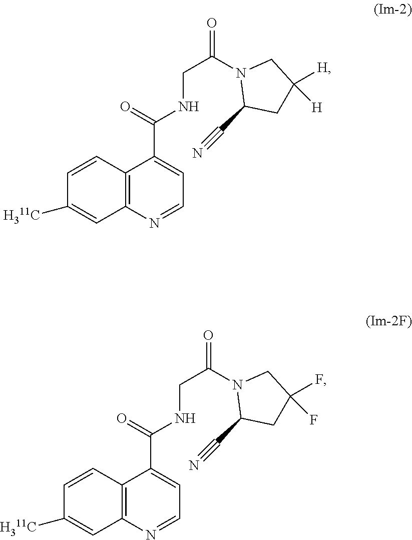

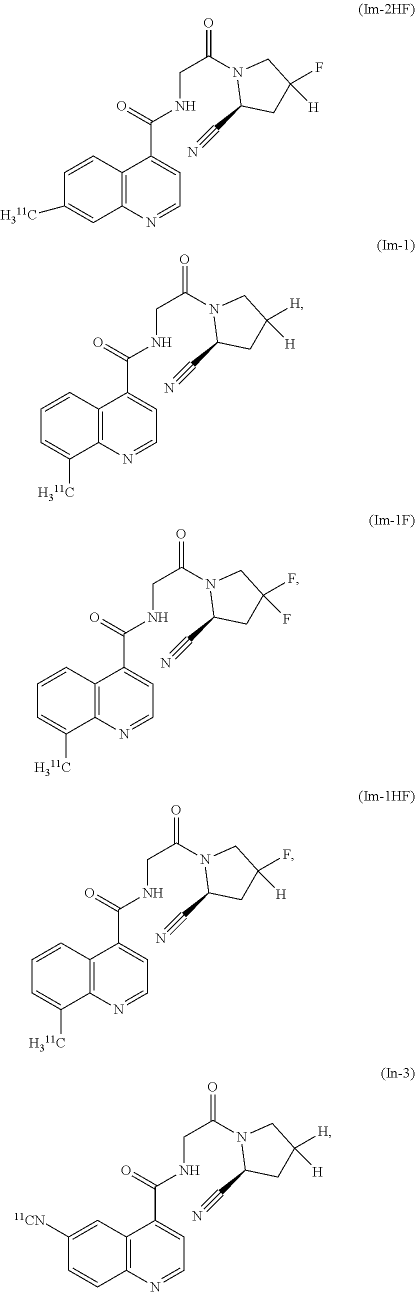

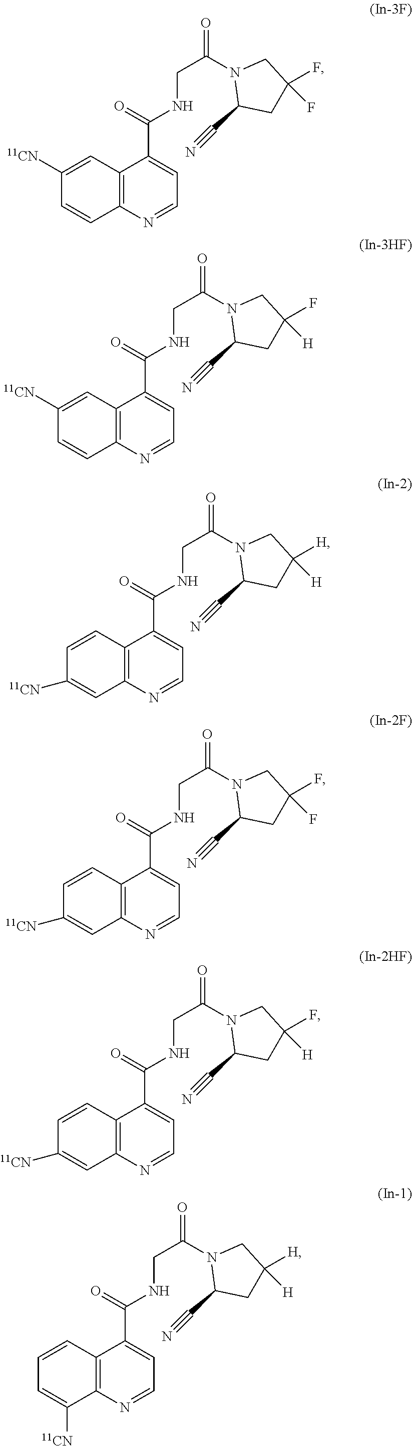

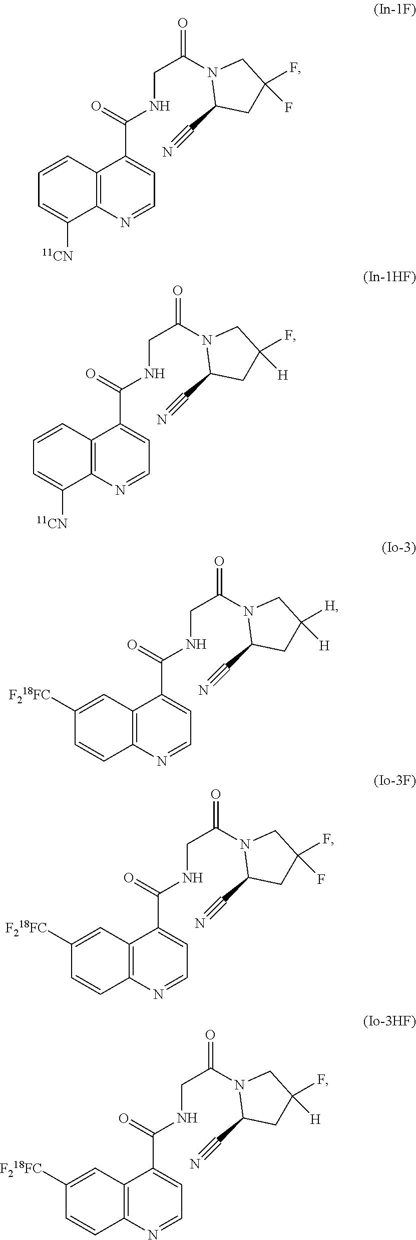

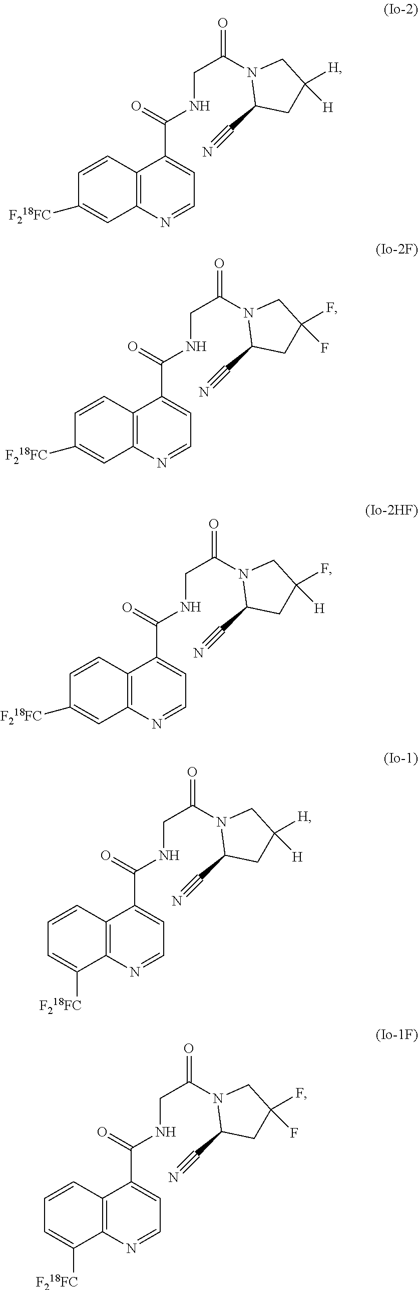

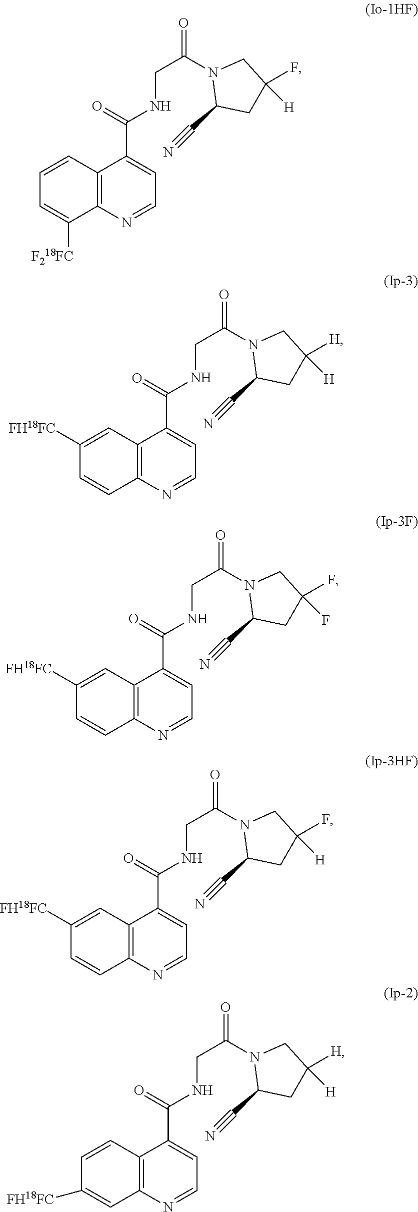

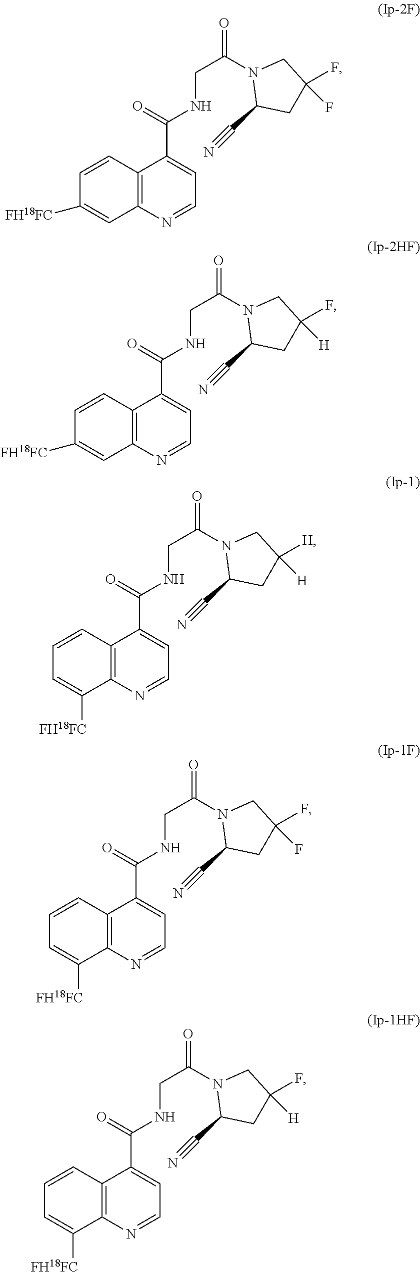

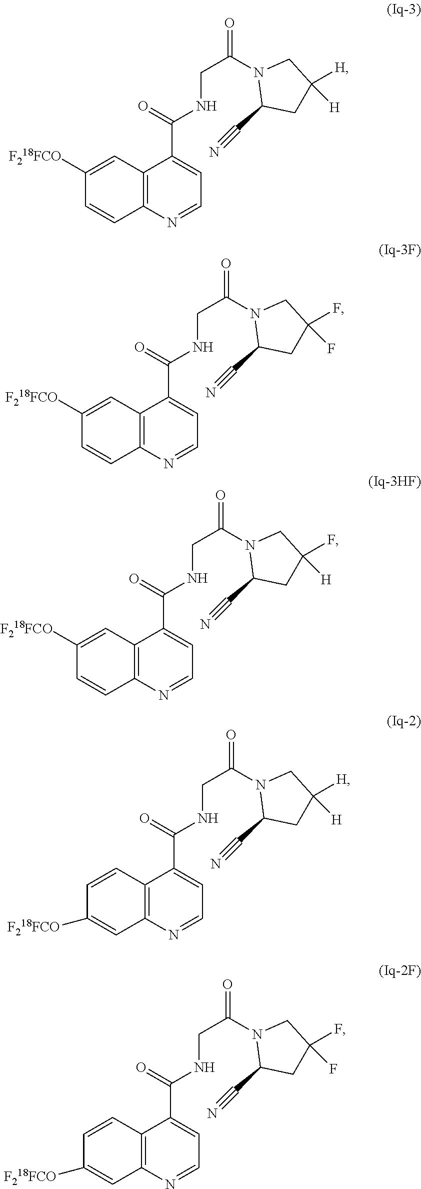

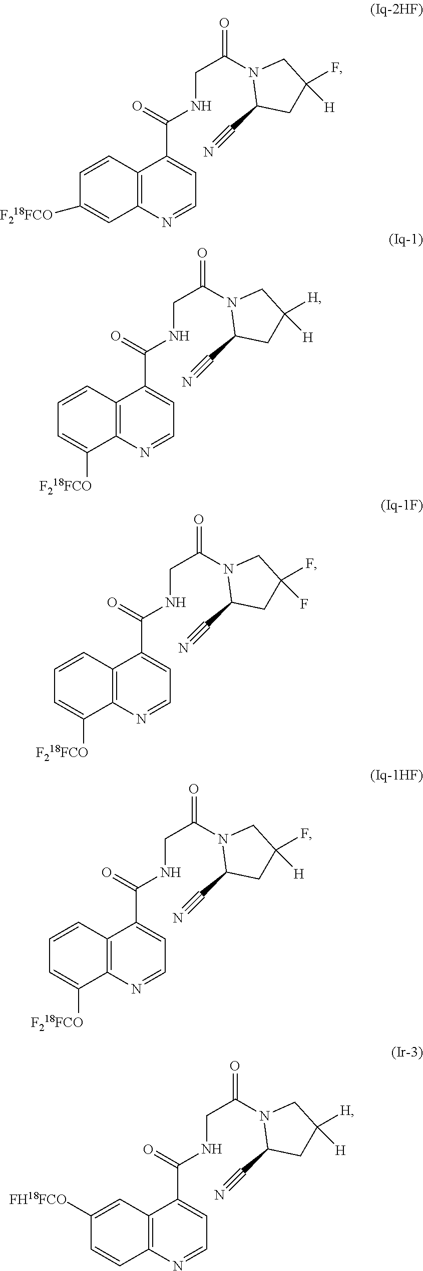

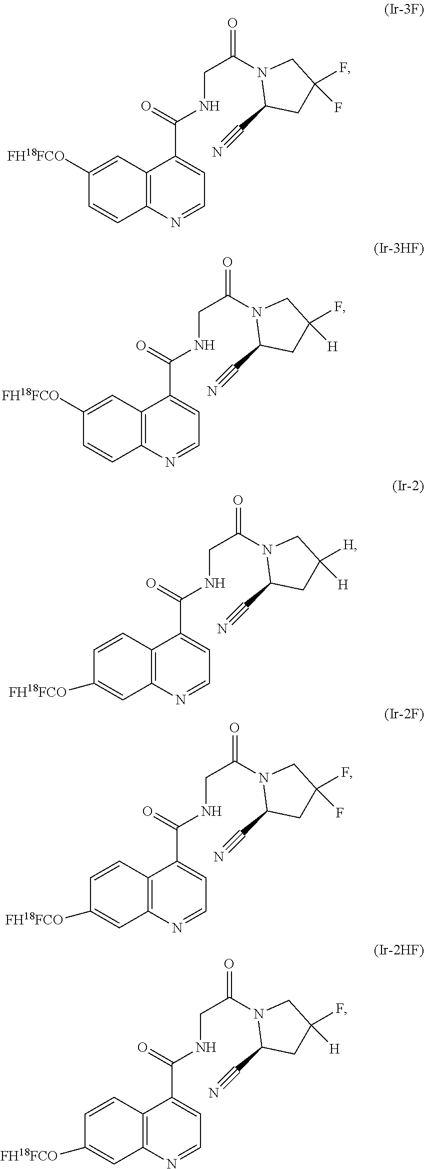

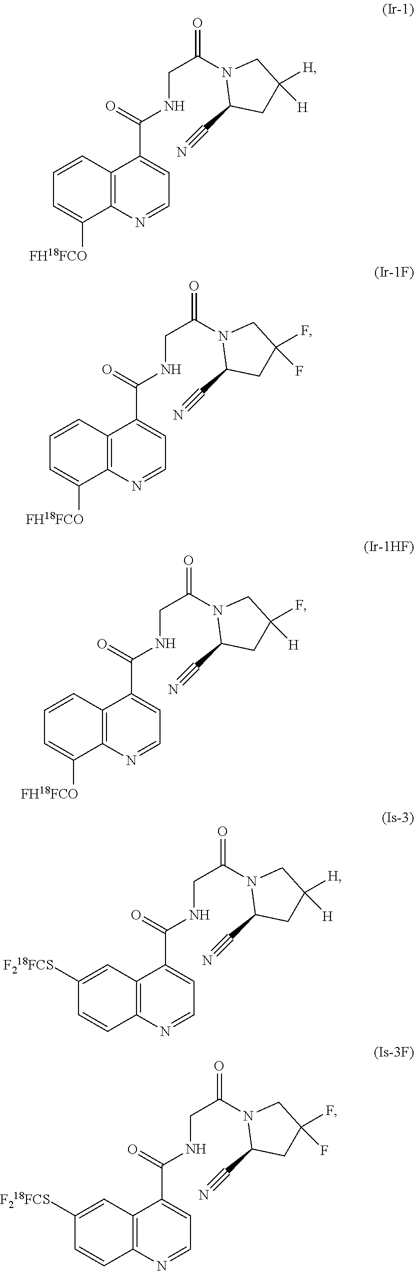

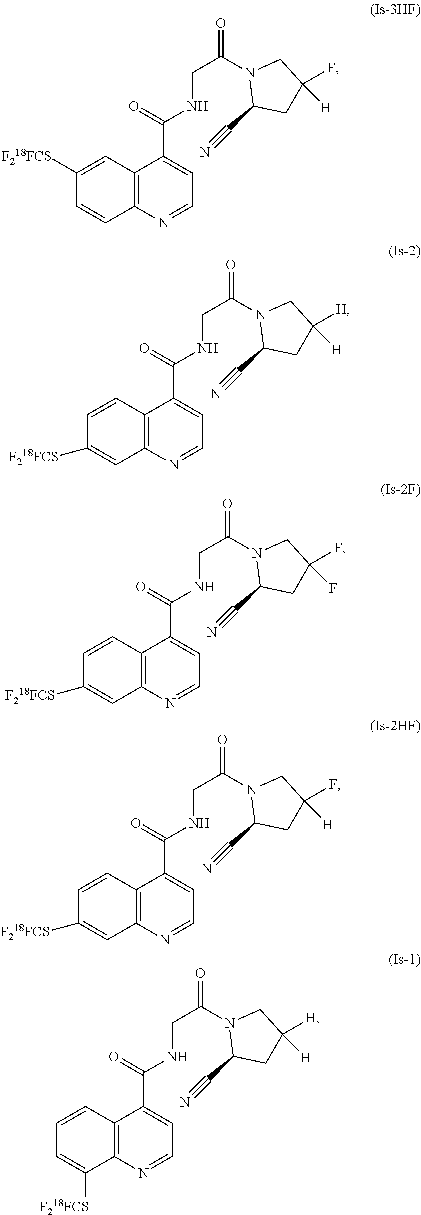

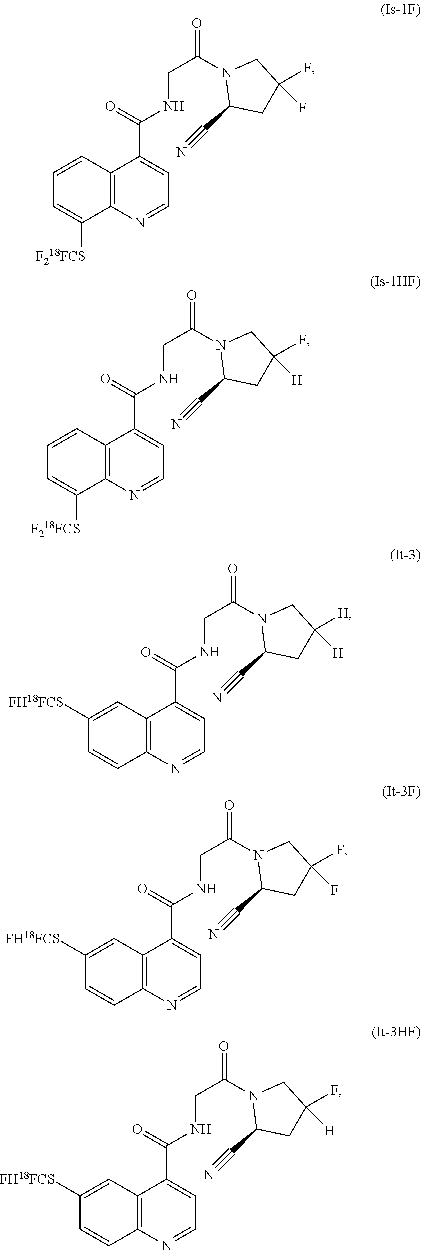

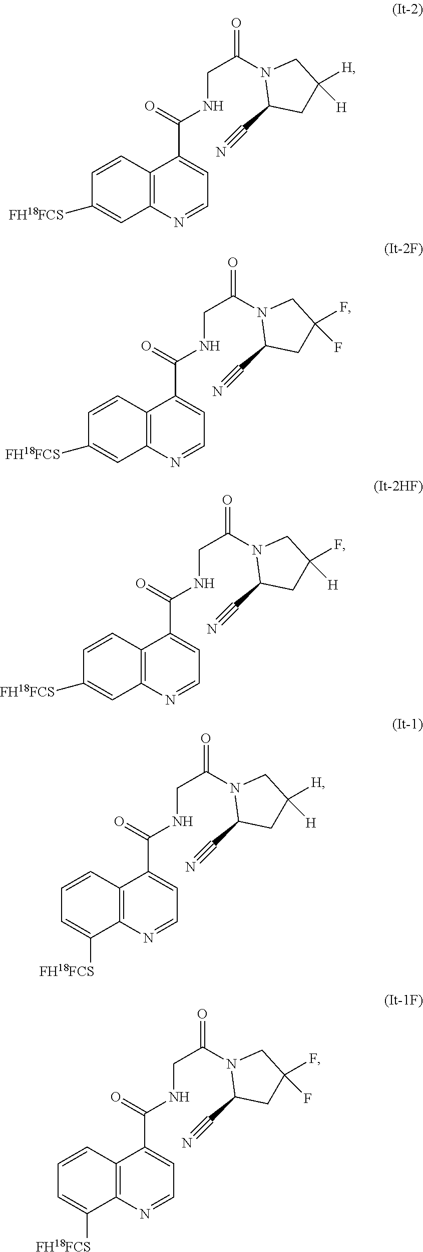

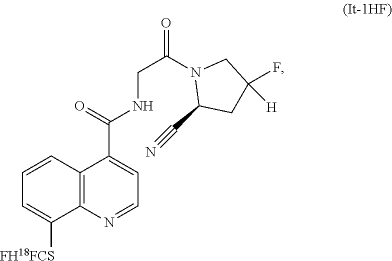

26. The fibroblast activation protein inhibitor of claim 1 having a structure according to one of the following formulae:

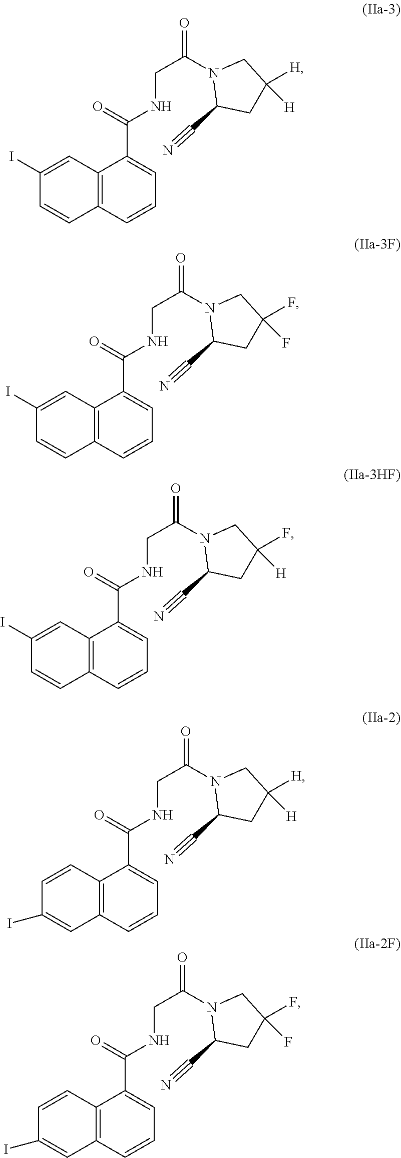

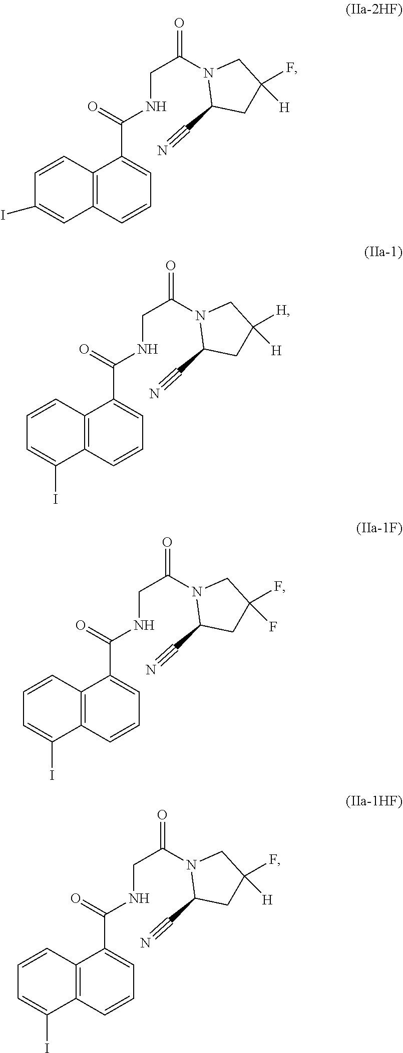

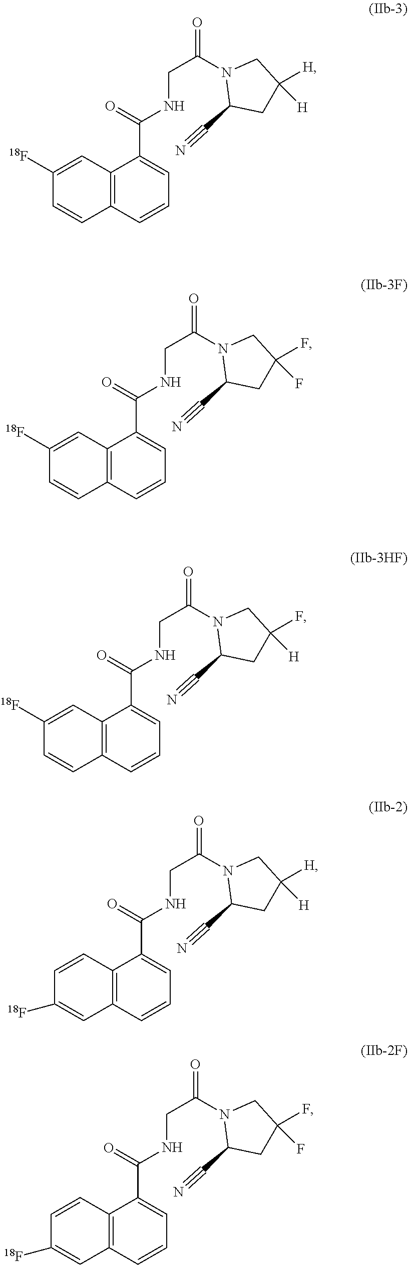

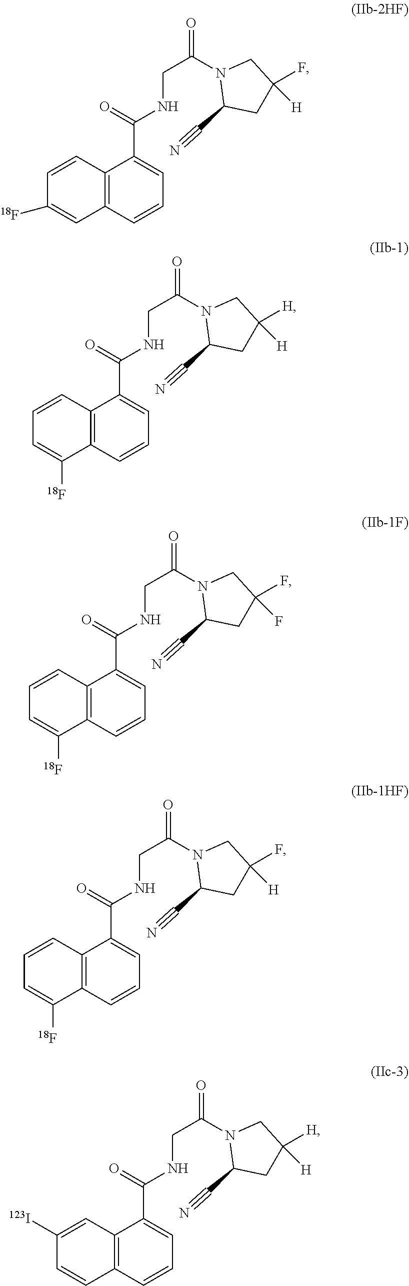

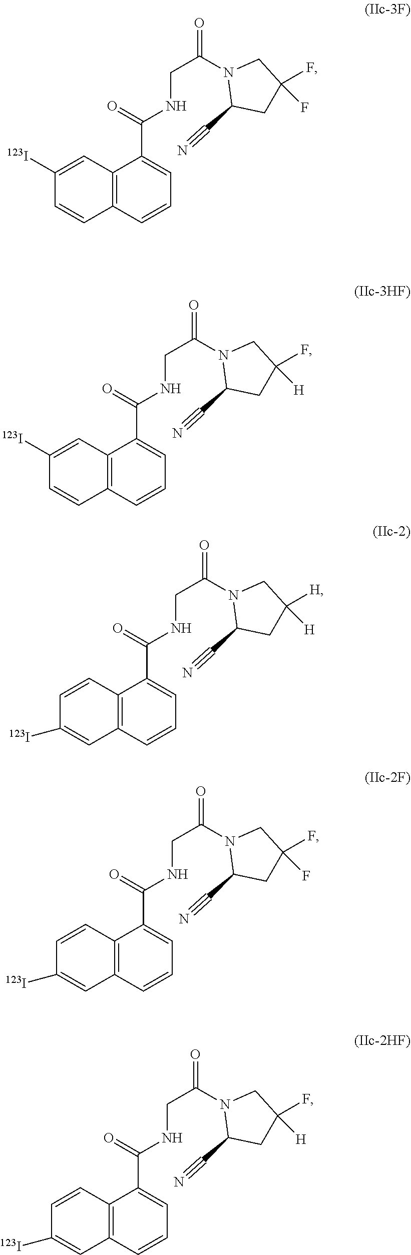

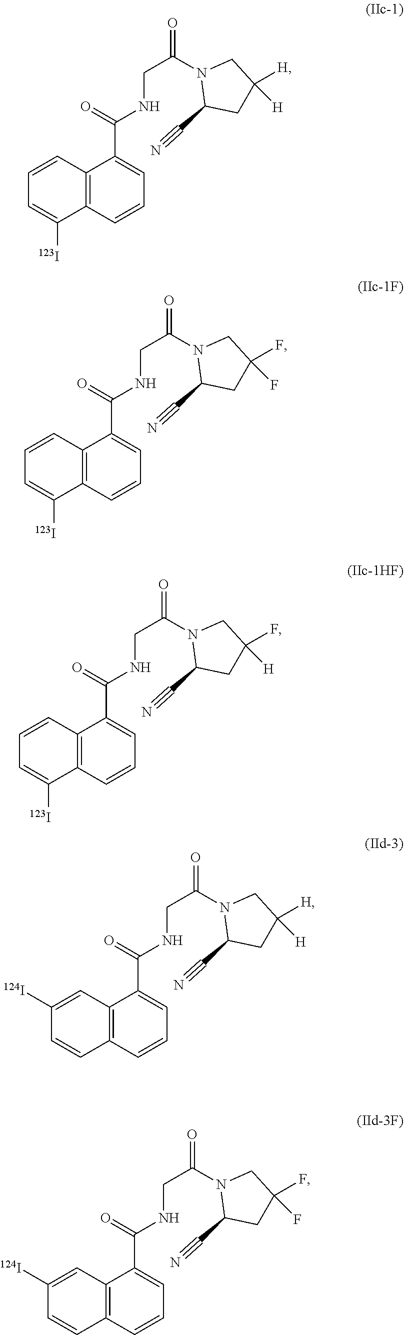

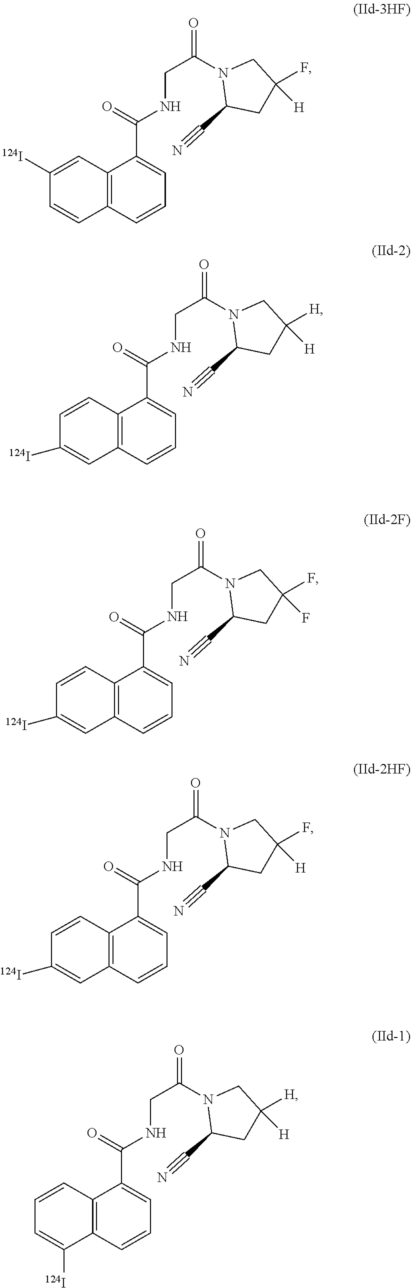

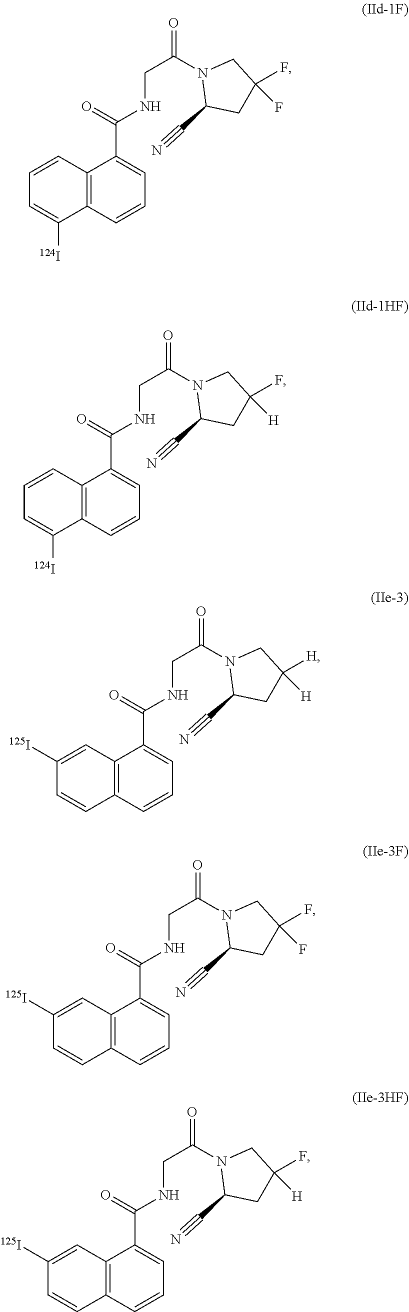

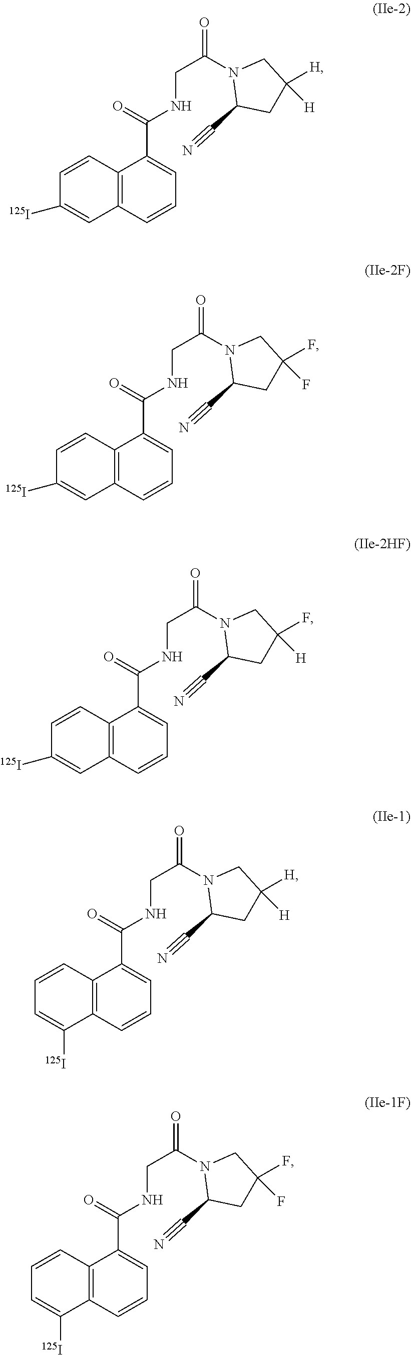

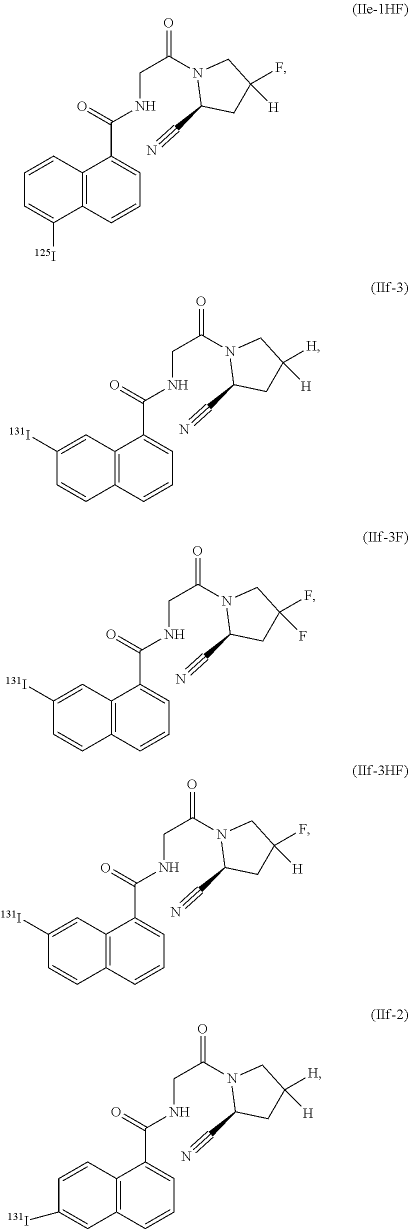

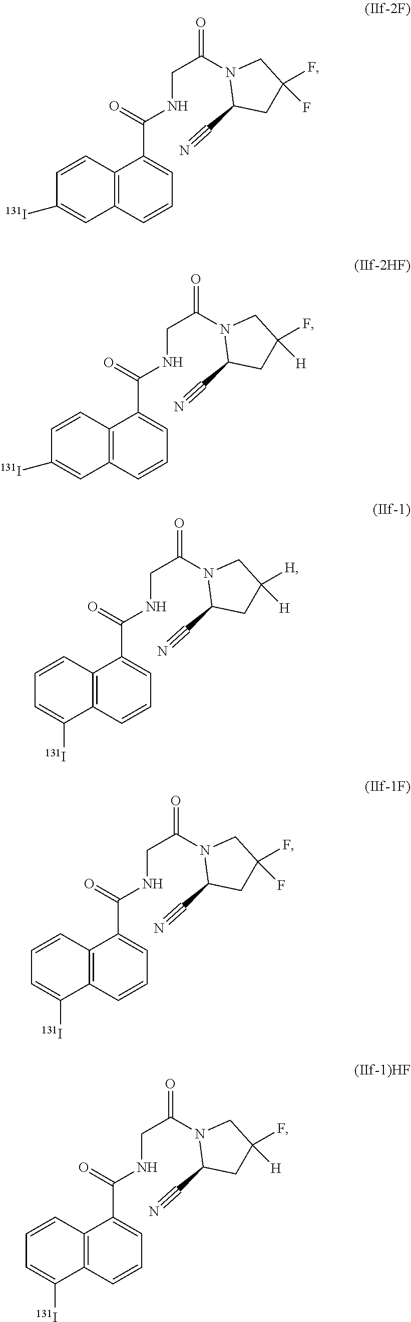

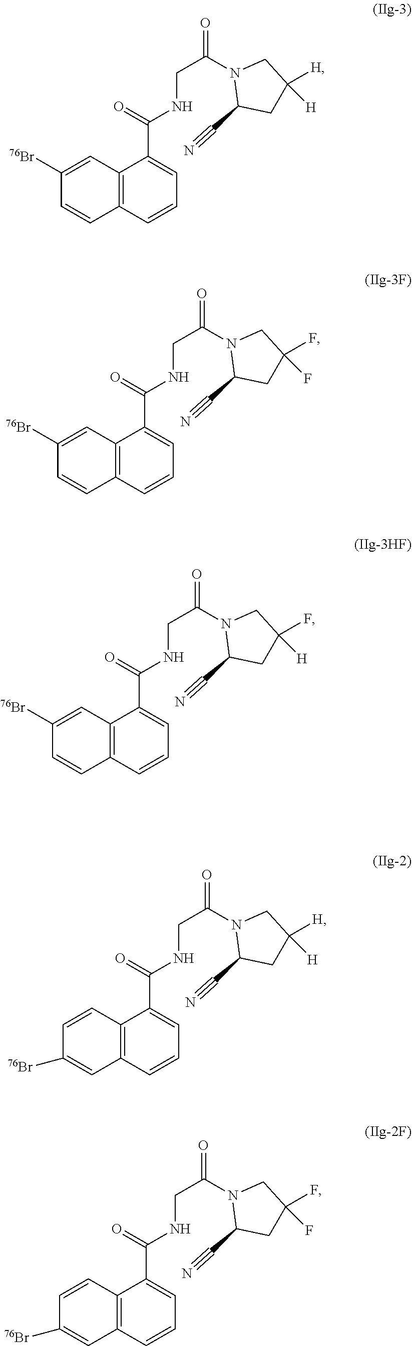

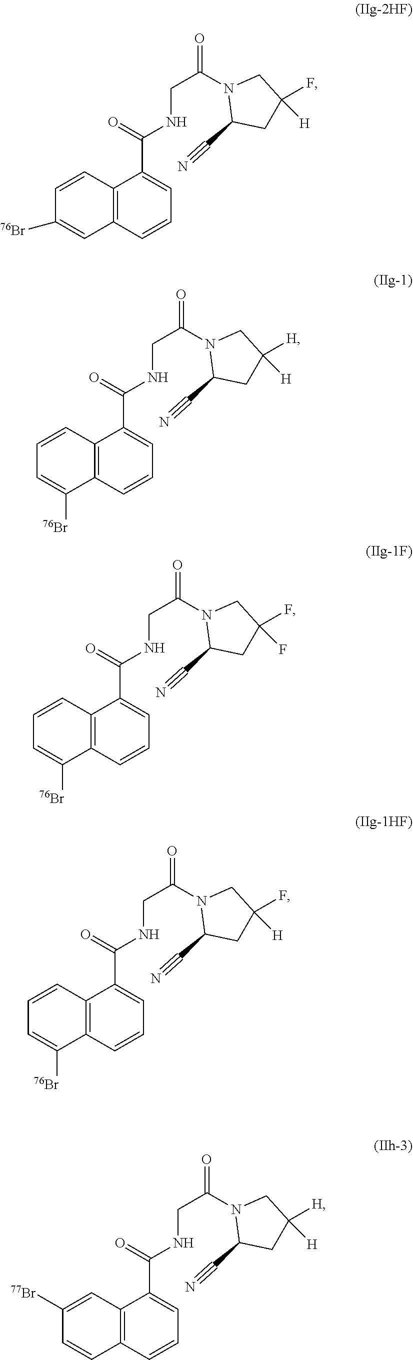

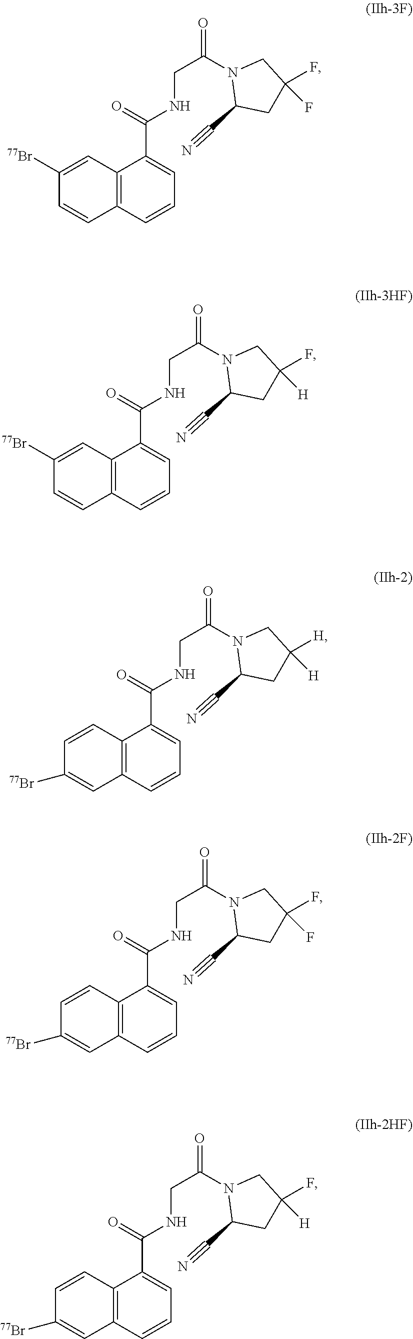

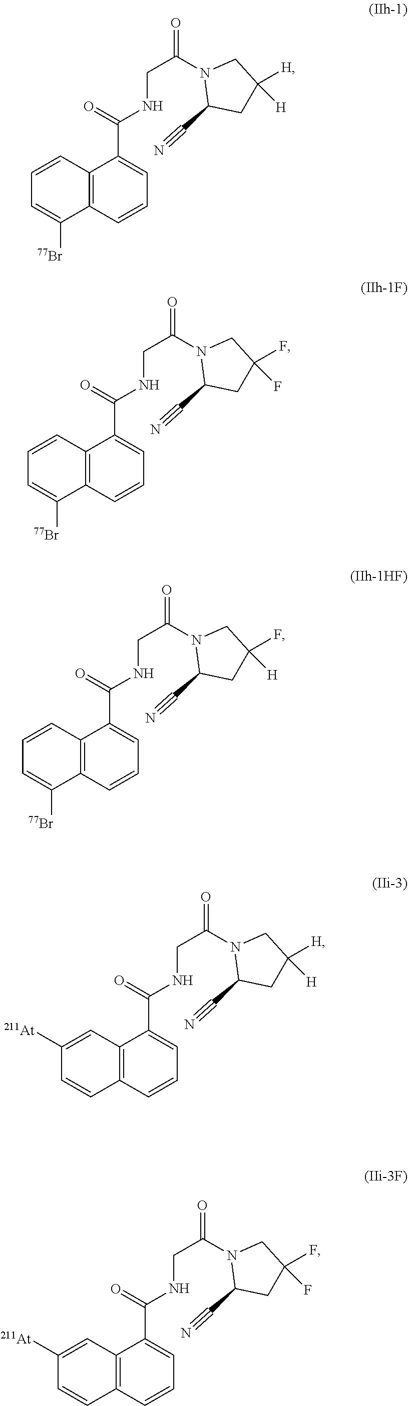

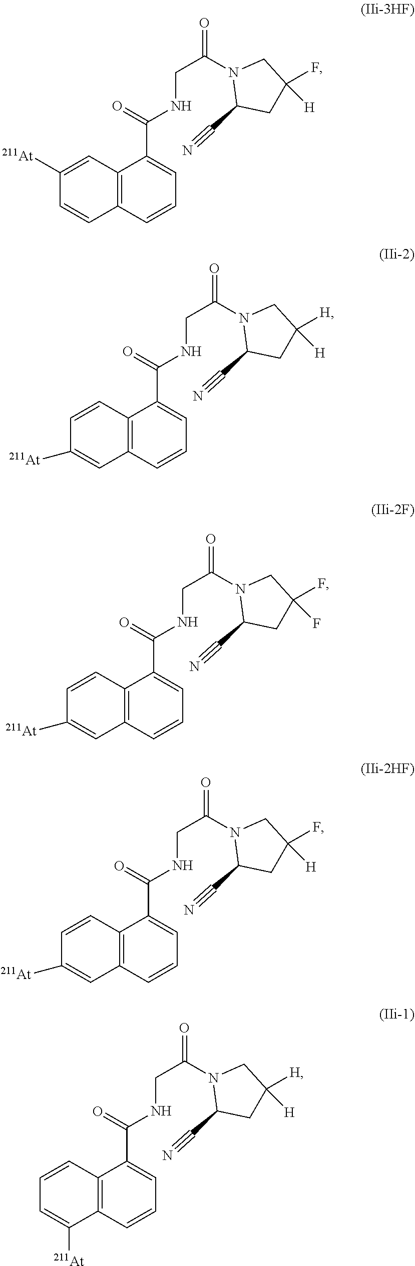

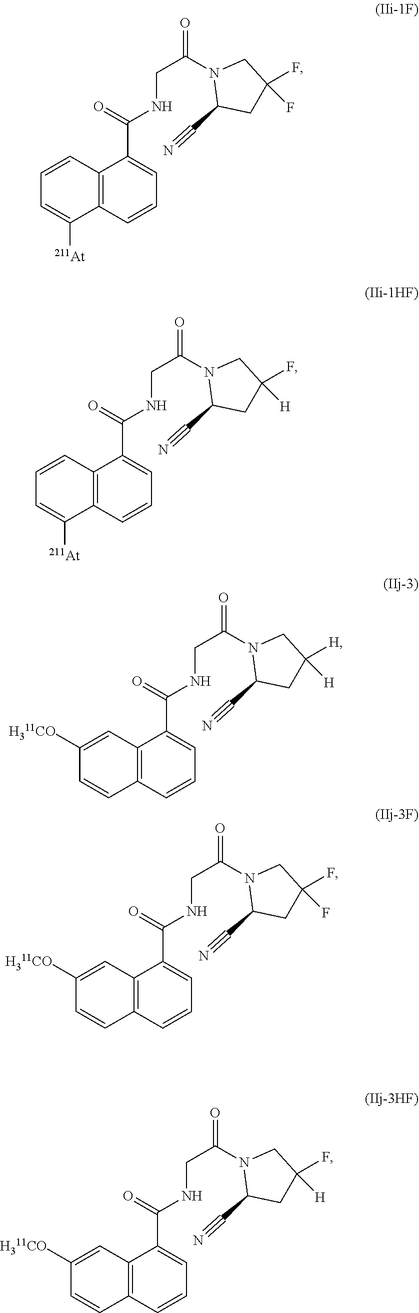

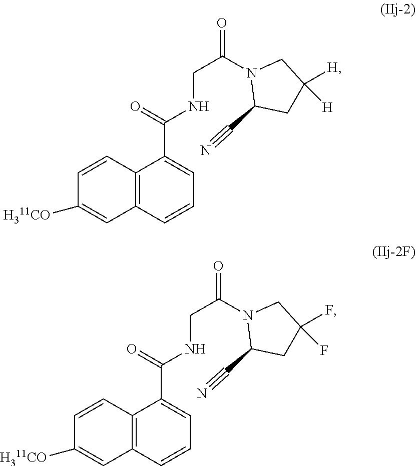

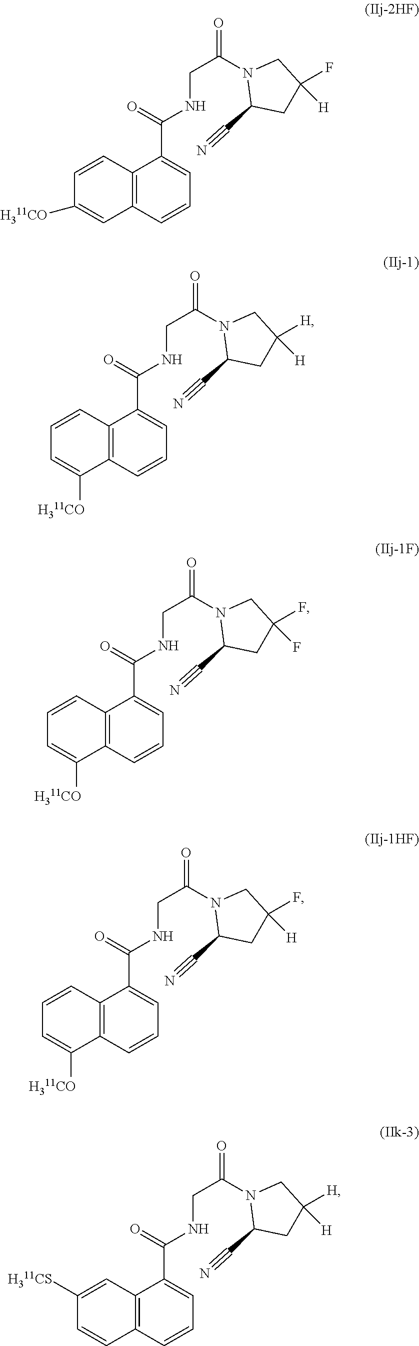

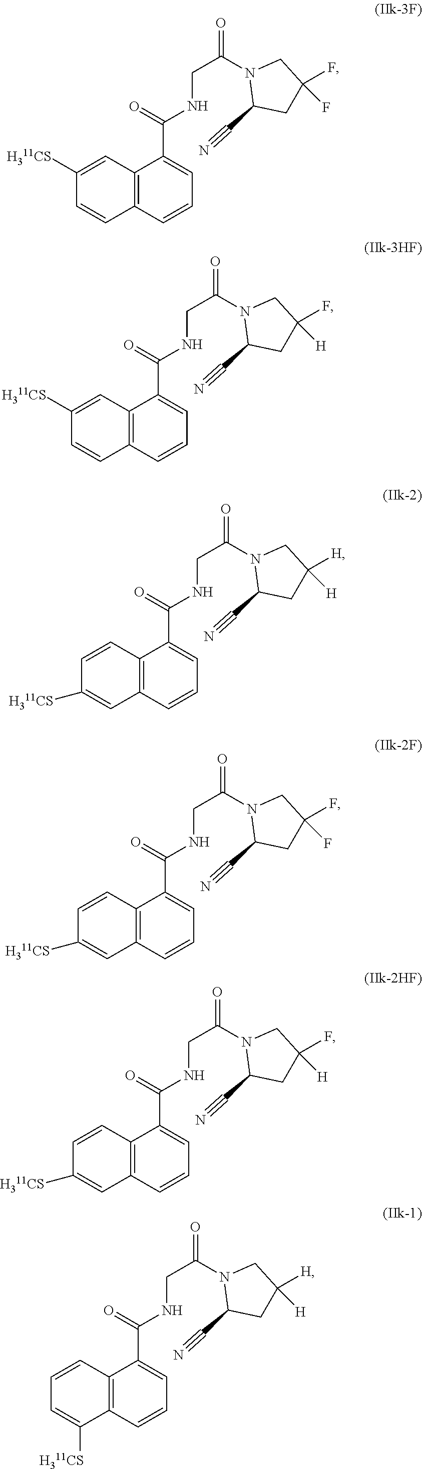

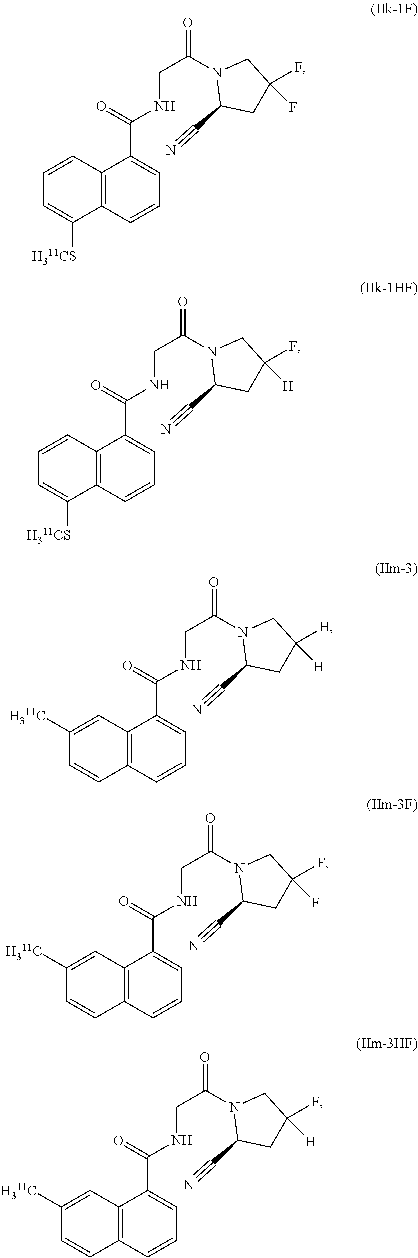

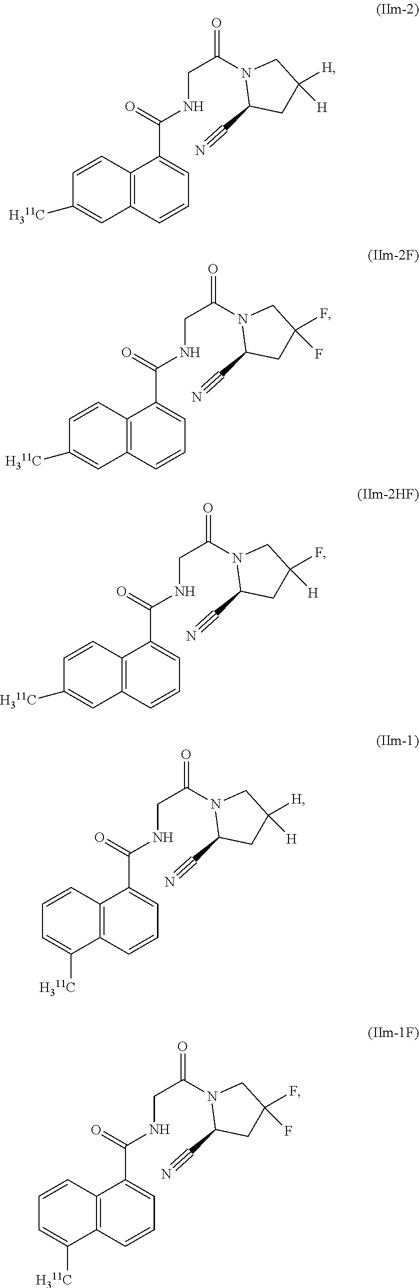

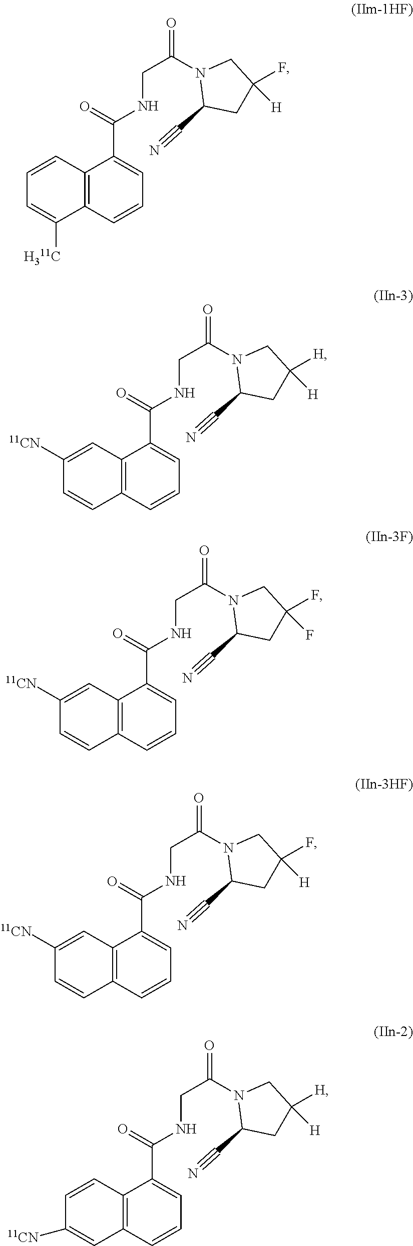

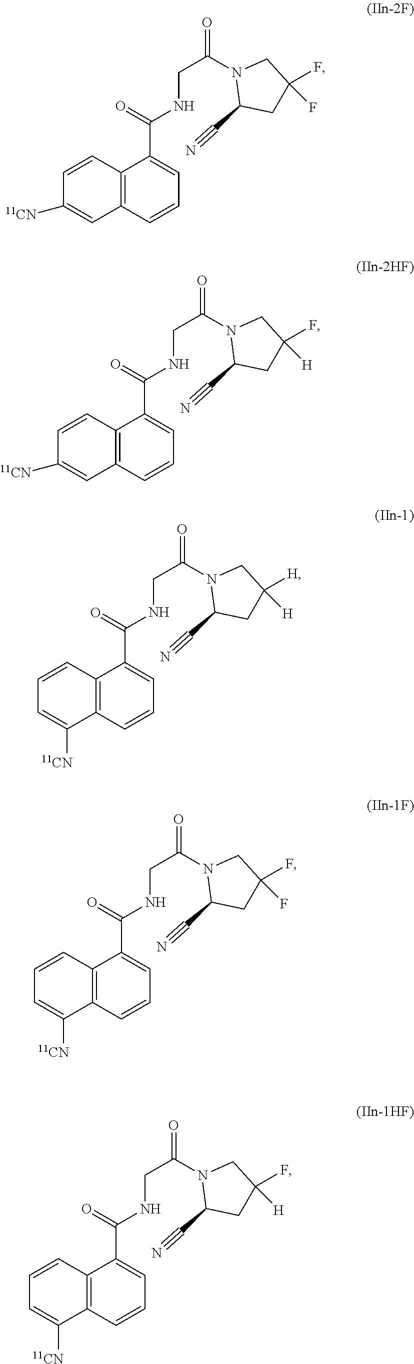

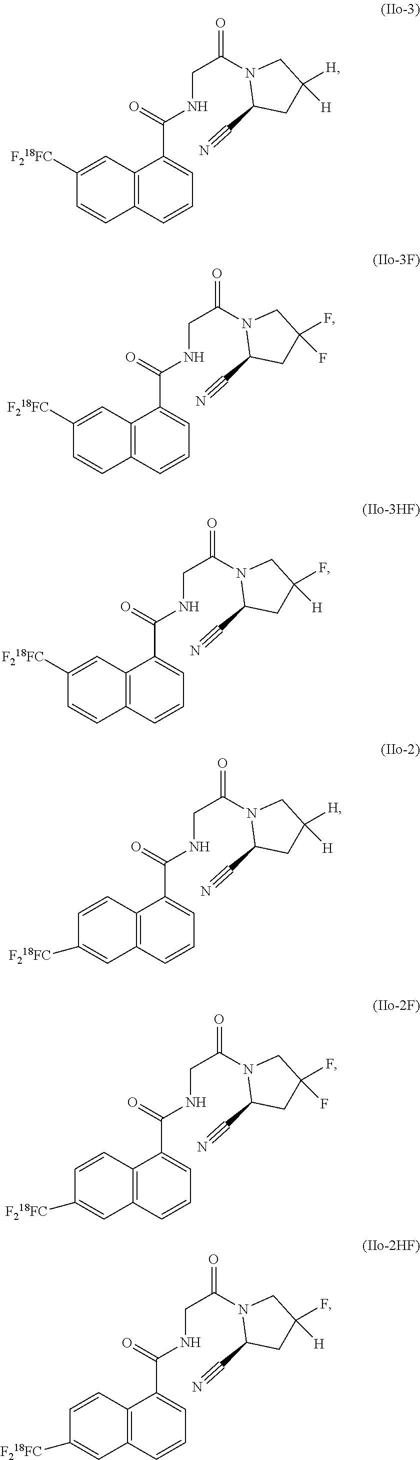

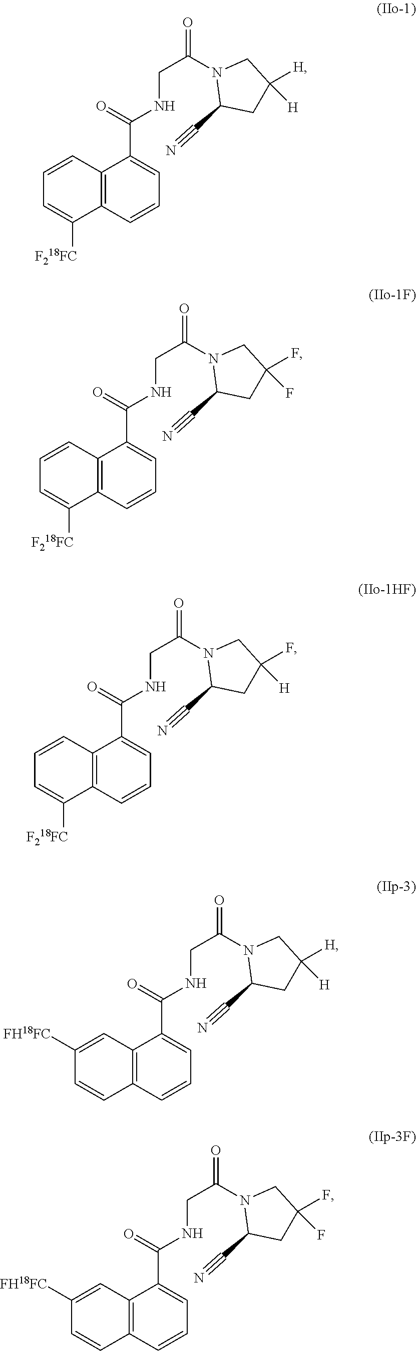

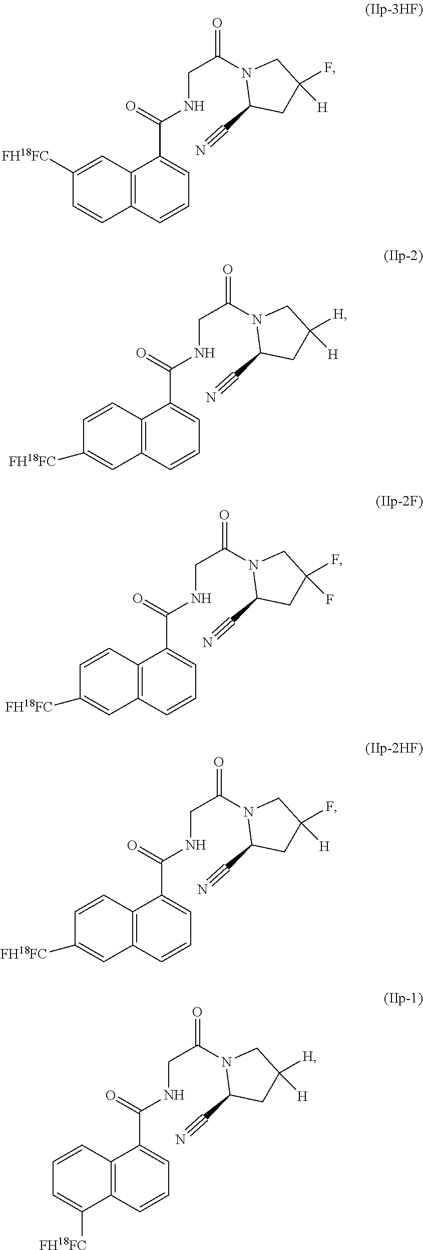

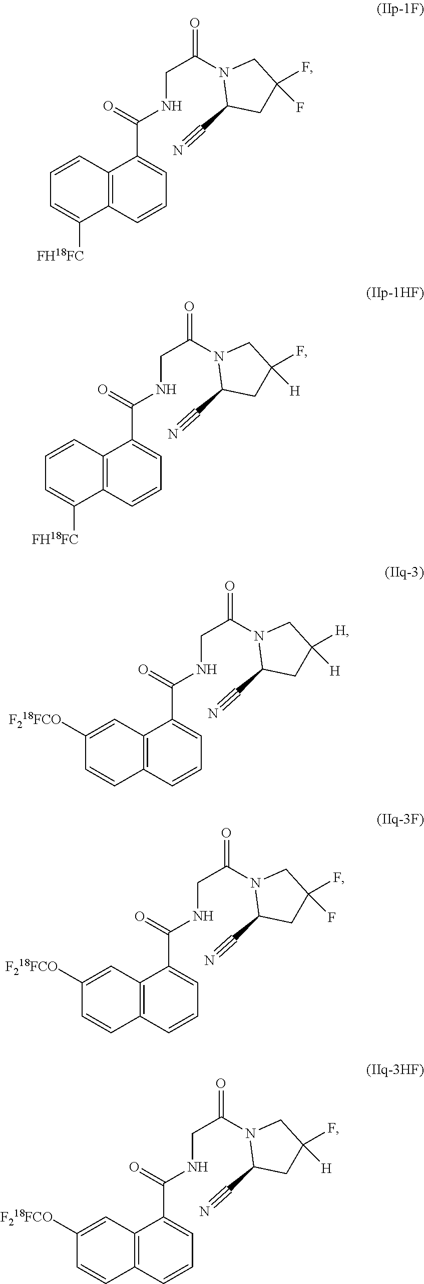

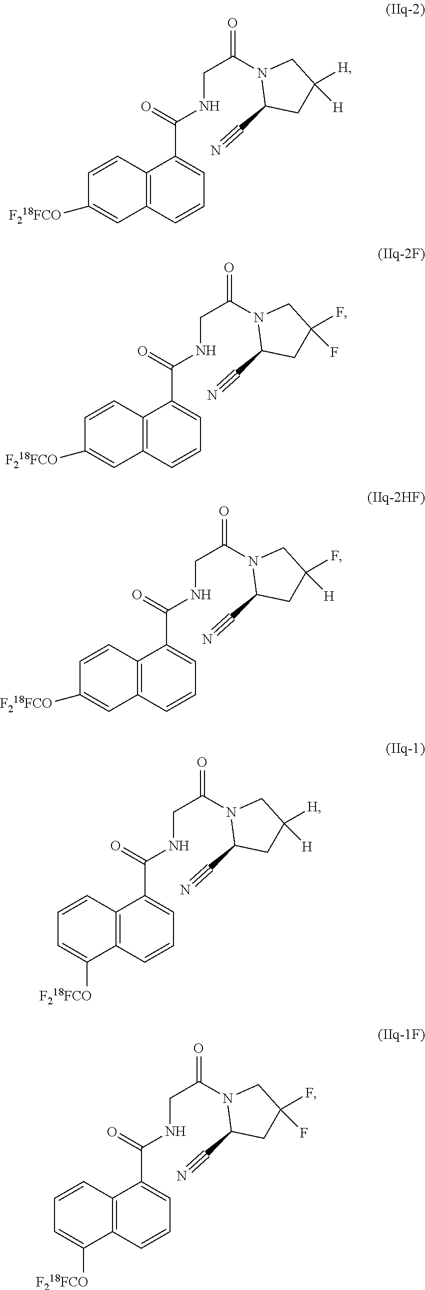

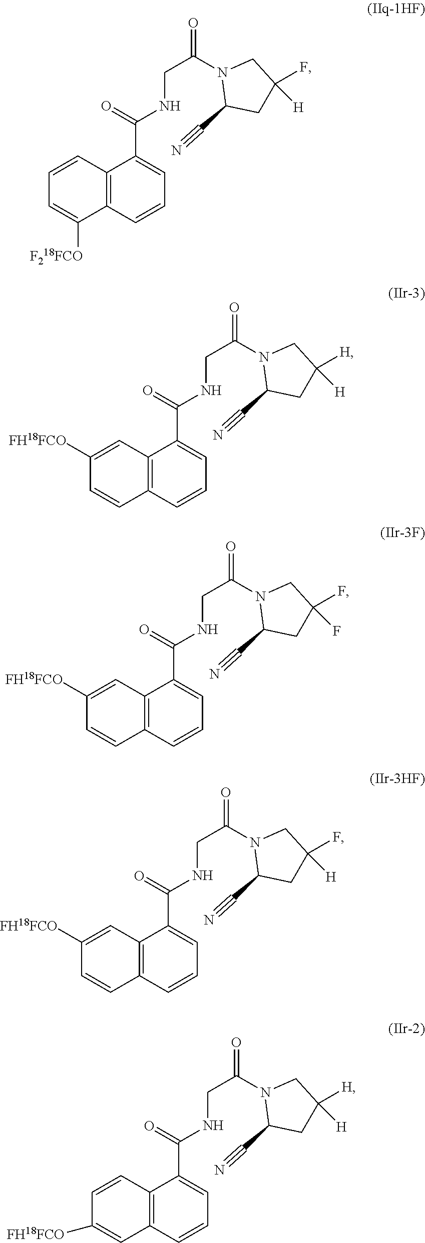

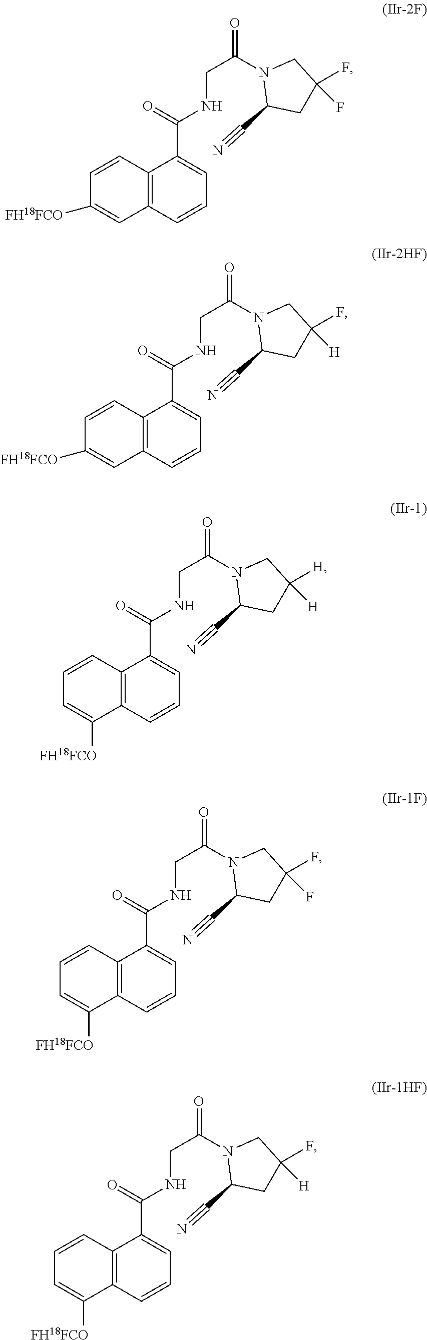

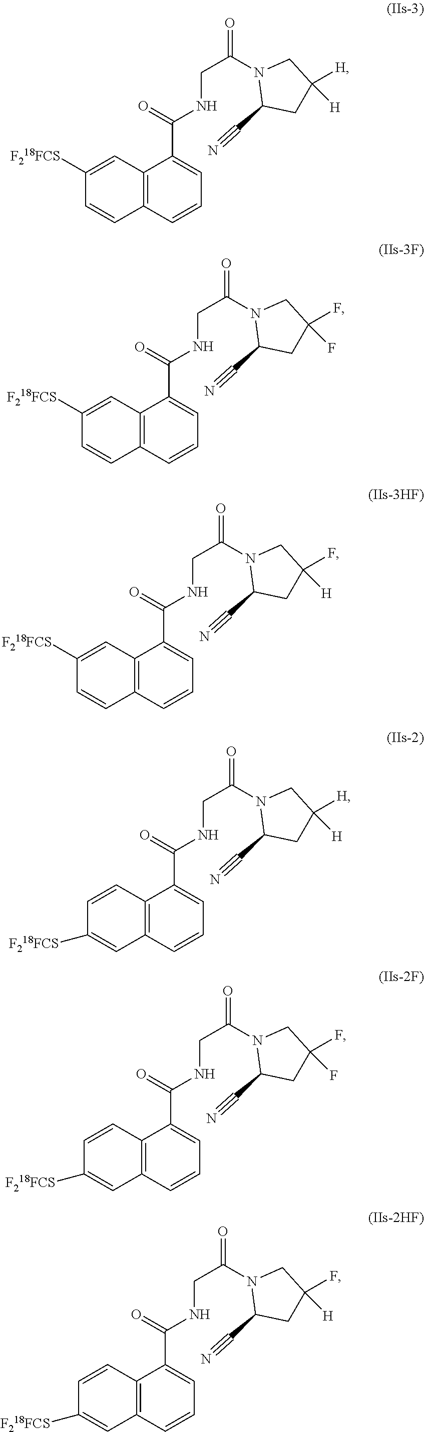

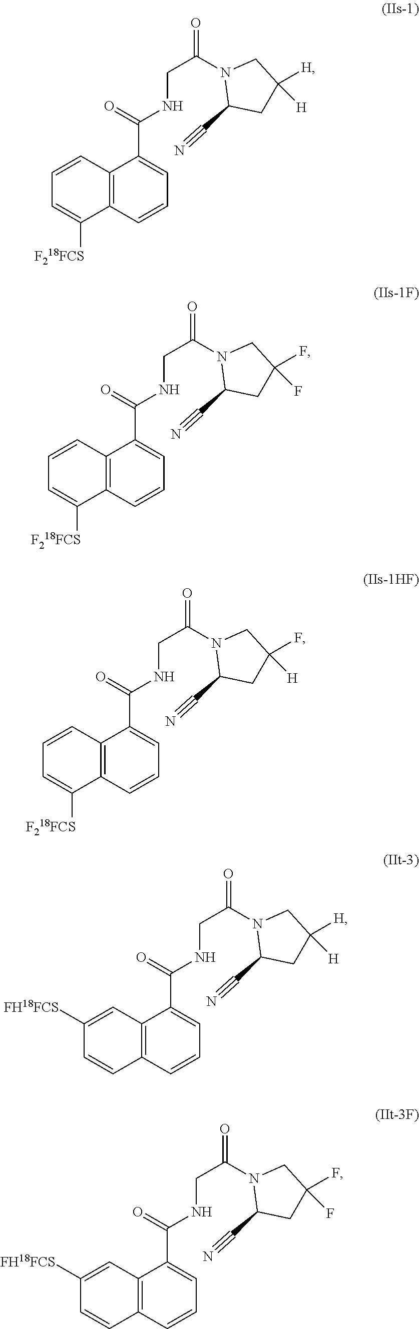

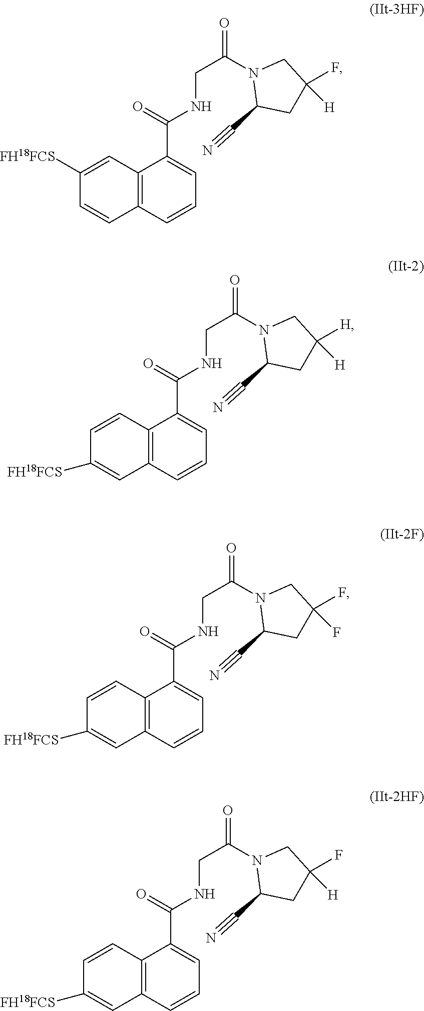

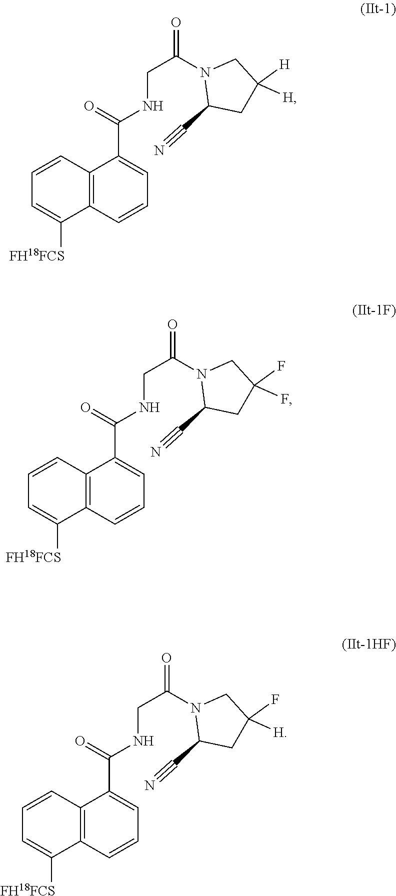

27. The fibroblast activation protein inhibitor of claim 1 having a structure according to one of the following formulae:

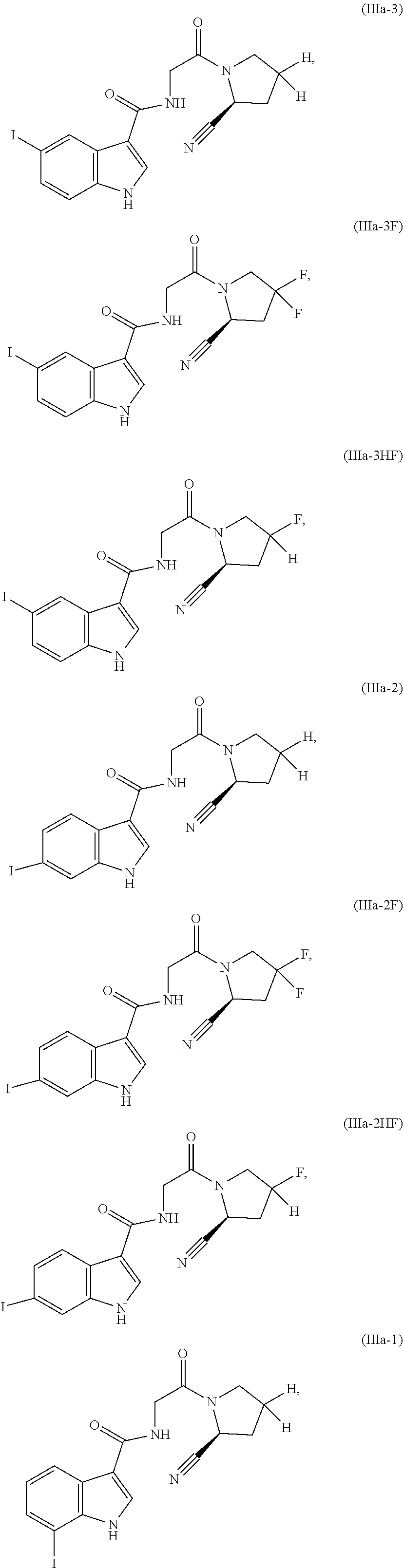

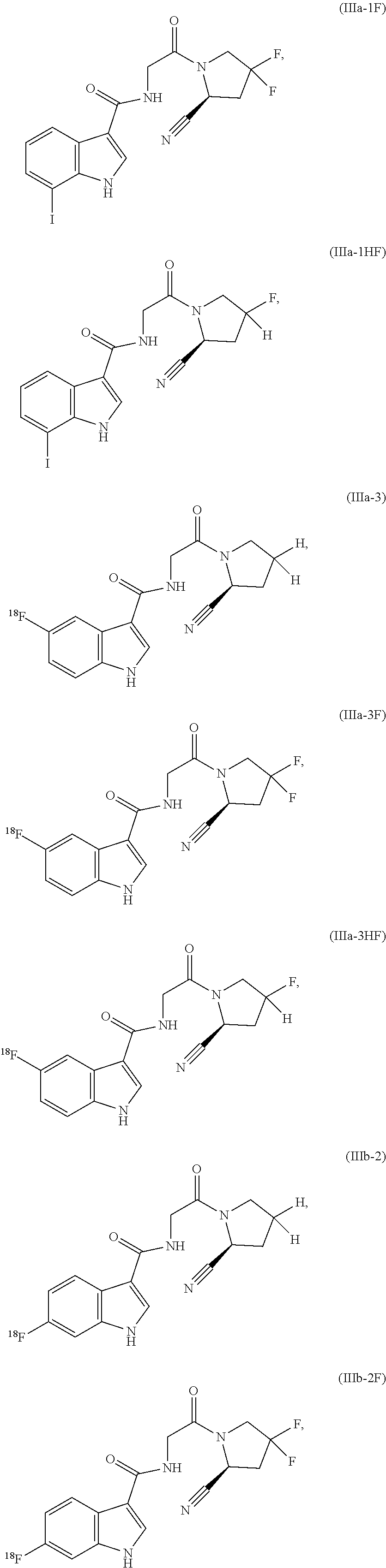

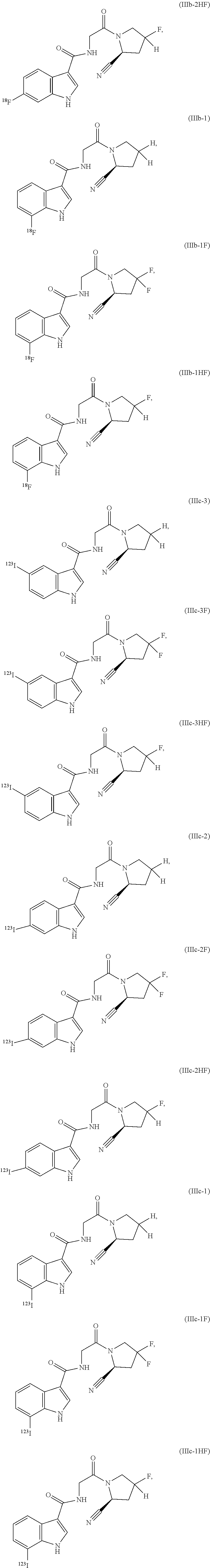

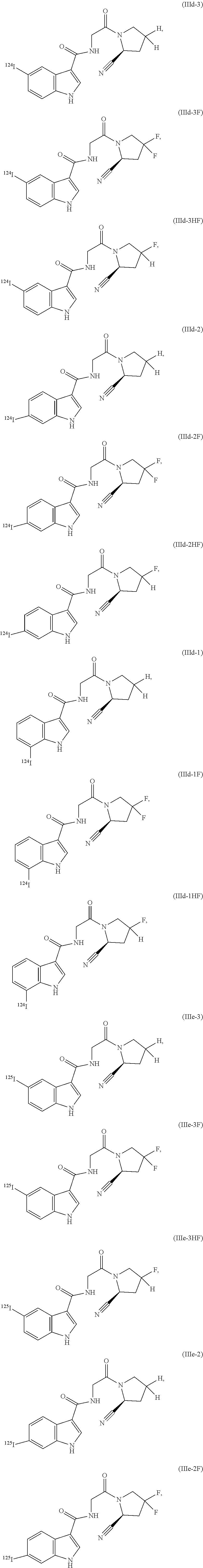

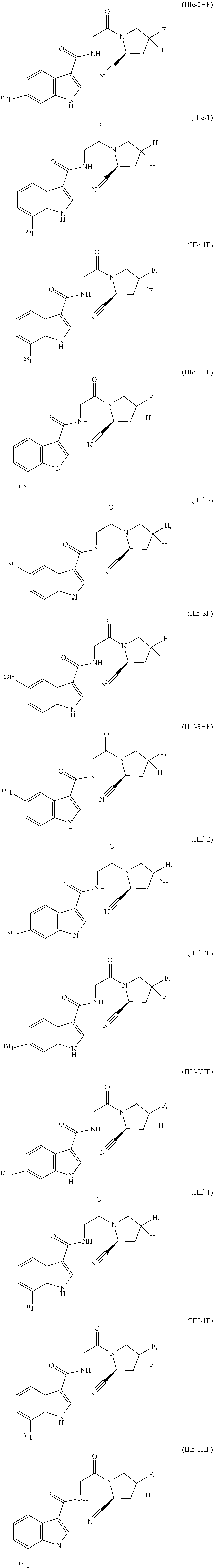

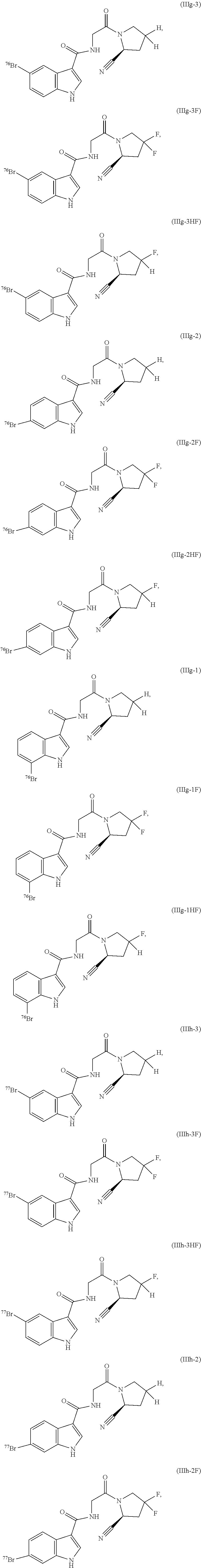

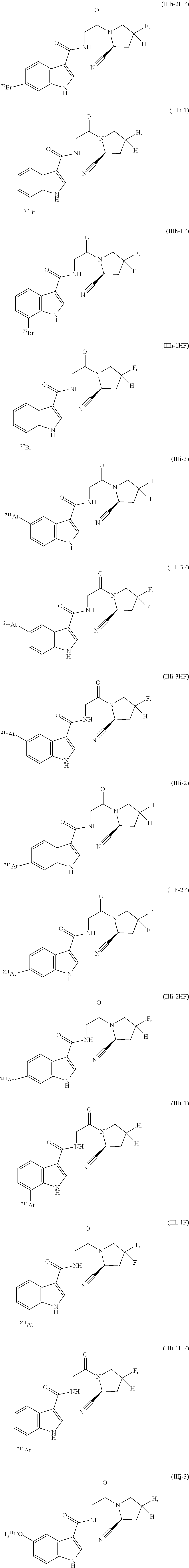

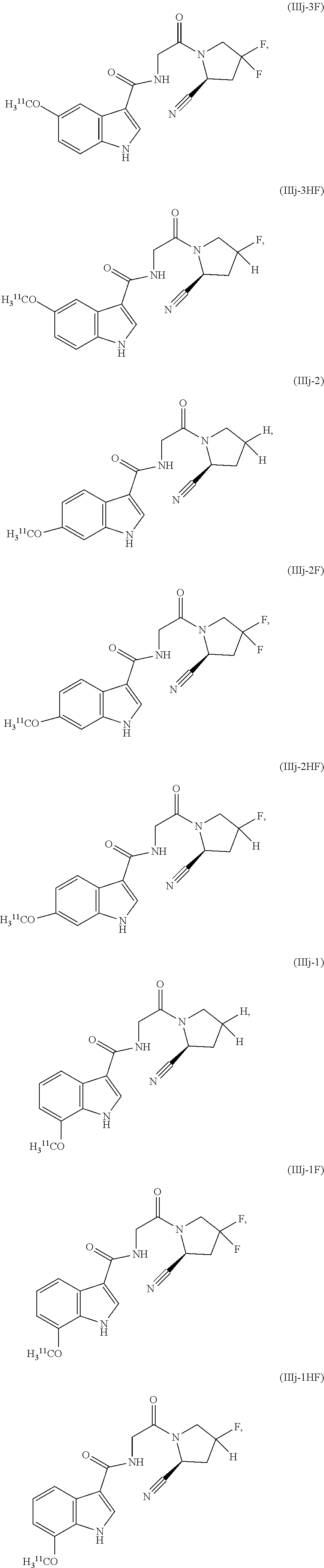

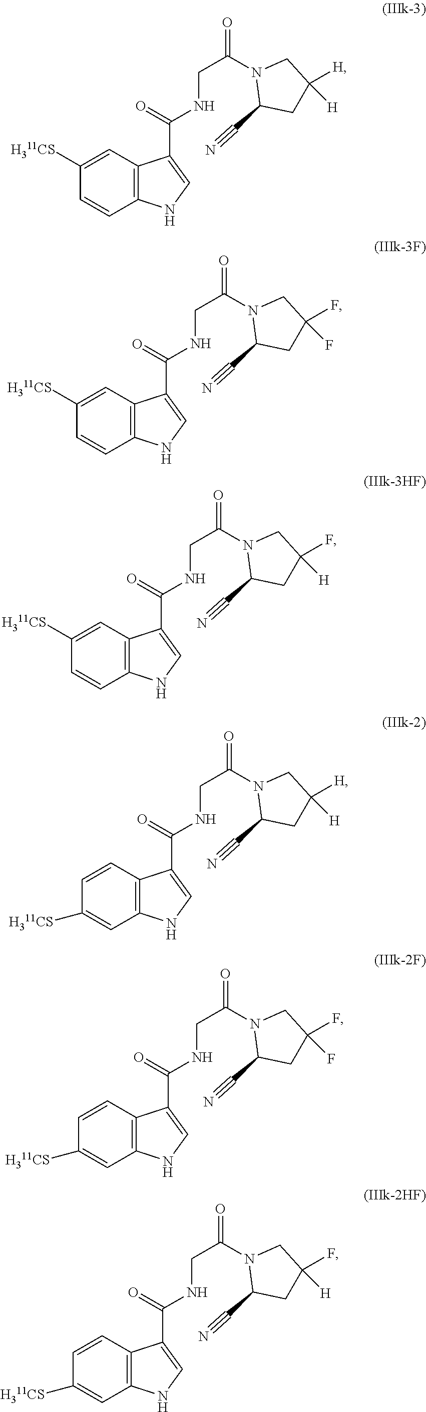

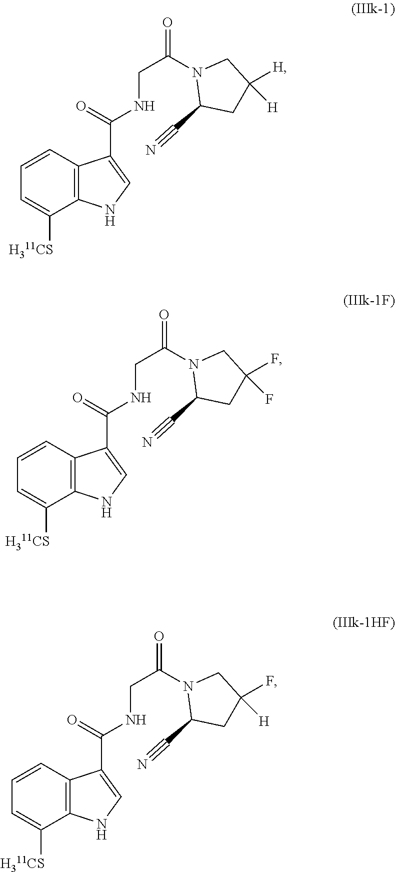

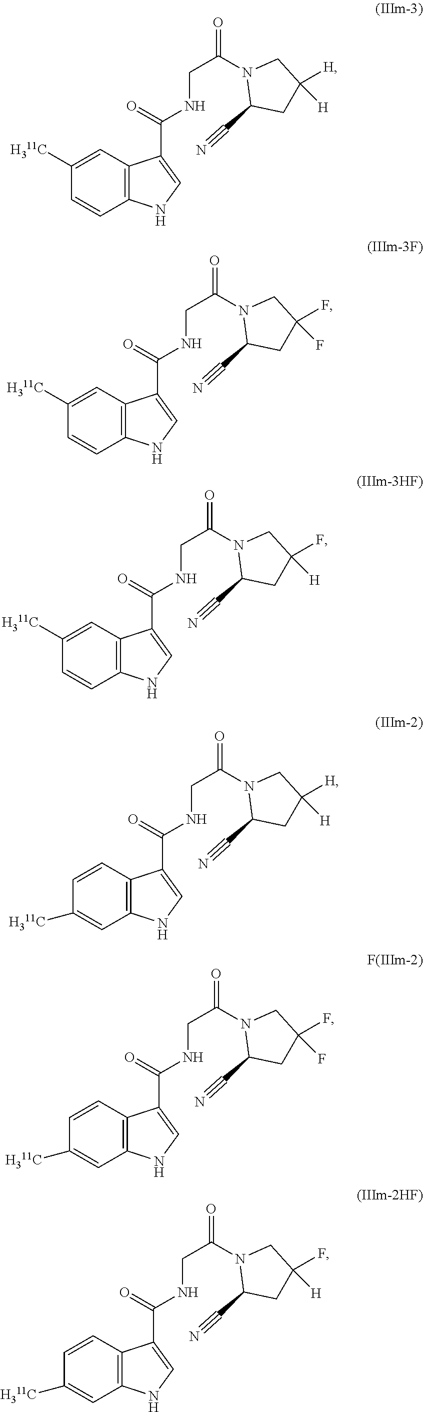

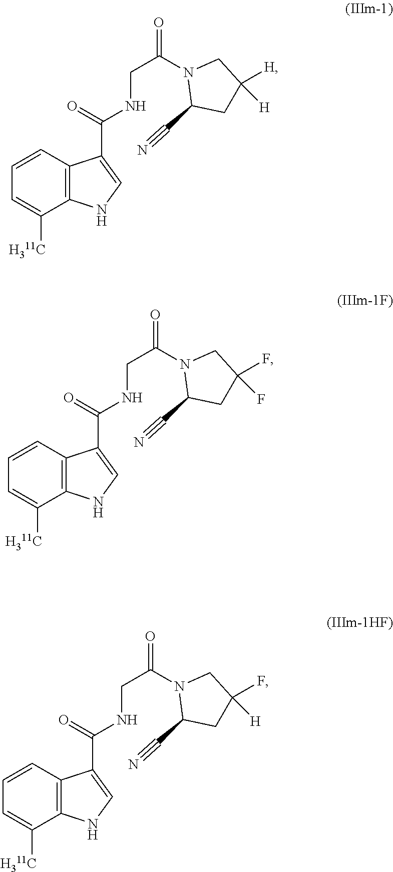

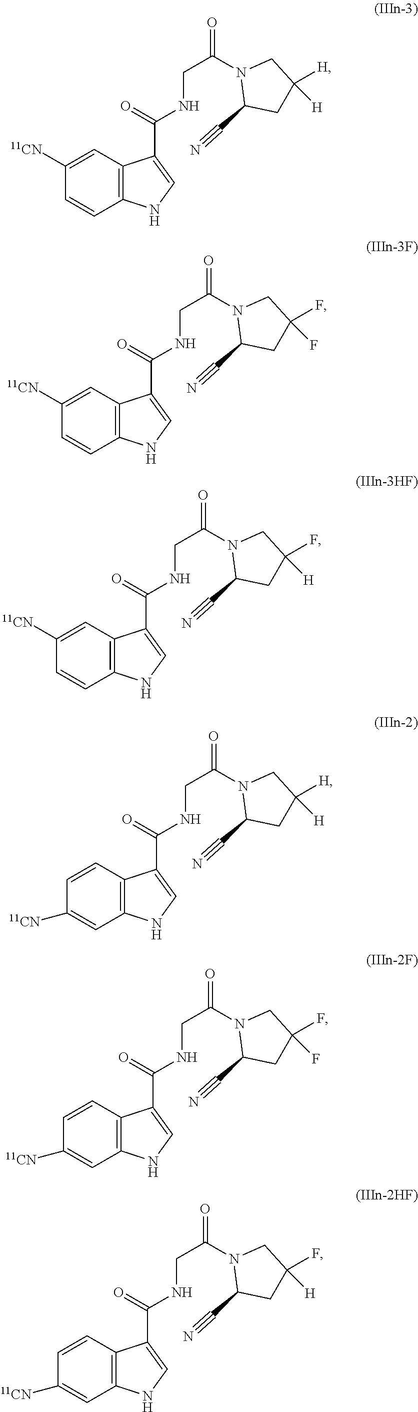

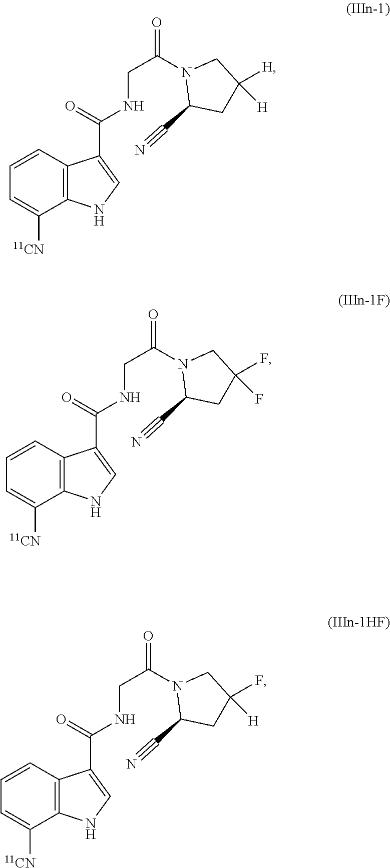

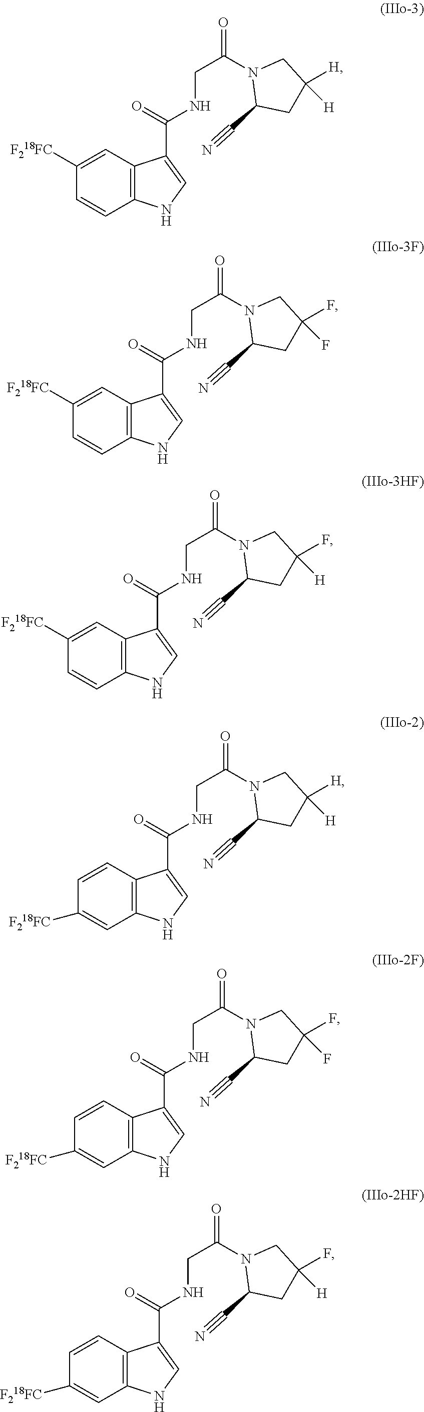

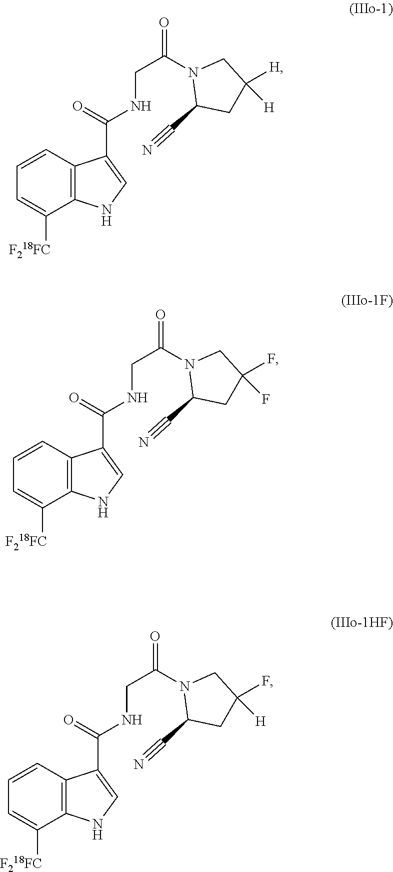

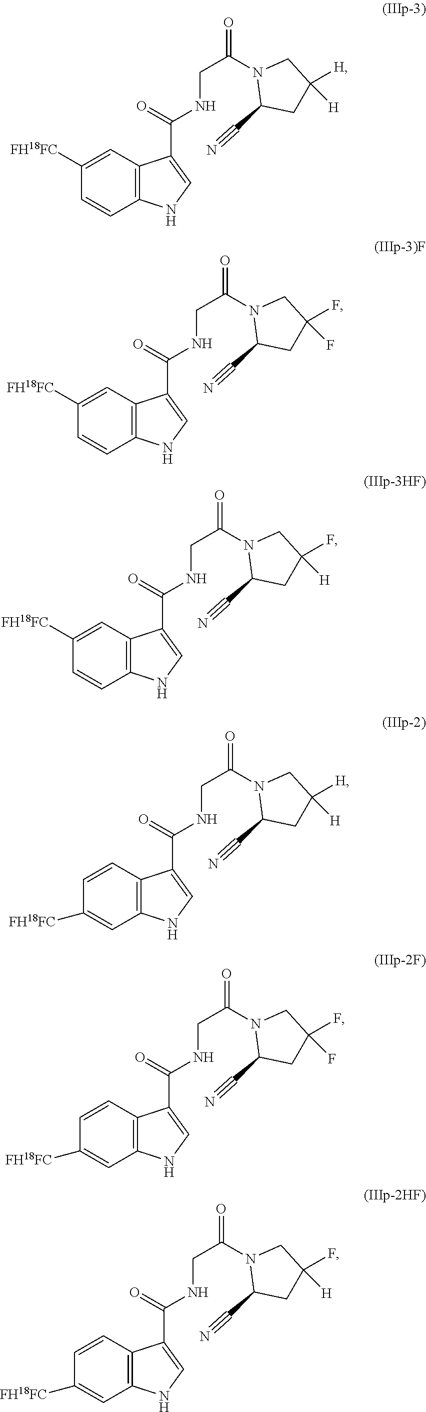

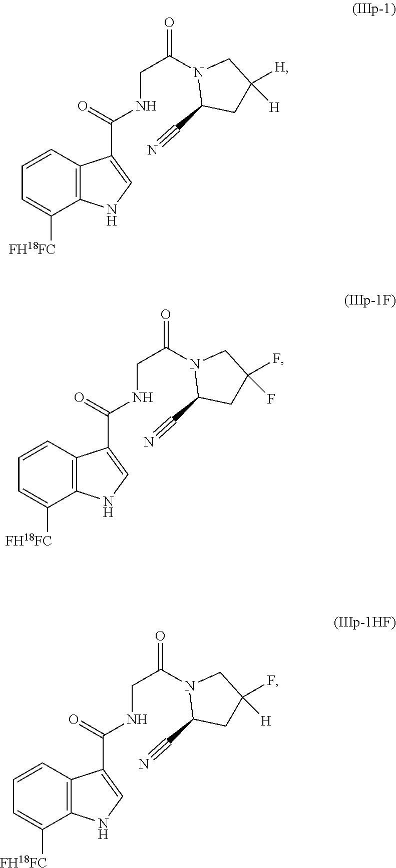

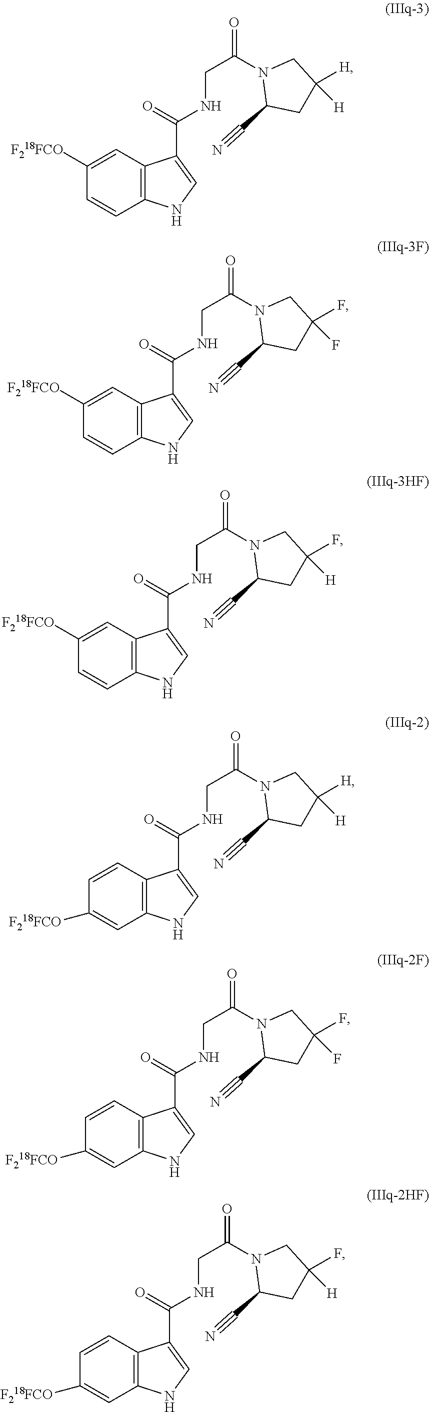

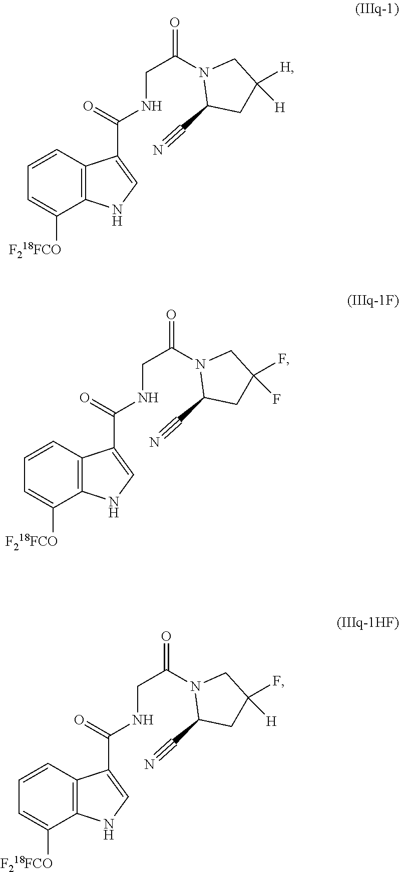

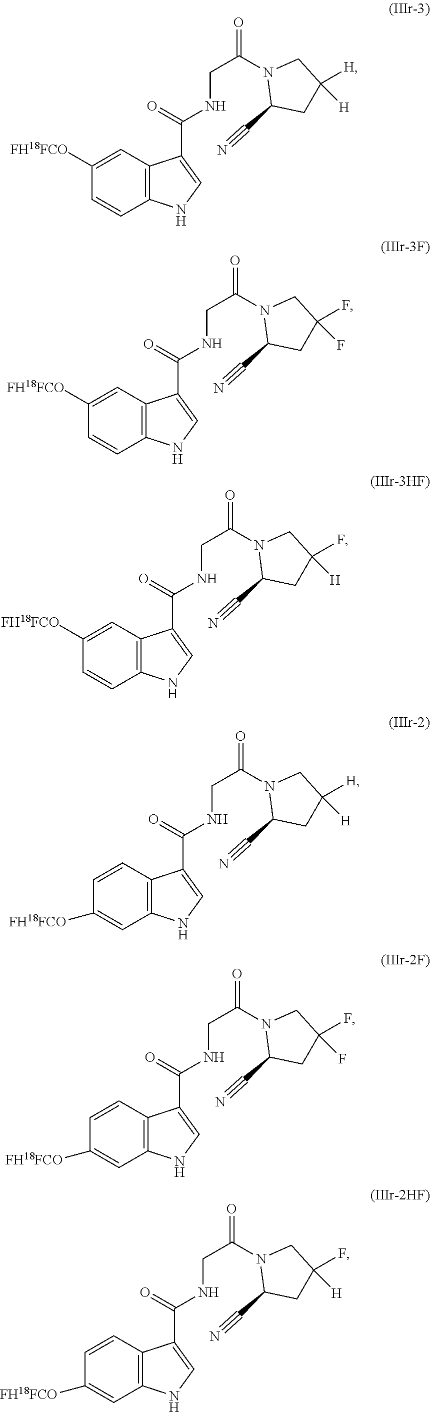

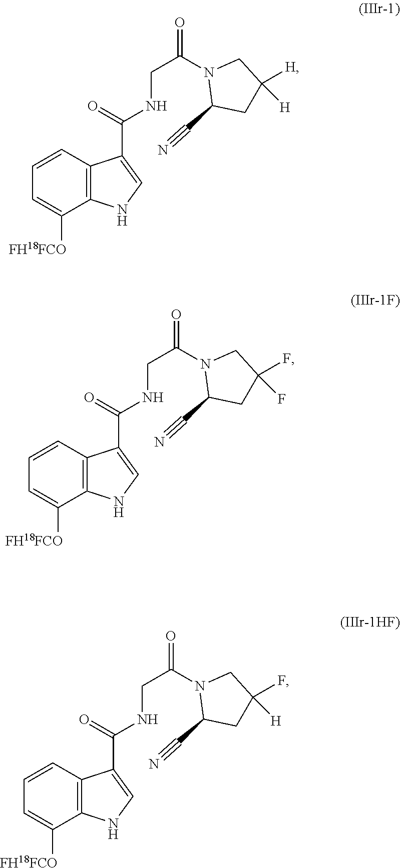

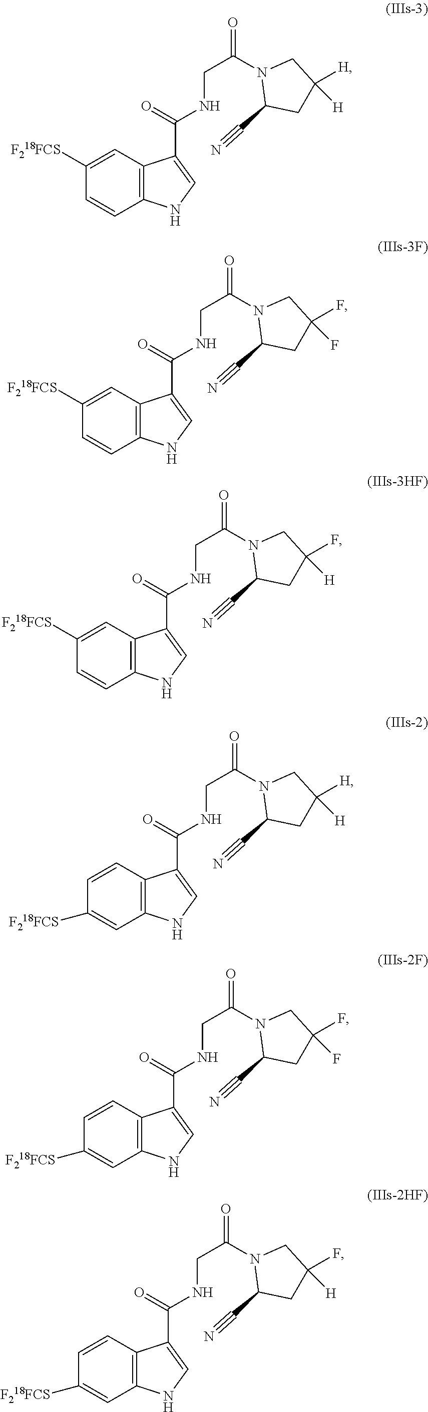

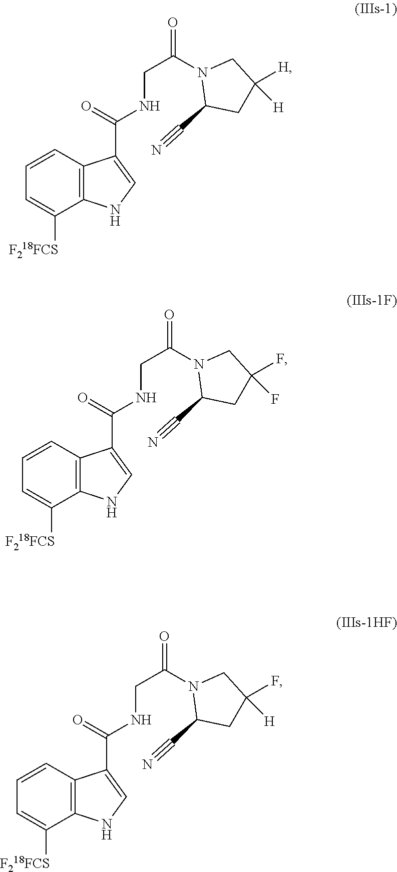

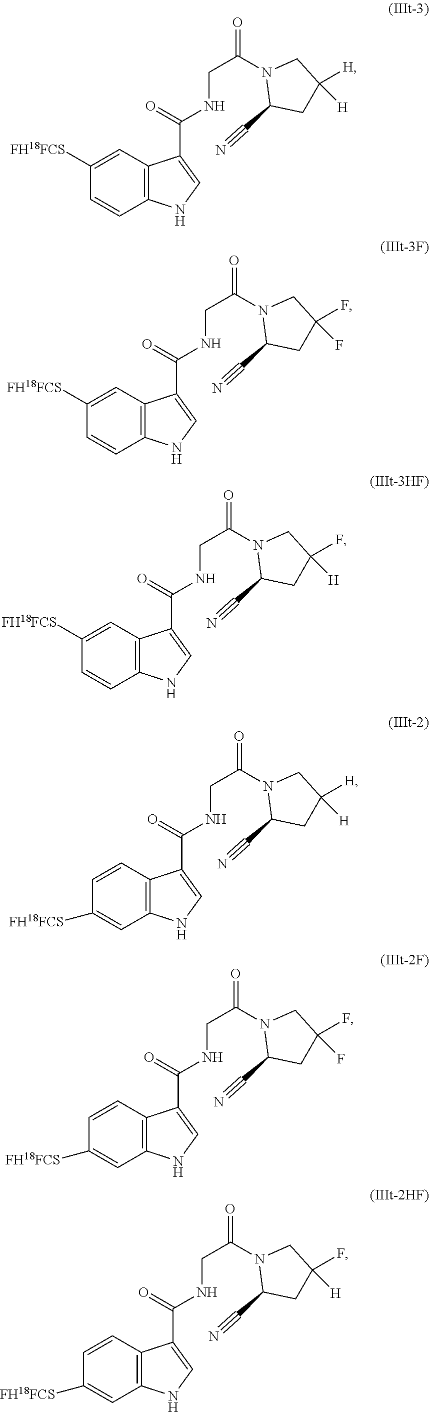

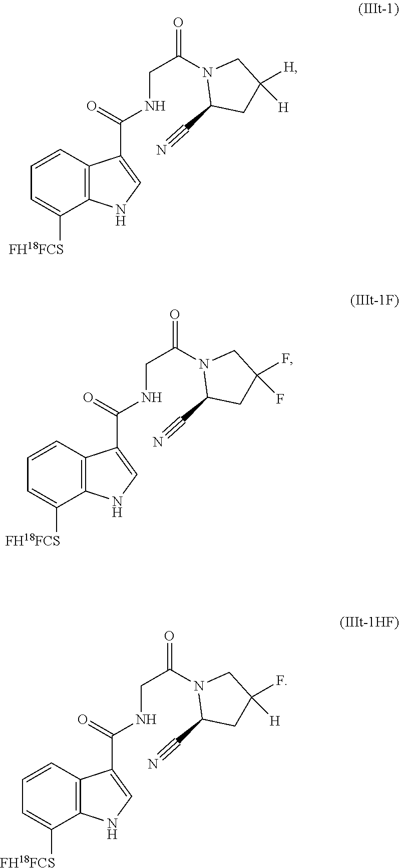

28. A method of preparing a fibroblast activation protein inhibitor comprising:
admixing a compound having a structure according to formula (IV) with a salt, MX, to form a compound having a structure according to formula (I): 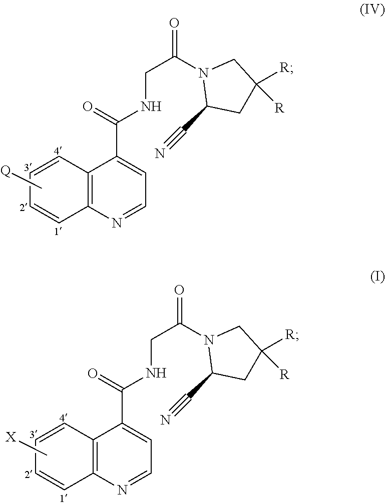
wherein
M is selected from the group of K, Li, Na, Rb, Cs, tetraethylammonium, and tetrabutylammonium;
in the salt, KX, and in formula (I), X is selected from the group of ¹²³I, ¹²⁴I, ¹²⁵I, ¹³¹I, ²¹¹At, ⁷⁶Br, ⁷⁷Br, ¹⁸F, H₃¹¹CO, CF₂¹⁸F, CHF¹⁸F, OCF₂¹⁸F, OCHF¹⁸F, SCF₂¹⁸F, SCHF¹⁸F, ¹¹CN, ¹¹CH₃, and H₃¹¹CS; and X is the same in the salt and in formula (I);
Q is selected from trialkyl stannyl, trialkyl germyl, and trialkyl silyl;
in formula (IV) and formula (I), Q and X are located at carbon 1′, 2′, or 3′, and the location of Q in formula (IV) and the location of X in formula (I) are the same; and
each R is independently selected from H, F, ²H, CH₃, and cyclopentyl, and the R in formula (I) are the same as the R in formula (IV).

29. The method of claim 28, wherein the admixing is performed
(a) in the presence of Cu(OTf)₂, (CH₃CN)₄CuOTf, or CuOTf-toluene;
(b) in the presence of pyridine and DMF or DMA; and/or
(c) at a temperature in a range of about 20° C. to about 140° C. ° C. for about 5 minutes to about 72 hrs.

30. The method of any one of claim 28 or 29, further comprising preparing the compound having a structure according to formula (IV), by admixing a compound having a structure according to formula (Ia) with one of hexaalkyldistannane, hexaalkyldigermane, or hexaalkyldisilane: 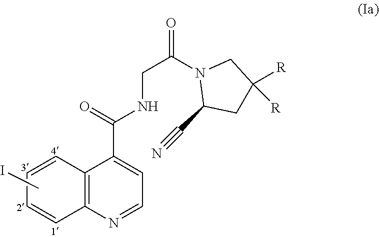
wherein the R in formula (Ia) are the same R as in the compound according to formula (IV) and the I is located at the same carbon as the Q in the compound according to formula (IV).

31. The method of claim 30, wherein the admixing of the compound having a structure according to formula (Ia) with one of hexaalkyldistannane, hexalkyldigermane, or hexaalkyldisilane is performed
(a) in the presence of LiCl, toluene, and Pd(PPh₃)₄; and/or
(b) at a temperature in a range of about 20° C. to about 140° C. ° C. for a time in a range of 5 minutes to 72 hrs.

32. The method of any one of claim 30 or 31, wherein the alkyl of the hexaalkyldistannane, hexaalkyldigermane, or hexaalkyldisilane is methyl or butyl.

33. The method of any one of claims 30 to 32, wherein the hexaalkyldistannane is hexamethyldistannane.

34. The method of any one of claims 30 to 32, wherein the hexaalkyldistannane is hexabutyldistannane.

35. The method of any one of claims 28 to 34, wherein Q and X are located at the 3′ carbon.

36. The method of any one of claims 28 to 34, wherein Q and X are located at the 2′ carbon.

37. The method of any one of claims 28 to 34, wherein Q and X are located at the 1′ carbon.

38. The method of any one of claims 28 to 37, wherein X is selected from the group of ¹⁸F, ²¹¹At, ¹²³I, ¹²⁴I, ¹²⁵I, ¹³¹I, ⁷⁶Br, ⁷⁷Br, and H₃¹¹CO.

39. The method of any one of claims 28 to 38, wherein X is ¹⁸F.

40. The method of any one of claims 28 to 39, wherein in the compounds having a structure according to formulae (IV), (I), and (Ia), at least one R is H or ²H.

41. The method of any one of claims 28 to 40, wherein both R are H or ²H.

42. The method of any one of claims 28 to 40, wherein at least one R is F.

43. The method of any one of claims 28 to 39, wherein both R are F.

44. The method of any one of claims 28 to 40, or 42, wherein at least one R is CH₃ or cyclopentyl.

45. The method of any one of claims 28 to 39, wherein both R are CH₃ or cyclopentyl.

46. The method of any one of claims 28 to 34, wherein Q and X are located at carbon 3′ and all R are H or F.

47. The method of any one of claims 28 to 34, wherein Q and X are located at carbon 2′ and all R are H or F.

48. The method of any one of claims 28 to 34, wherein Q and X are located at carbon 1′ and all R are H or F.

49. The method of any one of claims 28 to 48, wherein Q is trialkyl stannyl.

50. The method of any one of claims 28 to 48, wherein Q is trialkyl germyl.

51. The method of any one of claims 28 to 48, wherein Q is trialkyl silyl.

52. The method of any one of claims 28 to 51, wherein the alkyl of the trialkyl tin, trialkyl germanium, or trialkyl silyl is methyl or butyl.

53. A method of preparing a fibroblast activation protein inhibitor comprising:
admixing a compound having a structure according to formula (VI) with a salt, MX, to form a compound having a structure according to formula (II): 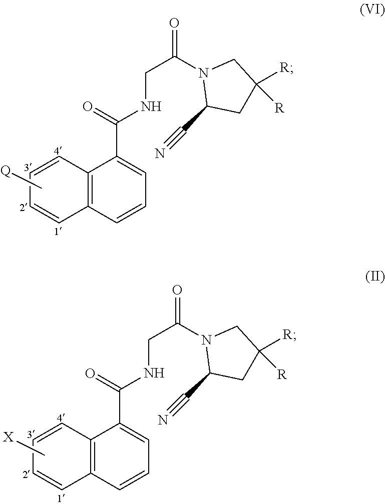
wherein
M is selected from the group of K, Li, Na, Rb, Cs, tetraethylammonium, and tetrabutylammonium;
in the salt, KX, and in formula (II), X is selected from the group of ¹²³I, ¹²⁴I, ¹²⁵I, ¹³¹I, ²¹¹At, ⁷⁶Br, ⁷⁷Br, ¹⁸F, H₃¹¹CO, CF₂¹⁸F, CHF¹⁸F, OCF₂¹⁸F, OCHF¹⁸F, SCF₂¹⁸F, SCHF¹⁸F, ¹¹CN, ¹¹CH₃, and H₃¹¹CS, and X is the same in the salt and in formula (II);
Q is selected from trialkyl stannyl, trialkyl germyl, or trialkyl silyl;
in formula (VI) and formula (II), Q and X are located at carbon 1′, 2′, or 3′, and the location of Q in formula (VI) and the location of X in formula (II) are the same; and
each R is independently selected from H, F, ²H, CH₃, and cyclopentyl, and the R in formula (II) are the same as the R in formula (VI).

54. The method of claim 53, wherein the admixing is performed
(a) in the presence of Cu(OTf)₂, (CH₃CN)₄CuOTf, or CuOTf-toluene;
(b) in the presence of pyridine and DMF or DMA; and/or
(c) at a temperature in a range of 20° C. to 140° C. for a time in a range of 5 minutes to 72 hrs.

55. The method of any one of claim 53 or 54, further comprising preparing the compound having a structure according to formula (VI), by admixing a compound having a structure according to formula (IIa) with one of hexaalkyldistannane, hexaalkyldigermane, or hexaalkyldisilane: 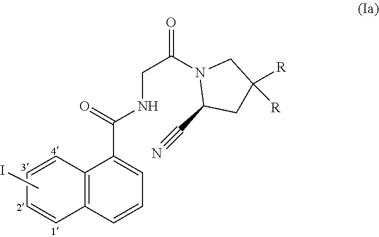
wherein the R in formula (IIa) are the same R as in the compound according to formula (VI) and the I is located at the same carbon as the Q in the compound according to formula (VI).

56. The method of claim 55, wherein the admixing of the compound having a structure according to formula (IIa) with one of hexaalkyldistannane, hexalkyldigermane, or hexaalkyldisilane is performed
(a) in the presence of LiCl, toluene, and Pd(PPh₃)₄; and/or
(b) at a temperature in a range of 20° C. to 140° C. for a time in a range of about 5 minutes to about 72 hrs.

57. The method of any one of claims 55 to 56, wherein the alkyl of the hexaalkyldistannane, hexaalkyldigermane, or hexaalkyldisilane is methyl or butyl.

58. The method of any one of claims 55 to 56, wherein the hexaalkyldistannane is hexamethyldistannane.

59. The method of any one of claims 55 to 56, wherein the hexaalkyldistannane is hexabutyldistannane.

60. The method of any one of claims 53 to 57, wherein Q and X are located at the 3′ carbon.

61. The method of any one of claims 53 to 57, wherein Q and X are located at the 2′ carbon.

62. The method of any one of claims 53 to 57, wherein Q and X are located at the 1′ carbon.

63. The method of any one of claims 53 to 62, wherein X is selected from the group of ¹⁸F, ²¹¹At, ¹²³I, ¹²⁴I, ¹²⁵I, ¹³¹I, ⁷⁶Br, ⁷⁷Br, and H₃¹¹CO.

64. The method of any one of claims 53 to 63, wherein X is ¹⁸F.

65. The method of any one of claims 53 to 64, wherein in the compounds having a structure according to formulae (IV), (I), and (la), at least one R is H or ²H.

66. The method of any one of claims 53 to 64, wherein both R are H or ²H.

67. The method of any one of claims 53 to 65, wherein at least one R is F.

68. The method of any one of claims 53 to 64, wherein both R are F.

69. The method of any one of claims 53 to 65, wherein at least one R is CH₃ or cyclopentyl.

70. The method of any one of claims 53 to 64, wherein both R are CH₃ or cyclopentyl.

71. The method of any one of claims 53 to 59, wherein Q and X are located at carbon 3′ and all R are H or F.

72. The method of any one of claims 53 to 59, wherein Q and X are located at carbon 2′ and all R are H or F.

73. The method of any one of claims 53 to 59, wherein Q and X are located at carbon 1′ and all R are H or F.

74. The method of any one of claims 53 to 73, wherein Q is trialkyl tin.

75. The method of any one of claims 53 to 73, wherein Q is trialkyl germanium.

76. The method of any one of claims 53 to 73, wherein Q is trialkyl silyl.

77. The method of any one of claims 53 to 73, wherein the alkyl of the trialkyl stannyl, trialkyl germyl, or trialkyl silyl is methyl or butyl.

78. The method of claim 77, wherein the alkyl of the trialkyl stannyl, trialkyl germyl, or trialkyl silyl is methyl.

79. The method of claim 77, wherein the alkyl of the trialkyl stannyl, trialkyl germyl, or trialkyl silyl is butyl.

80. A method of preparing a fibroblast activation protein inhibitor comprising:
admixing a compound having a structure according to formula (VII) with a salt, MX, to form a compound having a structure according to formula (III): 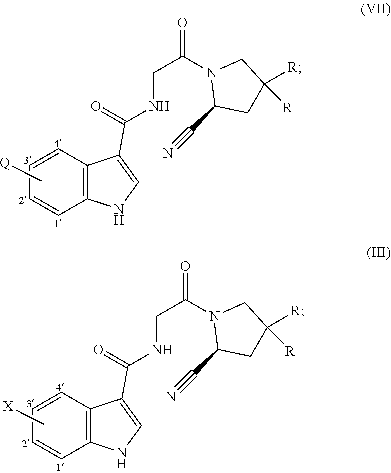
wherein
M is selected from the group of K, Li, Na, Rb, Cs, tetraethylammonium, and tetrabutylammonium;
in the salt, MX, and in formula (III), X is selected from the group of ¹²³I, ¹²⁴I, ¹²⁵I, ¹³¹I, ²¹¹At, ⁷⁶Br, ⁷⁷Br, ¹⁸F, H₃¹¹CO, CF₂¹⁸F, CHF¹⁸F, OCF₂¹⁸F, OCHF¹⁸F, SCF₂¹⁸F, SCHF¹⁸F, ¹¹CN, ¹¹CH₃, and H₃¹¹CS, and X is the same in the salt and in formula (III);
Q is selected from trialkyl stannyl, trialkyl germyl, or trialkyl silyl;
in formula (VII) and formula (III), Q and X are located at carbon 1′, 2′, or 3′, and the location of Q in formula (VII) and the location of X in formula (III) are the same; and
each R is independently selected from H, F, ²H, CH₃, and cyclopentyl, and the R in formula (III) are the same as the R in formula (VII).

81. The method of claim 80, wherein the admixing is performed
(a) in the presence of Cu(OTf)₂, (CH₃CN)₄CuOTf, or CuOTf-toluene;
(b) in the presence of pyridine and DMF or DMA; and/or
(c) at a temperature in a range of about 20° C. to about 140° C. for a time in a range of about 5 minutes to about 72 hrs.

82. The method of any one of claim 80 or 81, further comprising preparing the compound having a structure according to formula (VII), by admixing a compound having a structure according to formula (IIIa) with one of hexaalkyldistannane, hexaalkyldigermane, or hexaalkyldisilane: 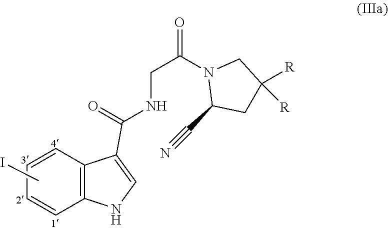
wherein the R in formula (IIIa) are the same R as in the compound according to formula (VII) and the I is located at the same carbon as the Q in the compound according to formula (VII).

83. The method of claim 82, wherein the admixing of the compound having a structure according to formula (IIIa) with one of hexaalkyldistannane, hexalkyldigermane, or hexaalkyldisilane is performed
(a) in the presence of LiCl, toluene, and Pd(PPh₃)₄; and/or
(b) at a temperature in a range of 20° C. to about 140° C. for a time in a range of about 5 minutes to about 72 hrs.

84. The method of any one of claims 82 to 83, wherein the alkyl of the hexaalkyldistannane, hexaalkyldigermane, or hexaalkyldisilane is methyl or butyl.

85. The method of any one of claims 82 to 84, wherein the hexaalkyldistannane is hexamethyldistannane.

86. The method of any one of claims 82 to 84, wherein the hexaalkyldistannane is hexabutyldistannane.

87. The method of any one of claims 80 to 86, wherein Q and X are located at the 3′ carbon.

88. The method of any one of claims 80 to 86, wherein Q and X are located at the 2′ carbon.

89. The method of any one of claims 80 to 86, wherein Q and X are located at the 1′ carbon.

90. The method of any one of claims 80 to 89, wherein X is selected from the group of ¹⁸F, ²¹¹At, ¹²³I, ¹²⁴I, ¹²⁵I, ¹³¹I, ⁷⁶Br, ⁷⁷Br, and H₃¹¹CO.

91. The method of any one of claims 80-90, wherein X is ¹⁸F.

92. The method of any one of claims 80 to 91, wherein in the compounds having a structure according to formulae (IV), (1), and (Ia), at least one R is H or ²H.

93. The method of any one of claims 80 to 91, wherein both R are H or ²H.

94. The method of any one of claims 80 to 92, wherein at least one R is F.

95. The method of any one of claims 80 to 91, wherein both R are F.

96. The method of any one of claims 80 to 92, or 94 wherein at least one R is CH₃ or cyclopentyl.

97. The method of any one of claims 80 to 91, wherein both R are CH₃ or cyclopentyl.

98. The method of any one of claims 80 to 86, wherein Q and X are located at carbon 3′ and all R are H or F.

99. The method of any one of claims 80 to 86, wherein Q and X are located at carbon 2′ and all R are H or F.

100. The method of any one of claims 80 to 86, wherein Q and X are located at carbon 1′ and all R are H or F.

101. The method of any one of claims 80 to 100, wherein Q is trialkyl stannyl.

102. The method of any one of claims 80 to 100, wherein Q is trialkyl germyl.

103. The method of any one of claims 80 to 100, wherein Q is trialkyl silyl.

104. The method of any one of claims 80 to 100, wherein the alkyl of the trialkyl stannyl, trialkyl germyl, or trialkyl silyl is methyl or butyl.

105. The method of claim 104, wherein the alkyl of the trialkyl stannyl, trialkyl germyl, or trialkyl silyl is methyl.

106. The method of claim 104, wherein the alkyl of the trialkyl stannyl, trialkyl germyl, or trialkyl silyl is butyl.

107. A method of preparing a fibroblast activation protein inhibitor comprising:
admixing a compound having a structure according to formula (VIII) with a radiolabeled salt, MX, to form a compound having a structure according to formula (I): 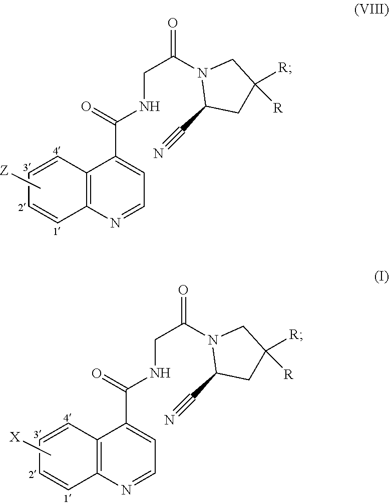
wherein
Z has a structure according selected from the group of: 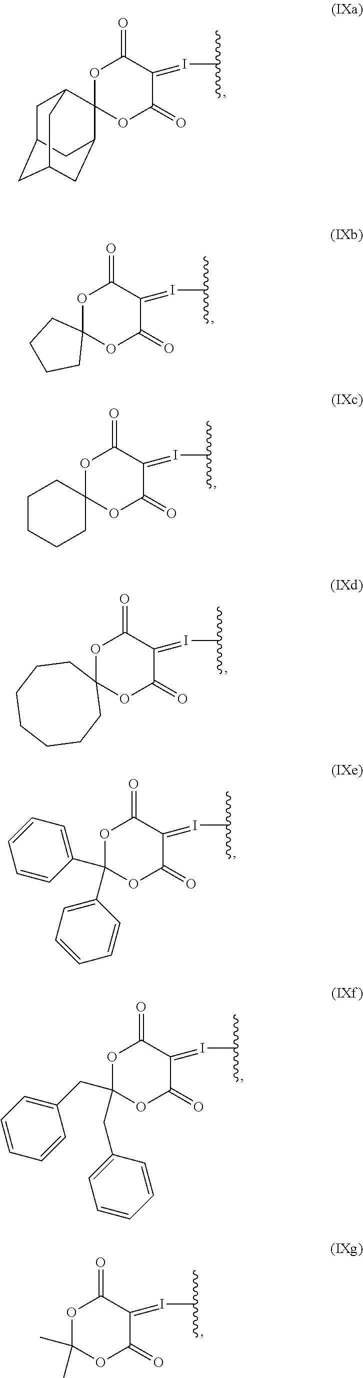 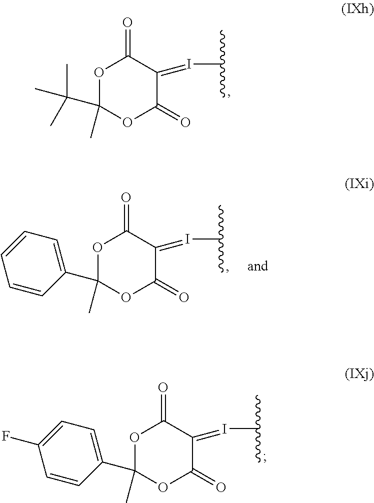 [SMILES: *I=C1C(=O)OC(C)(C(C)(C)C)OC1=O ; *I=C1C(=O)OC(C)(c2ccccc2)OC1=O ; *I=C1C(=O)OC(C)(c2ccc(F)cc2)OC1=O]
in formula (VIII) and formula (I), Z and X are located at carbon 1′, 2′, or 3′, and the location of Z in formula (VIII) and the location of X in formula (I) are the same;
M is selected from the group of K, Li, Na, Rb, Cs, tetraethylammonium, and tetrabutylammonium;
in the salt, MX, and in formula (I), X is selected from the group of ¹⁸F, ²¹¹At, ¹²³I, ¹²⁴I, ¹²⁵I, ¹³¹I, ⁷⁶Br, ⁷⁷Br; and
each R is independently selected from H, F, ²H, CH₃, and cyclopentyl, and the R in formula (I) are the same as the R in formula (VIII).

108. The method of claim 107, wherein the admixing is performed
(a) in the presence of dimethylsulfoxide (DMSO), dimethylformamide (DMF), dimethylacetamide (DMA), or a combination thereof;
(b) at a temperature in a range of 85° C. to 140° C. for a time in a range of about 5 minutes to about 12 hrs.

109. The method of any one of claims 217 to 219, further comprising preparing the compound having a structure according to formula (VIII), by admixing a compound having a structure according to formula (Ia): 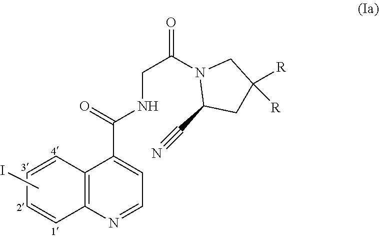
with 1-chloromethyl-4-fluoro-1,4-diazoniabicyclo[2.2.2]octanebis(tetrafluoroborate), and trimethylsilyl acetate to form a reaction product;
wherein the R in formula (Ia) are the same R as in the compound according to formula (VIII) and the I is located at the same carbon as the Z in the compound according to formula (VIII).

110. The method of claim 220, wherein the admixing is performed in the presence of acetonitrile.

111. The method of claim 220 or 221, wherein the admixing comprises dropwise addition of 1-chloromethyl-4-fluoro-1,4-diazoniabicyclo[2.2.2]octanebis(tetrafluoroborate) to a solution of the compound having a structure according to formula (Ia) and trimethylsilyl acetate at about 23° C. and stirring for about 16 hrs.

112. The method of any one of claims 220 to 222, further comprising
admixing (a) the reaction product of the compound having a structure according to formula (Ia) with 1-chloromethyl-4-fluoro-1,4-diazoniabicyclo[2.2.2]octanebis(tetrafluoroborate), and trimethylsilyl acetate with (b) a dioxane-dione and sodium carbonate to form the compound having a structure according to formula (VIII),
wherein the dioxane-dione is selected from the group of (1r,3r,5r,7r)-spiro[adamantan-2,2′-[1,3]-dioxane]-4′,6′-dione (SPIAd), 6,10-dioxaspiro[4.5]decane-7,9-dione, 1,5-dioxaspiro[5.5]undecane-2,4-dione, 1,5-dioxaspiro[5.7]tridecane-2,4-dione, 2,2-diphenyl-1,3-dioxane-4,6-dione, 2,2-dibenzyl-1,3-dioxane-4,6-dione, 5,5-dimethylcyclohexane-1,3-dione, 2-(tert-butyl)-2-methyl-1,3-dioxane-4,6-dione, 2-methyl-2-phenyl-1,3-dioxane-4,6-dione, and 2-(4-fluorophenyl)-2-methyl-1,3-dioxane-4,6-dione.

113. The method of claim 223, wherein the admixing is performed in the presence of ethanol.

114. The method of claim 223 or claim 224, wherein the admixing comprises adding the dioxane-dione and sodium carbonate to the reaction product of the compound having a structure according to formula (Ia) with 1-chloromethyl-4-fluoro-1,4-diazoniabicyclo[2.2.2]octanebis(tetrafluoroborate), methyl cyanide, and trimethylsilyl acetate dropwise at about 23° C. and stirring for about 4 hrs.

115. A method of preparing a fibroblast activation protein inhibitor comprising:
admixing a compound having a structure according to formula (X) with a radiolabeled salt, MX, to form a compound having a structure according to formula (II): 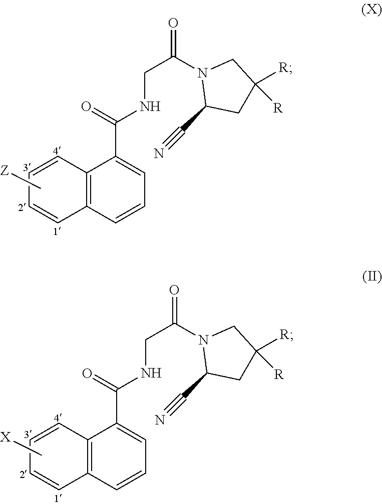
wherein
Z has a structure selected from the group of: 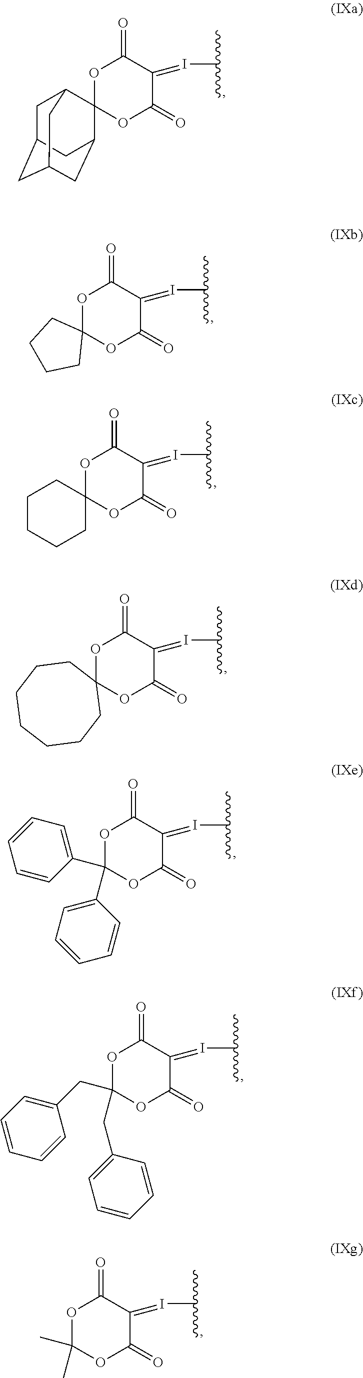 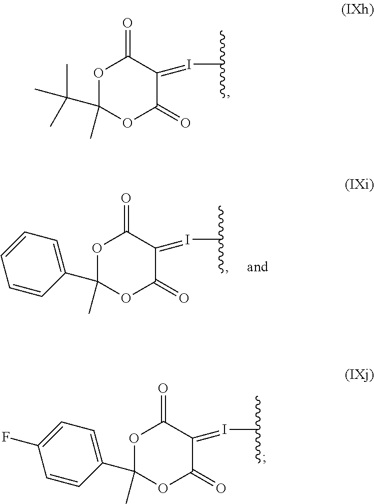 [SMILES: *I=C1C(=O)OC(C)(C(C)(C)C)OC1=O ; *I=C1C(=O)OC(C)(c2ccccc2)OC1=O ; *I=C1C(=O)OC(C)(c2ccc(F)cc2)OC1=O]
in formula (X) and formula (II), Z and X are located at carbon 1′, 2′, or 3′, and the location of Z in formula (X) and the location of X in formula (II) are the same;
M is selected from the group of K, Li, Na, Rb, Cs, tetraethylammonium, and tetrabutylammonium;
in the salt, MX, and in formula (II), X is selected from the group of ¹⁸F, ²¹¹At, ¹²³I, ¹²⁴I, ¹²⁵I, ¹³¹I, ⁷⁶Br, ⁷⁷Br; and
each R is independently selected from H, F, ²H, CH₃, and cyclopentyl, and the R in formula (II) are the same as the R in formula (X).

116. The method of claim 115, wherein the admixing is performed
(a) in the presence of dimethylsulfoxide (DMSO), dimethylformamide (DMF), dimethylacetamide (DMA), or a combination thereof; and/or
(b) at a temperature in a range of 85° C. to 140° C. for a time in a range of about 5 minutes to about 12 hrs.

117. The method of any one of claim 115 or 116, further comprising preparing the compound having a structure according to formula (X), by admixing a compound having a structure according to formula (IIa): 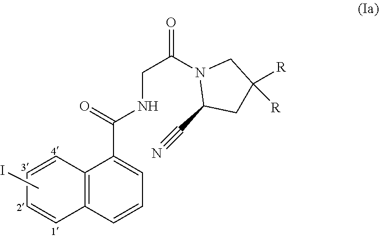
with 1-chloromethyl-4-fluoro-1,4-diazoniabicyclo[2.2.2]octanebis(tetrafluoroborate), and trimethylsilyl acetate to form a reaction product;
wherein the R in formula (IIa) are the same R as in the compound according to formula (X) and the I is located at the same carbon as the Z in the compound according to formula (X).

118. The method of claim 117, wherein the admixing is performed in the presence of acetonitrile.

119. The method of claim 117 or 118, wherein the admixing comprises dropwise addition of 1-chloromethyl-4-fluoro-1,4-diazoniabicyclo[2.2.2]octanebis(tetrafluoroborate) to a solution of the compound having a structure according to formula (IIa) and trimethylsilyl acetate at about 23° C. and stirring for about 16 hrs.

120. The method of any one of claims 1179 to 118, further comprising
admixing (a) the reaction product of the compound having a structure according to formula (IIa) with 1-chloromethyl-4-fluoro-1,4-diazoniabicyclo[2.2.2]octanebis(tetrafluoroborate), and trimethylsilyl acetate with (b) a dioxane-dione and sodium carbonate to form the compound having a structure according to formula (X),
wherein the dioxane-dione is selected from the group of (1r,3r,5r,7r)-spiro[adamantan-2,2′-[1,3]-dioxane]-4′,6′-dione (SPIAd), 6,10-dioxaspiro[4.5]decane-7,9-dione, 1,5-dioxaspiro[5.5]undecane-2,4-dione, 1,5-dioxaspiro[5.7]tridecane-2,4-dione, 2,2-diphenyl-1,3-dioxane-4,6-dione, 2,2-dibenzyl-1,3-dioxane-4,6-dione, 5,5-dimethylcyclohexane-1,3-dione, 2-(tert-butyl)-2-methyl-1,3-dioxane-4,6-dione, 2-methyl-2-phenyl-1,3-dioxane-4,6-dione, and 2-(4-fluorophenyl)-2-methyl-1,3-dioxane-4,6-dione.

121. The method of claim 120, wherein the admixing is performed in the presence of ethanol.

122. The method of claim 120 or claim 121, wherein the admixing comprises adding the SPIAd and sodium carbonate to the reaction product of the compound having a structure according to formula (IIa) with 1-chloromethyl-4-fluoro-1,4-diazoniabicyclo[2.2.2]octanebis(tetrafluoroborate), methyl cyanide, and trimethylsilyl acetate dropwise at about 23° C. and stirring for about 4 hrs.

123. A method of preparing a fibroblast activation protein inhibitor comprising:
admixing a compound having a structure according to formula (XI) with a radiolabeled salt, MX, to form a compound having a structure according to formula (III): 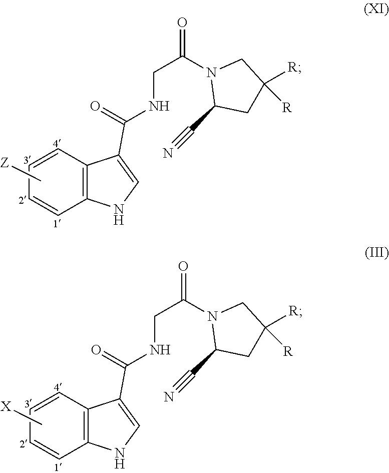
wherein
Z has a structure selected from the group of: 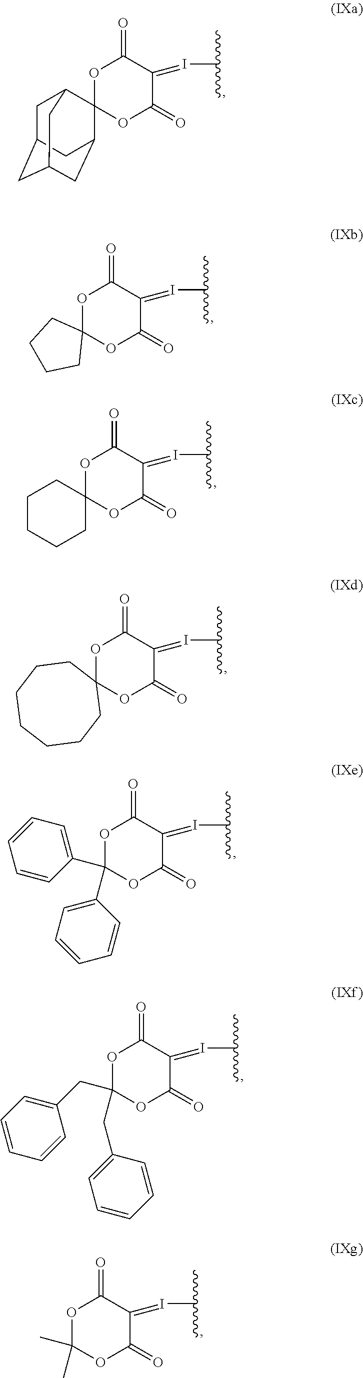 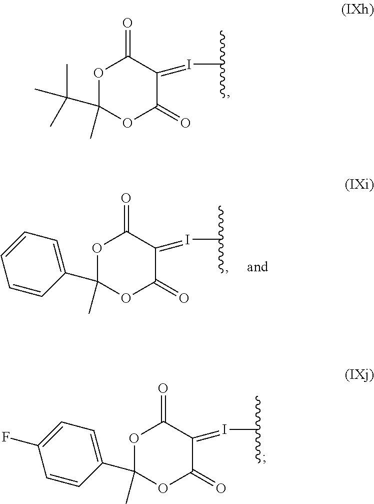 [SMILES: *I=C1C(=O)OC(C)(C(C)(C)C)OC1=O ; *I=C1C(=O)OC(C)(c2ccccc2)OC1=O ; *I=C1C(=O)OC(C)(c2ccc(F)cc2)OC1=O]
in formula (XI) and formula (III), Z and X are located at carbon 1′, 2′, or 3′, and the location of Z in formula (XI) and the location of X in formula (III) are the same;
M is selected from the group of K, Li, Na, Rb, Cs, tetraethylammonium, and tetrabutylammonium;
in the salt, MX, and in formula (III), X is selected from the group of ¹⁸F, ²¹¹At, ¹²³I, ¹²⁴I, ¹²⁵I, ¹³¹I, ⁷⁶Br, ⁷⁷Br; and
each R is independently selected from H, F, ²H, CH₃, and cyclopentyl, and the R in formula (III) are the same as the R in formula (XI).

124. The method of claim 123, wherein the admixing is performed
(a) in the presence of dimethylsulfoxide (DMSO), dimethylformamide (DMF), dimethylacetamide (DMA), or a combination thereof; and/or
(b) at a temperature in a range of 85° C. to 140° C. for a time in a range of about 5 minutes to about 12 hrs.

125. The method of any one of claim 123 or 124, further comprising preparing the compound having a structure according to formula (XI), by admixing a compound having a structure according to formula (IIIa): 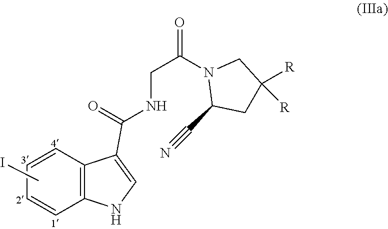
with 1-chloromethyl-4-fluoro-1,4-diazoniabicyclo[2.2.2]octanebis(tetrafluoroborate), and trimethylsilyl acetate to form a reaction product;
wherein the R in formula (IIIa) are the same R as in the compound according to formula (XI) and the I is located at the same carbon as the Z in the compound according to formula (XI).

126. The method of claim 125, wherein the admixing is performed in the presence of acetonitrile.

127. The method of claim 125 or 126, wherein the admixing comprises dropwise addition of 1-chloromethyl-4-fluoro-1,4-diazoniabicyclo[2.2.2]octanebis(tetrafluoroborate) to a solution of the compound having a structure according to formula (IIa) and trimethylsilyl acetate at about 23° C. and stirring for about 16 hrs.

128. The method of any one of claims 125 to 127, further comprising
admixing (a) the reaction product of the compound having a structure according to formula (IIIa) with 1-chloromethyl-4-fluoro-1,4-diazoniabicyclo[2.2.2]octanebis(tetrafluoroborate), and trimethylsilyl acetate with (b) a dioxane-dione and sodium carbonate to form the compound having a structure according to formula (XI),
wherein the dioxane-dione is selected from the group of (1r,3r,5r,7r)-spiro[adamantan-2,2′-[1,3]-dioxane]-4′,6′-dione (SPIAd), 6,10-dioxaspiro[4.5]decane-7,9-dione, 1,5-dioxaspiro[5.5]undecane-2,4-dione, 1,5-dioxaspiro[5.7]tridecane-2,4-dione, 2,2-diphenyl-1,3-dioxane-4,6-dione, 2,2-dibenzyl-1,3-dioxane-4,6-dione, 5,5-dimethylcyclohexane-1,3-dione, 2-(tert-butyl)-2-methyl-1,3-dioxane-4,6-dione, 2-methyl-2-phenyl-1,3-dioxane-4,6-dione, and 2-(4-fluorophenyl)-2-methyl-1,3-dioxane-4,6-dione.

129. The method of claim 128, wherein the admixing is performed in the presence of ethanol.

130. The method of claim 128 or claim 129, wherein the admixing comprises adding the SPIAd and sodium carbonate to the reaction product of the compound having a structure according to formula (IIIa) with 1-chloromethyl-4-fluoro-1,4-diazoniabicyclo[2.2.2]octanebis(tetrafluoroborate), methyl cyanide, and trimethylsilyl acetate dropwise at about 23° C. and stirring for about 4 hrs.

131. A method treating, detecting, or imaging cancer comprising, administering to a patient a fibroblast activation protein inhibitor according to any one of claims 1 to 27.

132. Use of a fibroblast activation protein inhibitor according to any one of claims 1 to 27 in the treatment, detection, or imaging of cancer.

133. Use in the manufacture of a medicament for the treatment, detection, or imaging of cancer, of a fibroblast activation protein inhibitor according to any one of claims 1 to 27.

134. The method or use of any one of claims 131 to 133, wherein the cancer comprises an epithelial tumor, sarcoma, or mesothelioma.

135. A method of detecting fibroblast activation protein expression in human development, growth, wound healing, or a combination thereof comprising administering to a patient a fibroblast activation protein inhibitor according to any one of claims 1 to 27.

136. Use of a fibroblast activation protein inhibitor according to any one of claims 1 to 27 in the detection of the expression of fibroblast activation protein or the lack of expression of fibroblast activation protein in a human.

137. Use of a fibroblast activation protein inhibitor according to any one of claims 1 to 27 in the manufacture of a medicament for the detection of the expression of fibroblast activation protein.

138. A method of detecting a cardiovascular pathology comprising, administering to a patient a fibroblast activation protein inhibitor according to any one of claims 1 to 27.

139. Use of a fibroblast activation protein inhibitor according to any one of claims 1 to 27 in the detection of a cardiovascular pathology in a human.

140. Use of a fibroblast activation protein inhibitor according to any one of claims 1 to 27 in the manufacture of a medicament for the detection of a cardiovascular pathology.

141. The method of any one of claims 138 to 140, wherein the cardiovascular pathology comprises a myocardial infarction, cardiac remodeling after infarction, or aortic remodeling.

142. Use of a fibroblast activation protein inhibitor according to any one of claims 1 to 27 in the detection of a pulmonary pathology in a human.

143. Use of a fibroblast activation protein inhibitor according to any one of claims 1 to 27 in the manufacture of a medicament for the detection of a pulmonary pathology.

144. The method of claim 132 or 143, wherein the pulmonary pathology is the result of an auto immune or rheumatologic response, primary pulmonary pathology such as interstitial lung disease, or post treatment effects from chemotherapy, radiation therapy, or a combination thereof.
